(19) United States
(12) Patent Application Publication
(10) Pub. No.: US 2025/0082753 A1
(43) Pub. Date: Mar. 13, 2025

(54) CHIMERIC RECEPTORS

(71) Applicant: Hoffmann-La Roche Inc., Little Falls, NJ (US)

(72) Inventors: Christian KLEIN, Bonstetten (CH); Simone LANG, Zurich (CH); Ekkehard MOESSNER, Kreuzlingen (CH); Dario VENETZ, Bonstetten (CH)

(73) Assignee: Hoffmann-La Roche Inc., Little Falls, NJ (US)

(21) Appl. No.: 18/894,979

(22) Filed: Sep. 24, 2024

Related U.S. Application Data
(63) Continuation of application No. PCT/EP2023/057599, filed on Mar. 24, 2023.

(30) Foreign Application Priority Data
Mar. 25, 2022 (EP) 22164300.0

(51) Int. Cl. A61K 39/00; C07K 14/725; C07K 16/42

(52) U.S. Cl. CPC A61K 40/41; A61K 39/0005; A61K 40/11; A61K 40/31; C07K 14/7051; C07K 16/4283; C07K 2317/24; C07K 2317/92; C07K 2319/30

(57) ABSTRACT

The present invention generally relates to chimeric receptors comprising an extracellular domain comprising a mutated Fc domain. The invention also relates to transduced immune cells expressing the chimeric receptors of the invention and/or nucleic acid molecules encoding the chimeric receptors of the present invention. Further provided are kits comprising such cells and/or nucleic acid molecules encoding the chimeric receptors, and antibodies capable of binding to the chimeric receptors.

### CROSS-REFERENCE TO RELATED APPLICATIONS

This application is a Continuation of International Application No. PCT/EP2023/057599, filed Mar. 24, 2023, which claims benefit of priority to European Application No. 22164300.0, filed Mar. 25, 2022, each of which is incorporated herein by reference in its entirety.

### SEQUENCE LISTING

This application contains a Sequence Listing which has been submitted electronically in XML format and is hereby incorporated by reference in its entirety. Said XML copy, created on Aug. 14, 2024, is named P37388-US sequence listing.xml and is 157,866 bytes in size.

### FIELD OF THE INVENTION

The present invention generally relates to chimeric receptors comprising an extracellular domain comprising a mutated Fc domain. The invention also relates to transduced immune cells expressing the chimeric receptors of the invention and/or nucleic acid molecules encoding the chimeric receptors of the present invention. Further provided are kits comprising such cells and/or nucleic acid molecules encoding the chimeric receptors, and antibodies capable of binding to the chimeric receptors.

### BACKGROUND

Adoptive T cell therapy (ACT) is a powerful treatment approach using cancer-specific T cells (Rosenberg and Restifo, Science 348 (6230) 2015:62-68). ACT may use naturally occurring tumor-specific cells or T cells rendered tumor specific by genetic engineering using T cell or chimeric antigen receptors (Rosenberg and Restifo, Science 348 (6230) 2015:62-68). ACT can successfully treat and induce remission in patients suffering even from advanced and otherwise treatment refractory diseases such as acute lymphatic leukemia, non-hodgkins lymphoma or melanoma (Dudley et al., J Clin Oncol 26 (32) 2008:5233-5239; Grupp et al., N Engl J Med 368 (16) 2013:1509-1518: Kochenderfer et al., J Clin Oncol. 33 (6) 2015:540-549). However, despite impressive clinical efficacy, ACT is limited by treatment-related toxicities.

The specificity, and resulting on-target and off-target effects, of engineered T cells used in ACT is mainly driven by the tumor targeting antigen binding moiety implemented in the antigen binding receptors (Hinrichs et al., Nat Biotechnol. 31 (11) 2013, 999-1008). Non-exclusive expression of the tumor antigen or temporal difference in the expression level can result with serious side effects or even abortion of ACT due to non-tolerable toxicity of the treatment. Indeed, apart from few lineage specific antigens such as CD19, CD20 or BCMA progress with conventional CAR-T cells has been slow, particularly in solid tumors where epithelial tumor antigens are targeted that frequently are expressed in normal tissues resulting in on-target off-tumor toxicities, this has been shown for example for CAR-T cells targeting HER2 (Morgan et al., Mol Ther 18 (4) 1020:843-851). Thus, ways to make CAR-T cells more tumor specific are of great therapeutic potential.

Additionally, the availability of tumor-specific T cells for efficient tumor cells lysis is dependent on the long-term survival and proliferation capacity of engineered T cells in vivo. On the other hand, in vivo survival and proliferation of T cells may also result in unwanted long-term effects due to the persistence of an uncontrolled T cell response which can result in damage of healthy tissue (Grupp et al. 2013 N Engl J Med 368 (16): 1509-18, Maude et al. 2014 2014 N Engl J Med 371 (16): 1507-17).

One approach for limiting serious treatment-related toxicities and to improve safety of ACT is to restrict the activation and proliferation of T cells by introducing adaptor molecules which control the formation of an immunological synapse. Such adaptor molecules comprise small molecular bimodular switches as e.g. recently described folate-FITC switch (Kim et al. J Am Chem Soc 2015:137:2832-2835). A further approach included artificially modified antibodies comprising a tag to guide and direct the specificity of the T cells to target tumor cells (Ma et al. PNAS 2016; 113 (4): E450-458, Cao et al. Angew Chem 2016:128:1-6, Rogers et al. PNAS 2016; 113 (4): E459-468. Tamada et al. Clin Cancer Res 2012; 18 (23): 6436-6445).

However, existing approaches have several limitations. Immunological synapses relying on molecular switches require introduction of additional elements that might elicit an immune response or result with non-specific off-target effects. On the other hand, the introduction of tag structure in existing therapeutic monoclonal antibodies may affect the efficacy and safety profile of these constructs. Further, adding tags require additional modification and purification steps making the production of such antibodies more complex and further require additional safety testing.

Another limitation of existing approaches is the possibility of on-target off-tumor effects. In most indications, clean targets are missing because the antigen of interest, although highly expressed on tumor cells, is also expressed on healthy tissue. To reduce side effects and increase the choice of druggable target antigens, it is favorable to have only locally active cancer specific T cells. To improve the selectivity of CAR T cells to be only active in tumor, different approaches have been developed e.g an oxygen sensitive CAR (Juillerat A et al. Sci Rep. 2017:7:39833. Published 2017 Jan. 20. doi: 10.1038/srep39833). Also direct CARs possessing a protease-sensitive linker have been developed that aim to improve safety of conventional ACT (Han X et al. Mol Ther. 2017; 25 (1): 274-284. doi: 10.1016/j.ymthe.2016.10.011). However, tumor-activated CARs are complex and often rely on tumor-intrinsic activation triggers such as tumor-associated proteases. Due to the heterogenic nature of cancer, the presence of these activation triggers may vary widely between different tumor lesions, cancer types and patients. Adaptor molecules, which are otherwise biologically inert but specifically interact with an engineered chimeric receptor, could allow control of the T cell activation in a dose-dependent manner. Therefore, such adaptor approaches provide an alternative generic strategy to control therapy-related side effects. However, engineered orthogonal ligand/receptor systems are often based on a mutated ligand receptor interface which bears the risk of immunogenicity when administered to patients. This can lead to the formation of anti-drug antibodies which can potentially result in a loss of exposure of the therapeutically active component of the cell therapy.

Hence, there is a need for improved therapies to address the challenges of ACT.

### SUMMARY OF THE INVENTION

The present invention provides chimeric receptors with improved properties as described herein below:

Provided herein is a chimeric receptor comprising
(i) an extracellular domain comprising a mutated Fc domain or a fragment thereof, and
(ii) a transmembrane domain.

In one embodiment the chimeric receptor further comprises (iii) at least one intracellular stimulatory signaling domain and/or at least one co-stimulatory signaling domain.

In one embodiment, the extracellular domain comprises a mutated CH2 domain and/or a mutated CH3 domain.

In one embodiment, the extracellular domain comprises a mutated CH2 domain and/or a mutated CH3 domain, wherein the mutated CH2 domain and/or the mutated CH3 domain comprises the amino acid substitution (numbering according to Kabat EU index):
(a) I253A, H310A and H435A,
(b) I253A,
(c) I253A and H310A,
(d) I253A and H435A,
(e) H310A and H435A,
(f) H310A,
(g) H435A,
(h) L234A, L235A, and P329G, or
(i) L234A, L235A, I253A, H310A, P329G and H435A.

In one embodiment, the transmembrane domain is selected from the group consisting of the CD8, the CD4, the CD3z, the FCGR3A, the NKG2D, the CD27, the CD28, the CD137, the OX40, the ICOS, the DAP10 or the DAP12 transmembrane domain or a fragment thereof.

In one embodiment, the stimulatory signaling domain is selected from the group consisting of the intracellular domain of CD3z, of FCGR3A and of NKG2D, or a fragment thereof that retains stimulatory signaling activity.

In one embodiment, the co-stimulatory signaling domain is selected from the group consisting of the intracellular domain of CD27, of CD28, of CD137, of OX40, of ICOS, of DAP10 and of DAP12, or a fragment thereof that retain co-stimulatory signaling activity.

In one embodiments, the co-stimulatory signaling domain is the CD28 intracellular domain or a fragment thereof that retains CD28 co-stimulatory activity.

In one embodiment, the chimeric receptor comprises one stimulatory signaling domain comprising the intracellular domain of CD3z, or a fragment thereof that retains CD3z stimulatory signaling activity, and wherein the chimeric receptor comprises one co-stimulatory signaling domain comprising the intracellular domain of CD137, or a fragment thereof that retains CD137 co-stimulatory signaling activity.

In one embodiment, the stimulatory signaling domain comprises the amino acid sequence of SEQ ID NO: 13 and the co-stimulatory signaling domain comprises the amino acid sequence of SEQ ID NO:12.

In one embodiment, the chimeric receptor comprises an amino acid sequence that is at least about 95%, 96%, 97%, 98%, 99% or 100% identical to an amino acid sequence selected from the group consisting of SEQ ID NO: 87, SEQ ID NO: 106, SEQ ID NO: 125 and SEQ ID NO:127.

In one embodiment, provided is a chimeric receptor comprising or consisting of an amino acid sequence selected from the group consisting of SEQ ID NO:87, SEQ ID NO:106, SEQ ID NO: 125 and SEQ ID NO:127.

In one embodiment, provided is an isolated polynucleotide encoding the chimeric receptor as described herein before.

In one embodiment, provided is a polypeptide encoded by the isolated polynucleotide as described herein before.

In one embodiment, provided is a vector, particularly an expression vector, comprising the polynucleotide as described herein before.

In one embodiment, provided is a transduced T cell comprising the polynucleotide as described herein before or the vector as described herein before.

In one embodiment, provided is a transduced T cell capable of expressing the chimeric receptor as described herein before.

In one embodiment, provided is a kit comprising
(A) a transduced T cell capable of expressing the chimeric receptor as described herein before, wherein the chimeric receptor comprises an extracellular domain comprising a mutated Fc domain or a fragment thereof; and
(B) an antibody that comprises at least one antigen binding moiety capable of binding to the mutated Fc domain but not capable of binding to the non-mutated parent Fc domain, wherein the antibody comprises at least one effector moiety.

In one embodiment, provided is a kit comprising
(A) an isolated polynucleotide encoding the chimeric receptor as described herein before, wherein the chimeric receptor comprises an extracellular domain comprising a mutated Fc domain or a fragment thereof; and
(B) an antibody that comprises at least one antigen binding moiety capable of binding to the mutated Fc domain but not capable of binding to the non-mutated parent Fc domain, wherein the antibody comprises at least one effector moiety. In one embodiment, said effector moiety is (i) an antigen binding moiety capable of binding to a target cell antigen, or (ii) and immune activating moiety, in particular a cytokine.

In one embodiment, provided is the kit as described herein before for use as a medicament.

In one embodiment, provided is the chimeric receptor as described herein before or the transduced T cell as described herein before for use as a medicament, wherein the chimeric receptor comprises an extracellular domain comprising a mutated Fc domain or a fragment thereof, wherein a transduced T cell expressing the chimeric receptor is administered before, simultaneously with or after administration of an antibody that comprises at least one antigen binding moiety capable of binding to a target cell antigen, and at least one antigen binding moiety capable of binding to the mutated Fc domain but not capable of binding to the non-mutated parent Fc domain.

In one embodiment, provided is the kit as described herein before for use in the treatment of a disease, in particular for use in the treatment of a cancer.

In one embodiment, provided is a method of treating a disease in a subject, comprising administering to the subject a transduced T cell capable of expressing the chimeric receptor as described herein before, wherein the chimeric receptor comprises an extracellular domain comprising a mutated Fc domain or a fragment thereof, and administering before, simultaneously with or after administration of the transduced T cell a therapeutically effective amount of an an antibody that comprises at least one antigen binding moiety capable of binding to the mutated Fc domain but not capable of binding to the non-mutated parent Fc domain, wherein the antibody comprises at least one effector moiety. In one embodiment said effector moiety is (i) an antigen binding moiety capable of binding to a target cell antigen, or (ii) and immune activating moiety, in particular a cytokine.

In one embodiment, provided is the use of the chimeric receptor as described herein before, the polynucleotide as described herein before or the transduced T cell as described herein before for the manufacture of a medicament.

In one embodiment, the medicament is for treatment of cancer. In one embodiment said cancer is selected from cancer of epithelial, endothelial or mesothelial origin and cancer of the blood.

### SHORT DESCRIPTION OF THE FIGURES

FIGS. 1A-1D: Schematic representation of chimeric receptors of the invention comprising a CH2 domain containing the P329G substitution (FIG. 1A) or CH2-CH3 domains containing substitutions 1253A. H310A. P329R (CH2 domain) and H435A (CH3 domain) (FIG. 1B). Schematic representation of corresponding DNA constructs (FIGS. 1C-1D).

FIGS. 2A-2C: Expression of CH2 P329G construct in Jurkat TCRab KO CD4+ cells (Promega) after lentiviral transduction and pool sorting for eGFP positive cells. The construct was detected with an anti-P329G VH3×VL1 (clone M-1.7.24) humanized IgG labelled with AF647. Schematic representation of the assay (FIG. 2A). CH2 P329G construct surface expression (FIG. 2B) and corresponding eGFP expression (FIG. 2C). Jurkat TCRab KO CD4+ wild type cells served as negative control.

FIGS. 3A-3D Activation of sorted pool of Jurkat TCRab KO CD4+ cells transduced with CH2 P329G construct (SEQ ID NO:87) in the presence of FolR1⁺ target cells with high (HeLa), medium (Ovcar3) and low (HT-29) target expression levels upon stimulation with anti-FolR1× anti-CH2_P329G bispecific) antibody (SEQ ID Nos: 108, 109, 110, 111). Activation was assessed by quantification of the intensity of CD3 downstream signaling reported by NFAT promoter-controlled luciferase expression. Schematic representation of the assay (FIG. 3A). Dose-depended activation of Jurkat cells in the presence of HT29 (FIG. 3B), Ovcar-3 (FIG. 3C) or HeLa (FIG. 3D) as target cells. Mock-transduced cells were used as negative control. Depicted are technical average values from triplicates, error bars indicate SD.

FIGS. 4A, 4B: Binding of anti-FolR1× anti-CH2-CH3 AAA P329R bispecific antibody (SEQ ID Nos: SEQ ID Nos: 108, 112, 113, 114) on non-sorted Jurkat NFAT cells (Promega) that were transduced via lentivirus with the CH2-CH3 AAA P329R construct (SEQ ID NO:105). Binding was assessed via secondary R-Phycoerythrin (PE) AffiniPure F(ab′)₂ Fragment Goat Anti-Human IgG (F(ab′)₂ fragment specific). Schematic representation of the assay (FIG. 4A). Dose-depended binding to the CH2-CH3 AAA P329R construct transduced Jurkat cells (FIG. 4B). Jurkat NFAT wildtype cells were used as negative control.

FIGS. 5A-5C: Expression of CH2-CH3 AAA P329R construct (SEQ ID NO:105) in Jurkat NFAT cells after lentiviral transduction. The construct was detected with anti-AAA×FolR1 2+1 bispecific antibody (SEQ ID Nos: SEQ ID Nos: 108, 112, 113, 114) and secondary PE AffiniPure F(ab′)₂ Fragment Goat Anti-Human IgG (F(ab′)₂ fragment specific). Schematic representation of the assay (FIG. 5A). Surface expression of the CH2-CH3 AAA P329R construct (FIG. 5B) and corresponding eGFP expression (FIG. 5C). Jurkat NFAT wild type cells served as negative control.

FIGS. 6A, 6B: Activation of non-sorted Jurkat NFAT cells transduced with CH2-CH3 AAA P329R construct (SEQ ID NO:105) in the presence of FolR1⁺ target cells with high target expression levels (HeLa) in combination with anti-FolR1×AAA 2+1 bispecific antibody (SEQ ID Nos: SEQ ID Nos: 108, 112, 113, 114). Activation was assessed by quantification of the intensity of CD3 downstream signaling reported by NFAT promoter-controlled luciferase expression. Schematic representation of the assay (FIG. 6A). Dose-depended activation of Jurkat cells in the presence HeLa as target cells (FIG. 6B). Jurkat NFAT wildtype cells served as negative control. Depicted are technical average values from triplicates, error bars indicate SD.

FIG. 7: depict a schematic of use of a combination of the chimeric receptor of the invention with an exemplary immune activating Fc binding molecules with an IL2v (cytokine) effector moiety.

FIGS. 8A-8D: FIGS. 8A-8B depict schematic representation of chimeric receptors of the invention comprising (FIG. 8A) a CH2 domain containing the P329G mutation or (FIG. 8B) CH2-CH3 domains containing mutations I253A, H310A, P329G (CH2 domain) and H435A (CH3 domain). FIGS. 8C-8D depict schematic representation of corresponding DNA constructs. The DNA constructs used in the appended Example in addition contained an eGFP encoding gene separated from the chimeric receptors via a 2A self-cleaving peptide.

FIGS. 9A-9B: Primary T cells expressing the chimeric receptors of the invention. Human CD4 and CD8 T cells were transduced with either CH2-CH3 AAA P329G CAR (SEQ ID NO: 127), CH2-CH3 AAA P329R CAR (SEQ ID NO:125) or CH2 P329G CAR (SEQ ID NO:87), respectively. The expression of the constructs was assessed by eGFP expression and surface staining of the CARs. For the latter, a staining with either anti-PG VH3×VL1 huIgG-AF647 (CH2-CH3 AAA P329G CAR, CH2 P329G CAR) (FIG. 9A) or anti-AAA×FolRI 2+1 TCB and secondary APC-F(ab)₂ Fragment anti-huIgG (Jackson ImmunoResearch, #109-136-097) (CH2-CH3 AAA P329G CAR. CH2-CH3 AAA P329R CAR) (FIG. 9B) was performed. Between 86-89% of the primary T cells were expressing the chimeric receptors.

FIGS. 10A-10F: T cell mediated killing. CH2-CH3 AAA P329G CAR (SEQ ID NO: 127. FIGS. 10A-10B)/CH2-CH3 AAA P329R CAR (SEQ ID NO:125. FIGS. 10C-10D) or CH2 P329G CAR T cells (SEQ ID NO:87. FIGS. 10E-10F) were incubated with HeLa NLR (NucLight Red) cells. As adaptor molecules served anti-AAA×FolRI 2+1/anti-AAA×FolRI 1+1 or anti-PG×FolRI 2+1/anti-PG×FolRI 1+1 antibodies. The red cell count was tracked with the Incucyte® system over 6 days. As negative control served wells without adaptor molecule and as positive control 10 nM anti-FolR1 TCB. Dose-dependent red cell count reduction (cancer cell killing) or cancer cell growth was observed. Depicted are technical average values from duplicates, error bars indicate SD.

FIGS. 11A-11C: T cell mediated killing. CH2-CH3 AAA P329G CAR (SEQ ID NO: 127. FIG. 11A)/CH2-CH3 AAA P329R CAR (SEQ ID NO: 125. FIG. 11B) or CH2 P329G CAR T cells (SEQ ID NO:87. FIG. 11C) were incubated with MKN45 NLR (NucLight Red) cells. As adaptor molecules served anti-AAA×CEA 2+1 or anti-PG×CEA 2+1 antibodies. As negative control served wells without adaptor molecule and as positive control 10 nM anti-CEA TCB. Dose-dependent red cell count reduction (cancer cell killing) or cancer cell growth was observed. Depicted are technical average values from duplicates, error bars indicate SD.

FIGS. 12A-12B: CH2-CH3 AAA P329G CAR (SEQ ID NO: 127)/CH2-CH3 AAA P329R CAR (SEQ ID NO: 125) or CH2 P329G CAR (SEQ ID NO:87) T cells were stained with anti-PG and anti-AAA antibodies to assess whether the AAA and P329G mutations can be bound simultaneously (FIG. 12A). FIG. 12B shows double positive (BV421+, AF647+) CAR-T cells, showing that AAA and P329G mutations can be bound at the same time. The CH2-CH3 AAA P329R CAR or CH2 P329G CAR T cells only showed the respective single staining as expected.

FIGS. 13A-13D: STAT5 phosphorylation (pSTAT5) of primary T cells transduced with CH2-CH3 AAA P329G CAR (SEQ ID NO:127)/CH2-CH3 AAA P329R CAR (SEQ ID NO:125) or CH2 P329G CAR (SEQ ID NO:87) after stimulation with anti-P329G-IL2v. Shown is the pSTAT5 median fluorescence intensity after gating on eGFP+ (CAR+) or eGFP− (CAR−) cells. Targeting of anti-P329G-IL2v via the P329G mutation to CAR T cells leads to a ˜50-fold (CH2 PG CAR. FIG. 13A) or 225-fold (CH2 CH3 AAA PG CAR, FIG. 13B) difference in the EC50) compared to eGFP-cells in the same well. This effect was not observed with CH2 CH3 AAA PR CAR T cells (FIG. 13C) as expected. pSTAT5 MFI of the cells incubated with Proleukin was comparable between the cell products and between eGFP+ and eGFP− (FIG. 13D). Depicted are technical average values from duplicates, error bars indicate SD. The EC50 values were calculated with GraphPad Prism 8.4.2 (log (agonist) vs. response—Variable slope (four parameters)).

### DETAILED DESCRIPTION

### Definitions

Terms are used herein as generally used in the art, unless otherwise defined in the following. An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

“Antibody-dependent cell-mediated cytotoxicity” (“ADCC”) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831).

An “effective amount” of an agent, e.g., a pharmaceutical composition, refers to an amount effective, at dosages and for periods of time necessary, to achieve the desired therapeutic or prophylactic result.

“Affinity” refers to the strength of the sum total of noncovalent interactions between a single binding site of a molecule (e.g., an antibody) and its binding partner (e.g., an antigen). Unless indicated otherwise, as used herein. “binding affinity” refers to intrinsic binding affinity which reflects a 1:1 interaction between members of a binding pair (e.g., antibody and antigen). The affinity of a molecule X for its partner Y can generally be represented by the dissociation constant (K_{D})). Affinity can be measured by common methods known in the art, including those described herein. Specific illustrative and exemplary methods for measuring binding affinity are described in the following.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g. hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid. Amino acids may be referred to herein by either their commonly known three letter symbols or by the one-letter symbols recommended by the IUPAC-IUB Biochemical Nomenclature Commission.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor, or increased association with another peptide. Amino acid sequence deletions and insertions include amino- and/or carboxy-terminal deletions and insertions of amino acids. Particular amino acid mutations are amino acid substitutions. For the purpose of altering e.g. the binding characteristics of an Fc region, non-conservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art.

Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc domain to glycine can be indicated as 329G, G329, G₃₂₉, P329G, or Pro329Gly.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “antigen binding domain” refers to the part of an antibody that comprises the area which specifically binds to and is complementary to part or all of an antigen. An antigen binding domain may be provided by, for example, one or more antibody variable domains (also called antibody variable regions). Particularly, an antigen binding domain comprises an antibody light chain variable domain (VL) and an antibody heavy chain variable domain (VH).

As used herein, the term “antigen binding molecule” refers in its broadest sense to a molecule that specifically binds an antigenic determinant. Examples of antigen binding molecules are immunoglobulins and derivatives, e.g., fragments, thereof as well as antigen binding receptors and derivatives thereof.

As used herein, the term “antigen binding moiety” refers to a polypeptide molecule that specifically binds to an antigenic determinant. In one embodiment, an antigen binding moiety is able to direct the entity to which it is attached to a target site, for example to a specific type of tumor cell or tumor stroma bearing the antigenic determinant. Antigen binding moieties include antibodies and fragments thereof as further defined herein. Particular antigen binding moieties include an antigen binding domain of an antibody, comprising an antibody heavy chain variable region and an antibody light chain variable region (e.g. a scFv fragment). In certain embodiments, the antigen binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ.

In the context of the present invention the term “chimeric receptor” relates to an engineered receptor comprising an extracellular domain comprising a mutated Fc domain, in particular a mutated CH2 and/or CH3 domain, a transmembrane domain, and at least one intracellular stimulatory signaling domain and/or at least one co-stimulatory signaling domain. A chimeric receptor can be made of polypeptide parts from different sources. Accordingly, it may be also understood as a “fusion protein” and/or a “chimeric protein”. Usually, fusion proteins are proteins created through the joining of two or more genes (or preferably cDNAs) that originally coded for separate proteins. Translation of this fusion gene (or fusion cDNA) results in a single polypeptide, preferably with functional properties derived from each of the original proteins.

Recombinant fusion proteins are created artificially by recombinant DNA technology for use in biological research or therapeutics. Further details to the chimeric receptors of the present invention are described herein below.

An “antigen binding site” refers to the site. i.e. one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

The term “antigen binding domain” refers to the part of an antibody or an antigen binding receptor that comprises the area which specifically binds to and is complementary to part or all of an antigen. An antigen binding domain may be provided by, for example, one or more immunoglobuling variable domains (also called variable regions). Particularly, an antigen binding domain comprises an immunoglobulin light chain variable domain (VL) and an immunoglobulin heavy chain variable domain (VH).

As used herein, the term “antigenic determinant” is synonymous with “antigen” and “epitope” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

“Antibodies” according to the present invention. i.e. therapeutic antibodies may have one, two, three or more binding domains and may be monospecific, bispecific or multispecific. The antibodies can be full length from a single species, or be chimerized or humanized. For an antibody with more than two antigen binding domains, some binding domains may be identical and/or have the same specificity.

The term “transmembrane domain” or abbreviated “TD” as used herein defines a polypeptide stretch capable of integrating in the cellular membrane (or plasma membrane) of a cell. The transmembrane domain can be fused to extracellular and/or intracellular polypeptide domains wherein these extracellular and/or intracellular polypeptide domains will be confined to the cell membrane. In the context of the chimeric receptors of the present invention the transmembrane domain confers membrane attachment and confinement of the chimeric receptor of the present invention. The chimeric receptors of the present invention comprise at least one transmembrane domain and an extracellular domain comprising a CH2 domain. Additionally, the transmembrane domain may be fused to intracellular signaling domains.

The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antigen binding molecule comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹M to 10⁻¹³ M).

The term “CDR” as employed herein relates to “complementary determining region”, which is well known in the art. The CDRs are parts of immunoglobulins or antigen binding receptors that determine the specificity of said molecules and make contact with a specific ligand. The CDRs are the most variable part of the molecule and contribute to the antigen binding diversity of these molecules. There are three CDR regions CDR1, CDR2 and CDR3 in each V domain. CDR-H depicts a CDR region of a variable heavy chain and CDR-L relates to a CDR region of a variable light chain. VH means the variable heavy chain and VL means the variable light chain. The CDR regions of an Ig-derived region may be determined as described in “Kabat” (Sequences of Proteins of Immunological Interest”, 5th edit. NIH Publication no. 91-3242 U.S. Department of Health and Human Services (1991); Chothia J. Mol. Biol. 196 (1987), 901-917) or “Chothia” (Nature 342 (1989), 877-883).

The term “CD3z” refers to T-cell surface glycoprotein CD3 zeta chain, also known as “T-cell receptor T3 zeta chain” and “CD247”.

The term “chimeric antigen receptor” or “CAR” refers to an antigen binding receptor constituted of an extracellular portion of an antigen binding moiety (e.g. a single chain antibody domain) fused by a spacer sequence to intracellular signaling/co-signalling domains (such as e.g. of CD3z and CD28). The chimeric receptor according to the invention comprises a different extracellular domain compared to a CAR. i.e. the extracellular domain of the chimeric receptor according to the invention does not comprise a target antigen binding moiety (e.g. the chimeric receptor of the invention does not comprise a scFv). However, the transmembrane domain and the intracellular signaling domains comprised in the chimeric receptors according to the invention are similar or identical to the transmembrane domains and intracellular signaling domains that may be used in a CAR.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. In certain aspects, the antibody is of the IgG₁ isotype. In certain aspects, the antibody is of the IgG₁ isotype with the P329G, L234A and L235A substitution to reduce Fc-region effector function. In other aspects, the antibody is of the IgG₂ isotype. In certain aspects, the antibody is of the IgG₄ isotype with the S228P substitution in the hinge region to improve stability of IgG₄ antibody. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

The terms “constant region derived from human origin” or “human constant region” as used in the current application denotes a constant heavy chain region of a human antibody of the subclass IgG1, IgG2, IgG3, or IgG4 and/or a constant light chain kappa or lambda region. Such constant regions can be used in human or humanized antibodies and are well known in the state of the art and e.g. described by Kabat, E. A., et al., Sequences of Proteins of Immunological Interest, 5th ed., Public Health Service. National Institutes of Health. Bethesda, MD (1991) (see also e.g. Johnson, G., and Wu, T. T., Nucleic Acids Res. 28 (2000) 214-218: Kabat, E. A., et al., Proc. Natl. Acad. Sci. USA 72 (1975) 2785-2788). Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service. National Institutes of Health. Bethesda. MD (1991), NIH Publication 91-3242.

By a “crossover” Fab molecule (also termed “Crossfab”) is meant a Fab molecule wherein the variable domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the crossover Fab molecule.

The term “CSD” as used herein refers to co-stimulatory signaling domain. An example of a co-stimulatory signaling domain is the intracellular domain of CD137, or fragments thereof.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment.

Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

A “Fab molecule” refers to a protein consisting of the VH and CH1 domain of the heavy chain (the “Fab heavy chain”) and the VL and CL domain of the light chain (the “Fab light chain”) of an immunoglobulin.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain.

Therefore, an antibody produced by a host cell by expression of a specific nucleic acid molecule encoding a full-length heavy chain may include the full-length heavy chain, or it may include a cleaved variant of the full-length heavy chain (also referred to herein as a “cleaved variant heavy chain”). This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Amino acid sequences of heavy chains including Fc domains (or a subunit of an Fc domain as defined herein) are denoted herein without C-terminal glycine-lysine dipeptide if not indicated otherwise. In one embodiment of the invention, a heavy chain including a subunit of an Fc domain as specified herein, comprises an additional C-terminal glycine-lysine dipeptide (G446 and K447, numbering according to EU index of Kabat). In one embodiment of the invention, a heavy chain including a subunit of an Fc domain as specified herein, comprises an additional C-terminal glycine residue (G446, numbering according to EU index of Kabat). Compositions of the invention, such as the pharmaceutical compositions described herein, comprise a population of antigen binding molecules of the invention. The population of antigen binding molecule may comprise molecules having a full-length heavy chain and molecules having a cleaved variant heavy chain. The population of antigen binding molecules may consist of a mixture of molecules having a full-length heavy chain and molecules having a cleaved variant heavy chain, wherein at least 50%, at least 60%, at least 70%, at least 80% or at least 90% of the antigen binding molecules have a cleaved variant heavy chain. In one embodiment of the invention a composition comprising a population of antigen binding molecules of the invention comprises an antigen binding molecule comprising a heavy chain including a subunit of an Fc domain as specified herein with an additional C-terminal glycine-lysine dipeptide (G446 and K447, numbering according to EU index of Kabat). In one embodiment of the invention a composition comprising a population of antigen binding molecules of the invention comprises an immune activating Fc domain binding molecule comprising a heavy chain including a subunit of an Fc domain as specified herein with an additional C-terminal glycine residue (G446, numbering according to EU index of Kabat). In one embodiment of the invention such a composition comprises a population of antigen binding molecules comprised of molecules comprising a heavy chain including a subunit of an Fc domain as specified herein: molecules comprising a heavy chain including a subunit of a Fc domain as specified herein with an additional C-terminal glycine residue (G446, numbering according to EU index of Kabat); and molecules comprising a heavy chain including a subunit of an Fc domain as specified herein with an additional C-terminal glycine-lysine dipeptide (G446 and K447, numbering according to EU index of Kabat). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service. National Institutes of Health, Bethesda, MD, 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1 (CDR-L1)-FR2-CDR-H2 (CDR-L2)-FR3-CDR-H3 (CDR-L3)-FR4.

The terms “full length antibody”, “intact antibody”, and “whole antibody” are used herein interchangeably to refer to an antibody having a structure substantially similar to a native antibody structure or having heavy chains that contain an Fc region as defined herein.

By “fused” is meant that the components (e.g., a Fab and a transmembrane domain) are linked by peptide bonds, either directly or via one or more peptide linkers.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al . . . supra. In one aspect, for the VH, the subgroup is subgroup III as in Kabat et al . . . supra.

A “humanized” antibody (e.g. a humanized scFv fragment) refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)).

Unless otherwise indicated, the CDRs are determined according to Kabat et al . . . supra. One of skill in the art will understand that the CDR designations can also be determined according to Chothia, supra, McCallum, supra, or any other scientifically accepted nomenclature system.

An “immune activating moiety” as used herein refers to one or more polypeptide(s) inducing activation of an immune cell (e.g. a T cell) upon interaction with an antigen, receptor or ligand (or other elements of the cells inducing activation) on the immune cell. Exemplary immune activating moieties are cytokines (e.g. IL2), antigen binding moieties capable of binding to a costimulatory T cell antigen (e.g. CD28, 4-1BB) or costimulatory ligands (e.g. 4-1BBL) as described herein and in WO2021/255138.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

By “isolated nucleic acid” molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed below for polypeptides (e.g., ALIGN-2).

By an “isolated polypeptide” or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus. (hetero) dimerization occurs between a polypeptide comprising the first Fc domain subunit and a polypeptide comprising the second Fc domain subunit, which might be non-identical in the sense that further components fused to each of the subunits (e.g. antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical composition.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST. BLAST-2. Clustal W. Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2.

The ALIGN-2 sequence comparison computer program was authored by Genentech, Inc., and the source code has been filed with user documentation in the U.S. Copyright Office. Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”. PNAS 85:2444-2448: W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or http://www.ebi.ac.uk/Tools/sss/fasta. Alternatively, a accessible at fasta.bioch.virginia.edu/fasta_www2/index.cgi can be used to compare the sequences, using the ggsearch (global protein: protein) program and default options (BLOSUM50; open: −10: ext: −2: Ktup=2) to ensure a global, rather than local, alignment is performed. Percent amino acid identity is given in the output alignment header. The term “nucleic acid molecule” relates to the sequence of bases comprising purine- and pyrimidine bases which are comprised by polynucleotides, whereby said bases represent the primary structure of a nucleic acid molecule.

Herein, the term nucleic acid molecule includes DNA, cDNA, genomic DNA, RNA, synthetic forms of DNA and mixed polymers comprising two or more of these molecules. In addition, the term nucleic acid molecule includes both, sense and antisense strands. Moreover, the herein described nucleic acid molecule may contain non-natural or derivatized nucleotide bases, as will be readily appreciated by those skilled in the art.

The term “package insert” is used to refer to instructions customarily included in commercial packages of therapeutic products, that contain information about the indications, usage, dosage, administration, combination therapy, contraindications and/or warnings concerning the use of such therapeutic products.

The term “pharmaceutical composition” refers to a preparation which is in such form as to permit the biological activity of an active ingredient contained therein to be effective, and which contains no additional components which are unacceptably toxic to a subject to which the formulation would be administered. A pharmaceutical composition usually comprises one or more pharmaceutically acceptable carrier(s).

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, term “polypeptide” refers to a molecule composed of monomers (amino acids) linearly linked by amide bonds (also known as peptide bonds).

The term “polypeptide” refers to any chain of two or more amino acids, and does not refer to a specific length of the product. Thus, peptides, dipeptides, tripeptides, oligopeptides, “protein”, “amino acid chain”, or any other term used to refer to a chain of two or more amino acids, are included within the definition of “polypeptide”, and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

The term “polynucleotide” refers to an isolated nucleic acid molecule or construct, e.g., messenger RNA (mRNA), virally-derived RNA, or plasmid DNA (pDNA). A polynucleotide may comprise a conventional phosphodiester bond or a non-conventional bond (e.g., an amide bond, such as found in peptide nucleic acids (PNA). The term nucleic acid molecule refers to any one or more nucleic acid segments, e.g., DNA or RNA fragments, present in a polynucleotide.

“Reduced binding”, for example reduced binding to an Fc receptor, refers to a decrease in affinity for the respective interaction, as measured for example by SPR. For clarity the term includes also reduction of the affinity to zero (or below the detection limit of the analytic method), i.e. complete abolishment of the interaction. Conversely, “increased binding” refers to an increase in binding affinity for the respective interaction.

The term “regulatory sequence” refers to DNA sequences, which are necessary to effect the expression of coding sequences to which they are ligated. The nature of such control sequences differs depending upon the host organism. In prokaryotes, control sequences generally include promoter, ribosomal binding site, and terminators. In eukaryotes generally control sequences include promoters, terminators and, in some instances, enhancers, transactivators or transcription factors. The term “control sequence” is intended to include, at a minimum, all components the presence of which are necessary for expression, and may also include additional advantageous components.

As used herein, the term “single-chain” refers to a molecule comprising amino acid monomers linearly linked by peptide bonds. In certain embodiments, one of the antigen binding moieties is a single-chain Fab molecule, i.e. a Fab molecule wherein the Fab light chain and the Fab heavy chain are connected by a peptide linker to form a single peptide chain. In a particular such embodiment, the C-terminus of the Fab light chain is connected to the N-terminus of the Fab heavy chain in the single-chain Fab molecule.

The term “SSD” as used herein refers to “stimulatory signaling domain”. An example of a stimulatory signaling domain is the intracellular domain of CD3z, or fragments thereof.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The immune activating Fc domain binding molecules of the invention are capable of inducing T cell activation. Suitable assays to measure T cell activation are known in the art described herein.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

The term “valent” as used herein denotes the presence of a specified number of antigen binding sites in an antigen binding molecule. As such, the term “monovalent binding to an antigen” denotes the presence of one (and not more than one) antigen binding site specific for the antigen in the antigen binding molecule.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

### Chimeric Receptors of the Invention

The present invention relates to chimeric receptors comprising an extracellular domain comprising a mutated Fc domain or fragment thereof (such as a CH2 and/or a CH3 domain). Provided herein is a chimeric receptor comprising a mutated Fc domain or a fragment thereof and a transmembrane domain.

The mutated Fc domain can specifically interact with antigen binding molecules, for example antibodies according to the invention to target cell expressing the chimeric receptors of the invention to target cells (e.g. tumor cells) or to deliver effector molecules (e.g. cytokines) to cells expressing the chimeric receptors of the invention.

In one embodiment, the extracellular domain comprises a mutated CH2 domain and/or a mutated CH3 domain. In one embodiment, the extracellular domain comprises a mutated CH2 domain.

The intracellular part of the chimeric receptor may provide stimulatory and co-stimulatory activity to the cells (e.g. T cells) comprising the chimeric receptor. Hence, further provided herein is a chimeric receptor comprising at least one intracellular stimulatory signaling domain and/or at least one co-stimulatory signaling domain.

The chimeric receptors of the present invention are useful is multiple therapeutic and non-therapeutic applications.

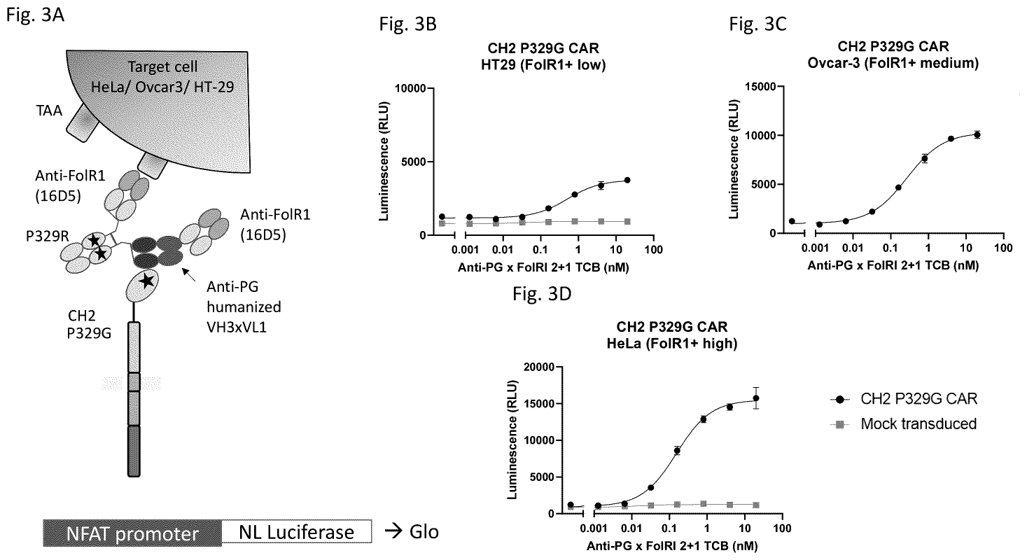

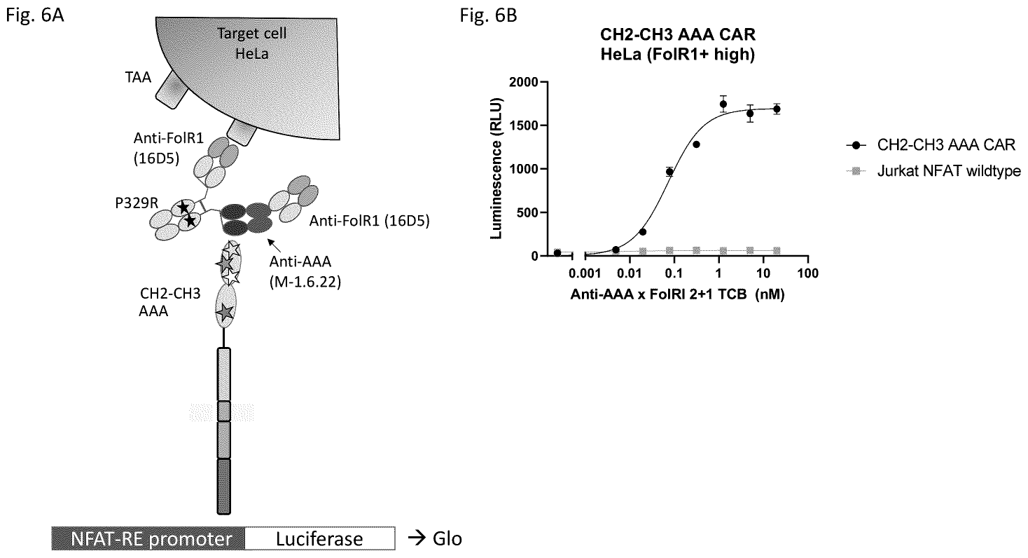

For example, the chimeric receptors of the present invention can be used to direct (or target) immune cell (such as T cells) to target cells through therapeutic antibodies comprising at least one antigen binding moiety capable of binding to the mutated Fc domain of the chimeric receptor but not capable of binding to the non-mutated parent Fc domain. In a preferred embodiment, the antibody is a multispecific antibody, i.e the antibody comprises at least one antigen binding moiety capable of binding to a target cell antigen and at least one antigen binding moiety capable of binding to the mutated Fc domain of the chimeric receptor. The therapeutic antibody can simultaneously bind to the target cell and the immune cell comprising the chimeric receptor on the immune cell whereupon the immune cell becomes activated (see e.g. FIGS. 3A and 6A).

According to this concept, the chimeric receptor comprising the mutated Fc domain is used to direct effector cells to target cells based on the target specificity of the therapeutic antibody.

The chimeric receptor is inactive in the absence of the therapeutic antibody and, importantly, exhibits an improved safety profile. The chimeric receptor of the invention comprising a mutated Fc domain is not capable of binding, e.g. in the tissue of a patient. This is in contrast to CARs comprising an antigen binding moiety (e.g. a scFv) which might exhibit off-target or on-target off-tumor binding which might result in unwanted toxicity.

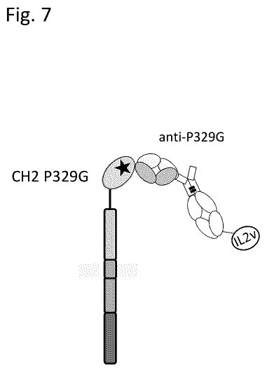

A further exemplary use of the chimeric receptors of the present invention is to specifically target effector molecules (e.g. cytokines) to cells, particularly T cells, comprising the chimeric receptors of the present invention (see e.g FIG. 7).

Of note, the CH2 domain comprising the P329G substitution (EU numbering) has been tested in clinical trials without inducing anti-drug antibodies. Moreover, the P329G substitution prevents interactions with activating Fc gamma receptors which are abundantly expressed by a variety of innate immune cells.

Hence, the present invention provides an improved chimeric receptors for improved treatment of e.g. cancer.

The present invention further relates to the transduction of T cells, such as CD8+ T cells, CD4+ T cells, CD3+ T cells, γδ T cells, natural killer (NK) cells, NKT cells or macrophages, preferably CD8+ T cells, with a chimeric receptor as described herein and their targeted recruitment, e.g., to a tumor, by an antibody molecule, e.g. a therapeutic antibody capable of binding to the mutated Fc domain.

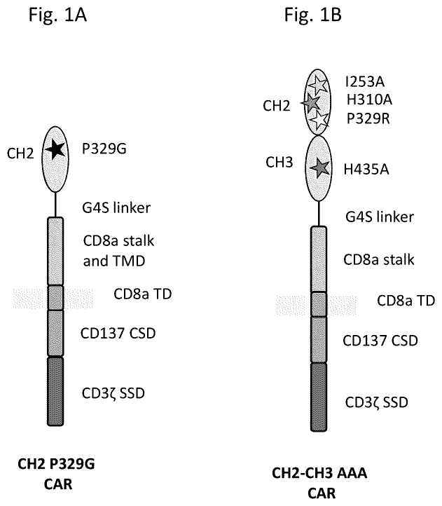

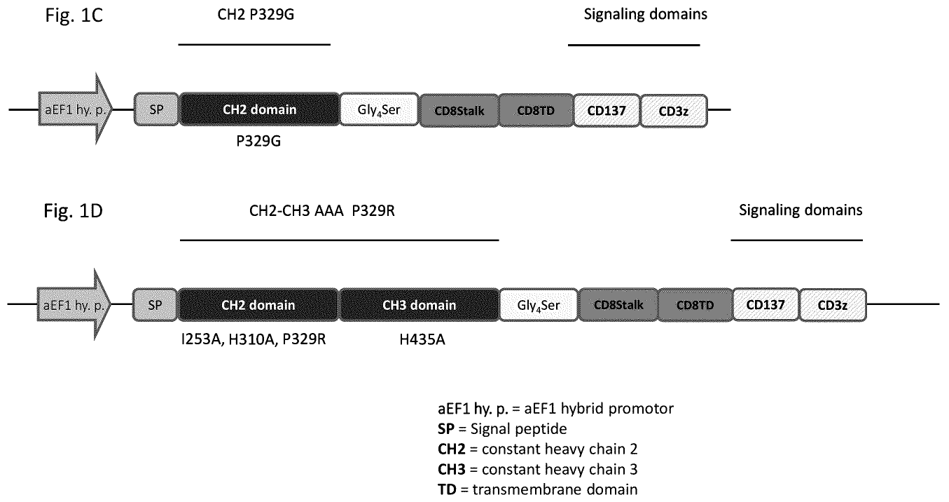

As shown in the appended Examples, as a proof of concept, a chimeric receptor comprising a CH2 domain comprising the P329G substitution according to the invention (SEQ ID NO:87 as encoded by the DNA sequence shown in SEQ ID NO:96 and as shown in FIG. 1A) was constructed which is capable of binding to a therapeutic antibody as herein described. Transduced T cells (Jurkat NFAT T cells) expressing the construct according to SEQ ID NO:87 could be strongly activated by co-incubation with an antibody that comprises two antigen binding moieties capable of binding to FolR1 and one antigen binding moiety capable of binding a CH2 domain comprising the P329G substitution but not capable of binding to the non-mutated parent Fc domain (see e.g. FIG. 3). The antibody may additionally comprise a substitution in the Fc domain at the position P329 other than glycine (G) to prevent multimerization of the therapeutic antibody (e.g. the antibody may additionally comprise the P329R substitution).

As a further proof of concept, a chimeric receptor comprising a CH2-CH3 domain comprising the AAA substitution according to the invention (SEQ ID NO: 105 as encoded by the DNA sequence shown in SEQ ID NO: 107 and as shown in FIG. 1B) was constructed which is capable of binding to a therapeutic antibody as herein described. Transduced T cells (Jurkat NFAT T cells) expressing the construct according to SEQ ID NO: 105) could be strongly activated by co-incubation with an antibody that comprises two antigen binding moieties capable of binding to FolR1 and one antigen binding moiety capable of binding a CH2-CH3 domain comprising the I253A, H310A, H435A substitutions but not capable of binding to the non-mutated parent Fc domain (see e.g. FIG. 6).

The concept of the present invention and its components are further described in detail herein below.

In one aspect of the invention, pairing of a tumor-specific antibody as herein described with T cells transduced with a chimeric receptor as herein described results in a specific activation of the T cells and subsequent lysis of the tumor cell. As hereinbefore described, this approach bears significant safety advantages over conventional T cell based approaches. Importantly, the T cell are inert in the absence of the antibody capable of binding to the mutated Fc domain.

For many antigen binding moieties, unspecific binding in a patient is difficult to avoid completely which might lead to unwanted toxicity. The present invention provides a two-component system wherein the antigen binding moiety bearing molecule (e.g. the therapeutic antibody) has a defined half-life time in vivo. This is in contrast to e.g. CAR-T cells which are permanently present in the patient after application. Hence, treating unwanted toxicities in CAR-T approaches is limited. In contrast, the chimeric receptors as provided herein does not comprise an antigen binding moiety, but a mutated Fc domain exhibiting a very well-known activity profile in vivo. This approach offers the potential of greatly reducing side effects and increasing the safety of treatment based thereupon.

The degree of T cell activation can further be adjusted by adjusting the dosage of the co-applied therapeutic antibody or by changing to different antibody specificities or formats.

### Mutated Fc Domain or a Fragment Thereof

In one aspect, provided are chimeric receptors comprising a mutated Fc domain or a fragment thereof. The Fc domain and its subdomains are well known in the art and are also herein described. The Fc domain consists of a pair of polypeptide chains comprising heavy chain constant domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. The extracellular domain according to the present invention comprises one or both subunits of an IgG Fc domain or a fragment thereof (or a dimer of a fragment thereof such as a CH2 dimer). In one aspect, the extracellular domain comprises an IgG Fc domain or a fragment thereof. In a particular embodiment, the extracellular domain comprises an IgG₁ Fc domain or a fragment thereof, or an IgG₄ Fc domain or a fragment thereof. In one embodiment, the mutated Fc domain is a mutated CH2 domain and/or a mutated CH3 domain. In one embodiment, the mutated Fc domain is a mutated CH2 domain. In one embodiment, the extracellular domain comprises a mutated CH2 domain and/or a mutated CH3 domain. In one embodiment, the extracellular domain comprises a mutated CH2 domain.

In one embodiment, the mutated Fc domain comprise at least one amino acid substitution at a position selected from the group consisting of L234, L235, 1253, H310, P331, P329 and H435 (numbering according to Kabat EU index), in particular wherein the amino acid substitution is L234A, L235A, 1253A, N297A, H310A, P329G and/or H435A.

In a preferred embodiment, the extracellular domain comprising a mutated CH2 domain, in particular wherein the mutated CH2 domain comprises the amino acid substitution P329G (numbering according to Kabat EU index).

In another preferred embodiment, the extracellular domain comprises a mutated CH2 domain and/or a mutated CH3 domain, in particular wherein the mutated CH2 domain and/or the mutated CH3 domain comprises the amino acid substitution (numbering according to Kabat EU index):
(a) I253A, H310A and H435A,
(b) I253A
(c) I253A and H310A
(d) I253A and H435A
(e) H310A and H435A
(f) H310A, or
(g) H435A.

In another preferred embodiment, the extracellular domain comprises a mutated CH2 domain and/or a mutated CH3 domain, in particular wherein the mutated CH2 domain and/or the mutated CH3 domain comprises the amino acid substitution (numbering according to Kabat EU index):
(a) I253A, H310A and H435A,
(b) I253A
(c) I253A and H310A
(d) I253A and H435A
(e) H310A and H435A
(f) H310A,
(g) H435A,
(h) L234A, L235A, and P329G, or
(i) L234A, L235A, I253A, H310A, P329G and H435A.

In one embodiment, the extracellular domain comprising a mutated CH2 domain and/or a mutated CH3 domain further comprises an amino acid substitution selected from the group consisting of P329G, P329L, P329I, P329R, and P329A (numbering according to Kabat EU index).

In a preferred embodiment, the extracellular domain comprises a mutated CH2 domain and a mutated CH3 domain, wherein the mutated CH2 domain and the mutated CH3 domain comprise the amino acid substitution I253A, H310A, P329G and H435A (numbering according to Kabat EU index). In one such embodiment, the chimeric receptor is combined with a therapeutic antibody as herein described comprising the P329R substitution (numbering according to Kabat) as well as an antigen binding moiety capable of binding to a CH2 domain comprising the P329R substitution.

In another preferred embodiment, the extracellular domain comprises a mutated CH2 domain and a mutated CH3 domain, wherein the mutated CH2 domain and the mutated CH3 domain comprise the amino acid substitution 1253A, H310A, P329R and H435A (numbering according to Kabat EU index). In one such embodiment, the chimeric receptor is combined with a therapeutic antibody as herein described comprising the P329G substitution (numbering according to Kabat) as well as an antigen binding moiety capable of binding to a CH2 domain comprising the P329R substitution.

In one embodiment, the extracellular domain does not comprise a VH and/or VL domain.

In the context of the present invention, the antigen binding receptor comprises an extracellular domain that does not naturally occur in or on T cells. Thus, the antigen binding receptor is capable of providing tailored binding specificity to cells expressing the antigen binding receptor according to the invention. In one embodiment, the extracellular domain comprises a mutated CH2 domain. In one embodiment, the extracellular domain comprises a mutated Fc domain or fragment thereof, wherein the mutated Fc domain or fragment thereof comprises or consists of one of the below mutated CH2 domains (1)-(18).

(1) In one embodiment, the mutated CH2 domain comprises or consist of an amino acid sequence that is at least about 95%, 96%, 97%, 98%, 99% or 100% identical to the amino acid sequence of SEQ ID NO:20, wherein the mutated CH2 domain comprises the amino acid G329 (numbering according to Kabat).

(2) In one embodiment, the mutated CH2 domain comprises or consist of an amino acid sequence that is at least about 95%, 96%, 97%, 98%, 99% or 100% identical to the amino acid sequence of SEQ ID NO:20, wherein the mutated CH2 domain comprises glycine (G) at position 99 wherein numbering is relative to the sequence of SEQ ID NO: 20.

(3) In one embodiment, the mutated CH2 domain comprises or consists of the amino acid sequence of SEQ ID NO:20.

(4) In one embodiment, the mutated CH2 domain comprises or consist of an amino acid sequence that is at least about 95%, 96%, 97%, 98%, 99% or 100% identical to the amino acid sequence of SEQ ID NO:21, wherein the mutated CH2 domain comprises the amino acid L329 (numbering according to Kabat).

(5) In one embodiment, the mutated CH2 domain comprises or consist of an amino acid sequence that is at least about 95%, 96%, 97%, 98%, 99% or 100% identical to the amino acid sequence of SEQ ID NO:21, wherein the mutated CH2 domain comprises leucine (L) at position 99 wherein numbering is relative to the sequence of SEQ ID NO: 21.

(6) In one embodiment, the mutated CH2 domain comprises or consists of the amino acid sequence of SEQ ID NO:21.

(7) In one embodiment, the mutated CH2 domain comprises or consist of an amino acid sequence that is at least about 95%, 96%, 97%, 98%, 99% or 100% identical to the amino acid sequence of SEQ ID NO:22, wherein the mutated CH2 domain comprises the amino acid 1329 (numbering according to Kabat).

(8) In one embodiment, the mutated CH2 domain comprises or consist of an amino acid sequence that is at least about 95%, 96%, 97%, 98%, 99% or 100% identical to the amino acid sequence of SEQ ID NO:22, wherein the mutated CH2 domain comprises isoleucine (I) at position 99 wherein numbering is relative to the sequence of SEQ ID NO: 22.

(9) In one embodiment, the mutated CH2 domain comprises or consists of the amino acid sequence of SEQ ID NO:22.

(10) In one embodiment, the mutated CH2 domain comprises or consist of an amino acid sequence that is at least about 95%, 96%, 97%, 98%, 99% or 100% identical to the amino acid sequence of SEQ ID NO:23, wherein the mutated CH2 domain comprises the amino acid R329 (numbering according to Kabat).

(11) In one embodiment, the mutated CH2 domain comprises or consist of an amino acid sequence that is at least about 95%, 96%, 97%, 98%, 99% or 100% identical to the amino acid sequence of SEQ ID NO:23, wherein the mutated CH2 domain comprises arginine (R) at position 99 wherein numbering is relative to the sequence of SEQ ID NO: 23.

(12) In one embodiment, the mutated CH2 domain comprises or consists of the amino acid sequence of SEQ ID NO:23.

(13) In one embodiment, the mutated CH2 domain comprises or consist of an amino acid sequence that is at least about 95%, 96%, 97%, 98%, 99% or 100% identical to the amino acid sequence of SEQ ID NO:24, wherein the mutated CH2 domain comprises the amino acid A329 (numbering according to Kabat).

(14) In one embodiment, the mutated CH2 domain comprises or consist of an amino acid sequence that is at least about 95%, 96%, 97%, 98%, 99% or 100% identical to the amino acid sequence of SEQ ID NO:24, wherein the mutated CH2 domain comprises alanine (A) at position 99 wherein numbering is relative to the sequence of SEQ ID NO: 24.

(15) In one embodiment, the mutated CH2 domain comprises or consists of the amino acid sequence of SEQ ID NO:24.

(16) In one embodiment, the mutated CH2 domain comprises or consist of an amino acid sequence that is at least about 95%, 96%, 97%, 98%, 99% or 100% identical to the amino acid sequence of SEQ ID NO:86, wherein the mutated CH2 domain comprises the amino acids A234, A235 and G329 (numbering according to Kabat).

(17) In one embodiment, the mutated CH2 domain comprises or consist of an amino acid sequence that is at least about 95%, 96%, 97%, 98%, 99% or 100% identical to the amino acid sequence of SEQ ID NO:86, wherein the mutated CH2 domain comprises alanine (A) at position 4 and 5, and glycine (G) at position 99 wherein numbering is relative to the sequence of SEQ ID NO: 86.

(18) In a preferred embodiment, the mutated CH2 domain comprises or consists of the amino acid sequence of SEQ ID NO:86.

In a specific preferred embodiment, provided is a chimeric receptor comprising an extracellular domain comprising a mutated CH2 domain comprising or consisting of the amino acid sequence of SEQ ID NO:86, and a transmembrane domain.

Optionally, the mutated CH2 domain as hereinabove described further comprises a CH3 domain. As can be seen in Example 7, the inclusion of a CH3 domain may lead to a stronger effect (even if the antibody capable of binding to the chimeric receptor of the invention is directed against the mutated CH2 domain). Without being bound to theory, the inventors believe that inclusion of a CH2-CH3 domain in the extracellular domain leads to stronger expression of the chimeric receptor. Expression of the chimeric receptor can be measured using methods known in the art and as described in the appended Examples.

In one embodiment, the CH3 domain is a mutated CH3 domain. In one embodiment, the extracellular domain comprises a mutated CH2-CH3 domain (i.e. a CH2 domain fused to a CH3 domain, optionally through a peptide linker).

In one embodiment, the extracellular domain comprises a mutated Fc domain or fragment thereof, wherein the mutated Fc domain or fragment thereof comprises or consists of one of the below mutated CH2-CH3 domains (1)-(4).

(1) In one embodiment, the mutated CH2-CH3 domain comprises or consist of the amino acid sequence of SEQ ID NO:27.

(2) In one embodiment, the mutated CH2-CH3 domain comprises or consist of the amino acid sequence of SEQ ID NO: 105.

(3) In one embodiment, the mutated CH2-CH3 domain comprises or consist of the amino acid sequence of SEQ ID NO: 124.

(4) In one embodiment, the mutated CH2-CH3 domain comprises or consist of the amino acid sequence of SEQ ID NO: 126.

### Transmembrane Domain (TD)

In the context of the present invention any transmembrane domain of a transmembrane protein as laid down among others by the CD-nomenclature may be used to generate the chimeric receptors of the invention.

Accordingly, in the context of the present invention, the transmembrane domain may comprise part of a murine/mouse or preferably of a human transmembrane domain. An example for such an transmembrane domain is a transmembrane domain of CD8, for example, having the amino acid sequence as shown herein in SEQ ID NO:89 (as encoded by the DNA sequence shown in SEQ ID NO:98). In the context of the present invention, the transmembrane domain of the chimeric receptor of the present invention may comprise/consist of an amino acid sequence as shown in SEQ ID NO:89 (as encoded by the DNA sequence shown in SEQ ID NO:98).

In another embodiment, the herein provided chimeric receptor may comprise the transmembrane domain of CD28 which is located at amino acids 153 to 179, 154 to 179, 155 to 179, 156 to 179, 157 to 179, 158 to 179, 159 to 179, 160 to 179, 161 to 179, 162 to 179, 163 to 179, 164 to 179, 165 to 179, 166 to 179, 167 to 179, 168 to 179, 169 to 179, 170 to 179, 171 to 179, 172 to 179, 173 to 179, 174 to 179, 175 to 179, 176 to 179, 177 to 179 or 178 to 179 of the human full length CD28 protein as shown in SEQ ID NO:41 (as encoded by the cDNA shown in SEQ ID NO:40).

Alternatively, any protein having a transmembrane domain, as provided among others by the CD nomenclature, may be used as a transmembrane domain of the chimeric receptor protein of the invention.

In some embodiments, the transmembrane domain comprises the transmembrane domain of any one of the group consisting of CD27 (SEQ ID NO:37 as encoded by SEQ ID NO:36), CD137 (SEQ ID NO:45 as encoded by SEQ ID NO:44), OX40 (SEQ ID NO:49, as encoded by SEQ ID NO: 48), ICOS (SEQ ID NO:53 as encoded by SEQ ID NO:52), DAP10 (SEQ ID NO:57 as encoded by SEQ ID NO:56), DAP12 (SEQ ID NO:61 as encoded by SEQ ID NO:60), CD3% (SEQ ID NO:64 as encoded by SEQ ID NO:65), FCGR3A (SEQ ID NO:68 as encoded by SEQ ID NO: 69), NKG2D (SEQ ID NO: 72 as encoded by SEQ ID NO: 73), CD8 (SEQ ID NO:82 as encoded by SEQ ID NO:83), or a fragment of the transmembrane thereof that retains the capability to anchor the chimeric receptor to the membrane.

Human sequences might be beneficial in the context of the common invention, for example because (parts) of the transmembrane domain might be accessible from the extracellular space and hence to the immune system of a patient.

### Stimulatory Signaling Domain (SSD) and Co-Stimulatory Signaling Domain (CSD)

Preferably, the chimeric receptor of the present invention comprises at least one stimulatory signaling domain and/or at least one co-stimulatory signaling domain. Accordingly, the herein provided chimeric receptor preferably comprises a stimulatory signaling domain, which provides T cell activation. The herein provided chimeric receptor may comprise a stimulatory signaling domain which is a fragment/polypeptide part of murine/mouse or human CD3% (the UniProt Entry of the human CD3z is P20963 (version number 177 with sequence number 2: the UniProt Entry of the murine/mouse CD3% is P24161 (primary citable accession number) or Q9D3G3 (secondary citable accession number) with the version number 143 and the sequence number 1)), FCGR3A (the UniProt Entry of the human FCGR3A is P08637 (version number 178 with sequence number 2)), or NKG2D (the UniProt Entry of the human NKG2D is P26718 (version number 151 with sequence number 1); the UniProt Entry of the murine/mouse NKG2D is 054709 (version number 132 with sequence number 2)).

Thus, the stimulatory signaling domain which is comprised in the herein provided chimeric receptor may be a fragment/polypeptide part of the full length of CD3%. FCGR3A or NKG2D.

The amino acid sequences of the murine/mouse full length of CD3%, or NKG2D are shown herein as SEQ ID NOs: 66 (CD3z), 70 (FCGR3A) or 74 (NKG2D) (murine/mouse as encoded by the DNA sequences shown in SEQ ID NOs: 67 (CD32), 71 (FCGR3A) or 75 (NKG2D). The amino acid sequences of the human full length CD3%. FCGR3A or NKG2D are shown herein as SEQ ID NOs: 64 (CD3z), 68 (FCGR3A) or 72 (NKG2D) (human as encoded by the DNA sequences shown in SEQ ID NOs: 65 (CD32), 69 (FCGR3A) or 73 (NKG2D)). The chimeric receptor of the present invention may comprise fragments of CD32. FCGR3A or NKG2D as stimulatory domain, provided that at least one signaling domain is comprised. In particular, any part/fragment of CD3z, FCGR3A, or NKG2D is suitable as stimulatory domain as long as at least one signaling motive is comprised. However, more preferably, the chimeric receptor of the present invention comprises polypeptides which are derived from human origin.

In a preferred embodiment, stimulatory signaling domain(s) which is (are) comprised in the chimeric receptor comprises or consists of the amino acid sequence shown in SEQ ID NO:91 (as encoded by the DNA sequence shown in SEQ ID NO: 100). In further embodiments the chimeric receptor comprises the sequence as shown in SEQ ID NO:91 or a sequence which has up to 1, 2, 3, 4, 5, 6, 7, 8, 9, 10, 11, 12, 13, 14, 15, 16, 17, 18, 19, 20, 21, 22, 23, 23, 24, 25, 26, 27, 28, 29 or 30 substitutions, deletions or insertions in comparison to SEQ ID NO:91 and which is characterized by having (or retaining) a stimulatory signaling activity. Specific configurations of chimeric receptors comprising a stimulatory signaling domain (SSD) are provided herein below and in the Examples and Figures. The stimulatory signaling activity can be determined; e.g., by enhanced cytokine release, as measured by ELISA (IL-2, IFNγ, TNFα), enhanced proliferative activity (as measured by enhanced cell numbers), or enhanced lytic activity as measured by LDH release assays.

Furthermore, the herein provided chimeric receptor preferably comprises at least one co-stimulatory signaling domain which provides additional activity to the T cell. The herein provided chimeric receptor may comprise a co-stimulatory signaling domain which is a fragment/polypeptide part of murine/mouse or human CD28 (the UniProt Entry of the human CD28 is P10747 (version number 173 with sequence number 1); the UniProt Entry of the murine/mouse CD28 is P31041 (version number 134 with sequence number 2)), CD137 (the UniProt Entry of the human CD137 is Q07011 (version number 145 with sequence number 1); the UniProt Entry of murine/mouse CD137 is P20334 (version number 139 with sequence number 1)), OX40 (the UniProt Entry of the human OX40 is P23510 (version number 138 with sequence number 1); the UniProt Entry of murine/mouse OX40 is P43488 (version number 119 with sequence number 1)), ICOS (the UniProt Entry of the human ICOS is Q9Y6W8 (version number 126 with sequence number 1)); the UniProt Entry of the murine/mouse ICOS is Q9WV40 (primary citable accession number) or Q9JL17 (secondary citable accession number) with the version number 102 and sequence version 2)), CD27 (the UniProt Entry of the human CD27 is P26842 (version number 160 with sequence number 2); the Uniprot Entry of the murine/mouse CD27 is P41272 (version number 137 with sequence version 1)), 4-1-BB (the UniProt Entry of the murine/mouse 4-1-BB is P20334 (version number 140 with sequence version 1); the UniProt Entry of the human 4-1-BB is Q07011 (version number 146 with sequence version)), DAP10 (the UniProt Entry of the human DAP10 is Q9UBJ5 (version number 25 with sequence number 1); the UniProt entry of the murine/mouse DAP10 is Q9QUJ0 (primary citable accession number) or Q9RIE7 (secondary citable accession number) with the version number 101 and the sequence number 1)) or DAP12 (the UniProt Entry of the human DAP12 is 043914 (version number 146 and the sequence number 1); the UniProt entry of the murine/mouse DAP12 is 0054885 (primary citable accession number) or Q9RIE7 (secondary citable accession number) with the version number 123 and the sequence number 1). In certain embodiments of the present invention the chimeric receptor of the present invention may comprise one or more. i.e. 1, 2, 3, 4, 5, 6 or 7 of the herein defined co-stimulatory signaling domains. Accordingly, in the context of the present invention, the chimeric receptor of the present invention may comprise a fragment/polypeptide part of a murine/mouse or preferably of a human CD137 as first co-stimulatory signaling domain and the second co-stimulatory signaling domain is selected from the group consisting of the murine/mouse or preferably of the human CD27, CD28, CD137, OX40, ICOS, DAP10 and DAP12, or fragments thereof.

Preferably, the chimeric receptor of the present invention comprises a co-stimulatory signaling domain which is derived from a human origin.

In a preferred embodiment, the co-stimulatory signaling domain(s) which is (are) comprised in the chimeric receptor of the present invention may comprise or consist of the amino acid sequence as shown in SEQ ID NO:90 (as encoded by the DNA sequence shown in SEQ ID NO: 99).

Thus, the co-stimulatory signaling domain which may be optionally comprised in the herein provided chimeric receptor is a fragment/polypeptide part of the full length CD27, CD28, CD137, OX40, ICOS, DAP10 or DAP12, The amino acid sequences of the murine/mouse full length CD27, CD28, CD137, OX40, ICOS, CD27, DAP10 and DAP12 are shown herein as SEQ ID NOs: 39 (CD27), 43 (CD28), 47 (CD137), 51 (OX40), 55 (ICOS), 59 (DAP10) or 63 (DAP12) (murine/mouse as encoded by the DNA sequences shown in SEQ ID NOs: 38 (CD27), 42 (CD28), 46 (CD137), 50 (OX40), 54 (ICOS), 58 (DAP10) or 62 (DAP12)). However, because human sequences are most preferred in the context of the present invention, the co-stimulatory signaling domain which may be optionally comprised in the herein provided chimeric receptor protein is a fragment/polypeptide part of the human full length CD27, CD28, CD137, OX40, ICOS, DAP10 or DAP12. The amino acid sequences of the human full length CD27, CD28, CD137, OX40, ICOS, DAP10 or DAP12 are shown herein as SEQ ID NOs: 37, (CD27), 41 (CD28), 45 (CD137), 49 (OX40), 53 (ICOS), 57 (DAP10) or 61 (DAP12) (human as encoded by the DNA sequences shown in SEQ ID NOs: 36 (CD27), 40 (CD28), 44 (CD137), 48 (OX40), 52 (ICOS), 56 (DAP10) or 60 (DAP12)).

In one embodiment, the chimeric receptor comprises CD28 or a fragment thereof as co-stimulatory signaling domain. The herein provided chimeric receptor may comprise a fragment of CD28 as co-stimulatory signaling domain, provided that at least one signaling domain of CD28 is comprised. In particular, any part/fragment of CD28 is suitable for the chimeric receptor of the invention as long as at least one of the signaling motives of CD28 is comprised. The co-stimulatory signaling domains PYAP (AA 208 to 211 of CD28) and YMNM (AA 191 to 194 of CD28) are beneficial for the function of the CD28 polypeptide and the functional effects enumerated above. The amino acid sequence of the YMNM domain is shown in SEQ ID NO: 76; the amino acid sequence of the PYAP domain is shown in SEQ ID NO:77.

Accordingly, in the chimeric receptor of the present invention, the CD28 polypeptide preferably comprises a sequence derived from intracellular domain of a CD28 polypeptide having the sequences YMNM (SEQ ID NO: 76) and/or PYAP (SEQ ID NO:77). In other embodiments, in the chimeric receptor of the present invention, one or both of these domains are mutated to FMNM (SEQ ID NO:78) and/or AYAA (SEQ ID NO:79), respectively. Either of these mutations reduces the ability of a transduced cell comprising the chimeric receptor to release cytokines without affecting its ability to proliferate and can advantageously be used to prolong the viability and thus the therapeutic potential of the transduced cells. Or, in other words, such a non-functional mutation preferably enhances the persistence of the cells which are transduced with the herein provided chimeric receptor in vivo. These signaling motives may, however, be present at any site within the intracellular domain of the herein provided chimeric receptor. In a preferred embodiment, the chimeric receptor comprises CD137 or a fragment thereof as co-stimulatory signaling domain. The herein provided chimeric receptor may comprise a fragment of CD137 as co-stimulatory signaling domain, provided that at least one signaling domain of CD137 is comprised. In particular, any part/fragment of CD137 is suitable for the chimeric receptor of the invention as long as at least one of the signaling motives of CD137 is comprised. In a preferred embodiment, the CD137 polypeptide which is comprised in the chimeric receptor protein of the present invention comprises or consists of the amino acid sequence shown in SEQ ID NO:90 (as encoded by the DNA sequence shown in SEQ ID NO: 99).

Specific configurations of chimeric receptors comprising a co-stimulatory signaling domain (CSD) are provided herein below and in the Examples and Figures. The co-stimulatory signaling activity can be determined: e.g., by enhanced cytokine release, as measured by ELISA (IL-2, IFNγ, TNFα), enhanced proliferative activity (as measured by enhanced cell numbers), or enhanced lytic activity as measured by LDH release assays. CD28 and/or CD137 activity can be measured by release of cytokines by ELISA or flow cytometry of cytokines such as interferon-gamma (IFN-γ) or interleukin 2 (IL-2), proliferation of T cells measured e.g. by ki67-measurement, cell quantification by flow cytometry, or lytic activity as assessed by real time impedence measurement of the target cell (by using e.g. an ICELLligence instrument as described e.g. in Thakur et al., Biosens Bioelectron. 35 (1) (2012), 503-506; Krutzik et al., Methods Mol Biol. 699 (2011), 179-202: Ekkens et al., Infect Immun. 75 (5) (2007), 2291-2296; Ge et al., Proc Natl Acad Sci USA. 99 (5) (2002), 2983-2988: Düwell et al., Cell Death Differ. 21 (12) (2014), 1825-1837. Erratum in: Cell Death Differ. 21 (12) (2014), 161).

### Additional Linkers and Signal Peptides

Moreover, the herein provided chimeric receptor may comprise at least one linker (or “spacer”). A linker is usually a peptide having a length of up to 20 amino acids. Accordingly, in the context of the present invention the linker may have a length of 1, 2, 3, 4, 5, 6, 7, 8, 9, 10, 11, 12, 13, 14, 15, 16, 17, 18, 19 or 20 amino acids. For example, the herein provided chimeric receptor may comprise a linker between the extracellular domain, the transmembrane domain, the co-stimulatory signaling domain and/or the stimulatory signaling domain. Such linkers may have the advantage that they may increase the probability that the different polypeptides of the chimeric receptor (i.e. the extracellular domain, the transmembrane domain, the co-stimulatory signaling domain and/or the stimulatory signaling domain) fold independently and behave as expected. In the context of the present invention, the extracellular domain, the transmembrane domain, the co-stimulatory signaling domain and the stimulatory signaling domain may be comprised in a single-chain multi-functional polypeptide. A single-chain fusion construct e.g. may consist of (a) polypeptide(s) comprising (an) extracellular domain(s), (an) transmembrane domain(s), (a) co-stimulatory signaling domain(s) and/or (a) stimulatory signaling domain(s).

Accordingly, the extracellular domain, the transmembrane domain, the co-stimulatory signaling domain and the stimulatory signaling domain may be connected by one or more identical or different peptide linker as described herein. For example, in the herein provided chimeric receptor the linker between the extracellular domain and the transmembrane domain may comprise or consist of the amino and amino acid sequence as shown in SEQ ID NO:97.

Accordingly, the transmembrane domain, the co-stimulatory signaling domain and/or the stimulatory domain may be connected to each other by peptide linkers or alternatively, by direct fusion of the domains.

The herein provided chimeric receptor or parts thereof may comprise a signal peptide. Such a signal peptide will bring the protein to the surface of the T cell membrane. For example, in the herein provided chimeric receptor the signal peptide may have the amino and amino acid sequence as shown in SEQ ID NO:80 (as encoded by the DNA sequence shown in SEQ ID NO: 81).

### Specific Chimeric Receptors Capable of Binding to Mutated Fc Domains

The components of the chimeric receptors as described herein above can be fused to each other in a variety of configurations.

In some embodiments, the chimeric receptor comprises an extracellular domain comprising a mutated Fc domain. In one embodiment, the chimeric receptor comprises a mutated CH2 domain and/or a mutated CH3 domain as hereinbefore described. In preferred embodiments, the Fc domain is fused at the C-terminus to the N-terminus of the transmembrane domain, optionally through a peptide linker. In other embodiments, the chimeric receptor further comprises a stimulatory signaling domain and/or a co-stimulatory signaling domain.

In a specific such embodiment, the chimeric receptor comprises (or essentially consists of) an extracellular domain comprising a mutated Fc domain (e.g. a CH2 and/or CH3 domain) fused at the C-terminus to the N-terminus of the transmembrane domain, wherein the transmembrane domain is fused at the C-terminus to the N-terminus of the stimulatory signaling domain.

Optionally, the chimeric receptor further comprises a co-stimulatory signaling domain. In one such specific embodiment, the chimeric receptor comprises (or essentially consists of) an extracellular domain comprising a mutated Fc domain (e.g. a CH2 and/or CH3 domain) fused at the C-terminus to the N-terminus of the transmembrane domain, wherein the transmembrane domain is fused at the C-terminus to the N-terminus of the stimulatory signaling domain, wherein the stimulatory signaling domain is fused at the C-terminus to the N-terminus of the co-stimulatory signaling domain.

In an alternative embodiment, the co-stimulatory signaling domain is connected to the transmembrane domain instead of the stimulatory signaling domain. In a specific such embodiment, the chimeric receptor comprises (or essentially consists of) an extracellular domain comprising a mutated Fc domain (e.g. a CH2 and/or CH3 domain) fused at the C-terminus to the N-terminus of the transmembrane domain, wherein the transmembrane domain is fused at the C-terminus to the N-terminus of the co-stimulatory signaling domain, wherein the co-stimulatory signaling domain is fused at the C-terminus to the N-terminus of the stimulatory signaling domain.

In a preferred embodiment, the chimeric receptor comprises (or essentially consists of) from N-terminus to C-terminus in this order an extracellular domain comprising a mutated Fc domain (e.g. a CH2 and/or CH3 domain), a transmembrane domain, a co-stimulatory signaling domain and a stimulatory signaling domain connected by one or more peptide linkers. In a preferred embodiment, the mutated Fc domain is a CH2 domain.

The Fc domain, the transmembrane domain and the stimulatory signaling and/or co-stimulatory signaling domains may be fused to each other directly or through one or more peptide linker, comprising one or more amino acids, typically about 2-20 amino acids. Peptide linkers are known in the art and are described herein. Suitable, non-immunogenic peptide linkers include, for example, (G₄S)ₙ, (SG₄)ₙ, (G₄S)ₙ or G₄(SG₄)ₙ peptide linkers, wherein “n” is generally a number between 1 and 10, typically between 2 and 4. A preferred peptide linker for connecting the antigen binding moiety and the anchoring transmembrane moiety is (G₄S). Another preferred peptide linker for connecting the antigen binding moiety and the anchoring transmembrane moiety is the amino acid sequence of SEQ ID NO: 88 (CD8stalk). Additionally, linkers may comprise (a portion of) an immunoglobulin hinge region.

In one embodiment, the Fc domain (e.g. the mutated CH2 domain) is an IgG Fc domain or fragment thereof, specifically an IgG₁ or IgG₄ Fc domain or fragment thereof. In one embodiment, the extracellular domain comprises a CH2 domain, a CH3 domain and/or a CH4 domain, preferably a CH2 domain. In one embodiment the Fc domain comprises at least one amino acid substitution compared to a native Fc domain. In one embodiment, the at least one amino acid substitution reduce binding to an Fc receptor and/or reduce effector function. In one embodiment, the at least one amino acid substitution is at a position selected from the list consisting of 233, 234, 235, 238, 253, 265, 269, 270, 297, 310, 331, 327, 329 and 435 (numberings according to Kabat EU index). In one embodiment, the at least one amino acid substitution comprises a substitution at position P329 (numbering according to Kabat EU index). In one embodiment, the at least one amino acid substitution comprises a substitution at position P329 (numbering according to Kabat EU index) by an amino acid selected from the list consisting of alanine (A) arginine (R), leucine (L), isoleucine (I), and proline (P). In one embodiment, the at least one amino acid substitution comprises the amino acid substitution P329G (numbering according to Kabat EU index).

In one particular embodiment, the chimeric receptor comprises a mutated Fc domain, in particular an IgG1 Fc domain, comprising the P329G substitution (according to EU numbering). In one embodiment, the Fc domain comprises the P329G substitution (according to EU numbering). In one embodiment, the Fc domain is fused at the C-terminus to the N-terminus of a transmembrane domain, optionally through a peptide linker. In one embodiment the peptide linker comprises the amino acid sequence of SEQ ID NO:88. In one embodiment, the transmembrane domain is a transmembrane domain selected from the group consisting of the CD8, the CD4, the CD32, the FCGR3A, the NKG2D, the CD27, the CD28, the CD137, the OX40, the ICOS, the DAP10 or the DAP12 transmembrane domain or a fragment thereof. In a preferred embodiment, the transmembrane domain is the CD8 transmembrane domain or a fragment thereof. In a particular embodiment, the transmembrane domain comprises or consist of the amino acid sequence of SEQ ID NO:89. In one embodiment, the chimeric receptor further comprises a co-stimulatory signaling domain (CSD). In one embodiment, the transmembrane domain of the chimeric receptor is fused at the C-terminus to the N-terminus of a co-stimulatory signaling domain. In one embodiment, the co-stimulatory signaling domain is individually selected from the group consisting of the intracellular domain of CD27, of CD28, of CD137, of OX40, of ICOS, of DAP10 and of DAP12, or fragments thereof as described herein before. In one embodiment, the co-stimulatory signaling domain is the intracellular domain of CD28 or a fragment thereof. In one embodiment, the co-stimulatory signaling domain comprises the intracellular domain of CD28 or a fragment thereof that retains CD28 signaling. In a preferred embodiment, the co-stimulatory signaling domain comprises the intracellular domain of CD137 or a fragment thereof that retains CD137 signaling. In a particular such embodiment the co-stimulatory signaling domain comprises or consists of SEQ ID NO:90. In one embodiment, the chimeric receptor further comprises a stimulatory signaling domain. In one embodiment, the co-stimulatory signaling domain of the chimeric receptor is fused at the C-terminus to the N-terminus of the stimulatory signaling domain. In one embodiment, the at least one stimulatory signaling domain is individually selected from the group consisting of the intracellular domain of CD3%. FCGR3A and NKG2D. or fragments thereof. In a preferred embodiment, the stimulatory signaling domain is the intracellular domain of CD3% or a fragment thereof that retains CD3% signaling. In a particular embodiment the stimulatory signaling domain comprises or consists of SEQ ID NO:91.

In one embodiment, the chimeric receptor is fused to a reporter protein, particularly to GFP or enhanced analogs thereof. In one embodiment, the chimeric receptor is fused at the C-terminus to the N-terminus of eGFP (enhanced green fluorescent protein), optionally through a peptide linker as described herein. In a preferred embodiment, the peptide linker is GEGRGSLLTCGDVEENPGP (T2A) according to SEQ ID NO:95.

In a particular embodiment, the chimeric receptor comprises an transmembrane domain and an extracellular domain comprising a mutated CH2 domain, wherein the mutated CH2 domain comprises the P329G substitution (according to EU numbering). The P329G substitution reduces Fcγ receptor binding. In one embodiment, the chimeric receptor of the invention comprises an transmembrane domain (TD), a co-stimulatory signaling domain (CSD) and a stimulatory signaling domain (SSD). In one such embodiment, the chimeric receptor has the configuration CH2-TD-CSD-SSD. In another embodiment, the chimeric receptor has the configuration CH2-TD-SSD-CSD. In a preferred embodiment, the chimeric receptor has the configuration CH2 (P329G)-TD-CSD-SSD. In a more specific such embodiment, the chimeric receptor has the configuration CH2 (P329G)-linker-TD-CSD-SSD wherein “linker” is a linker as herein before described.

In one embodiment the present invention provides a chimeric receptor comprising or consisting of in order from the N-terminus to the C-terminus an extracellular domain comprising a CH2 domain, wherein the CH2 domain comprises the amino acid substitution P329G (numbering according to Kabat EU index), and a transmembrane domain. In a particular embodiment, the CH2 domain comprises or consists of a sequence of SEQ ID NO:86.

In one embodiment the present invention provides a chimeric receptor comprising in order from the N-terminus to the C-terminus:
(i) an extracellular domain comprising a CH2 domain, wherein the CH2 domain comprises the amino acid substitution P329G (numbering according to Kabat EU index),
(ii) a transmembrane domain, and
(iii) at least one intracellular stimulatory signaling domain and/or at least one co-stimulatory signaling domain.

In one embodiment the present invention provides a chimeric receptor comprising in order from the N-terminus to the C-terminus:
(i) an extracellular domain comprising a CH2 domain of SEQ ID NO:86; and optionally a linker, in particular the linker of SEQ ID NO:88,
(ii) a transmembrane domain: in particular the transmembrane domain of SEQ ID NO:89,
(iii) a co-stimulatory signaling domain, in particular the co-stimulatory signaling domain of SEQ ID NO:90, and
(iv) a stimulatory signaling domain, in particular the stimulatory signaling domain of SEQ ID NO: 91.

In one embodiment, provided is a chimeric receptor comprising an amino acid sequence that is at least about 95%, 96%, 97%, 98%, 99% or 100% identical to the amino acid sequence of: SEQ ID NO:87. In one embodiment, provided is a chimeric receptor comprising or consisting of the amino acid sequence of: SEQ ID NO:87.

In one embodiment the present invention provides a chimeric receptor comprising in order from the N-terminus to the C-terminus an extracellular domain comprising a CH2-CH3 domain, wherein the CH2-CH3 domain comprises the amino acid substitutions I253A, H310A and H435A (numbering according to Kabat EU index), and a transmembrane domain. In a particular embodiment, the CH2-CH3 domain comprises or consists of the amino acid sequence sequence of SEQ ID NO: 105.

In one embodiment the present invention provides a chimeric receptor comprising in order from the N-terminus to the C-terminus:
(i) an extracellular domain comprising a CH2-CH3 domain, wherein the CH2-CH3 domain comprises the amino acid substitutions I253A, H310A and H435A (numbering according to Kabat EU index),
(ii) a transmembrane domain, and
(iii) at least one intracellular stimulatory signaling domain and/or at least one co-stimulatory signaling domain.

In one embodiment the present invention provides a chimeric receptor comprising in order from the N-terminus to the C-terminus:
(i) an extracellular domain comprising a CH2-CH3 domain of SEQ ID NO: 105; and optionally a linker, in particular the linker of SEQ ID NO:88,
(ii) a transmembrane domain: in particular the transmembrane domain of SEQ ID NO:89,
(iii) a co-stimulatory signaling domain, in particular the co-stimulatory signaling domain of SEQ ID NO:90, and
(iv) a stimulatory signaling domain, in particular the stimulatory signaling domain of SEQ ID NO: 91.

In one embodiment, provided is a chimeric receptor comprising an amino acid sequence that is at least about 95%, 96%, 97%, 98%, 99% or 100% identical to the amino acid sequence of: SEQ ID NO:106. In one embodiment, provided is a chimeric receptor comprising or consisting of the amino acid sequence of: SEQ ID NO:106.

In one embodiment, provided is a chimeric receptor comprising an amino acid sequence that is at least about 95%, 96%, 97%, 98%, 99% or 100% identical to the amino acid sequence of: SEQ ID NO:125. In one embodiment, provided is a chimeric receptor comprising or consisting of the amino acid sequence of: SEQ ID NO: 125.

In one embodiment, provided is a chimeric receptor comprising an amino acid sequence that is at least about 95%, 96%, 97%, 98%, 99% or 100% identical to the amino acid sequence of: SEQ ID NO:126. In one embodiment, provided is a chimeric receptor comprising or consisting of the amino acid sequence of: SEQ ID NO: 126.

### Transduced Cells Capable of Expressing Chimeric Receptors of the Invention

A further aspect of the present invention are transduced T cells capable of expressing a chimeric receptor of the present invention. The chimeric receptors as described herein relate to molecules which are naturally not comprised in and/or on the surface of T cells and which are not (endogenously) expressed in or on normal (non-transduced) T cells. Thus, the chimeric receptor of the invention in and/or on T cells is artificially introduced into T cells. In the context of the present invention said T cells, preferably CD8+ T cells, may be isolated/obtained from a subject to be treated as defined herein. In the context of the present invention, the chimeric receptor is presented in and/or on the surface of said T cells after (retroviral, lentiviral or non-viral) transduction as described herein below. Accordingly, after transduction. T cells according to the invention can be activated by immunoglobulins, preferably (therapeutic) antibodies comprising at least one antigen binding moiety capable of binding to a target cell antigen and at least one antigen binding moiety capable of binding to a mutated Fc domain but not capable of binding to the respective non-mutated parent Fc domain.

The invention also relates to transduced T cells expressing a chimeric receptor encoded by (a) nucleic acid molecule(s) encoding the chimeric receptor of the present invention. Accordingly, in the context of the present invention, the transduced cell may comprise a nucleic acid molecule encoding the chimeric receptor of the present invention or a vector of the present invention which expresses a chimeric receptor of the present invention.

In the context of the present invention, the term “transduced T cell” relates to a genetically modified T cell (i.e. a T cell wherein a nucleic acid molecule has been introduced deliberately). The herein provided transduced T cell may comprise the vector of the present invention.

Preferably, the herein provided transduced T cell comprises the nucleic acid molecule encoding the chimeric receptor of the present invention and/or the vector of the present invention. The transduced T cell of the invention may be a T cell which transiently or stably expresses the foreign DNA (i.e. the nucleic acid molecule which has been introduced into the T cell). In particular, the nucleic acid molecule encoding the chimeric receptor of the present invention can be stably integrated into the genome of the T cell by using a retroviral or lentiviral transduction. By using mRNA transfection, the nucleic acid molecule encoding the chimeric receptor of the present invention may be expressed transiently. Preferably, the herein provided transduced T cell has been genetically modified by introducing a nucleic acid molecule in the T cell via a viral vector (e.g. a retroviral vector or a lentiviral vector). Accordingly, the expression of the chimeric receptors may be constitutive and the extracellular domain (and the mutated Fc domain) chimeric receptor may be detectable on the cell surface.

The expression may also be conditional or inducible in the case that the chimeric receptor is introduced into T cells under the control of an inducible or repressible promoter. Examples for such inducible or repressible promoters can be a transcriptional system containing the alcohol dehydrogenase I (alcA) gene promoter and the transactivator protein AlcR. Different agricultural alcohol-based formulations are used to control the expression of a gene of interest linked to the alcA promoter. Furthermore, tetracycline-responsive promoter systems can function either to activate or repress gene expression system in the presence of tetracycline.

Some of the elements of the systems include a tetracycline repressor protein (TetR), a tetracycline operator sequence (tetO) and a tetracycline transactivator fusion protein (tTA), which is the fusion of TetR and a herpes simplex virus protein 16 (VP16) activation sequence. Further, steroid-responsive promoters, metal-regulated or pathogenesis-related (PR) protein related promoters can be used.

The expression can be constitutive or constitutional, depending on the system used. The chimeric receptors of the present invention can be expressed on the surface of the herein provided transduced T cell. The extracellular portion of the chimeric receptor (i.e. the extracellular domain of the chimeric receptor can be detected on the cell surface, while the intracellular portion (i.e. the co-stimulatory signaling domain(s) and the stimulatory signaling domain) are not detectable on the cell surface. The detection of the extracellular domain of the chimeric receptor can be carried out by using an antibody which specifically binds to this extracellular domain and/or the mutated Fc domain, e.g. by flow cytometry or microscopy.

Other cells can also be transduced with the chimeric receptors of the invention and thereby be directed against target cells. These further cells include but are not limited to B-cells. Natural Killer (NK) cells, innate lymphoid cells, macrophages, monocytes, dendritic cells, or neutrophils. Preferentially, said immune cell would be a lymphocyte. Triggering of the chimeric receptor of the present invention on the surface of the leukocyte will render the cell cytotoxic against a target cell in conjunction with a therapeutic antibody as herein described irrespective of the lineage the cell originated from. Cytotoxicity will happen irrespective of the stimulatory signaling domain or co-stimulatory signaling domain chosen for the chimeric receptor and is not dependent on the exogenous supply of additional cytokines. Accordingly, the transduced cell of the present invention may be, e.g., a CD4+ T cell, a CD8+-T cell, a γδ T cell, a Natural Killer (NK) T cell, a Natural Killer (NK) cell, a tumor-infiltrating lymphocyte (TIL) cell, a myeloid cell, or a mesenchymal stem cell. Preferably, the herein provided transduced cell is a T cell (e.g. an autologous T cell), more preferably, the transduced cell is a CD8+ T cell.

Accordingly, in the context of the present invention, the transduced cell is a CD8+ T cell. Further, in the context of the present invention, the transduced cell is an autologous T cell.

Accordingly, in the context of the present invention, the transduced cell is preferably an autologous CD8+ T cell. In addition to the use of autologous cells (e.g. T cells) isolated from the subject, the present invention also comprehends the use of allogeneic cells. Accordingly, in the context of the present invention the transduced cell may also be an allogeneic cell, such as an allogeneic CD8+ T cell. The term allogeneic refers to cells coming from an unrelated donor individual/subject which is human leukocyte antigen (HLA) compatible to the individual/subject which will be treated by e.g. the herein described chimeric receptor expressing transduced cell. Autologous cells refer to cells which are isolated/obtained as described herein above from the subject to be treated with the transduced cell described herein.

The transduced cell of the invention may be co-transduced with further nucleic acid molecules. e.g. with a nucleic acid molecule encoding a cytokine.

The present invention also relates to a method for the production of a transduced T cell expressing a chimeric receptor of the invention, comprising the steps of transducing a T cell with a vector of the present invention, culturing the transduced T cell under conditions allowing the expressing of the chimeric receptor in or on said transduced cell and recovering said transduced T cell.

In the context of the present invention, the transduced cell of the present invention is preferably produced by isolating cells (e.g., T cells, preferably CD8+ T cells) from a subject (preferably a human patient). Methods for isolating/obtaining cells (e.g. T cells, preferably CD8+ T cells) from patients or from donors are well known in the art and in the context of the present cells (e.g. T cells, preferably CD8+ T cells) from patients or from donors, e.g. cells may be isolated by blood draw or removal of bone marrow. After isolating/obtaining cells as a sample of the patient, the cells (e.g. T cells) are separated from the other ingredients of the sample. Several methods for separating cells (e.g. T cells) from the sample are known and include, without being limiting, e.g. leukapheresis for obtaining cells from the peripheral blood sample from a patient or from a donor, isolating/obtaining cells by using a FACS cell sorting apparatus. The isolated/obtained cells T cells, are subsequently cultivated and expanded, e.g., by using an anti-CD3 antibody, by using anti-CD3 and anti-CD28 monoclonal antibodies and/or by using an anti-CD3 antibody, an anti-CD28 antibody and interleukin-2 (IL-2) (see, e.g., Dudley, Immunother. 26 (2003), 332-342 or Dudley, Clin. Oncol. 26 (2008), 5233-5239).

In a subsequent step the cells (e.g. T cells) are artificially/genetically modified/transduced by methods known in the art (see, e.g., Lemoine, J Gene Med 6 (2004), 374-386). Methods for transducing cells (e.g. T cells) are known in the art and include, without being limited, in a case where nucleic acid or a recombinant nucleic acid is transduced, for example, an electroporation method, calcium phosphate method, cationic lipid method or liposome method. The nucleic acid to be transduced can be conventionally and highly efficiently transduced by using a commercially available transfection reagent, for example. Lipofectamine (manufactured by Invitrogen, catalogue no.: 11668027). In a case where a vector is used, the vector can be transduced in the same manner as the above-mentioned nucleic acid as long as the vector is a plasmid vector (i.e. a vector which is not a viral vector In the context of the present invention, the methods for transducing cells (e.g. T cells) include retroviral or lentiviral T cell transduction, non-viral vectors (e.g., sleeping beauty minicircle vector) as well as mRNA transfection. “mRNA transfection” refers to a method well known to those skilled in the art to transiently express a protein of interest, like in the present case the chimeric receptor of the present invention, in a cell to be transduced. In brief cells may be electroporated with the mRNA coding for the chimeric receptor of the present by using an electroporation system (such as e.g. Gene Pulser, Bio-Rad) and thereafter cultured by standard cell (e.g. T cell) culture protocol as described above (see Zhao et al., Mol Ther. 13 (1) (2006), 151-159.) The transduced cell of the invention can be generated by lentiviral, or most preferably retroviral transduction.

In this context, suitable retroviral vectors for transducing cells are known in the art such as SAMEN CMV/SRa (Clay et al., J. Immunol. 163 (1999), 507-513), LZRS-id3-IHRES (Heemskerk et al., J. Exp. Med. 186 (1997), 1597-1602), FeLV (Neil et al., Nature 308 (1984), 814-820), SAX (Kantoff et al., Proc. Natl. Acad. Sci. USA 83 (1986), 6563-6567), pDOL (Desiderio, J. Exp. Med. 167 (1988), 372-388), N2 (Kasid et al., Proc. Natl. Acad. Sci. USA 87 (1990), 473-477), LNL6 (Tiberghien et al., Blood 84 (1994), 1333-1341), pZipNEO (Chen et al., J. Immunol. 153 (1994), 3630-3638), LASN (Mullen et al., Hum. Gene Ther. 7 (1996), 1123-1129), pGIXsNa (Taylor et al., J. Exp. Med. 184 (1996), 2031-2036), LCNX (Sun et al., Hum. Gene Ther. 8 (1997), 1041-1048), SFG (Gallardo et al., Blood 90 (1997), and LXSN (Sun et al., Hum. Gene Ther. 8 (1997), 1041-1048), SFG (Gallardo et al., Blood 90 (1997), 952-957), HMB-Hb-Hu (Vieillard et al., Proc. Natl. Acad. Sci. USA 94 (1997), 11595-11600), pMV7 (Cochlovius et al., Cancer Immunol. Immunother. 46 (1998), 61-66), pSTITCH (Weitjens et al., Gene Ther 5 (1998), 1195-1203), pLZR (Yang et al., Hum. Gene Ther. 10 (1999), 123-132), pBAG (Wu et al., Hum. Gene Ther. 10 (1999), 977-982), rKat.43.267bn (Gilham et al., J. Immunother. 25 (2002), 139-151), pLGSN (Engels et al., Hum. Gene Ther. 14 (2003), 1155-1168), pMP71 (Engels et al., Hum. Gene Ther. 14 (2003), 1155-1168), pGCSAM (Morgan et al., J. Immunol. 171 (2003), 3287-3295), pMSGV (Zhao et al., J. Immunol. 174 (2005), 4415-4423), or pMX (de Witte et al., J. Immunol. 181 (2008), 5128-5136). In the context of the present invention, suitable lentiviral vector for transducing cells (e.g. T cells) are, e.g. PL-SIN lentiviral vector (Hotta et al., Nat Methods. 6 (5) (2009). 370-376), p156RRL-sinPPT-CMV-GFP-PRE/NheI (Campeau et al., PLOS One 4 (8) (2009), e6529), pCMVR8.74 (Addgene Catalogoue No.: 22036), FUGW (Lois et al., Science 295 (5556) (2002), 868-872, pLVX-EF1 (Addgene Catalogue No.: 64368), pLVE (Brunger et al., Proc Natl Acad Sci USA 111 (9) (2014), E798-806), pCDH1-MCS1-EF1 (Hu et al., Mol Cancer Res. 7 (11) (2009), 1756-1770), pSLIK (Wang et al., Nat Cell Biol. 16 (4) (2014), 345-356), pLJMI (Solomon et al., Nat Genet. 45 (12) (2013), 1428-30), pLX302 (Kang et al., Sci Signal. 6 (287) (2013), rs13), pHR-IG (Xie et al., J Cereb Blood Flow Metab. 33 (12) (2013), 1875-85), pRRLSIN (Addgene Catalogoue No.: 62053), pLS (Miyoshi et al., J Virol. 72 (10) (1998), 8150-8157), pLL3.7 (Lazebnik et al., J Biol Chem. 283 (7) (2008), 11078-82), FRIG (Raissi et al., Mol Cell Neurosci. 57 (2013), 23-32), pWPT (Ritz-Laser et al., Diabetologia. 46 (6) (2003), 810-821), pBOB (Marr et al., J Mol Neurosci. 22 (1-2) (2004), 5-11), or pLEX (Addgene Catalogue No.: 27976).

The transduced cells of the present invention is/are preferably grown under controlled conditions, outside of their natural environment. In particular, the term “culturing” means that cells (e.g. the transduced cell(s) of the invention) which are derived from multi-cellular eukaryotes (preferably from a human patient) are grown in vitro. Culturing cells is a laboratory technique of keeping cells alive which are separated from their original tissue source. Herein, the transduced cell of the present invention is cultured under conditions allowing the expression of the chimeric receptor of the present invention in or on said transduced cells. Conditions which allow the expression or a transgene (i.e. of the chimeric receptor of the present invention) are commonly known in the art and include, e.g., agonistic anti-CD3- and anti-CD28 antibodies and the addition of cytokines such as interleukin 2 (IL-2), interleukin 7 (IL-7), interleukin 12 (IL-12) and/or interleukin 15 (IL-15). After expression of the chimeric receptor of the present invention in the cultured transduced cell (e.g., a CD8+ T), the transduced cell is recovered (i.e. re-extracted) from the culture (i.e. from the culture medium).

Accordingly, also encompassed by the invention is a transduced cell, preferably a T cell, in particular a CD8+ T expressing a chimeric receptor encoded by a nucleic acid molecule of the invention obtainable by the method of the present invention.

### Nucleic Acid Molecules

A further aspect of the present invention are nucleic acids and vectors encoding one or several chimeric receptors of the present invention. An exemplary nucleic acid molecules encoding the chimeric receptors of the present invention is shown in SEQ ID NO:96 or SEQ ID NO: 107. The nucleic acid molecules of the invention may be under the control of regulatory sequences. For example, promoters, transcriptional enhancers and/or sequences which allow for induced expression of the chimeric receptor of the invention may be employed. In the context of the present invention, the nucleic acid molecules are expressed under the control of constitutive or inducible promoter. Suitable promoters are e.g. the CMV promoter (Qin et al., PLOS One 5 (5) (2010), e10611), the UBC promoter (Qin et al., PLOS One 5 (5) (2010), e10611). PGK (Qin et al., PLOS One 5 (5) (2010), e10611), the EF1A promoter (Qin et al., PLOS One 5 (5) (2010), e10611), the CAGG promoter (Qin et al., PLOS One 5 (5) (2010), e10611), the SV40 promoter (Qin et al., PLOS One 5 (5) (2010), e10611), the COP1A promoter (Qin et al., PLOS One 5 (5) (2010), e10611), the ACT5C promoter (Qin et al., PLOS One 5 (5) (2010), e10611), the TRE promoter (Qin et al., PLOS One. 5 (5) (2010), e10611), the Oct3/4 promoter (Chang et al., Molecular Therapy 9 (2004). S367-S367 (doi: 10.1016/j.ymthe.2004.06.904)), or the Nanog promoter (Wu et al., Cell Res. 15 (5) (2005), 317-24). The present invention therefore also relates to (a) vector(s) comprising the nucleic acid molecule(s) described in the present invention. Herein the term vector relates to a circular or linear nucleic acid molecule which can autonomously replicate in a cell into which it has been introduced. Many suitable vectors are known to those skilled in molecular biology, the choice of which would depend on the function desired and include plasmids, cosmids, viruses, bacteriophages and other vectors used conventionally in genetic engineering. Methods which are well known to those skilled in the art can be used to construct various plasmids and vectors: see, for example, the techniques described in Sambrook et al. (loc cit.) and Ausubel, Current Protocols in Molecular Biology. Green Publishing Associates and Wiley Interscience. N.Y. (1989), (1994). Alternatively, the polynucleotides and vectors of the invention can be reconstituted into liposomes for delivery to target cells. As discussed in further details below, a cloning vector was used to isolate individual sequences of DNA. Relevant sequences can be transferred into expression vectors where expression of a particular polypeptide is required. Typical cloning vectors include pBluescript SK, pGEM, pUC9, pBR322, pGA18 and pGBT9. Typical expression vectors include pTRE, pCAL-n-EK, pESP-1, pOP13CAT.

The invention also relates to (a) vector(s) comprising (a) nucleic acid molecule(s) which is (are) a regulatory sequence operably linked to said nucleic acid molecule(s) encoding a chimeric receptor as defined herein. In the context of the present invention the vector can be polycistronic. Such regulatory sequences (control elements) are known to the skilled person and may include a promoter, a splice cassette, translation initiation codon, translation and insertion site for introducing an insert into the vector(s). In the context of the present invention, said nucleic acid molecule(s) is (are) operatively linked to said expression control sequences allowing expression in eukaryotic or prokaryotic cells. It is envisaged that said vector(s) is (are) (an) expression vector(s) comprising the nucleic acid molecule(s) encoding the chimeric receptor as defined herein. Operably linked refers to a juxtaposition wherein the components so described are in a relationship permitting them to function in their intended manner. A control sequence operably linked to a coding sequence is ligated in such a way that expression of the coding sequence is achieved under conditions compatible with the control sequences. In case the control sequence is a promoter, it is obvious for a skilled person that double-stranded nucleic acid is preferably used.

In the context of the present invention the recited vector(s) is (are) an expression vector(s). An expression vector is a construct that can be used to transform a selected cell and provides for expression of a coding sequence in the selected cell. An expression vector(s) can for instance be cloning (a) vector(s). (a) binary vector(s) or (a) integrating vector(s). Expression comprises transcription of the nucleic acid molecule preferably into a translatable mRNA. Regulatory elements ensuring expression in prokaryotes and/or eukaryotic cells are well known to those skilled in the art. In the case of eukaryotic cells they comprise normally promoters ensuring initiation of transcription and optionally poly-A signals ensuring termination of transcription and stabilization of the transcript. Possible regulatory elements permitting expression in prokaryotic host cells comprise, e.g., the PL, lac, trp or tac promoter in E. coli, and examples of regulatory elements permitting expression in eukaryotic host cells are the AOX1 or GAL1 promoter in yeast or the CMV-·SV40, RSV-promoter (Rous sarcoma virus), CMV-enhancer, SV40-enhancer or a globin intron in mammalian and other animal cells.

Beside elements which are responsible for the initiation of transcription such regulatory elements may also comprise transcription termination signals, such as the SV40-poly-A site or the tk-poly-A site, downstream of the polynucleotide. Furthermore, depending on the expression system used leader sequences encoding signal peptides capable of directing the polypeptide to a cellular compartment or secreting it into the medium may be added to the coding sequence of the recited nucleic acid sequence and are well known in the art: see also, e.g., appended Examples.

The leader sequence(s) is (are) assembled in appropriate phase with translation, initiation and termination sequences, and preferably, a leader sequence capable of directing secretion of translated protein, or a portion thereof, into the periplasmic space or extracellular medium.

Optionally, the heterologous sequence can encode a chimeric receptor including an N-terminal identification peptide imparting desired characteristics, e.g., stabilization or simplified purification of expressed recombinant product: see supra. In this context, suitable expression vectors are known in the art such as Okayama-Berg cDNA expression vector pcDV1 (Pharmacia), pCDM8, pRc/CMV, pcDNA1, pcDNA3 (In-vitrogene), pEF-DHFR, pEF-ADA or pEF-neo (Raum et al. Cancer Immunol Immunother 50 (2001). 141-150) or pSPORT1 (GIBCO BRL).

In the context of the present invention, the expression control sequences will be eukaryotic promoter systems in vectors capable of transforming or transfecting eukaryotic cells, but control sequences for prokaryotic cells may also be used. Once the vector has been incorporated into the appropriate cell, the cell is maintained under conditions suitable for high level expression of the nucleotide sequences, and as desired. Additional regulatory elements may include transcriptional as well as translational enhancers. Advantageously, the above-described vectors of the invention comprise a selectable and/or scorable marker. Selectable marker genes useful for the selection of transformed cells and, e.g., plant tissue and plants are well known to those skilled in the art and comprise, for example, antimetabolite resistance as the basis of selection for dhfr, which confers resistance to methotrexate (Reiss. Plant Physiol. (Life Sci. Adv.) 13 (1994). 143-149), npt, which confers resistance to the aminoglycosides neomycin, kanamycin and paromycin (Herrera-Estrella, EMBO J. 2 (1983), 987-995) and hygro, which confers resistance to hygromycin (Marsh, Gene 32 (1984), 481-485). Additional selectable genes have been described, namely trpB, which allows cells to utilize indole in place of tryptophan: hisD, which allows cells to utilize histinol in place of histidine (Hartman, Proc. Natl. Acad. Sci. USA 85 (1988), 8047); mannose-6-phosphate isomerase which allows cells to utilize mannose (WO 94/20627) and ODC (ornithine decarboxylase) which confers resistance to the ornithine decarboxylase inhibitor, 2-(difluoromethyl)-DL-ornithine, DFMO (McConlogue, 1987, In: Current Communications in Molecular Biology, Cold Spring Harbor Laboratory ed.) or deaminase from Aspergillus terreus which confers resistance to Blasticidin S (Tamura, Biosci, Biotechnol. Biochem. 59 (1995), 2336-2338).

Useful scorable markers are also known to those skilled in the art and are commercially available. Advantageously, said marker is a gene encoding luciferase (Giacomin, Pl. Sci. 116 (1996), 59-72; Scikantha, J. Bact. 178 (1996), 121), green fluorescent protein (Gerdes, FEBS Lett. 389 (1996), 44-47) or β-glucuronidase (Jefferson, EMBO J. 6 (1987), 3901-3907). This embodiment is particularly useful for simple and rapid screening of cells, tissues and organisms containing a recited vector.

As described above, the recited nucleic acid molecule(s) can be used alone or as part of (a) vector(s) to express the chimeric receptors of the invention in cells, for, e.g., adoptive T cell therapy but also for gene therapy purposes. The nucleic acid molecules or vector(s) containing the DNA sequence(s) encoding any one of the herein described chimeric receptors is introduced into the cells which in turn produce the polypeptide of interest. Gene therapy, which is based on introducing therapeutic genes into cells by ex-vivo or in-vivo techniques is one of the most important applications of gene transfer. Suitable vectors, methods or gene-delivery systems for in methods or gene-delivery systems for in-vitro or in-vivo gene therapy are described in the literature and are known to the person skilled in the art: see, e.g., Giordano, Nature Medicine 2 (1996), 534-539; Schaper, Circ. Res. 79 (1996), 911-919; Anderson, Science 256 (1992), 808-813; Verma, Nature 389 (1994), 239; Isner, Lancet 348 (1996), 370-374; Muhlhauser, Circ. Res. 77 (1995), 1077-1086; Onodera, Blood 91 (1998), 30-36; Verma, Gene Ther. 5 (1998), 692-699; Nabel, Ann. N.Y. Acad. Sci. 811 (1997), 289-292; Verzeletti, Hum. Gene Ther. 9 (1998), 2243-51; Wang, Nature Medicine 2 (1996), 714-716: WO 94/29469; WO 97/00957; U.S. Pat. Nos. 5,580,859; 5,589,466; or Schaper, Current Opinion in Biotechnology 7 (1996), 635-640).

The recited nucleic acid molecule(s) and vector(s) may be designed for direct introduction or for introduction via liposomes, or viral vectors (e.g., adenoviral, retroviral) into the cell. In the context of the present invention, said cell is a T cells, such as CD8+ T cells, CD4+ T cells, CD3+ T cells, γδ T cells or natural killer (NK) T cells, preferably CD8+ T cells.

In accordance with the above, the present invention relates to methods to derive vectors, particularly plasmids, cosmids and bacteriophages used conventionally in genetic engineering that comprise a nucleic acid molecule encoding the polypeptide sequence of a chimeric receptor defined herein. In the context of the present invention, said vector is an expression vector and/or a gene transfer or targeting vector. Expression vectors derived from viruses such as retroviruses, vaccinia virus, adeno-associated virus, herpes virus, or bovine papilloma virus, may be used for delivery of the recited polynucleotides or vector into targeted cell populations.

Methods which are well known to those skilled in the art can be used to construct (a) recombinant vector(s); see, for example, the techniques described in Sambrook et al. (loc cit.), Ausubel (1989, loc cit.) or other standard text books. Alternatively, the recited nucleic acid molecules and vectors can be reconstituted into liposomes for delivery to target cells. The vectors containing the nucleic acid molecules of the invention can be transferred into the host cell by well-known methods, which vary depending on the type of cellular host. For example, calcium chloride transfection is commonly utilized for prokaryotic cells, whereas calcium phosphate treatment or electroporation may be used for other cellular hosts: see Sambrook, supra. The recited vector may, inter alia, be the pEF-DHFR, pEF-ADA or pEF-neo. The vectors pEF-DHFR, pEF-ADA and pEF-neo have been described in the art, e.g. in Mack et al. Proc. Natl. Acad. Sci. USA 92 (1995). 7021-7025 and Raum et al. Cancer Immunol Immunother 50) (2001), 141-150.

The invention also provides for a T cell transduced with a vector as described herein. Said T cell may be produced by introducing at least one of the above described vector or at least one of the above described nucleic acid molecules into the T cell or its precursor cell. The presence of said at least one vector or at least one nucleic acid molecule in the T cell mediates the expression of a gene encoding the above described chimeric receptor. The vector of the present invention can be polycistronic.

The described nucleic acid molecule(s) or vector(s) which is (are) introduced in the T cell or its precursor cell may either integrate into the genome of the cell or it may be maintained extrachromosomally.

### Antibodies Capable of Binding to the Chimeric Receptor of the Invention

The chimeric receptors of the present invention are useful for example in methods for treating or delaying progression of various diseases (e.g. cancer), in particular in combination with antibodies capable of binding to the chimeric receptors of the present invention.

In one embodiment, provided is a method of treating a disease in a subject, comprising administering to the subject a transduced T cell capable of expressing the chimeric receptor as herein described, wherein the chimeric receptor comprises an extracellular domain comprising a mutated Fc domain or a fragment thereof, and administering before, simultaneously with or after administration of the transduced T cell a therapeutically effective amount of a molecule (e.g. an antibody) that comprises at least one antigen binding moiety capable of binding to the mutated Fc domain but not capable of binding to the non-mutated parent Fc domain. In one embodiment, the antibody comprises at least one effector moiety.

In one embodiment, the effector moiety is an antigen binding moiety capable of binding to a target cell antigen.

In one embodiment, the effector moiety is an immune activating moiety. In one embodiment, the immune activating moiety is a cytokine (e.g. IL2). In a particular embodiment, the cytokine is IL2 or a derivative thereof. In one embodiment, the immune activating moiety is a costimulatory T cell antigen (e.g. CD28, 4-1BB) or a costimulatory ligand (e.g. 4-1BBL).

Antigen binding moieties capable of binding to a mutated Fc domain, target antigens, and immune activating moieties are further described herein below:

### Antigen Binding Moieties Capable of Binding to a Mutated Fc Domain

The invention further provides antibodies that comprise at least one antigen binding moiety capable of binding to a mutated Fc domain (e.g. comprising the P329G substitution according to EU numbering). Antigen binding moieties capable of binding to a (modified/engineered) Fc domain or fragment thereof may be generated for example by immunization of e.g. a mammalian immune system with the Fc domain or fragment thereof comprising the relevant mutation. Such methods are known in the art and e.g. are described in Burns in Methods in Molecular Biology 295:1-12 (2005). Alternatively, antigen binding moieties of the invention may be isolated by screening combinatorial libraries for antigen binding moieties with the desired activity or activities. Methods for screening combinatorial libraries are reviewed, e.g., in Lerner et al. in Nature Reviews 16:498-508 (2016). For example, a variety of methods are known in the art for generating phage display libraries and screening such libraries for antigen binding moieties possessing the desired binding characteristics. Such methods are reviewed. e.g., in Frenzel et al. in mAbs 8:1177-1194 (2016); Bazan et al. in Human Vaccines and Immunotherapeutics 8:1817-1828 (2012) and Zhao et al. in Critical Reviews in Biotechnology 36:276-289 (2016) as well as in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352:624-628 (1991); Marks et al., J. Mol. Biol. 222:581-597 (1992) and in Marks and Bradbury in Methods in Molecular Biology 248:161-175 (Lo, ed., Human Press, Totowa, NJ, 2003); Sidhu et al., J. Mol. Biol. 338 (2); 299-310 (2004); Lee et al., J. Mol. Biol. 340 (5); 1073-1093 (2004); Fellouse, Proc. Natl. Acad. Sci. USA 101 (34); 12467-12472 (2004); and Lee et al., J. Immunol. Methods 284 (1-2); 119-132 (2004). In certain phage display methods, repertoires of VH and VL genes are separately cloned by polymerase chain reaction (PCR) and recombined randomly in phage libraries, which can then be screened for antigen-binding phage as described in Winter et al. in Annual Review of Immunology 12:433-455 (1994). Phage typically display antibody fragments, either as single-chain Fv (scFv) fragments or as Fab fragments. Libraries from immunized sources provide high-affinity antigen binding moieties without the requirement of constructing hybridomas. Alternatively, the naive repertoire can be cloned (e.g., from human) to provide a single source of antigen binding moieties to a wide range antigens without any immunization as described by Griffiths et al. in EMBO Journal 12:725-734 (1993).

Furthermore, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter in Journal of Molecular Biology 227:381-388 (1992). Patent publications describing human antibody phage libraries include, for example: U.S. Pat. Nos. 5,750,373; 7,985,840; 7,785,903 and 8,679,490 as well as US Patent Publication Nos. 2005/0079574, 2007/0117126, 2007/0237764 and 2007/0292936, and 2009/0002360. Further examples of methods known in the art for screening combinatorial libraries for antigen binding moieties with a desired activity or activities include ribosome and mRNA display, as well as methods for antibody display and selection on bacteria, mammalian cells, insect cells or yeast cells. Methods for yeast surface display are reviewed, e.g., in Scholler et al. in Methods in Molecular Biology 503:135-56 (2012) and in Cherf et al. in Methods in Molecular biology 1319:155-175 (2015) as well as in the Zhao et al. in Methods in Molecular Biology 889:73-84 (2012). Methods for ribosome display are described, e.g., in He et al. in Nucleic Acids Research 25:5132-5134 (1997) and in Hanes et al. in PNAS 94:4937-4942 (1997).

In an illustrative embodiment of the present invention, as a proof of concept, provided are antigen binding moieties capable of binding to a mutated Fc domain comprising the amino acid substitution P329G.

More generally, provided are antibodies capable of binding to a mutated Fc domain comprising a substitution at position P329 (numbering according to Kabat EU index). In one embodiment, the at least one amino acid substitution comprises a substitution at position P329 (numbering according to Kabat EU index) by an amino acid selected from the list consisting of alanine (A) arginine (R), leucine (L), isoleucine (I), and proline (P). In one embodiment, the at least one amino acid substitution comprises the amino acid substitution P329G (numbering according to Kabat EU index).

In one embodiment the antigen binding moiety is capable of binding to a mutated Fc domain. In one embodiment the Fc domain is an IgG, specifically an IgG₁ or IgG₄, Fc domain. In one embodiment the Fc domain is a human Fc domain. In one embodiment the mutated Fc domain exhibits reduced binding affinity to an Fc receptor and/or reduced effector function, as compared to a native IgG₁ Fc domain. In one embodiment the Fc domain comprises one or more amino acid mutations that reduce binding to an Fc receptor and/or effector function.

In a preferred embodiment, the mutated Fc domain comprises the P329G substitution.

In one embodiment, provided is an antibody that at least one antigen binding moiety capable of binding to a mutated Fc domain comprising the P329G substitution (EU numbering) but not capable of binding to the non-mutated parent Fc domain.

In one embodiment, the antigen binding moiety comprises a heavy chain variable domain (VH) comprising at least one of:
(a) a heavy chain complementarity determining region (CDR H) 1 amino acid sequence of SEQ ID NO:1;
(b) a CDR H2 amino acid sequence selected from the group consisting of SEQ ID NO: 2, SEQ ID NO:9, SEQ ID NO: 12, or SEQ ID NO: 15; and
(c) a CDR H3 amino acid sequence of SEQ ID NO:3.

In one embodiment, the antigen binding moiety comprises a light chain variable domain (VL) comprising at least one of:
(d) a light chain (CDR L) 1 amino acid sequence of SEQ ID NO:4 or SEQ ID NO: 18;
(e) a CDR L2 amino acid sequence of SEQ ID NO:5; and
(f) a CDR L3 amino acid sequence of SEQ ID NO:6.

In a preferred embodiment, the antigen binding moiety comprises a heavy chain variable domain (VH) comprising:
(a) a heavy chain complementarity determining region (CDR H) 1 amino acid sequence of SEQ ID NO:1;
(b) a CDR H2 amino acid sequence selected from the group consisting of SEQ ID NO: 2, SEQ ID NO:9, SEQ ID NO: 12, or SEQ ID NO: 15; and
(c) a CDR H3 amino acid sequence of SEQ ID NO:3;
   and a light chain variable domain (VL) comprising:
(d) a light chain (CDR L) 1 amino acid sequence of SEQ ID NO:4 or SEQ ID NO: 18;
(e) a CDR L2 amino acid sequence of SEQ ID NO:5; and
(f) a CDR L3 amino acid sequence of SEQ ID NO:6.

In one particular embodiment, the antigen binding moiety comprises a heavy chain variable domain (VH) comprising:
(a) a heavy chain complementarity determining region (CDR H) 1 amino acid sequence of SEQ ID NO:1;
(b) a CDR H2 amino acid sequence of SEQ ID NO:2;
(c) a CDR H3 amino acid sequence of SEQ ID NO:3;
   and a light chain variable domain (VL) comprising:
(d) a light chain (CDR L) 1 amino acid sequence of SEQ ID NO:4;
(e) a CDR L2 amino acid sequence of SEQ ID NO:5; and
(f) a CDR L3 amino acid sequence of SEQ ID NO:6.

In another particular embodiment, the antigen binding moiety comprises a heavy chain variable domain (VH) comprising:
(a) a heavy chain complementarity determining region (CDR H) 1 amino acid sequence of SEQ ID NO:1;
(b) a CDR H2 amino acid sequence of SEQ ID NO:9;
(c) a CDR H3 amino acid sequence of SEQ ID NO:3;
   and a light chain variable domain (VL) comprising:
(d) a light chain (CDR L) 1 amino acid sequence of SEQ ID NO:4;
(e) a CDR L2 amino acid sequence of SEQ ID NO:5; and
(f) a CDR L3 amino acid sequence of SEQ ID NO:6.

In another particular embodiment, the antigen binding moiety comprises a heavy chain variable domain (VH) comprising:
(a) a heavy chain complementarity determining region (CDR H) 1 amino acid sequence of SEQ ID NO:1;
(b) a CDR H2 amino acid sequence of SEQ ID NO:12;
(c) a CDR H3 amino acid sequence of SEQ ID NO:3;
   and a light chain variable domain (VL) comprising:
(d) a light chain (CDR L) 1 amino acid sequence of SEQ ID NO:4;
(e) a CDR L2 amino acid sequence of SEQ ID NO:5; and
(f) a CDR L3 amino acid sequence of SEQ ID NO:6.

In another particular embodiment, the antigen binding moiety comprises a heavy chain variable domain (VH) comprising:
(g) a heavy chain complementarity determining region (CDR H) 1 amino acid sequence of SEQ ID NO:1;
(h) a CDR H2 amino acid sequence of SEQ ID NO:15;
(i) a CDR H3 amino acid sequence of SEQ ID NO:3;
   and a light chain variable domain (VL) comprising:
(j) a light chain (CDR L) 1 amino acid sequence of SEQ ID NO:4;
(k) a CDR L2 amino acid sequence of SEQ ID NO:5; and
(l) a CDR L3 amino acid sequence of SEQ ID NO:6.

In another particular embodiment, the antigen binding moiety comprises a heavy chain variable domain (VH) comprising:
(a) a heavy chain complementarity determining region (CDR H) 1 amino acid sequence of SEQ ID NO: 1;
(b) a CDR H2 amino acid sequence of SEQ ID NO:9;
(c) a CDR H3 amino acid sequence of SEQ ID NO:3;
   and a light chain variable domain (VL) comprising:
(d) a light chain (CDR L) 1 amino acid sequence of SEQ ID NO:18;
(e) a CDR L2 amino acid sequence of SEQ ID NO:5; and
(f) a CDR L3 amino acid sequence of SEQ ID NO:6.

In one embodiment the antigen binding moiety comprises a heavy chain variable domain (VH) comprising an amino acid sequence that is at least about 95%, 96%, 97%, 98%, 99% or 100% identical to an amino acid sequence selected from the group consisting of SEQ ID NO:7, SEQ ID NO: 10, SEQ ID NO: 13, SEQ ID NO: 14 and SEQ ID NO:16.

In one embodiment the antigen binding moiety comprises a heavy chain variable domain (VH) comprising an amino acid sequence that is at least about 95%, 96%, 97%, 98%, 99% or 100% identical to the amino acid sequence of SEQ ID NO:7.

In one embodiment the antigen binding moiety comprises a heavy chain variable domain (VH) comprising an amino acid sequence that is at least about 95%, 96%, 97%, 98%, 99% or 100% identical to the amino acid sequence of SEQ ID NO: 10.

In one embodiment the antigen binding moiety comprises a heavy chain variable domain (VH) comprising an amino acid sequence that is at least about 95%, 96%, 97%, 98%, 99% or 100% identical to the amino acid sequence of SEQ ID NO: 13.

In one embodiment the antigen binding moiety comprises a heavy chain variable domain (VH) comprising an amino acid sequence that is at least about 95%, 96%, 97%, 98%, 99% or 100% identical to the amino acid sequence of SEQ ID NO: 14.

In one embodiment the antigen binding moiety comprises a heavy chain variable domain (VH) comprising an amino acid sequence that is at least about 95%, 96%, 97%, 98%, 99% or 100% identical to the amino acid sequence of SEQ ID NO: 16.

In one embodiment the antigen binding moiety comprises a light chain variable domain (VL) comprising an amino acid sequence that is at least about 95%, 96%, 97%, 98%, 99% or 100% identical to a sequence selected from the group consisting of SEQ ID NO:8, SEQ ID NO:11, SEQ ID NO: 17 and SEQ ID NO: 19.

In one embodiment the antigen binding moiety comprises a light chain variable domain (VL) comprising an amino acid sequence that is at least about 95%, 96%, 97%, 98%, 99% or 100% identical to the amino acid sequence of SEQ ID NO:8.

In one embodiment the antigen binding moiety comprises a light chain variable domain (VL) comprising an amino acid sequence that is at least about 95%, 96%, 97%, 98%, 99% or 100% identical to the amino acid sequence of SEQ ID NO: 11.

In one embodiment the antigen binding moiety comprises a light chain variable domain (VL) comprising an amino acid sequence that is at least about 95%, 96%, 97%, 98%, 99% or 100% identical to the amino acid sequence of SEQ ID NO: 17.

In one embodiment the antigen binding moiety comprises a light chain variable domain (VL) comprising an amino acid sequence that is at least about 95%, 96%, 97%, 98%, 99% or 100% identical to the amino acid sequence of SEQ ID NO: 19.

In one embodiment, the antigen binding moiety comprises a heavy chain variable domain (VH) comprising an amino acid sequence that is at least about 95%, 96%, 97%, 98%, 99% or 100% identical to the amino acid sequence of SEQ ID NO:7 and a light chain variable domain (VL) comprising an amino acid sequence that is at least about 95%, 96%, 97%, 98%, 99% or 100% identical to the amino acid sequence of SEQ ID NO:8.

In one embodiment, the antigen binding moiety comprises a heavy chain variable domain (VH) comprising an amino acid sequence that is at least about 95%, 96%, 97%, 98%, 99% or 100% identical to the amino acid sequence of SEQ ID NO: 10 and a light chain variable domain (VL) comprising an amino acid sequence that is at least about 95%, 96%, 97%, 98%, 99% or 100% identical to the amino acid sequence of SEQ ID NO: 11.

In one embodiment, the antigen binding moiety comprises a heavy chain variable domain (VH) comprising an amino acid sequence that is at least about 95%, 96%, 97%, 98%, 99% or 100% identical to the amino acid sequence of SEQ ID NO: 13 and a light chain variable domain (VL) comprising an amino acid sequence that is at least about 95%, 96%, 97%, 98%, 99% or 100% identical to the amino acid sequence of SEQ ID NO: 11.

In one embodiment, the antigen binding moiety comprises a heavy chain variable domain (VH) comprising an amino acid sequence that is at least about 95%, 96%, 97%, 98%, 99% or 100% identical to the amino acid sequence of SEQ ID NO: 14 and a light chain variable domain (VL) comprising an amino acid sequence that is at least about 95%, 96%, 97%, 98%, 99% or 100% identical to the amino acid sequence of SEQ ID NO: 11.

In one embodiment, the antigen binding moiety comprises a heavy chain variable domain (VH) comprising an amino acid sequence that is at least about 95%, 96%, 97%, 98%, 99% or 100% identical to the amino acid sequence of SEQ ID NO: 16 and a light chain variable domain (VL) comprising an amino acid sequence that is at least about 95%, 96%, 97%, 98%, 99% or 100% identical to the amino acid sequence of SEQ ID NO: 11.

In one embodiment, the antigen binding moiety comprises a heavy chain variable domain (VH) comprising an amino acid sequence that is at least about 95%, 96%, 97%, 98%, 99% or 100% identical to the amino acid sequence of SEQ ID NO: 10 and a light chain variable domain (VL) comprising an amino acid sequence that is at least about 95%, 96%, 97%, 98%, 99% or 100% identical to the amino acid sequence of SEQ ID NO: 17.

In one embodiment, the antigen binding moiety comprises a heavy chain variable domain (VH) comprising an amino acid sequence that is at least about 95%, 96%, 97%, 98%, 99% or 100% identical to the amino acid sequence of SEQ ID NO: 10 and a light chain variable domain (VL) comprising an amino acid sequence that is at least about 95%, 96%, 97%, 98%, 99% or 100% identical to the amino acid sequence of SEQ ID NO: 19.

In a preferred embodiment the antigen binding moiety comprises a heavy chain variable domain (VH) comprising the amino acid sequence of SEQ ID NO: 14, and a light chain variable domain (VL) comprising the amino acid sequence of SEQ ID NO:11.

In one embodiment, the mutated Fc domain comprises the 1253A, H310A and H435A (“AAA”) substitutions. In one embodiment, provided is an antibody that comprises at least one antigen binding moiety capable of binding to a mutated Fc domain comprising the I253A, H310A and H435A substitutions (EU numbering) but not capable of binding to the non-mutated parent Fc domain.

In one embodiment, antigen binding moiety comprises a heavy chain variable region comprising at least one of:
(a) a heavy chain complementarity determining region (CDR H) 1 amino acid sequence of SEQ ID NO:28;
(b) a CDR H2 amino acid sequence of SEQ ID NO:29; and
(c) a CDR H3 amino acid sequence of SEQ ID NO:30).

In one embodiment, antigen binding moiety comprises at least one of:
(d) a light chain (CDR L) 1 amino acid sequence of SEQ ID NO:31;
(e) a CDR L2 amino acid sequence of SEQ ID NO:32; and
(f) a CDR L3 amino acid sequence of SEQ ID NO:33.

In one embodiment, the antigen binding moiety comprises at least one heavy chain complementarity determining region (CDR) comprising an amino acid sequence that is at least about 95%, 96%, 97%, 98%, 99% or 100% identical to an amino acid sequence selected from the group consisting of SEQ ID NO:28, SEQ ID NO:29 and SEQ ID NO:30 and at least one light chain CDR selected from the group of SEQ ID NO:31, SEQ ID NO:32 and SEQ ID NO:33.

In one embodiment, the antigen binding moiety comprises the heavy chain complementarity determining region (CDRs) of SEQ ID NO:28, SEQ ID NO:29 and SEQ ID NO:30 and the light chain CDRs of SEQ ID NO:31, SEQ ID NO:32 and SEQ ID NO:33.

In one embodiment, the antigen binding moiety comprises a heavy chain variable region comprising:
(a) a heavy chain complementarity determining region (CDR H) 1 amino acid sequence of SEQ ID NO:28;
(b) a CDR H2 amino acid sequence of SEQ ID NO:29;
(c) a CDR H3 amino acid sequence of SEQ ID NO:30; and a light chain variable region comprising:
(d) a light chain (CDR L) 1 amino acid sequence of SEQ ID NO:31;
(e) a CDR L2 amino acid sequence of SEQ ID NO:32; and
(f) a CDR L3 amino acid sequence of SEQ ID NO:33.

In one embodiment, the antigen binding moiety comprises a heavy chain variable region (VH) comprising an amino acid sequence that is at least about 95%, 96%, 97%, 98%, 99% or 100% identical to the amino acid sequence of SEQ ID NO:34 and a light chain variable region (VL) comprising an amino acid sequence that is at least about 95%, 96%, 97%, 98%, 99% or 100% identical to the amino acid sequence selected of SEQ ID NO:35.

In one embodiment, the antigen binding moiety comprises a heavy chain variable region (VH) comprising the amino acid sequence of SEQ ID NO:34, and a light chain variable region (VL) comprising the amino acid sequence of SEQ ID NO:35.

In one embodiment, antigen binding moiety comprises a heavy chain variable region comprising at least one of:
(d) a heavy chain complementarity determining region (CDR H) 1 amino acid sequence of SEQ ID NO: 130;
(e) a CDR H2 amino acid sequence of SEQ ID NO: 131; and
(f) a CDR H3 amino acid sequence of SEQ ID NO: 132).

In one embodiment, antigen binding moiety comprises at least one of:
(g) a light chain (CDR L) 1 amino acid sequence of SEQ ID NO: 133;
(h) a CDR L2 amino acid sequence of SEQ ID NO: 134; and
(i) a CDR L3 amino acid sequence of SEQ ID NO: 135.

In one embodiment, the antigen binding moiety comprises at least one heavy chain complementarity determining region (CDR) comprising an amino acid sequence that is at least about 95%, 96%, 97%, 98%, 99% or 100% identical to an amino acid sequence selected from the group consisting of SEQ ID NO: 130, SEQ ID NO: 131 and SEQ ID NO: 132 and at least one light chain CDR selected from the group of SEQ ID NO: 133, SEQ ID NO: 134 and SEQ ID NO: 135.

In one embodiment, the antigen binding moiety comprises the heavy chain complementarity determining region (CDRs) of SEQ ID NO: 130, SEQ ID NO: 131 and SEQ ID NO: 132 and the light chain CDRs of SEQ ID NO:133, SEQ ID NO:134 and SEQ ID NO:135.

In one embodiment, the antigen binding moiety comprises a heavy chain variable region comprising:
(g) a heavy chain complementarity determining region (CDR H) 1 amino acid sequence of SEQ ID NO:130;
(h) a CDR H2 amino acid sequence of SEQ ID NO:131;
(i) a CDR H3 amino acid sequence of SEQ ID NO:132; and a light chain variable region comprising;
(j) a light chain (CDR L) 1 amino acid sequence of SEQ ID NO: 133;
(k) a CDR L2 amino acid sequence of SEQ ID NO: 134; and
(l) a CDR L3 amino acid sequence of SEQ ID NO: 135.

In one embodiment, the antigen binding moiety comprises a heavy chain variable region (VH) comprising an amino acid sequence that is at least about 95%, 96%, 97%, 98%, 99% or 100% identical to the amino acid sequence of SEQ ID NO: 136 and a light chain variable region (VL) comprising an amino acid sequence that is at least about 95%, 96%, 97%, 98%, 99% or 100% identical to the amino acid sequence selected of SEQ ID NO:35.

In one embodiment, the antigen binding moiety comprises a heavy chain variable region (VH) comprising the amino acid sequence of SEQ ID NO: 136, and a light chain variable region (VL) comprising the amino acid sequence of SEQ ID NO:35.

### Target Cell Antigens

As mentioned above, the herein described (therapeutic) antibody may comprise at least one antigen binding moiety capable of binding to a target cell antigen (e.g. a tumor-specific antigen that naturally occurs on the surface of a tumor cell) in addition to the antigen binding moiety capable of binding to a mutated Fc domain but not capable of binding to the non-mutated parent Fc domain (i.e. capable of binding to the chimeric receptor of the invention). In the context of the present invention, such antibodies will bring transduced T cells as described herein comprising the chimeric receptor of the invention in physical contact with a target cell (e.g. a tumor cell), wherein the transduced T cell becomes activated. Activation of transduced T cells of the present invention preferentially results in lysis of the target cell as described herein.

Examples of target cell antigens (e.g., tumor markers) that naturally occur on the surface of target (e.g. tumor) cells are given herein below and comprise, but are not limited to FAP (fibroblast activation protein), CEA (carcinoembryonic antigen), p95 (p95HER2), BCMA (B-cell maturation antigen), EpCAM (epithelial cell adhesion molecule), MSLN (mesothelin), MCSP (melanoma chondroitin sulfate proteoglycan), HER-1 (human epidermal growth factor 1), HER-2 (human epidermal growth factor 2), HER-3 (human epidermal growth factor 3), CD19, CD20, CD22, CD33, CD38, CD52Flt3, folate receptor 1 (FOLR1), human trophoblast cell-surface antigen 2 (Trop-2) cancer antigen 12-5 (CA-12-5), human leukocyte antigen-antigen D related (HLA-DR), MUC-1 (Mucin-1), A33-antigen, PSMA (prostate-specific membrane antigen), FMS-like tyrosine kinase 3 (FLT-3), PSMA (prostate specific membrane antigen), PSCA (prostate stem cell antigen), transferrin-receptor, TNC (tenascin), carbon anhydrase IX (CA-IX), and/or peptides bound to a molecule of the human major histocompatibility complex (MHC).

The sequence(s) of the (human) members of the A33-antigen, BCMA (B-cell maturation antigen), cancer antigen 12-5 (CA-12-5), carbon anhydrase IX (CA-IX), CD19, CD20, CD22, CD33, CD38, CEA (carcinoembryonic antigen), EpCAM (epithelial cell adhesion molecule), FAP (fibroblast activation protein), FMS-like tyrosine kinase 3 (FLT-3), folate receptor 1 (FOLR1), HER-1 (human epidermal growth factor 1), HER-2 (human epidermal growth factor 2), HER-3 (human epidermal growth factor 3), human leukocyte antigen-antigen D related (HLA-DR), MSLN (mesothelin), MCSP (melanoma chondroitin sulfate proteoglycan), MUC-1 (Mucin-1), PSMA (prostate specific membrane antigen), PSMA (prostate-specific membrane antigen), PSCA (prostate stem cell antigen), p95 (p95HER2), transferrin-receptor, TNC (tenascin), human trophoblast cell-surface antigen 2 (Trop-2) are available in the UniProtKB/Swiss-Prot database and can be retrieved from http://www.uniprot.org/uniprot/?query=reviewed%3Ayes. These (protein) sequences also relate to annotated modified sequences. The present invention also provides techniques and methods wherein homologous sequences, and also genetic allelic variants and the like of the concise sequences provided herein are used. Preferably such variants and the like of the concise sequences herein are used. Preferably, such variants are genetic variants. The skilled person may easily deduce the relevant coding region of these (protein) sequences in these databank entries, which may also comprise the entry of genomic DNA as well as mRNA/cDNA. The sequence(s) of the (human) FAP (fibroblast activation protein) can be obtained from the Swiss-Prot database entry Q12884 (entry version 168, sequence version 5); The sequence(s) of the (human) CEA (carcinoembryonic antigen) can be obtained from the Swiss-Prot database entry P06731 (entry version 171, sequence version 3); the sequence(s) of the (human) EpCAM (Epithelial cell adhesion molecule) can be obtained from the Swiss-Prot database entry P16422 (entry version 117, sequence version 2); the sequence(s) of the (human) MSLN (mesothelin) can be obtained from the UniProt Entry number Q13421 (version number 132; sequence version 2); the sequence(s) of the (human) FMS-like tyrosine kinase 3 (FLT-3) can be obtained from the Swiss-Prot database entry P36888 (primary citable accession number) or Q13414 (secondary accession number) with the version number 165 and the sequence version 2; the sequences of (human) MCSP (melanoma chondroitin sulfate proteoglycan) can be obtained from the UniProt Entry number Q6UVK1 (version number 118; sequence version 2); the sequence(s) of the (human) folate receptor 1 (FOLR1) can be obtained from the UniProt Entry number P15328 (primary citable accession number) or Q53EW2 (secondary accession number) with the version number 153 and the sequence version 3; the sequence(s) of the (human) trophoblast cell-surface antigen 2 (Trop-2) can be obtained from the UniProt Entry number P09758 (primary citable accession number) or Q15658 (secondary accession number) with the version number 172 and the sequence version 3; the sequence(s) of the (human) PSCA (prostate stem cell antigen) can be obtained from the UniProt Entry number 043653 (primary citable accession number) or Q6UW92 (secondary accession number) with the version number 134 and the sequence version 1: the sequence(s) of the (human) HER-1 (Epidermal growth factor receptor) can be obtained from the Swiss-Prot database entry P00533 (entry version 177, sequence version 2); the sequence(s) of the (human) HER-2 (Receptor tyrosine-protein kinase erbB-2) can be obtained from the Swiss-Prot database entry P04626 (entry version 161, sequence version 1); the sequence(s) of the (human) HER-3 (Receptor tyrosine-protein kinase erbB-3) can be obtained from the Swiss-Prot database entry P21860 (entry version 140, sequence version 1); the sequence(s) of the (human) CD20 (B-lymphocyte antigen CD20) can be obtained from the Swiss-Prot database entry P11836 (entry version 117, sequence version 1); the sequence(s) of the (human) CD22 (B-lymphocyte antigen CD22) can be obtained from the Swiss-Prot database entry P20273 (entry version 135, sequence version 2); the sequence(s) of the (human) CD33 (B-lymphocyte antigen CD33) can be obtained from the Swiss-Prot database entry P20138 (entry version 129, sequence version 2); the sequence(s) of the (human) CA-12-5 (Mucin 16) can be obtained from the Swiss-Prot database entry Q8WX17 (entry version 66, sequence version 2); the sequence(s) of the (human) HLA-DR can be obtained from the Swiss-Prot database entry Q29900 (entry version 59, sequence version 1); the sequence(s) of the (human) MUC-1 (Mucin-1) can be obtained from the Swiss-Prot database entry P15941 (entry version 135, sequence version 3); the sequence(s) of the (human) A33 (cell surface A33 antigen) can be obtained from the Swiss-Prot database entry Q99795 (entry version 104, sequence version 1); the sequence(s) of the (human) PSMA (Glutamate carboxypeptidase 2) can be obtained from the Swiss-Prot database entry Q04609 (entry version 133, sequence version 1), the sequence(s) of the (human) transferrin receptor can be obtained from the Swiss-Prot database entries Q9UP52 (entry version 99, sequence version 1) and P02786 (entry version 152, sequence version 2); the sequence of the (human) TNC (tenascin) can be obtained from the Swiss-Prot database entry P24821 (entry version 141, sequence version 3); or the sequence(s) of the (human) CA-IX (carbonic anhydrase IX) can be obtained from the Swiss-Prot database entry Q16790 (entry version 115, sequence version 2).

In a specific embodiment, the target cell antigen is selected from the group consisting of fibroblast activation protein (FAP), carcinoembryonic antigen (CEA), mesothelin (MSLN), CD20, folate receptor 1 (FOLR1), and tenascin (TNC).

Antibodies capable of binding to any of the above mentioned target cell antigens can be generated using methods well known in the art such as immunizing a mammalian immune system and/or phage display using recombinant libraries as herein before described.

### Immune Activating Fe Binding Molecules

The chimeric receptor of the present invention can be combined with immune activating Fc binding molecules capable of binding to the mutated Fc domain. Suitable molecules are described herein below and also in WO2021/255138.

In one embodiment provided is a chimeric receptor as hereinbefore described for use in a method for treating or delaying progression of cancer, wherein the chimeric receptor is used in combination with any of the immune activating Fc binding molecules as described in WO2021/255138.

In one embodiment, provided is the chimeric receptor as hereinbefore described for use in a method for treating or delaying progression of cancer, wherein the chimeric receptor is used in combination with a molecule as described in FIG. 29A and Example 5 of WO2021/255138.

### Kits

A further aspect of the present invention are kits comprising or consisting of a nucleic acid encoding a chimeric receptor of the invention and/or cells, preferably T cells for transduction/transduced with chimeric receptors of the invention and, optionally, one or several (therapeutic) antibodies as hereinbefore described.

Accordingly, provided is a kit comprising a transduced T cell capable of expressing the chimeric receptor according to the invention, wherein the chimeric receptor comprises an extracellular domain comprising a mutated Fc domain or a fragment thereof; and an antibody, in particular a therapeutic antibody that comprises at least one antigen binding moiety capable of binding to the mutated Fc domain but not capable of binding to the non-mutated parent Fc domain, wherein the antibody comprises at least one effector moiety as described hereinbefore.

Further provided is a kit comprising an isolated polynucleotide encoding the chimeric receptor according to the invention.

In one embodiment, the effector moiety is an antigen binding moiety capable of binding to a target cell antigen as described hereinbefore.

In one embodiment, the effector moiety is an immune activating moiety as described hereinbefore, in particular a cytokine (e.g. IL2 or derivatives thereof).

In one embodiment, the chimeric receptor comprises a mutated Fc domain comprising the P329G substitution (numbering according to Kabat EU index). In a preferred such embodiment, the therapeutic antibody does not comprise the P329G substitution (numbering according to Kabat EU index). This prevents dimerization of the therapeutic antibody which comprises an antigen binding moiety capable of specific binding to the P329G substitution. In one embodiment, the therapeutic antibody comprises an amino acid substitution selected from the group consisting of P329G, P329L, P329I, P329R, and P329A (numbering according to Kabat EU index). In a preferred embodiment, the therapeutic antibody comprises the P329R substitution (numbering according to Kabat EU index).

In another preferred embodiment, the chimeric receptor comprises a mutated Fc domain comprising the amino acid substitutions I253A, H310A and H435A (numbering according to Kabat EU index). In a preferred such embodiment, the therapeutic antibody comprises the P329G substitution (numbering according to Kabat EU index).

In particular embodiments, the antibody is a therapeutic antibody, e.g. a tumor specific antibody or an immune activating antibody as hereinbefore described. Tumor specific antigens and immune activating moieties are known in the art and hereinbefore described. In the context of the present invention, the antibody is administered before, simultaneously with or after administration of transduced T cell expressing a chimeric receptor of the invention.

In one embodiment of the present invention, provided is a kit comprising a transduced T cell capable of expressing the amino acid sequence of SEQ ID NO:87, or alternatively, the kit comprises a polynucleotide encoding the amino acid sequence of SEQ ID NO:87 (for example the kit comprises a polynucleotide comprising the sequence of SEQ ID NO:96), combined with an antibody comprising a heavy chain of SEQ ID NO: 109, a heavy chain of SEQ ID NO: 110, a light chain of SEQ ID NO: 11 and two light chains of SEQ ID NO:108. This kit can be used for the treatment of FolR positive cancer.

In one embodiment of the present invention, provided is a kit comprising a transduced T cell capable of expressing the amino acid sequence of SEQ ID NO:106, or alternatively, the kit comprises a polynucleotide encoding the amino acid sequence of SEQ ID NO: 106 (for example the kit comprises a polynucleotide comprising the sequence of SEQ ID NO: 107), combined with an antibody comprising a heavy chain of SEQ ID NO: 112, a heavy chain of SEQ ID NO: 113, a light chain of SEQ ID NO: 114 and two light chains of SEQ ID NO: 108. This kit can be used for the treatment of FolR1 positive cancer.

In one embodiment of the present invention, provided is a kit comprising a transduced T cell capable of expressing the amino acid sequence of SEQ ID NO:87, or alternatively, the kit comprises a polynucleotide encoding the amino acid sequence of SEQ ID NO:87 (for example the kit comprises a polynucleotide comprising the sequence of SEQ ID NO:96), combined with an antibody comprising a heavy chain of SEQ ID NO: 109, a heavy chain of SEQ ID NO: 115, a light chain of SEQ ID NO: 108 and a light chain of SEQ ID NO: 111. This kit can be used for the treatment of FolR1 positive cancer.

In one embodiment of the present invention, provided is a kit comprising a transduced T cell capable of expressing the amino acid sequence of SEQ ID NO:106, or alternatively, the kit comprises a polynucleotide encoding the amino acid sequence of SEQ ID NO: 106 (for example the kit comprises a polynucleotide comprising the sequence of SEQ ID NO: 107), combined with an antibody comprising a heavy chain of SEQ ID NO: 112, a heavy chain of SEQ ID NO: 116, a light chain of SEQ ID NO:114 and a light chain of SEQ ID NO:108. This kit can be used for the treatment of FolR1 positive cancer.

In one embodiment of the present invention, provided is a kit comprising a transduced T cell capable of expressing the amino acid sequence of SEQ ID NO:87, or alternatively, the kit comprises a polynucleotide encoding the amino acid sequence of SEQ ID NO:87 (for example the kit comprises a polynucleotide comprising the sequence of SEQ ID NO:96), combined with an antibody comprising a heavy chain of SEQ ID NO: 118, a heavy chain of SEQ ID NO: 119, a light chain of SEQ ID NO:117 and two light chains of SEQ ID NO: 120. This kit can be used for the treatment of CEA positive cancer.

In one embodiment of the present invention, provided is a kit comprising a transduced T cell capable of expressing the amino acid sequence of SEQ ID NO:106, or alternatively, the kit comprises a polynucleotide encoding the amino acid sequence of SEQ ID NO: 106 (for example the kit comprises a polynucleotide comprising the sequence of SEQ ID NO:107), combined with an antibody comprising a heavy chain of SEQ ID NO: 122, a heavy chain of SEQ ID NO: 123, a light chain of SEQ ID NO: 121 and two light chains of SEQ ID NO:114. This kit can be used for the treatment of CEA positive cancer.

Furthermore, parts of the kit of the invention can be packaged individually in vials or bottles or in combination in containers or multicontainer units. Additionally, the kit of the present invention may comprise a (closed) bag cell incubation system where patient cells, preferably T cells as described herein, can be transduced with (an) chimeric receptor(s) of the invention and incubated under GMP (good manufacturing practice, as described in the guidelines for good manufacturing practice published by the European Commission under http://ec.europa.eu/health/documents/eudralex/index_en.htm) conditions. Furthermore, the kit of the present invention comprises a (closed) bag cell incubation system where isolated/obtained patients T cells can be transduced with (an) chimeric receptor(s) of the invention and incubated under GMP. Furthermore, in the context of the present invention, the kit may also comprise a vector encoding (the) chimeric receptor(s) as described herein. The kit of the present invention may be advantageously used, inter alia, for carrying out the method of the invention and could be employed in a variety of applications referred herein, e.g., as research tools or medical tools.

The manufacture of the kits preferably follows standard procedures which are known to the person skilled in the art.

In this context, patient derived cells, preferably T cells, can be transduced with a chimeric receptor as described herein using the kit as described above. The extracellular domain comprising the mutated Fc domain does not naturally occur in or on T cells. Accordingly, the patient derived cells transduced with the kits of the invention will acquire the capability to interact with the therapeutic antibody, e and will become capable of inducing elimination/lysis of target cells via interaction with a therapeutic antibody, wherein the therapeutic antibody is able to bind to a tumor-specific antigen naturally occurring (that is endogenously expressed) on the surface of a tumor cell. Binding of the extracellular domain of the chimeric receptor as described herein activates that T cell and brings it into physical contact with the tumor cell through the therapeutic antibody. Non-transduced or endogenous T cells (e.g. CD8+ T cells) are unable to bind to therapeutic antibody. Accordingly. T cells expressing the inventive chimeric receptor molecule have the ability to lyse target cells in the presence of an antibody as described herein in vivo and/or in vitro. Corresponding target cells comprise cells expressing a surface molecule. i.e. a tumor-specific antigen naturally occurring on the surface of a tumor cell, which is recognized by at least one, preferably two, binding domains of the therapeutic antibody as described herein.

Lysis of the target cell can be detected by methods known in the art. Accordingly, such methods comprise, inter alia, physiological in vitro assays. Such physiological assays may monitor cell death, for example by loss of cell membrane integrity (e.g. FACS based propidium Iodide assay, trypan blue influx assay, photometric enzyme release assays (LDH), radiometric 51Cr release assay, fluorometric Europium release and CalceinAM release assays). Further assays comprise monitoring of cell viability, for example by photometric MTT, XTT, WST-1 and alamarBlue assays, radiometric 3H-Thd incorporation assay, clonogenic assay measuring cell division activity, and fluorometric Rhodamine123 assay measuring mitochondrial transmembrane gradient. In addition, apoptosis may be monitored for example by FACS-based phosphatidylserin exposure assay. ELISA-based TUNEL test, caspase activity assay (photometric, fluorometric or ELISA-based) or analyzing changed cell morphology (shrinking, membrane blebbing).

### Exemplary Therapeutic Use and Methods of Treatment

The molecules or constructs (e.g., chimeric receptors, transduced T cells and kits) provided herein are particularly useful in medical settings, in particular for treatment of cancer. For example a tumor may be treated with a transduced T cell expressing a chimeric receptor of the present invention in conjunction with a therapeutic antibody that comprises at least one antigen binding moiety capable of binding to a target cell antigen and at least one antigen binding moiety capable of binding to a mutated Fc domain (e.g. comprising the P329G substitution according to EU numbering) but not capable of binding to the non-mutated parent Fc domain.

Accordingly, in certain embodiments, the chimeric receptor, the transduced T cell or the kit are used in the treatment of cancer, in particular cancer of epithelial, endothelial or mesothelial origin and cancer of the blood.

The tumor specificity of the treatment is provided by the therapeutic antibody that binds to a target cell antigen, wherein the antibody is administered before, simultaneously with or after administration of transduced T cell expressing a chimeric receptor of the invention. In this context, the transduced T cells are universal T cells since they are not specific for a given tumor but can target any tumor depending on the specificity of the therapeutic antibody used according to the invention.

The cancer may be a cancer/carcinoma of epithelial, endothelial or mesothelial origin or a cancer of the blood. In one embodiment the cancer/carcinoma is selected from the group consisting of gastrointestinal cancer, pancreatic cancer, cholangiocellular cancer, lung cancer, breast cancer, ovarian cancer, skin cancer, oral cancer, gastric cancer, cervical cancer. B and T cell lymphoma, myeloid leukemia, ovarial cancer, leukemia, lymphatic leukemia, nasopharyngeal carcinoma, colon cancer, prostate cancer, renal cell cancer, head and neck cancer, skin cancer (melanoma), cancers of the genitourinary tract, e.g., testis cancer, ovarial cancer, endothelial cancer, cervix cancer and kidney cancer, cancer of the bile duct, esophagus cancer, cancer of the salivatory glands and cancer of the thyroid gland or other tumorous diseases like haematological tumors, gliomas, sarcomas or osteosarcomas.

For example, tumorous diseases and/or lymphomas may be treated with a specific construct directed against these medical indication(s). For example, gastrointestinal cancer, pancreatic cancer, cholangiocellular cancer, lung cancer, breast cancer, ovarian cancer, skin cancer and/or oral cancer may be treated with an antibody directed against (human) EpCAM (as the tumor-specific antigen naturally occurring on the surface of a tumor cell).

Gastrointestinal cancer, pancreatic cancer, cholangiocellular cancer, lung cancer, breast cancer, ovarian cancer, skin cancer and/or oral cancer may be treated with a transduced T cell of the present invention administered before, simultaneously with or after administration of a therapeutic antibody directed against HER1, preferably human HER1. Furthermore, gastrointestinal cancer, pancreatic cancer, cholangiocellular cancer, lung cancer, breast cancer, ovarian cancer, skin cancer, glioblastoma and/or oral cancer may be treated with a transduced T cell of the present invention administered before, simultaneously with or after administration of a therapeutic antibody directed against MCSP, preferably human MCSP. Gastrointestinal cancer, pancreatic cancer, cholangiocellular cancer, lung cancer, breast cancer, ovarian cancer, skin cancer, glioblastoma and/or oral cancer may be treated with a transduced T cell of the present invention administered before, simultaneously with or after administration of a therapeutic antibody directed against FOLR1, preferably human FOLR1. Gastrointestinal cancer, pancreatic cancer, cholangiocellular cancer, lung cancer, breast cancer, ovarian cancer, skin cancer, glioblastoma and/or oral cancer may be treated with a transduced T cell of the present invention administered before, simultaneously with or after administration of a therapeutic antibody directed against Trop-2, preferably human Trop-2. Gastrointestinal cancer, pancreatic cancer, cholangiocellular cancer, lung cancer, breast cancer, ovarian cancer, skin cancer, glioblastoma and/or oral cancer may be treated with a transduced T cell of the present invention administered before, simultaneously with or after administration of a therapeutic antibody directed against PSCA, preferably human PSCA. Gastrointestinal cancer, pancreatic cancer, cholangiocellular cancer, lung cancer, breast cancer, ovarian cancer, skin cancer, glioblastoma and/or oral cancer may be treated with a transduced T cell of the present invention administered before, simultaneously with or after administration of a therapeutic antibody directed against EGFRvIII, preferably human EGFRvIII. Gastrointestinal cancer, pancreatic cancer, cholangiocellular cancer, lung cancer, breast cancer, ovarian cancer, skin cancer, glioblastoma and/or oral cancer may be treated with a transduced T cell of the present invention administered before, simultaneously with or after administration of a therapeutic antibody directed against MSLN, preferably human MSLN. Gastric cancer, breast cancer and/or cervical cancer may be treated with a transduced T cell of the present invention administered before, simultaneously with or after administration of a therapeutic antibody directed against HER2, preferably human HER2. Gastric cancer and/or lung cancer may be treated with a transduced T cell of the present invention administered before, simultaneously with or after administration of a therapeutic antibody directed against HER3, preferably human HER3. B-cell lymphoma and/or T cell lymphoma may be treated with a transduced T cell of the present invention administered before, simultaneously with or after administration of a therapeutic antibody directed against CD20, preferably human CD20. B-cell lymphoma and/or T cell lymphoma May be treated with a transduced T cell of the present invention administered before, simultaneously with or after administration of a therapeutic antibody directed against CD22, preferably human CD22. Myeloid leukemia may be treated with a transduced T cell of the present invention administered before, simultaneously with or after administration of a therapeutic antibody directed against CD33, preferably human CD33. Ovarian cancer, lung cancer, breast cancer and/or gastrointestinal cancer may be treated with a transduced T cell of the present invention administered before, simultaneously with or after administration of a therapeutic antibody directed against CA12-5, preferably human CA12-5. Gastrointestinal cancer, leukemia and/or nasopharyngeal carcinoma may be treated with a transduced T cell of the present invention administered before, simultaneously with or after administration of a therapeutic antibody directed against HLA-DR, preferably human HLA-DR. Colon cancer, breast cancer, ovarian cancer, lung cancer and/or pancreatic cancer may be with a transduced T cell of the present invention administered before, simultaneously with or after administration of a therapeutic antibody directed against MUC-1, preferably human MUC-1. Colon cancer may be treated with a transduced T cell of the present invention administered before, simultaneously with or after administration of a therapeutic antibody directed against A33, preferably human A33. Prostate cancer may be treated with a transduced T cell of the present invention administered before, simultaneously with or after administration of a therapeutic antibody directed against PSMA, preferably human PSMA. Gastrointestinal cancer, pancreatic cancer, cholangiocellular cancer, lung cancer, breast cancer, ovarian cancer, skin cancer and/or oral cancer may be treated with a transduced T cell of the present invention administered before, simultaneously with or after administration of a therapeutic directed against the transferrin receptor, preferably the human transferring receptor. Pancreatic cancer, lunger cancer and/or breast cancer may be treated with a transduced T cell of the present invention administered before, simultaneously with or after administration of a therapeutic antibody directed against the transferrin receptor, preferably the human transferring receptor. Renal cancer may be with a transduced T cell of the present invention administered before, simultaneously with or after administration of a therapeutic antibody directed against CA-IX, preferably human CA-IX.

The invention also relates to a method for the treatment of a disease, a malignant disease such as cancer of epithelial, endothelial or mesothelial origin and/or cancer of blood. In the context of the present invention, said subject is a human.

In the context of the present invention a particular method for the treatment of a disease comprises the steps of
(a) isolating T cells, preferably CD8+ T cells, from a subject;
(b) transducing said isolated T cells, preferably CD8+ T cells, with a chimeric receptor as described herein; and
(c) administering the transduced T cells, preferably CD8+ T cells, to said subject.

In the context of the present invention, said transduced T cells, preferably CD8+ T cells, and/or therapeutic antibody/antibodies are co-administered to said subject by intravenous infusion.

Moreover, in the context of the present invention the present invention, provides a method for the treatment of a disease comprising the steps of
(a) isolating T cells, preferably CD8+ T cells, from a subject;
(b) transducing said isolated T cells, preferably CD8+ T cells, with a chimeric receptor as described herein;
(c) expanding the T cells, preferably CD8+ T cells, by anti-CD3 and anti-CD28 antibodies; and
(d) administering the transduced T cells, preferably CD8+ T cells, to said subject.

The above mentioned step (c) (referring to the expanding step of the T cells such as TIL by anti-CD3 and/or anti-CD28 antibodies) may also be performed in the presence of (stimulating) cytokines such as interleukin-2 and/or interleukin-15 (IL-15). In the context of the present invention, the above mentioned step (d) (referring to the expanding step of the T cells such as TIL by anti-CD3 and/or anti-CD28 antibodies) may also be performed in the presence of interleukin-12 (IL-12), interleukin-7 (IL-7) and/or interleukin-21 (IL-21).

The method for the treatment, in addition, comprise the administration of the antibody used according to the present invention. Said antibody may be administered before, simultaneously with or after the transduced T cells are to be administered. In the context of the present invention the administration of the transduced T cells will be performed by intravenous infusion. In the context of the present invention that transduced T cells are isolated/obtained from the subject to be treated.

The invention further envisages the co-administration protocols with other compounds, e.g., molecules capable of providing an activation signal for immune effector cells, for cell proliferation or for cell stimulation. Said molecule may be, e.g., a further primary activation signal for T cells (e.g. a further costimulatory molecule: molecules of B7 family, Ox40L, 4.1 BBL, CD40L, anti-CTLA-4, anti-PD-1), or a further cytokine interleukin (e.g., IL-2).

The composition of the invention as described above may also be a diagnostic composition further comprising, optionally, means and methods for detection.

### Compositions

Furthermore, the invention provides compositions (medicaments) comprising (an) antibody molecule(s), and/or (a) transduced T cell(s) comprising a chimeric receptor of the invention, and/or (a) nucleic acid molecule(s) and (a) vector(s) encoding the chimeric receptors according to the invention. Furthermore, the invention provides kits comprising one or more of said compositions. In the context of the present invention, the composition is a pharmaceutical composition further comprising, optionally, suitable formulations of carrier, stabilizers and/or excipients. Accordingly, in the context of the present invention a pharmaceutical composition (medicament) is provided that comprises an antibody molecule as herein described which is to be administered in combination with a transduced T cell comprising a chimeric receptor as herein described and/or a composition comprising said transduced T cell, wherein said antibody molecule is to be administered before, simultaneously with or after administration of transduced T cells comprising a chimeric receptor of the invention.

The use of the term “in combination” does not restrict the order in which the components of the treatment regimen are to be administered to the subject. Accordingly, the pharmaceutical composition/medicament described herein encompass the administration of an antibody as defined herein before, simultaneously with or after administration of transduced T cells comprising a chimeric receptor of the present invention. “In combination” as used herein also does not restrict the timing between the administration of an antibody as defined herein before and the transduced T cells comprising a chimeric receptor as defined herein. Thus, when the two components are not administered simultaneously with/concurrently, the administrations may be separated by 1 minute, 5 minutes, 15 minutes, 30 minutes, 45 minutes, 1 hour, 2 hours, 4 hours, 6 hours, 12 hours, 24 hours, 48 hours or 72 hours or by any suitable time differential readily determined by one of skill in art and/or described herein.

In the context of the present invention the term “in combination” also encompasses the situation where the antibody as defined herein and the transduced T cells comprising a chimeric receptor according to the invention are pre-incubated together before administration to the subject. Thus, the two components may be pre-incubated before administration, for example, for 1 minute, 5 minutes, 10 minutes, 15 minutes, 30 minutes, 45 minutes or 1 hour or for any suitable time readily determined by one skilled in the art. The invention, in another embodiment, relates to a treatment regimen, in which the antibody as defined herein and the transduced T cells comprising a chimeric receptor as defined herein, are to be administered simultaneously with/concurrently. In the context of the present invention, the antibody as defined herein may be administered after the transduced T cells comprising a chimeric receptor has been administered.

Further, “in combination” as used herein does not restrict the disclosed treatment regimens to the administration of an antibody as defined herein and transduced T cells, preferably CD8+ T cells, comprising a chimeric receptor of the invention in immediate sequence (i.e., the administration of one of the two components, followed (after a certain time interval) by the administration of the other without the administration and/or practice of any other treatment protocol in between. Therefore, the present treatment regimens also encompass the separate administration of an antibody molecule as defined herein and transduced T cells, preferably CD8+ T cells, comprising a chimeric receptor according to the invention, wherein the administrations are separated by one or more treatment protocols necessary and/or suitable for the treatment or prevention of the disease, or a symptom thereof. Examples of such intervening treatment protocols include but are not limited to, administration of pain medications: administration of chemotherapeutics, surgical handling of the disease or a symptom thereof.

Accordingly, the treatment regimens as disclosed herein encompass the administration of an antibody as defined herein and transduced T cells, preferably CD8+ T cells, comprising a chimeric receptor as defined herein together with none, one, or more than one treatment protocol suitable for the treatment or prevention of a disease, or a symptom thereof, as described herein or as known in the art.

It is particular envisaged, that said pharmaceutical composition(s)/medicament(s) is (are) to be administered to a patient via infusion or injection. In the context of the present invention the transduced T cells comprising a chimeric receptor as described herein is to be administered to a patient via infusion or injection. Administration of the suitable compositions/medicaments may be effected by different ways, intravenous, intraperitoneal, subcutaneous, intramuscular, topical or intradermal administration.

The pharmaceutical composition/medicament of the present invention may further comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, etc. Compositions comprising such carriers can be formulated by well-known conventional methods. These pharmaceutical compositions can be administered to the subject at a suitable dose. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently. Generally, the regimen as a regular administration of the pharmaceutical composition should be in the range of 1 μg to 5 g units per day. However, a more preferred dosage for continuous infusion might be in the range of 0.01 μg to 2 mg, preferably 0.01 μg to 1 mg, more preferably 0.01 μg to 100 μg, even more preferably 0.01 μg to 50 μg and most preferably 0.01 μg to 10 μg units per kilogram of body weight per hour. Particularly preferred dosages are recited herein below.

Progress can be monitored by periodic assessment. Dosages will vary but a preferred dosage for intravenous administration of DNA is from approximately 106 to 1012 copies of the DNA molecule.

The compositions of the invention may be administered locally or systematically. Administration will generally be parenterally, e.g., intravenously: transduced T cells may also be administered directed to the target site, e.g., by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present invention might comprise proteinaceous carriers, like, e.g., serum albumine or immunoglobuline, preferably of human origin. It is envisaged that the pharmaceutical composition of the invention might comprise, in addition to the proteinaceous antibody constructs or nucleic acid molecules or vectors encoding the same (as described in this invention), and/or cells, further biologically active agents, depending on the intended use of the pharmaceutical composition. Such agents might be drugs acting on the gastro-intestinal system, drugs acting as cytostatica, drugs preventing hyperurikemia, drugs inhibiting immunereactions (e.g. corticosteroids), drugs acting on the circulatory system and/or agents such as T cell co-stimulatory molecules or cytokines known in the art.

### FURTHER EXEMPLARY EMBODIMENTS

1. A chimeric receptor comprising
(i) an extracellular domain comprising a mutated Fc domain or a fragment thereof, and
(ii) a transmembrane domain.

2. The chimeric receptor of embodiment 1, further comprising (iii) at least one intracellular stimulatory signaling domain and/or at least one co-stimulatory signaling domain.

3. The chimeric receptor of embodiment 1 or 2, wherein the extracellular domain comprises a mutated CH2 domain and/or a mutated CH3 domain.

4. The chimeric receptor of any one of embodiments 1-3, wherein the extracellular domain comprises a mutated CH2 domain.

5. The chimeric receptor of any one of embodiments 1-4, wherein the mutated Fc domain comprise at least one amino acid substitution at a position selected from the group consisting of L234, L235, 1253, H310, P331, P329 and H435 (numbering according to Kabat EU index), in particular wherein the amino acid substitution is selected from the group consisting of L234A, L235A, I253A, N297A, H310A, P329G and/or H435A.

6. The chimeric receptor of any one of embodiments 1-5, wherein the extracellular domain comprises a mutated CH2 domain, wherein the mutated CH2 domain comprises the amino acid substitution P329G (numbering according to Kabat EU index).

7. The chimeric receptor of any one of embodiments 1-6, wherein the extracellular domain comprises a mutated CH2 domain and/or a mutated CH3 domain, wherein the mutated CH2 domain and/or the mutated CH3 domain comprises the amino acid substitution (numbering according to Kabat EU index):
(a) I253A, H310A and H435A,
(b) I253A
(c) I253A and H310A
(d) I253A and H435A
(e) H310A and H435A
(f) H310A,
(g) H435A,
(h) L234A, L235A, and P329G, or
(i) L234A, L235A, I253A, H310A, P329G and H435A.

8. The chimeric receptor of any one of embodiments 1-7, wherein the extracellular domain does not comprise a heavy chain variable domain (VH) and/or light chain variable domain (VL).

9. The chimeric receptor of any one of embodiments 1-8, wherein the transmembrane domain is selected from the group consisting of the CD8, the CD4, the CD3z, the FCGR3A, the NKG2D, the CD27, the CD28, the CD137, the OX40, the ICOS, the DAP10 or the DAP12 transmembrane domain or a fragment thereof.

10. The chimeric receptor of any one of embodiments 2-9, wherein the transmembrane domain is the CD8 transmembrane domain, in particular wherein the transmembrane domain comprises the amino acid sequence of SEQ ID NO: 11.

11. The chimeric receptor of any one of embodiments 2-10, wherein the stimulatory signaling domain is selected from the group consisting of the intracellular domain of CD3z, of FCGR3A and of NKG2D, or a fragment thereof that retains stimulatory signaling activity.

12. The chimeric receptor of any one of embodiments 2-11, wherein the stimulatory signaling domain is the intracellular domain of CD3z or a fragment thereof that retains stimulatory signaling activity, in particular wherein the stimulatory signaling domain comprises the amino acid sequence of SEQ ID NO: 13.

13. The chimeric receptor of any one of embodiments 2-12, wherein the co-stimulatory signaling domain is selected from the group consisting of the intracellular domain of CD27, of CD28, of CD137, of OX40, of ICOS, of DAP10 and of DAP12, or a fragment thereof that retain co-stimulatory signaling activity.

14. The chimeric receptor of any one of embodiments 2-13, wherein the co-stimulatory signaling domain is a CD137 intracellular domain or a fragment thereof that retains CD137 co-stimulatory activity, in particular wherein the co-stimulatory signaling domain comprises the amino acid sequence of SEQ ID NO: 12.

15. The chimeric receptor of any one of embodiments 2-14, wherein the co-stimulatory signaling domain is the CD28 intracellular domain or a fragment thereof that retains CD28 co-stimulatory activity.

16. The chimeric receptor of any one of embodiments 2-15, wherein the chimeric receptor comprises one stimulatory signaling domain comprising the intracellular domain of CD3z, or a fragment thereof that retains CD3z stimulatory signaling activity, and wherein the chimeric receptor comprises one co-stimulatory signaling domain comprising the intracellular domain of CD137, or a fragment thereof that retains CD137 co-stimulatory signaling activity.

17. The chimeric receptor of any one of embodiments 2-16, wherein the stimulatory signaling domain comprises the amino acid sequence of SEQ ID NO:13 and the co-stimulatory signaling domain comprises the amino acid sequence of SEQ ID NO: 12.

18. The chimeric receptor of any one of embodiments 1-17, wherein the extracellular domain is connected to the transmembrane domain, optionally through a peptide linker.

19. The chimeric receptor of any one of embodiments 1-18, wherein the transmembrane domain is connected at the N-terminus to the C-terminus of the extracellular domain.

20. The chimeric receptor of any one of embodiments 2-19, wherein the transmembrane domain is connected to the co-stimulatory signaling domain or to the stimulatory signaling domain, optionally through a peptide linker.

21. The chimeric receptor of any one of embodiments 2-20, wherein the co-stimulatory signaling domain is connected at the N-terminus to the C-terminus of the transmembrane domain.

22. The chimeric receptor of embodiment 21, wherein the chimeric receptor comprises a stimulatory signaling domain, wherein the stimulatory signaling domain is connected at the N-terminus to the C-terminus of the co-stimulatory signaling domain.

23. The chimeric receptor of any one of embodiments 1-22, wherein the chimeric receptor comprises an amino acid sequence that is at least about 95%, 96%, 97%, 98%, 99% or 100% identical to the amino acid sequence of SEQ ID NO:7 or SEQ ID NO:125.

24. A chimeric receptor comprising the amino acid sequence of SEQ ID NO:7; or a chimeric receptor comprising the amino acid sequence of SEQ ID NO: 125, 25. The chimeric receptor of any one of embodiments 1-22, wherein the chimeric receptor comprises an amino acid sequence that is at least about 95%, 96%, 97%, 98%, 99% or 100% identical to an amino acid sequence selected from the group consisting of SEQ ID NO: 87, SEQ ID NO: 106, SEQ ID NO: 125 and SEQ ID NO:126.

26. A chimeric receptor comprising or consisting of the amino acid sequence of SEQ ID NO:87.

27. A chimeric receptor comprising or consisting of the amino acid sequence of SEQ ID NO: 106.

28. A chimeric receptor comprising or consisting of the amino acid sequence of SEQ ID NO: 125.

29. A chimeric receptor comprising or consisting of the amino acid sequence of SEQ ID NO: 127.

30. An isolated polynucleotide encoding the chimeric receptor of any one of embodiments 1-29.

31. A polypeptide encoded by the isolated polynucleotide of embodiments 30.

32. A vector, particularly an expression vector, comprising the polynucleotide of embodiment 30.

33. A transduced T cell comprising the polynucleotide of embodiment 30 or the vector of embodiment 32.

34. A transduced T cell capable of expressing the chimeric receptor of any one of embodiments 1-29.

35. A kit comprising
(A) a transduced T cell capable of expressing the chimeric receptor of any one of embodiments 1-30, wherein the chimeric receptor comprises an extracellular domain comprising a mutated Fc domain or a fragment thereof; and
(B) an antibody that comprises at least one antigen binding moiety capable of binding to the mutated Fc domain but not capable of binding to the non-mutated parent Fc domain, wherein the antibody comprises at least one effector moiety.

36. A kit comprising
(A) an isolated polynucleotide encoding the chimeric receptor of any one of embodiments 1-30, wherein the chimeric receptor comprises an extracellular domain comprising a mutated Fc domain or a fragment thereof; and
(B) an antibody that comprises at least one antigen binding moiety capable of binding to the mutated Fc domain but not capable of binding to the non-mutated parent Fc domain, wherein the antibody comprises at least one effector moiety.

37. The kit of embodiment 36 or 37, wherein the effector moiety is (i) an antigen binding moiety capable of binding to a target cell antigen, or (ii) and immune activating moiety, in particular a cytokine.

38. The kit of any one of embodiments 35-37 for use as a medicament.

39. The chimeric receptor of any one of embodiments 1-29 or the transduced T cell of any one of embodiments 33 or 34 for use as a medicament, wherein the chimeric receptor comprises an extracellular domain comprising a mutated Fc domain or a fragment thereof, wherein a transduced T cell expressing the chimeric receptor is administered before, simultaneously with or after administration of an antibody that comprises at least one antigen binding moiety capable of binding to a target cell antigen, and at least one antigen binding moiety capable of binding to the mutated Fc domain but not capable of binding to the non-mutated parent Fc domain.

40. The kit of any one of embodiments 35-36 for use in the treatment of a disease, in particular for use in the treatment of a cancer.

41. A method of treating a disease in a subject, comprising administering to the subject a transduced T cell capable of expressing the chimeric receptor of any one of embodiments 1-30, wherein the chimeric receptor comprises an extracellular domain comprising a mutated Fc domain or a fragment thereof, and administering before, simultaneously with or after administration of the transduced T cell a therapeutically effective amount of an an antibody that comprises at least one antigen binding moiety capable of binding to the mutated Fc domain but not capable of binding to the non-mutated parent Fc domain, wherein the antibody comprises at least one effector moiety.

42. The method of embodiment 41, wherein the effector moiety is (i) an antigen binding moiety capable of binding to a target cell antigen, or (ii) and immune activating moiety, in particular a cytokine.

43. Use of the chimeric receptor of any one of embodiments 1-29, the polynucleotide of embodiment 30 or the transduced T cell of embodiment 33 or 34 for the manufacture of a medicament.

44. The use of embodiment 43, wherein the medicament is for treatment of cancer.

45. The use of embodiment 44, characterized in that said cancer is selected from cancer of epithelial, endothelial or mesothelial origin and cancer of the blood.

46. The invention as hereinbefore described.

These and other embodiments are disclosed and encompassed by the description and Examples of the present invention. Further literature concerning any one of the antibodies, methods, uses and compounds to be employed in accordance with the present invention may be retrieved from public libraries and databases, using for example electronic devices. For example, the public database “Medline”, available on the Internet, may be utilized, for example under http://www.ncbi.nlm.nih.gov/PubMed/medline.html. Further databases and addresses, such as http://www.ncbi.nlm.nih.gov/, http://www.infobiogen.fr/, http://www.fmi.ch/biology/research_tools.html, http://www.tigr.org/, are known to the person skilled in the art and can also be obtained using, e.g., http://www.lycos.com.

### Exemplary Sequences

CDR definition in the sequence tables is according to Kabat.

TABLE 1
Exemplary anti-P329G antigen binding moieties

| | | SEQ ID |
|---|---|---|
| Construct | Amino acid sequence | NO |
| M-1.7.24 | RYWMN | 1 |
| HCDR1 | | |
| M-1.7.24 | EITPDSSTINYTPSLKD | 2 |
| HCDR2 | | |
| M-1.7.24 | PYDYGAWFAS | 3 |
| HCDR3 | | |
| M-1.7.24 | RSSTGAVTTSNYAN | 4 |
| LCDR1 | | |
| M-1.7.24 | GTNKRAP | 5 |
| LCDR2 | | |
| M-1.7.24 | ALWYSNHWV | 6 |
| LCDR3 | | |
| M-1.7.24 VH | EVKLLESGGGLVQPGGSLKLSCAASGFDFSRYWMNWVRQ | 7 |
| | APGKGLEWIGEITPDSSTINYTPSLKDKFIISRDNAKNTLYLQ | |
| | MIKVRSEDTALYYCVRPYDYGAWFASWGQGTLVTVSA | |
| M-1.7.24 VL | QAVVTQESALTTSPGETVTLTCRSSTGAVTTSNYANWVQE | 8 |
| | KPDHLFTGLIGGTNKRAPGVPARFSGSLIGDKAALTITGAQ | |
| | TEDEAIYFCALWYSNHWVFGGGTKLTVL | |
| VH1VL1 | RYWMN | 1 |
| HCDR1 | | |
| VH1VL1 | EITPDSSTINYTPSLKG | 9 |
| HCDR2 | | |
| VH1VL1 | PYDYGAWFAS | 3 |
| HCDR3 | | |
| VH1VL1 | RSSTGAVTTSNYAN | 4 |
| LCDR1 | | |
| VH1VL1 | GTNKRAP | 5 |
| LCDR2 | | |
| VH1VL1 | ALWYSNHWV | 6 |
| LCDR3 | | |
| VH1VL1 VH | EVQLVESGGGLVQPGGSLRLSCAASGFDFSRYWMNWVRQ | 10 |
| | APGKGLEWVGEITPDSSTINYTPSLKGRFTISRDNAKNSLYL | |
| | QMNSLRAEDTAVYYCVRPYDYGAWFASWGQGTLVTVSS | |
| VH1VLI VL | QAVVTQEPSLTVSPGGTVTLTCRSSTGAVTTSNYANWVQE | 11 |
| | KPDHLFTGLIGGTNKRAPGTPARFSGSLLGGKAALTLSGAQ | |
| | PEDEAEYYCALWYSNHWVFGGGTKLTVL | |
| VH2VL1 | RYWMN | 1 |
| HCDR1 | | |
| VH2VL1 | EITPDSSTINYAPSLKG | 12 |
| HCDR2 | | |
| VH2VL1 | PYDYGAWFAS | 3 |
| HCDR3 | | |
| VH2VL1 | RSSTGAVTTSNYAN | 4 |
| LCDR1 | | |
| VH2VL1 | GTNKRAP | 5 |
| LCDR2 | | |
| VH2VL1 | ALWYSNHWV | 6 |
| LCDR3 | | |
| VH2VL1 VH | EVQLVESGGGLVQPGGSLRLSCAASGFDFSRYWMNWVRQ | 13 |
| | APGKGLEWVGEITPDSSTINYAPSLKGRFTISRDNAKNSLYL | |
| | QMNSLRAEDTAVYYCVRPYDYGAWFASWGQGTLVTVSS | |
| VH2VL1 VL | QAVVTQEPSLTVSPGGTVTLTCRSSTGAVTTSNYANWVQE | 11 |
| | KPDHLFTGLIGGTNKRAPGTPARFSGSLLGGKAALTLSGAQ | |
| | PEDEAEYYCALWYSNHWVFGGGTKLTVL | |
| VH3VL1 | RYWMN | 1 |
| HCDR1 | | |
| VH3VL1 | EITPDSSTINYAPSLKG | 12 |
| HCDR2 | | |
| VH3VL1 | PYDYGAWFAS | 3 |
| HCDR3 | | |
| VH3VL1 | RSSTGAVTTSNYAN | 4 |
| LCDR1 | | |
| VH3V1 | GTNKRAP | 5 |
| LCDR2 | | |
| VH3VL1 | ALWYSNHWV | 6 |
| LCDR3 | | |
| VH3VL1 VH | EVQLVESGGGLVQPGGSLRLSCAASGFTFSRYWMNWVRQ | 14 |
| | APGKGLEWVGEITPDSSTINYAPSLKGRFTISRDNAKNSLYL | |
| | QMNSLRAEDTAVYYCARPYDYGAWFASWGQGTLVTVSS | |
| VH3VL1 VL | QAVVTQEPSLTVSPGGTVTLTCRSSTGAVTTSNYANWVQE | 11 |
| | KPDHLFTGLIGGTNKRAPGTPARFSGSLLGGKAALTLSGAQ | |
| | PEDEAEYYCALWYSNHWVFGGGTKLTVL | |
| VH4VL1 | RYWMN | 1 |
| HCDR1 | | |
| VH4VL1 | EITPDSSTINYADSVKG | 15 |
| HCDR2 | | |
| VH4VL1 | PYDYGAWFAS | 3 |
| HCDR3 | | |
| VH4VL1 | RSSTGAVTTSNYAN | 4 |
| LCDR1 | | |
| VH4VL1 | GTNKRAP | 5 |
| LCDR2 | | |
| VH4VL1 | ALWYSNHWV | 6 |
| LCDR3 | | |
| VH4VL1 VH | EVQLVESGGGLVQPGGSLRLSCAASGFDFSRYWMNWVRQ | 16 |
| | APGKGLEWVSEITPDSSTINYADSVKGRFTISRDNAKNSLY | |
| | LQMNSLRAEDTAVYYCARPYDYGAWFASWGQGTLVTVSS | |
| VH4VL1 VL | QAVVTQEPSLTVSPGGTVTLTCRSSTGAVTTSNYANWVQE | 11 |
| | KPDHLFTGLIGGTNKRAPGTPARFSGSLLGGKAALTLSGAQ | |
| | PEDEAEYYCALWYSNHWVFGGGTKLTVL | |
| VH1VL2 | RYWMN | 1 |
| HCDR1 | | |
| VH1VL2 | EITPDSSTINYTPSLKG | 9 |
| HCDR2 | | |
| VH1VL2 | PYDYGAWFAS | 3 |
| HCDR3 | | |
| VH1VL2 | RSSTGAVTTSNYAN | 4 |
| LCDR1 | | |
| VH1VL2 | GTNKRAP | 5 |
| LCDR2 | | |
| VH1VL2 | ALWYSNHWV | 6 |
| LCDR3 | | |
| VH1VL2 VH | EVQLVESGGGLVQPGGSLRLSCAASGFDFSRYWMNWVRQ | 10 |
| | APGKGLEWVGEITPDSSTINYTPSLKGRFTISRDNAKNSLYL | |
| | QMNSLRAEDTAVYYCVRPYDYGAWFASWGQGTLVTVSS | |
| VH1VL2 VL | QAVVTQEPSLTVSPGGTVTLTCRSSTGAVTTSNYANWFQQ | 17 |
| | KPGQAFTGLIGGTNKRAPGTPARFSGSLLGGKAALTLSGAQ | |
| | PEDEAEYYCALWYSNHWVFGGGTKLTVL | |
| VH1VL3 | RYWMN | 1 |
| HCDR1 | | |
| VH1VL3 | EITPDSSTINYTPSLKG | 9 |
| HCDR2 | | |
| VH1VL3 | PYDYGAWFAS | 3 |
| HCDR3 | | |
| VH1VL3 | GSSTGAVTTSNYAN | 18 |
| LCDR1 | | |
| VH1VL3 | GTNKRAP | 5 |
| LCDR2 | | |
| VH1VL3 | ALWYSNHWV | 6 |
| LCDR3 | | |
| VH1VL3 VH | EVQLVESGGGLVQPGGSLRLSCAASGFDFSRYWMNWVRQ | 10 |
| | APGKGLEWVGEITPDSSTINYTPSLKGRFTISRDNAKNSLYL | |
| | QMNSLRAEDTAVYYCVRPYDYGAWFASWGQGTLVTVSS | |
| VH1VL3 VL | QAVVTQEPSLTVSPGGTVTLTCGSSTGAVTTSNYANWFQQ | 19 |
| | KPGQAPRTLIGGTNKRAPGTPARFSGSLLGGKAALTLSGAQ | |
| | PEDEAEYYCALWYSNHWVFGGGTKLTVL | |

TABLE 2
Exemplary mutated CH2 domains

| | | SEQ ID |
|---|---|---|
| Construct | Amino acid sequence | NO |
| CH2 P329G | APELLGGPSVFLFPPKPKDTLMISRTPEVTCVVVDVSHEDPE | 20 |
| | VKFNWYVDGVEVHNAKTKPREEQYNSTYRVVSVLTVLHQ | |
| | DWLNGKEYKCKVSNKALGAPIEKTISKAK | |
| CH2 Fc | APELLGGPSVFLFPPKPKDTLMISRTPEVTCVVVDVSHEDPE | 21 |
| P329L | VKFNWYVDGVEVHNAKTKPREEQYNSTYRVVSVLTVLHQ | |
| | DWLNGKEYKCKVSNKALLAPIEKTISKAK | |
| CH2 Fc | APELLGGPSVFLFPPKPKDTLMISRTPEVTCVVVDVSHEDPE | 22 |
| P329I | VKFNWYVDGVEVHNAKTKPREEQYNSTYRVVSVLTVLHQ | |
| | DWLNGKEYKCKVSNKALIAPIEKTISKAK | |
| CH2 Fc | APELLGGPSVFLFPPKPKDTLMISRTPEVTCVVVDVSHEDPE | 23 |
| P329R | VKFNWYVDGVEVHNAKTKPREEQYNSTYRVVSVLTVLHQ | |
| | DWLNGKEYKCKVSNKALRAPIEKTISKAK | |
| CH2 Fc | APELLGGPSVFLFPPKPKDTLMISRTPEVTCVVVDVSHEDPE | 24 |
| P329A | VKFNWYVDGVEVHNAKTKPREEQYNSTYRVVSVLTVLHQ | |
| | DWLNGKEYKCKVSNKALAAPIEKTISKAK | |

TABLE 3
Exemplary mutated CH2-CH3 domains

| | | SEQ ID |
|---|---|---|
| Construct | Amino acid sequence | NO |
| CH2 P329R | AAPEAAGGPSVFLFPPKPKDTLMASRTPEVTCVVVDVSHE | 25 |
| LALA, | DPEVKFNWYVDGVEVHNAKTKPREEQYNSTYRVVSVLTV | |
| I253A, | LAQDWLNGKEYKCKVSNKALRAPIEKTISKAK | |
| H310A | | |
| CH3 H435A | GQPREPQVYTLPPSRDELTKNQVSLTCLVKGFYPSDIAVEW | 26 |
| | ESNGQPENNYKTTPPVLDSDGSFFLYSKLTVDKSRWQQGN | |
| | VFSCSVMHEALHNAYTQKSLSLSPGK | |
| CH2-CH3 | AAPELLGGPSVFLFPPKPKDTLMASRTPEVTCVVVDVSHED | 27 |
| I253A | PEVKFNWYVDGVEVHNAKTKPREEQYNSTYRVVSVLTVL | |
| H310A | AQDWLNGKEYKCKVSNKALRAPIEKTISKAKGQPREPQVY | |
| H435A | TLPPSRDELTKNQVSLTCLVKGFYPSDIAVEWESNGQPENN | |
| | YKTTPPVLDSDGSFFLYSKLTVDKSRWQQGNVFSCSVMHE | |
| | ALHNAYTQKSLSLSPGK | |

TABLE 4
Exemplary anti-AAA antigen binding moiety

| | | SEQ ID |
|---|---|---|
| Construct | Amino acid sequence | NO |
| HCDR1 | GYYWS | 28 |
| HCDR2 | EINHGGYVTYNPSLES | 29 |
| HCDR3 | DYGPGNYDWYFDL | 30 |
| LCDR1 | RASQSVSSYLA | 31 |
| LCDR2 | DASNRAT | 32 |
| LCDR3 | QQRSNWPPALT | 33 |
| VH | MNFGLSLVFLALILKGVQCEVQLVESGGDLVKPGGSLKLS | 34 |
| | CAASGFTFSSYGMSWVRQTPDKRLEWVATISSGGSYIYYP | |
| | DSVKGRFTISRDNAKNTLYLQMSSLKSEDTAMYYCARLG | |
| | MITTGYAMDYWGQGTSVTVSS | |
| VL | DVLMTQTPLSLPVSLGDQASISCRSSQTIVHSTGHTYLEWFL | 35 |
| | QKPGQSPKLLIYKVSNRFSGVPDRFSGSGSGTDFTLKISRVE | |
| | AEDLGVYYCFQGSHVPYTFGGGTKLEIK | |

TABLE 5
Exemplary human and murine sequences

| | | SEQ ID |
|---|---|---|
| Construct | Amino acid sequence | NO |
| Human CD27 | ATGGCGCGCCCGCATCCGTGGTGGCTGTGCGTGCTGGGC | 36 |
| | ACCCTGGTGGGCCTGAGCGCGACCCCGGCGCCGAAAAG | |
| | CTGCCCGGAACGCCATTATTGGGCGCAGGGCAAACTGTG | |
| | CTGCCAGATGTGCGAACCGGGCACCTTTCTGGTGAAAGA | |
| | TTGCGATCAGCATCGCAAAGCGGCGCAGTGCGATCCGTG | |
| | CATTCCGGGCGTGAGCTTTAGCCCGGATCATCATACCCG | |
| | CCCGCATTGCGAAAGCTGCCGCCATTGCAACAGCGGCCT | |
| | GCTGGTGCGCAACTGCACCATTACCGCGAACGCGGAATG | |
| | CGCGTGCCGCAACGGCTGGCAGTGCCGCGATAAAGAAT | |
| | GCACCGAATGCGATCCGCTGCCGAACCCGAGCCTGACCG | |
| | CGCGCAGCAGCCAGGCGCTGAGCCCGCATCCGCAGCCG | |
| | ACCCATCTGCCGTATGTGAGCGAAATGCTGGAAGCGCGC | |
| | ACCGCGGGCCATATGCAGACCCTGGCGGATTTTCGCCAG | |
| | CTGCCGGCGCGCACCCTGAGCACCCATTGGCCGCCGCAG | |
| | CGCAGCCTGTGCAGCAGCGATTTTATTCGCATTCTGGTG | |
| | ATTTTTAGCGGCATGTTTCTGGTGTTTACCCTGGCGGGCG | |
| | CGCTGTTTCTGCATCAGCGCCGCAAATATCGCAGCAACA | |
| | AAGGCGAAAGCCCGGTGGAACCGGCGGAACCGTGCCAT | |
| | TATAGCTGCCCGCGCGAAGAAGAAGGCAGCACCATTCC | |
| | GATTCAGGAAGATTATCGCAAACCGGAACCGGCGTGCA | |
| | GCCCG | |
| Human CD27 | MARPHPWWLCVLGTLVGLSATPAPKSCPERHYWAQGKLC | 37 |
| | CQMCEPGTFLVKDCDQHRKAAQCDPCIPGVSFSPDHHTRP | |
| | HCESCRHCNSGLLVRNCTITANAECACRNGWQCRDKECTE | |
| | CDPLPNPSLTARSSQALSPHPQPTHLPYVSEMLEARTAGHM | |
| | QTLADFRQLPARTLSTHWPPQRSLCSSDFIRILVIFSGMFLVF | |
| | TLAGALFLHQRRKYRSNKGESPVEPAEPCHYSCPREEEGST | |
| | IPIQEDYRKPEPACSP | |
| Murine CD27 | ATGGCGTGGCCGCCGCCGTATTGGCTGTGCATGCTGGGC | 38 |
| | ACCCTGGTGGGCCTGAGCGCGACCCTGGCGCCGAACAG | |
| | CTGCCCGGATAAACATTATTGGACCGGCGGCGGCCTGTG | |
| | CTGCCGCATGTGCGAACCGGGCACCTTTTTTGTGAAAGA | |
| | TTGCGAACAGGATCGCACCGCGGCGCAGTGCGATCCGTG | |
| | CATTCCGGGCACCAGCTTTAGCCCGGATTATCATACCCG | |
| | CCCGCATTGCGAAAGCTGCCGCCATTGCAACAGCGGCTT | |
| | TCTGATTCGCAACTGCACCGTGACCGCGAACGCGGAATG | |
| | CAGCTGCAGCAAAAACTGGCAGTGCCGCGATCAGGAAT | |
| | GCACCGAATGCGATCCGCCGCTGAACCCGGCGCTGACCC | |
| | GCCAGCCGAGCGAAACCCCGAGCCCGCAGCCGCCGCCG | |
| | ACCCATCTGCCGCATGGCACCGAAAAACCGAGCTGGCC | |
| | GCTGCATCGCCAGCTGCCGAACAGCACCGTGTATAGCCA | |
| | GCGCAGCAGCCATCGCCCGCTGTGCAGCAGCGATTGCAT | |
| | TCGCATTTTTGTGACCTTTAGCAGCATGTTTCTGATTTTT | |
| | GTGCTGGGCGCGATTCTGTTTTTTCATCAGCGCCGCAAC | |
| | CATGGCCCGAACGAAGATCGCCAGGCGGTGCCGGAAGA | |
| | ACCGTGCCCGTATAGCTGCCCGCGCGAAGAAGAAGGCA | |
| | GCGCGATTCCGATTCAGGAAGATTATCGCAAACCGGAAC | |
| | CGGCGTTTTATCCG | |
| Murine CD27 | MAWPPPYWLCMLGTLVGLSATLAPNSCPDKHYWTGGGLC | 39 |
| | CRMCEPGTFFVKDCEQDRTAAQCDPCIPGTSFSPDYHTRPH | |
| | CESCRHCNSGFLIRNCTVTANAECSCSKNWQCRDQECTEC | |
| | DPPLNPALTRQPSETPSPQPPPTHLPHGTEKPSWPLHRQLPN | |
| | STVYSQRSSHRPLCSSDCIRIFVTFSSMFLIFVLGAILFFHQRR | |
| | NHGPNEDRQAVPEEPCPYSCPREEEGSAIPIQEDYRKPEPAF | |
| | YP | |
| Human CD28 | ATGCTGCGCCTGCTGCTGGCGCTGAACCTGTTTCCGAGC | 40 |
| | ATTCAGGTGACCGGCAACAAAATTCTGGTGAAACAGAG | |
| | CCCGATGCTGGTGGCGTATGATAACGCGGTGAACCTGAG | |
| | CTGCAAATATAGCTATAACCTGTTTAGCCGCGAATTTCG | |
| | CGCGAGCCTGCATAAAGGCCTGGATAGCGCGGTGGAAG | |
| | TGTGCGTGGTGTATGGCAACTATAGCCAGCAGCTGCAGG | |
| | TGTATAGCAAAACCGGCTTTAACTGCGATGGCAAACTGG | |
| | GCAACGAAAGCGTGACCTTTTATCTGCAGAACCTGTATG | |
| | TGAACCAGACCGATATTTATTTTTGCAAAATTGAAGTGA | |
| | TGTATCCGCCGCCGTATCTGGATAACGAAAAAAGCAACG | |
| | GCACCATTATTCATGTGAAAGGCAAACATCTGTGCCCGA | |
| | GCCCGCTGTTTCCGGGCCCGAGCAAACCGTTTTGGGTGC | |
| | TGGTGGTGGTGGGCGGCGTGCTGGCGTGCTATAGCCTGC | |
| | TGGTGACCGTGGCGTTTATTATTTTTTGGGTGCGCAGCA | |
| | AACGCAGCCGCCTGCTGCATAGCGATTATATGAACATGA | |
| | CCCCGCGCCGCCCGGGCCCGACCCGCAAACATTATCAGC | |
| | CGTATGCGCCGCCGCGCGATTTTGCGGCGTATCGCAGC | |
| Human CD28 | MLRLLLALNLFPSIQVTGNKILVKQSPMLVAYDNAVNLSC | 41 |
| | KYSYNLFSREFRASLHKGLDSAVEVCVVYGNYSQQLQVYS | |
| | KTGFNCDGKLGNESVTFYLQNLYVNQTDIYFCKIEVMYPPP | |
| | YLDNEKSNGTIIHVKGKHLCPSPLFPGPSKPFWVLVVVGGV | |
| | LACYSLLVTVAFIIFWVRSKRSRLLHSDYMNMTPRRPGPTR | |
| | KHYQPYAPPRDFAAYRS | |
| Murine CD28 | ATGACCCTGCGCCTGCTGTTTCTGGCGCTGAACTTTTTTA | 42 |
| | GCGTGCAGGTGACCGAAAACAAAATTCTGGTGAAACAG | |
| | AGCCCGCTGCTGGTGGTGGATAGCAACGAAGTGAGCCT | |
| | GAGCTGCCGCTATAGCTATAACCTGCTGGCGAAAGAATT | |
| | TCGCGCGAGCCTGTATAAAGGCGTGAACAGCGATGTGG | |
| | AAGTGTGCGTGGGCAACGGCAACTTTACCTATCAGCCGC | |
| | AGTTTCGCAGCAACGCGGAATTTAACTGCGATGGCGATT | |
| | TTGATAACGAAACCGTGACCTTTCGCCTGTGGAACCTGC | |
| | ATGTGAACCATACCGATATTTATTTTTGCAAAATTGAATT | |
| | TATGTATCCGCCGCCGTATCTGGATAACGAACGCAGCAA | |
| | CGGCACCATTATTCATATTAAAGAAAAACATCTGTGCCA | |
| | TACCCAGAGCAGCCCGAAACTGTTTTGGGCGCTGGTGGT | |
| | GGTGGCGGGCGTGCTGTTTTGCTATGGCCTGCTGGTGAC | |
| | CGTGGCGCTGTGCGTGATTTGGACCAACAGCCGCCGCAA | |
| | CCGCCTGCTGCAGAGCGATTATATGAACATGACCCCGCG | |
| | CCGCCCGGGCCTGACCCGCAAACCGTATCAGCCGTATGC | |
| | GCCGGCGCGCGATTTTGCGGCGTATCGCCCG | |
| Murine CD28 | MTLRLLFLALNFFSVQVTENKILVKQSPLLVVDSNEVSLSC | 43 |
| | RYSYNLLAKEFRASLYKGVNSDVEVCVGNGNFTYQPQFRS | |
| | NAEFNCDGDFDNETVTFRLWNLHVNHTDIYFCKIEFMYPPP | |
| | YLDNERSNGTIIHIKEKHLCHTQSSPKLFWALVVVAGVLFC | |
| | YGLLVTVALCVIWTNSRRNRLLQSDYMNMTPRRPGLTRKP | |
| | YQPYAPARDFAAYRP | |
| Human | ATGGGAAACAGCTGTTACAACATAGTAGCCACTCTGTTG | 44 |
| CD137 | CTGGTCCTCAACTTTGAGAGGACAAGATCATTGCAGGAT | |
| | CCTTGTAGTAACTGCCCAGCTGGTACATTCTGTGATAAT | |
| | AACAGGAATCAGATTTGCAGTCCCTGTCCTCCAAATAGT | |
| | TTCTCCAGCGCAGGTGGACAAAGGACCTGTGACATATGC | |
| | AGGCAGTGTAAAGGTGTTTTCAGGACCAGGAAGGAGTG | |
| | TTCCTCCACCAGCAATGCAGAGTGTGACTGCACTCCAGG | |
| | GTTTCACTGCCTGGGGGCAGGATGCAGCATGTGTGAACA | |
| | GGATTGTAAACAAGGTCAAGAACTGACAAAAAAAGGTT | |
| | GTAAAGACTGTTGCTTTGGGACATTTAACGATCAGAAAC | |
| | GTGGCATCTGTCGACCCTGGACAAACTGTTCTTTGGATG | |
| | GAAAGTCTGTGCTTGTGAATGGGACGAAGGAGAGGGAC | |
| | GTGGTCTGTGGACCATCTCCAGCCGACCTCTCTCCGGGA | |
| | GCATCCTCTGTGACCCCGCCTGCCCCTGCGAGAGAGCCA | |
| | GGACACTCTCCGCAGATCATCTCCTTCTTTCTTGCGCTGA | |
| | CGTCGACTGCGTTGCTCTTCCTGCTGTTCTTCCTCACGCT | |
| | CCGTTTCTCTGTTGTTAAACGGGGCAGAAAGAAACTCCT | |
| | GTATATATTCAAACAACCATTTATGAGACCAGTACAAAC | |
| | TACTCAAGAGGAAGATGGCTGTAGCTGCCGATTTCCAGA | |
| | AGAAGAAGAAGGAGGATGTGAACTGTGA | |
| Human | MGNSCYNIVATLLLVLNFERTRSLQDPCSNCPAGTFCDNNR | 45 |
| CD137 | NQICSPCPPNSFSSAGGQRTCDICRQCKGVFRTRKECSSTSN | |
| | AECDCTPGFHCLGAGCSMCEQDCKQGQELTKKGCKDCCF | |
| | GTFNDQKRGICRPWTNCSLDGKSVLVNGTKERDVVCGPSP | |
| | ADLSPGASSVTPPAPAREPGHSPQIISFFLALTSTALLFLLFFL | |
| | TLRFSVVKRGRKKLLYIFKQPFMRPVQTTQEEDGCSCRFPE | |
| | EEEGGCEL | |
| Murine | ATGGGCAACAACTGCTATAACGTGGTGGTGATTGTGCTG | 46 |
| CD137 | CTGCTGGTGGGCTGCGAAAAAGTGGGCGCGGTGCAGAA | |
| | CAGCTGCGATAACTGCCAGCCGGGCACCTTTTGCCGCAA | |
| | ATATAACCCGGTGTGCAAAAGCTGCCCGCCGAGCACCTT | |
| | TAGCAGCATTGGCGGCCAGCCGAACTGCAACATTTGCCG | |
| | CGTGTGCGCGGGCTATTTTCGCTTTAAAAAATTTTGCAG | |
| | CAGCACCCATAACGCGGAATGCGAATGCATTGAAGGCTT | |
| | TCATTGCCTGGGCCCGCAGTGCACCCGCTGCGAAAAAGA | |
| | TTGCCGCCCGGGCCAGGAACTGACCAAACAGGGCTGCA | |
| | AAACCTGCAGCCTGGGCACCTTTAACGATCAGAACGGCA | |
| | CCGGCGTGTGCCGCCCGTGGACCAACTGCAGCCTGGATG | |
| | GCCGCAGCGTGCTGAAAACCGGCACCACCGAAAAAGAT | |
| | GTGGTGTGCGGCCCGCCGGTGGTGAGCTTTAGCCCGAGC | |
| | ACCACCATTAGCGTGACCCCGGAAGGCGGCCCGGGCGG | |
| | CCATAGCCTGCAGGTGCTGACCCTGTTTCTGGCGCTGAC | |
| | CAGCGCGCTGCTGCTGGCGCTGATTTTTATTACCCTGCTG | |
| | TTTAGCGTGCTGAAATGGATTCGCAAAAAATTTCCGCAT | |
| | ATTTTTAAACAGCCGTTTAAAAAAACCACCGGCGCGGCG | |
| | CAGGAAGAAGATGCGTGCAGCTGCCGCTGCCCGCAGGA | |
| | AGAAGAAGGCGGCGGCGGCGGCTATGAACTG | |
| Murine | MGNNCYNVVVIVLLLVGCEKVGAVQNSCDNCQPGTFCRK | 47 |
| CD137 | YNPVCKSCPPSTFSSIGGQPNCNICRVCAGYFRFKKFCSSTH | |
| | NAECECIEGFHCLGPQCTRCEKDCRPGQELTKQGCKTCSLG | |
| | TFNDQNGTGVCRPWTNCSLDGRSVLKTGTTEKDVVCGPPV | |
| | VSFSPSTTISVTPEGGPGGHSLQVLTLFLALTSALLLALIFITL | |
| | LFSVLKWIRKKFPHIFKQPFKKTTGAAQEEDACSCRCPQEE | |
| | EGGGGGYEL | |
| Human | ATGTGCGTGGGCGCGCGCCGCCTGGGCCGCGGCCCGTGC | 48 |
| OX40 | GCGGCGCTGCTGCTGCTGGGCCTGGGCCTGAGCACCGTG | |
| | ACCGGCCTGCATTGCGTGGGCGATACCTATCCGAGCAAC | |
| | GATCGCTGCTGCCATGAATGCCGCCCGGGCAACGGCATG | |
| | GTGAGCCGCTGCAGCCGCAGCCAGAACACCGTGTGCCG | |
| | CCCGTGCGGCCCGGGCTTTTATAACGATGTGGTGAGCAG | |
| | CAAACCGTGCAAACCGTGCACCTGGTGCAACCTGCGCAG | |
| | CGGCAGCGAACGCAAACAGCTGTGCACCGCGACCCAGG | |
| | ATACCGTGTGCCGCTGCCGCGCGGGCACCCAGCCGCTGG | |
| | ATAGCTATAAACCGGGCGTGGATTGCGCGCCGTGCCCGC | |
| | CGGGCCATTTTAGCCCGGGCGATAACCAGGCGTGCAAAC | |
| | CGTGGACCAACTGCACCCTGGCGGGCAAACATACCCTGC | |
| | AGCCGGCGAGCAACAGCAGCGATGCGATTTGCGAAGAT | |
| | CGCGATCCGCCGGCGACCCAGCCGCAGGAAACCCAGGG | |
| | CCCGCCGGCGCGCCCGATTACCGTGCAGCCGACCGAAGC | |
| | GTGGCCGCGCACCAGCCAGGGCCCGAGCACCCGCCCGG | |
| | TGGAAGTGCCGGGCGGCCGCGCGGTGGCGGCGATTCTG | |
| | GGCCTGGGCCTGGTGCTGGGCCTGCTGGGCCCGCTGGCG | |
| | ATTCTGCTGGCGCTGTATCTGCTGCGCCGCGATCAGCGC | |
| | CTGCCGCCGGATGCGCATAAACCGCCGGGCGGCGGCAG | |
| | CTTTCGCACCCCGATTCAGGAAGAACAGGCGGATGCGCA | |
| | TAGCACCCTGGCGAAAATT | |
| Human | MCVGARRLGRGPCAALLLLGLGLSTVTGLHCVGDTYPSND | 49 |
| OX40 | RCCHECRPGNGMVSRCSRSQNTVCRPCGPGFYNDVVSSKP | |
| | CKPCTWCNLRSGSERKQLCTATQDTVCRCRAGTQPLDSYK | |
| | PGVDCAPCPPGHFSPGDNQACKPWTNCTLAGKHTLQPASN | |
| | SSDAICEDRDPPATQPQETQGPPARPITVQPTEAWPRTSQGP | |
| | STRPVEVPGGRAVAAILGLGLVLGLLGPLAILLALYLLRRD | |
| | QRLPPDAHKPPGGGSFRTPIQEEQADAHSTLAKI | |
| Murine | ATGTATGTGTGGGTGCAGCAGCCGACCGCGCTGCTGCTG | 50 |
| OX40 | CTGGCGCTGACCCTGGGCGTGACCGCGCGCCGCCTGAAC | |
| | TGCGTGAAACATACCTATCCGAGCGGCCATAAATGCTGC | |
| | CGCGAATGCCAGCCGGGCCATGGCATGGTGAGCCGCTG | |
| | CGATCATACCCGCGATACCCTGTGCCATCCGTGCGAAAC | |
| | CGGCTTTTATAACGAAGCGGTGAACTATGATACCTGCAA | |
| | ACAGTGCACCCAGTGCAACCATCGCAGCGGCAGCGAAC | |
| | TGAAACAGAACTGCACCCCGACCCAGGATACCGTGTGCC | |
| | GCTGCCGCCCGGGCACCCAGCCGCGCCAGGATAGCGGC | |
| | TATAAACTGGGCGTGGATTGCGTGCCGTGCCCGCCGGGC | |
| | CATTTTAGCCCGGGCAACAACCAGGCGTGCAAACCGTGG | |
| | ACCAACTGCACCCTGAGCGGCAAACAGACCCGCCATCC | |
| | GGCGAGCGATAGCCTGGATGCGGTGTGCGAAGATCGCA | |
| | GCCTGCTGGCGACCCTGCTGTGGGAAACCCAGCGCCCGA | |
| | CCTTTCGCCCGACCACCGTGCAGAGCACCACCGTGTGGC | |
| | CGCGCACCAGCGAACTGCCGAGCCCGCCGACCCTGGTG | |
| | ACCCCGGAAGGCCCGGCGTTTGCGGTGCTGCTGGGCCTG | |
| | GGCCTGGGCCTGCTGGCGCCGCTGACCGTGCTGCTGGCG | |
| | CTGTATCTGCTGCGCAAAGCGTGGCGCCTGCCGAACACC | |
| | CCGAAACCGTGCTGGGGCAACAGCTTTCGCACCCCGATT | |
| | CAGGAAGAACATACCGATGCGCATTTTACCCTGGCGAAA | |
| | ATT | |
| Murine | MYVWVQQPTALLLLALTLGVTARRLNCVKHTYPSGHKCC | 51 |
| OX40 | RECQPGHGMVSRCDHTRDTLCHPCETGFYNEAVNYDTCK | |
| | QCTQCNHRSGSELKQNCTPTQDTVCRCRPGTQPRQDSGYK | |
| | LGVDCVPCPPGHFSPGNNQACKPWTNCTLSGKQTRHPASD | |
| | SLDAVCEDRSLLATLLWETQRPTFRPTTVQSTTVWPRTSEL | |
| | PSPPTLVTPEGPAFAVLLGLGLGLLAPLTVLLALYLLRKAW | |
| | RLPNTPKPCWGNSFRTPIQEEHTDAHFTLAKI | |
| Human ICOS | ATGAAAAGCGGCCTGTGGTATTTTTTTCTGTTTTGCCTGC | 52 |
| | GCATTAAAGTGCTGACCGGCGAAATTAACGGCAGCGCG | |
| | AACTATGAAATGTTTATTTTTCATAACGGCGGCGTGCAG | |
| | ATTCTGTGCAAATATCCGGATATTGTGCAGCAGTTTAAA | |
| | ATGCAGCTGCTGAAAGGCGGCCAGATTCTGTGCGATCTG | |
| | ACCAAAACCAAAGGCAGCGGCAACACCGTGAGCATTAA | |
| | AAGCCTGAAATTTTGCCATAGCCAGCTGAGCAACAACAG | |
| | CGTGAGCTTTTTTCTGTATAACCTGGATCATAGCCATGCG | |
| | AACTATTATTTTTGCAACCTGAGCATTTTTGATCCGCCGC | |
| | CGTTTAAAGTGACCCTGACCGGCGGCTATCTGCATATTT | |
| | ATGAAAGCCAGCTGTGCTGCCAGCTGAAATTTTGGCTGC | |
| | CGATTGGCTGCGCGGCGTTTGTGGTGGTGTGCATTCTGG | |
| | GCTGCATTCTGATTTGCTGGCTGACCAAAAAAAAATATA | |
| | GCAGCAGCGTGCATGATCCGAACGGCGAATATATGTTTA | |
| | TGCGCGCGGTGAACACCGCGAAAAAAAGCCGCCTGACC | |
| | GATGTGACCCTG | |
| Human ICOS | MKSGLWYFFLFCLRIKVLTGEINGSANYEMFIFHNGGVQIL | 53 |
| | CKYPDIVQQFKMQLLKGGQILCDLTKTKGSGNTVSIKSLKF | |
| | CHSQLSNNSVSFFLYNLDHSHANYYFCNLSIFDPPPFKVTLT | |
| | GGYLHIYESQLCCQLKFWLPIGCAAFVVVCILGCILICWLTK | |
| | KKYSSSVHDPNGEYMFMRAVNTAKKSRLTDVTL | |
| Murine ICOS | ATGAAACCGTATTTTTGCCGCGTGTTTGTGTTTTGCTTTC | 54 |
| | TGATTCGCCTGCTGACCGGCGAAATTAACGGCAGCGCGG | |
| | ATCATCGCATGTTTAGCTTTCATAACGGCGGCGTGCAGA | |
| | TTAGCTGCAAATATCCGGAAACCGTGCAGCAGCTGAAA | |
| | ATGCGCCTGTTTCGCGAACGCGAAGTGCTGTGCGAACTG | |
| | ACCAAAACCAAAGGCAGCGGCAACGCGGTGAGCATTAA | |
| | AAACCCGATGCTGTGCCTGTATCATCTGAGCAACAACAG | |
| | CGTGAGCTTTTTTCTGAACAACCCGGATAGCAGCCAGGG | |
| | CAGCTATTATTTTTGCAGCCTGAGCATTTTTGATCCGCCG | |
| | CCGTTTCAGGAACGCAACCTGAGCGGCGGCTATCTGCAT | |
| | ATTTATGAAAGCCAGCTGTGCTGCCAGCTGAAACTGTGG | |
| | CTGCCGGTGGGCTGCGCGGCGTTTGTGGTGGTGCTGCTG | |
| | TTTGGCTGCATTCTGATTATTTGGTTTAGCAAAAAAAAA | |
| | TATGGCAGCAGCGTGCATGATCCGAACAGCGAATATATG | |
| | TTTATGGCGGCGGTGAACACCAACAAAAAAAGCCGCCT | |
| | GGCGGGCGTGACCAGC | |
| Murine ICOS | MKPYFCRVFVFCFLIRLLTGEINGSADHRMFSFHNGGVQIS | 55 |
| | CKYPETVQQLKMRLFREREVLCELTKTKGSGNAVSIKNPM | |
| | LCLYHLSNNSVSFFLNNPDSSQGSYYFCSLSIFDPPPFQERN | |
| | LSGGYLHIYESQLCCQLKLWLPVGCAAFVVVLLFGCILIIW | |
| | FSKKKYGSSVHDPNSEYMFMAAVNTNKKSRLAGVTS | |
| Human | ATGATTCATCTGGGCCATATTCTGTTTCTGCTGCTGCTGC | 56 |
| DAP10 | CGGTGGCGGCGGCGCAGACCACCCCGGGCGAACGCAGC | |
| | AGCCTGCCGGCGTTTTATCCGGGCACCAGCGGCAGCTGC | |
| | AGCGGCTGCGGCAGCCTGAGCCTGCCGCTGCTGGCGGGC | |
| | CTGGTGGCGGCGGATGCGGTGGCGAGCCTGCTGATTGTG | |
| | GGCGCGGTGTTTCTGTGCGCGCGCCCGCGCCGCAGCCCG | |
| | GCGCAGGAAGATGGCAAAGTGTATATTAACATGCCGGG | |
| | CCGCGGC | |
| Human | MIHLGHILFLLLLPVAAAQTTPGERSSLPAFYPGTSGSCSGC | 57 |
| DAP10 | GSLSLPLLAGLVAADAVASLLIVGAVFLCARPRRSPAQEDG | |
| | KVYINMPGRG | |
| Murine | ATGGATCCGCCGGGCTATCTGCTGTTTCTGCTGCTGCTGC | 58 |
| DAP10 | CGGTGGCGGCGAGCCAGACCAGCGCGGGCAGCTGCAGC | |
| | GGCTGCGGCACCCTGAGCCTGCCGCTGCTGGCGGGCCTG | |
| | GTGGCGGCGGATGCGGTGATGAGCCTGCTGATTGTGGGC | |
| | GTGGTGTTTGTGTGCATGCGCCCGCATGGCCGCCCGGCG | |
| | CAGGAAGATGGCCGCGTGTATATTAACATGCCGGGCCGC | |
| | GGC | |
| Murine | MDPPGYLLFLLLLPVAASQTSAGSCSGCGTLSLPLLAGLVA | 59 |
| DAP10 | ADAVMSLLIVGVVFVCMRPHGRPAQEDGRVYINMPGRG | |
| Human | ATGGGGGGACTTGAACCCTGCAGCAGGCTCCTGCTCCTG | 60 |
| DAP12 | CCTCTCCTGCTGGCTGTAAGTGGTCTCCGTCCTGTCCAGG | |
| | CCCAGGCCCAGAGCGATTGCAGTTGCTCTACGGTGAGCC | |
| | CGGGCGTGCTGGCAGGGATCGTGATGGGAGACCTGGTG | |
| | CTGACAGTGCTCATTGCCCTGGCCGTGTACTTCCTGGGC | |
| | CGGCTGGTCCCTCGGGGGCGAGGGGCTGCGGAGGCAGC | |
| | GACCCGGAAACAGCGTATCACTGAGACCGAGTCGCCTTA | |
| | TCAGGAGCTCCAGGGTCAGAGGTCGGATGTCTACAGCG | |
| | ACCTCAACACACAGAGGCCGTATTACAAATGA | |
| Human | MGGLEPCSRLLLLPLLLAVSGLRPVQAQAQSDCSCSTVSPG | 61 |
| DAP12 | VLAGIVMGDLVLTVLIALAVYFLGRLVPRGRGAAEAATRK | |
| | QRITETESPYQELQGQRSDVYSDLNTQRPYYK | |
| Murine | ATGGGGGCTCTGGAGCCCTCCTGGTGCCTTCTGTTCCTTC | 62 |
| DAP12 | CTGTCCTCCTGACTGTGGGAGGATTAAGTCCCGTACAGG | |
| | CCCAGAGTGACACTTTCCCAAGATGCGACTGTTCTTCCG | |
| | TGAGCCCTGGTGTACTGGCTGGGATTGTTCTGGGTGACT | |
| | TGGTGTTGACTCTGCTGATTGCCCTGGCTGTGTACTCTCT | |
| | GGGCCGCCTGGTCTCCCGAGGTCAAGGGACAGCGGAAG | |
| | GGACCCGGAAACAACACATTGCTGAGACTGAGTCGCCTT | |
| | ATCAGGAGCTTCAGGGTCAGAGACCAGAAGTATACAGT | |
| | GACCTCAACACACAGAGGCAATATTACAGATGA | |
| Murine | MGALEPSWCLLFLPVLLTVGGLSPVQAQSDTFPRCDCSSVS | 63 |
| DAP12 | PGVLAGIVLGDLVLTLLIALAVYSLGRLVSRGQGTAEGTRK | |
| | QHIAETESPYQELQGQRPEVYSDLNTQRQYYR | |
| Human CD3z | MKWKALFTAAILQAQLPITEAQSFGLLDPKLCYLLDGILFIY | 64 |
| | GVILTALFLRVKFSRSADAPAYQQGQNQLYNELNLGRREE | |
| | YDVLDKRRGRDPEMGGKPQRRKNPQEGLYNELQKDKMA | |
| | EAYSEIGMKGERRRGKGHDGLYQGLSTATKDTYDALHMQ | |
| | ALPPR | |
| Human CD3z | ATGAAGTGGAAGGCGCTTTTCACCGCGGCCATCCTGCAG | 65 |
| | GCACAGTTGCCGATTACAGAGGCACAGAGCTTTGGCCTG | |
| | CTGGATCCCAAACTCTGCTACCTGCTGGATGGAATCCTC | |
| | TTCATCTATGGTGTCATTCTCACTGCCTTGTTCCTGAGAG | |
| | TGAAGTTCAGCAGGAGCGCAGAGCCCCCCGCGTACCAG | |
| | CAGGGCCAGAACCAGCTCTATAACGAGCTCAATCTAGG | |
| | ACGAAGAGAGGAGTACGATGTTTTGGACAAGAGACGTG | |
| | GCCGGGACCCTGAGATGGGGGGAAAGCCGAGAAGGAAG | |
| | AACCCTCAGGAAGGCCTGTACAATGAACTGCAGAAAGA | |
| | TAAGATGGCGGAGGCCTACAGTGAGATTGGGATGAAAG | |
| | GCGAGCGCCGGAGGGGCAAGGGGCACGATGGCCTTTAC | |
| | CAGGGTCTCAGTACAGCCACCAAGGACACCTACGACGC | |
| | CCTTCACATGCAGGCCCTGCCCCCTCGCTAA | |
| Murine CD3z | MKWKVSVLACILHVRFPGAEAQSFGLLDPKLCYLLDGILFI | 66 |
| | YGVIITALYLRAKFSRSAETAANLQDPNQLYNELNLGRREE | |
| | YDVLEKKRARDPEMGGKQQRRRNPQEGVYNALQKDKMA | |
| | EAYSEIGTKGERRRGKGHDGLYQGLSTATKDTYDALHMQ | |
| | TLAPR | |
| Murine CD3z | ATGAAGTGGAAAGTGTCTGTTCTCGCCTGCATCCTCCAC | 67 |
| | GTGCGGTTCCCAGGAGCAGAGGCACAGAGCTTTGGTCTG | |
| | CTGGATCCCAAACTCTGCTACTTGCTAGATGGAATCCTC | |
| | TTCATCTACGGAGTCATCATCACAGCCCTGTACCTGAGA | |
| | GCAAAATTCAGCAGGAGTGCAGAGACTGCTGCCAACCT | |
| | GCAGGACCCCAACCAGCTCTACAATGAGCTCAATCTAGG | |
| | GCGAAGAGAGGAATATGACGTCTTGGAGAAGAAGCGGG | |
| | CTCGGGATCCAGAGATGGGAGGCAAACAGCAGAGGAGG | |
| | AGGAACCCCCAGGAAGGCGTATACAATGCACTGCAGAA | |
| | AGACAAGATGGCAGAAGCCTACAGTGAGATCGGCACAA | |
| | AAGGCGAGAGGCGGAGAGGCAAGGGGCACGATGGCCTT | |
| | TACCAGGGTCTCAGCACTGCCACCAAGGACACCTATGAT | |
| | GCCCTGCATATGCAGACCCTGGCCCCTCGCTAA | |
| Human | MWQLLLPTALLLLVSAGMRTEDLPKAVVFLEPQWYRVLE | 68 |
| FCGR3A | KDSVTLKCQGAYSPEDNSTQWFHNESLISSQASSYFIDAAT | |
| | VDDSGEYRCQTNLSTLSDPVQLEVHIGWLLLQAPRWVFKE | |
| | EDPIHLRCHSWKNTALHKVTYLQNGKGRKYFHHNSDFYIP | |
| | KATLKDSGSYFCRGLFGSKNVSSETVNITITQGLAVSTISSFF | |
| | PPGYQVSFCLVMVLLFAVDTGLYFSVKTNIRSSTRDWKDH | |
| | KFKWRKDPQDK | |
| Human | ATGTGGCAGCTGCTGCTGCCGACCGCGCTGCTGCTGCTG | 69 |
| FCGR3A | GTGAGCGCGGGCATGCGCACCGAAGATCTGCCGAAAGC | |
| | GGTGGTGTTTCTGGAACCGCAGTGGTATCGCGTGCTGGA | |
| | AAAAGATAGCGTGACCCTGAAATGCCAGGGCGCGTATA | |
| | GCCCGGAAGATAACAGCACCCAGTGGTTTCATAACGAA | |
| | AGCCTGATTAGCAGCCAGGCGAGCAGCTATTTTATTGAT | |
| | GCGGCGACCGTGGATGATAGCGGCGAATATCGCTGCCA | |
| | GACCAACCTGAGCACCCTGAGCGATCCGGTGCAGCTGG | |
| | AAGTGCATATTGGCTGGCTGCTGCTGCAGGCGCCGCGCT | |
| | GGGTGTTTAAAGAAGAAGATCCGATTCATCTGCGCTGCC | |
| | ATAGCTGGAAAAACACCGCGCTGCATAAAGTGACCTATC | |
| | TGCAGAACGGCAAAGGCCGCAAATATTTTCATCATAACA | |
| | GCGATTTTTATATTCCGAAAGCGACCCTGAAAGATAGCG | |
| | GCAGCTATTTTTGCCGCGGCCTGTTTGGCAGCAAAAACG | |
| | TGAGCAGCGAAACCGTGAACATTACCATTACCCAGGGCC | |
| | TGGCGGTGAGCACCATTAGCAGCTTTTTTCCGCCGGGCT | |
| | ATCAGGTGAGCTTTTGCCTGGTGATGGTGCTGCTGTTTGC | |
| | GGTGGATACCGGCCTGTATTTTAGCGTGAAAACCAACAT | |
| | TCGCAGCAGCACCCGCGATTGGAAAGATCATAAATTTAA | |
| | ATGGCGCAAAGATCCGCAGGATAAA | |
| Murine | MFQNAHSGSQWLLPPLTILLLFAFADRQSAALPKAVVKLD | 70 |
| FCGR3A | PPWIQVLKEDMVTLMCEGTHNPGNSSTQWFHNGRSIRSQV | |
| | QASYTFKATVNDSGEYRCQMEQTRLSDPVDLGVISDWLLL | |
| | QTPQRVFLEGETITLRCHSWRNKLLNRISFFHNEKSVRYHH | |
| | YKSNFSIPKANHSHSGDYYCKGSLGSTQHQSKPVTITVQDP | |
| | ATTSSISLVWYHTAFSLVMCLLFAVDTGLYFYVRRNLQTPR | |
| | EYWRKSLSIRKHQAPQDK | |
| Murine | ATGTTTCAGAATGCACACTCTGGAAGCCAATGGCTACTT | 71 |
| FCGR3A | CCACCACTGACAATTCTGCTGCTGTTTGCTTTTGCAGACA | |
| | GGCAGAGTGCAGCTCTTCCGAAGGCTGTGGTGAAACTGG | |
| | ACCCCCCATGGATCCAGGTGCTCAAGGAAGACATGGTG | |
| | ACACTGATGTGCGAAGGGACCCACAACCCTGGGAACTCT | |
| | TCTACCCAGTGGTTCCACAACGGGAGGTCCATCCGGAGC | |
| | CAGGTCCAAGCCAGTTACACGTTTAAGGCCACAGTCAAT | |
| | GACAGTGGAGAATATCGGTGTCAAATGGAGCAGACCCG | |
| | CCTCAGCGACCCTGTAGATCTGGGAGTGATTTCTGACTG | |
| | GCTGCTGCTCCAGACCCCTCAGCGGGTGTTTCTGGAAGG | |
| | GGAAACCATCACGCTAAGGTGCCATAGCTGGAGGAACA | |
| | AACTACTGAACAGGATCTCATTCTTCCATAATGAAAAAT | |
| | CCGTGAGGTATCATCACTACAAAAGTAATTTCTCTATCC | |
| | CAAAAGCCAACCACAGTCACAGTGGGGACTACTACTGC | |
| | AAAGGAAGTCTAGGAAGTACACAGCACCAGTCCAAGCC | |
| | TGTCACCATCACTGTCCAAGATCCAGCAACTACATCCTC | |
| | CATCTCTCTAGTCTGGTACCACACTGCTTTCTCCCTAGTG | |
| | ATGTGCCTCCTGTTTGCAGTGGACACGGGCCTTTATTTCT | |
| | ACGTACGGAGAAATCTTCAAACCCCGAGGGAGTACTGG | |
| | AGGAAGTCCCTGTCAATCAGAAAGCACCAGGCTCCTCAA | |
| | GACAAGTGA | |
| Human | MGWIRGRRSRHSWEMSEFHNYNLDLKKSDFSTRWQKQRC | 72 |
| NKG2D | PVVKSKCRENASPFFFCCFIAVAMGIRFIIMVAIWSAVFLNS | |
| | LFNQEVQIPLTESYCGPCPKNWICYKNNCYQFFDESKNWY | |
| | ESQASCMSQNASLLKVYSKEDQDLLKLVKSYHWMGLVHI | |
| | PTNGSWQWEDGSILSPNLLTIIEMQKGDCALYASSFKGYIE | |
| | NCSTPNTYICMQRTV | |
| Human | ATGGGCTGGATTCGCGGCCGCCGCAGCCGCCATAGCTGG | 73 |
| NKG2D | GAAATGAGCGAATTTCATAACTATAACCTGGATCTGAAA | |
| | AAAAGCGATTTTAGCACCCGCTGGCAGAAACAGCGCTG | |
| | CCCGGTGGTGAAAAGCAAATGCCGCGAAAACGCGAGCC | |
| | CGTTTTTTTTTTGCTGCTTTATTGCGGTGGCGATGGGCAT | |
| | TCGCTTTATTATTATGGTGGCGATTTGGAGCGCGGTGTTT | |
| | CTGAACAGCCTGTTTAACCAGGAAGTGCAGATTCCGCTG | |
| | ACCGAAAGCTATTGCGGCCCGTGCCCGAAAAACTGGATT | |
| | TGCTATAAAAACAACTGCTATCAGTTTTTTGATGAAAGC | |
| | AAAAACTGGTATGAAAGCCAGGCGAGCTGCATGAGCCA | |
| | GAACGCGAGCCTGCTGAAAGTGTATAGCAAAGAAGATC | |
| | AGGATCTGCTGAAACTGGTGAAAAGCTATCATTGGATGG | |
| | GCCTGGTGCATATTCCGACCAACGGCAGCTGGCAGTGGG | |
| | AAGATGGCAGCATTCTGAGCCCGAACCTGCTGACCATTA | |
| | TTGAAATGCAGAAAGGCGATTGCGCGCTGTATGCGAGC | |
| | AGCTTTAAAGGCTATATTGAAAACTGCAGCACCCCGAAC | |
| | ACCTATATTTGCATGCAGCGCACCGTG | |
| Murine | MALIRDRKSHHSEMSKCHNYDLKPAKWDTSQEQQKQRLA | 74 |
| NKG2D | LTTSQPGENGIIRGRYPIEKLKISPMFVVRVLAIALAIRFTLN | |
| | TLMWLAIFKETFQPVLCNKEVPVSSREGYCGPCPNNWICH | |
| | RNNCYQFFNEEKTWNQSQASCLSQNSSLLKIYSKEEQDFLK | |
| | LVKSYHWMGLVQIPANGSWQWEDGSSLSYNQLTLVEIPK | |
| | GSCAVYGSSFKAYTEDCANLNTYICMKRAV | |
| Murine | ATGGCGCTGATTCGCGATCGCAAAAGCCATCATAGCGAA | 75 |
| NKG2D | ATGAGCAAATGCCATAACTATGATCTGAAACCGGCGAA | |
| | ATGGGATACCAGCCAGGAACAGCAGAAACAGCGCCTGG | |
| | CGCTGACCACCAGCCAGCCGGGCGAAAACGGCATTATTC | |
| | GCGGCCGCTATCCGATTGAAAAACTGAAAATTAGCCCGA | |
| | TGTTTGTGGTGCGCGTGCTGGCGATTGCGCTGGCGATTC | |
| | GCTTTACCCTGAACACCCTGATGTGGCTGGCGATTTTTA | |
| | AAGAAACCTTTCAGCCGGTGCTGTGCAACAAAGAAGTG | |
| | CCGGTGAGCAGCCGCGAAGGCTATTGCGGCCCGTGCCCG | |
| | AACAACTGGATTTGCCATCGCAACAACTGCTATCAGTTT | |
| | TTTAACGAAGAAAAAACCTGGAACCAGAGCCAGGCGAG | |
| | CTGCCTGAGCCAGAACAGCAGCCTGCTGAAAATTTATAG | |
| | CAAAGAAGAACAGGATTTTCTGAAACTGGTGAAAAGCT | |
| | ATCATTGGATGGGCCTGGTGCAGATTCCGGCGAACGGCA | |
| | GCTGGCAGTGGGAAGATGGCAGCAGCCTGAGCTATAAC | |
| | CAGCTGACCCTGGTGGAAATTCCGAAAGGCAGCTGCGC | |
| | GGTGTATGGCAGCAGCTTTAAAGCGTATACCGAAGATTG | |
| | CGCGAACCTGAACACCTATATTTGCATGAAACGCGCGGT | |
| | G | |
| CD28 | YMNM | 76 |
| YMNM | | |
| CD28 PYAP | PYAP | 77 |
| CD28 | FMNM | 78 |
| FMNM | | |
| CD28 AYAA | AYAA | 79 |
| Signal | ATMGWSCIILFLVATATGVHS | 80 |
| peptide | | |
| Signal | ATGGGATGGAGCTGTATCATCCTCTTCTTGGTAGCAACA | 81 |
| peptide DNA | GCTACCGGTGTGCACTCC | |
| sequence | | |
| Human CD8 | MRNQAPGRPKGATFPPRRPTGSRAPPLAPELRAKQRPGERV | 82 |
| | MALPVTALLLPLALLLHAARPSQFRVSPLDRTWNLGETVE | |
| | LKCQVLLSNPTSGCSWLFQPRGAAASPTFLLYLSQNKPKAA | |
| | EGLDTQRFSGKRLGDTFVLTLSDFRRENEGYYFCSALSNSI | |
| | MYFSHFVPVFLPAKPTTTPAPRPPTPAPTIASQPLSLRPEACR | |
| | PAAGGAVHTRGLDFACDIYIWAPLAGTCGVLLLSLVITLYC | |
| | NHRNRRRVCKCPRPVVKSGDKPSLSARYV | |
| Human CD8 | ATGCGCAACCAGGCGCCGGGCCGCCCGAAAGGCGCGAC | 83 |
| | CTTTCCGCCGCGCCGCCCGACCGGCAGCCGCGCGCCGCC | |
| | GCTGGCGCCGGAACTGCGCGCGAAACAGCGCCCGGGCG | |
| | AACGCGTGATGGCGCTGCCGGTGACCGCGCTGCTGCTGC | |
| | CGCTGGCGCTGCTGCTGCATGCGGCGCGCCCGAGCCAGT | |
| | TTCGCGTGAGCCCGCTGGATCGCACCTGGAACCTGGGCG | |
| | AAACCGTGGAACTGAAATGCCAGGTGCTGCTGAGCAAC | |
| | CCGACCAGCGGCTGCAGCTGGCTGTTTCAGCCGCGCGGC | |
| | GCGGCGGCGAGCCCGACCTTTCTGCTGTATCTGAGCCAG | |
| | AACAAACCGAAAGCGGCGGAAGGCCTGGATACCCAGCG | |
| | CTTTAGCGGCAAACGCCTGGGCGATACCTTTGTGCTGAC | |
| | CCTGAGCGATTTTCGCCGCGAAAACGAAGGCTATTATTT | |
| | TTGCAGCGCGCTGAGCAACAGCATTATGTATTTTAGCCA | |
| | TTTTGTGCCGGTGTTTCTGCCGGCGAAACCGACCACCAC | |
| | CCCGGCGCCGCGCCCGCCGACCCCGGCGCCGACCATTGC | |
| | GAGCCAGCCGCTGAGCCTGCGCCCGGAAGCGTGCCGCC | |
| | CGGCGGCGGGCGGCGCGGTGCATACCCGCGGCCTGGAT | |
| | TTTGCGTGCGATATTTATATTTGGGCGCCGCTGGCGGGC | |
| | ACCTGCGGCGTGCTGCTGCTGAGCCTGGTGATTACCCTG | |
| | TATTGCAACCATCGCAACCGCCGCCGCGTGTGCAAATGC | |
| | CCGCGCCCGGTGGTGAAAAGCGGCGATAAACCGAGCCT | |
| | GAGCGCGCGCTATGTG | |
| Murine CD8 | MASPLTRFLSLNLLLMGESIILGSGEAKPQAPELRIFPKKMD | 84 |
| | AELGQKVDLVCEVLGSVSQGCSWLFQNSSSKLPQPTFVVY | |
| | MASSHNKITWDEKLNSSKLFSAVRDTNNKYVLTLNKFSKE | |
| | NEGYYFCSVISNSVMYFSSVVPVLQKVNSTTTKPVLRTPSP | |
| | VHPTGTSQPQRPEDCRPRGSVKGTGLDFACDIYIWAPLAGI | |
| | CVAPLLSLIITLICYHRSRKRVCKCPRPLVRQEGKPRPSEKIV | |
| Murine CD8 | ATGGCGAGCCCGCTGACCCGCTTTCTGAGCCTGAACCTG | 85 |
| | CTGCTGATGGGCGAAAGCATTATTCTGGGCAGCGGCGAA | |
| | GCGAAACCGCAGGCGCCGGAACTGCGCATTTTTCCGAAA | |
| | AAAATGGATGCGGAACTGGGCCAGAAAGTGGATCTGGT | |
| | GTGCGAAGTGCTGGGCAGCGTGAGCCAGGGCTGCAGCT | |
| | GGCTGTTTCAGAACAGCAGCAGCAAACTGCCGCAGCCG | |
| | ACCTTTGTGGTGTATATGGCGAGCAGCCATAACAAAATT | |
| | ACCTGGGATGAAAAACTGAACAGCAGCAAACTGTTTAG | |
| | CGCGGTGCGCGATACCAACAACAAATATGTGCTGACCCT | |
| | GAACAAATTTAGCAAAGAAAACGAAGGCTATTATTTTTG | |
| | CAGCGTGATTAGCAACAGCGTGATGTATTTTAGCAGCGT | |
| | GGTGCCGGTGCTGCAGAAAGTGAACAGCACCACCACCA | |
| | AACCGGTGCTGCGCACCCCGAGCCCGGTGCATCCGACCG | |
| | GCACCAGCCAGCCGCAGCGCCCGGAAGATTGCCGCCCG | |
| | CGCGGCAGCGTGAAAGGCACCGGCCTGGATTTTGCGTGC | |
| | GATATTTATATTTGGGCGCCGCTGGCGGGCATTTGCGTG | |
| | GCGCCGCTGCTGAGCCTGATTATTACCCTGATTTGCTATC | |
| | ATCGCAGCCGCAAACGCGTGTGCAAATGCCCGCGCCCGC | |
| | TGGTGCGCCAGGAAGGCAAACCGCGCCCGAGCGAAAAA | |
| | ATTGTG | |

TABLE 6
Exemplary CH2 P329G chimeric receptor
amino acid sequences:

| | | SEQ |
|---|---|---|
| | | ID |
| Construct | Amino acid sequence | NO |
| CH2/P329G | APEAAGGPSVFLFPPKPKDTLMISRTPEVT | 86 |
| LALA | CVVVDVSHEDPEVKFNWYVDGVEVHNAKTK | |
| | PREEQYNSTYRVVSVLTVLHQDWLNGKEYK | |
| | CKVSNKALGAPIEKTISKAK | |
| CH2- | APEAAGGPSVFLFPPKPKDTLMISRTPEVT | 87 |
| CD8a_stalk- | CVVVDVSHEDPEVKFNWYVDGVEVHNAKTK | |
| CD8a_TD- | PREEQYNSTYRVVSVLTVLHQDWLNGKEYK | |
| CD137CSD- | CKVSNKALGAPIEKTISKAKGGGGSAKPTT | |
| CD3zSSD | TPAPRPPTPAPTIASQPLSLRPEACRPAAG | |
| fusion | GAVHTRGLDFACDIYIWAPLAGTCGVLLLS | |
| | LVITKRGRKKLLYIFKQPFMRPVQTTQEED | |
| | GCSCRFPEEEEGGCELRVKFSRSADAPAYQ | |
| | QGQNQLYNELNLGRREEYDVLDKRRGRDPE | |
| | MGGKPQRRKNPQEGLYNELQKDKMAEAYSE | |
| | IGMKGERRRGKGHDGLYQGLSTATKDTYDA | |
| | LHMQALPPR | |
| CD8a_stalk | AKPTTTPAPRPPTPAPTIASQPLSLRPEAC | 88 |
| | RPAAGGAVHTRGLDFACD | |
| CD8a_TD | IYIWAPLAGTCGVLLLSLVIT | 89 |
| CD137CSD | KRGRKKLLYIFKQPFMRPVQTTQEEDGCSC | 90 |
| | RFPEEEEGGCEL | |
| CD3zSSD | RVKFSRSADAPAYQQGQNQLYNELNLGRRE | 91 |
| | EYDVLDKRRGRDPEMGGKPQRRKNPQEGLY | |
| | NELQKDKMAEAYSEIGMKGERRRGKGHDGL | |
| | YQGLSTATKDTYDALHMQALPPR | |
| CD8a_stalk- | AKPTTTPAPRPPTPAPTIASQPLSLRPEAC | 92 |
| CD8a_TD- | RPAAGGAVHTRGLDFACDIYIWAPLAGTCG | |
| CD137CSD- | VLLLSLVITKRGRKKLLYIFKQPFMRPVQT | |
| CD3zSSD | TQEEDGCSCRFPEEEEGGCELRVKFSRSAD | |
| | APAYQQGQNQLYNELNLGRREEYDVLDKRR | |
| | GRDPEMGGKPQRRKNPQEGLYNELQKDKMA | |
| | EAYSEIGMKGERRRGKGHDGLYQGLSTATK | |
| | DTYDALHMQALPPR | |
| eGFP | VSKGEELFTGVVPILVELDGDVNGHKFSVS | 93 |
| | GEGEGDATYGKLTLKFICTTGKLPVPWPTL | |
| | VTTLTYGVQCFSRYPDHMKQHDFFKSAMPE | |
| | GYVQERTIFFKDDGNYKTRAEVKFEGDTLV | |
| | NRIELKGIDFKEDGNILGHKLEYNYNSHNV | |
| | YIMADKQKNGIKVNFKIRHNIEDGSVQLAD | |
| | HYQQNTPIGDGPVLLPDNHYLSTQSALSKD | |
| | PNEKRDHMVLLEFVTAAGITLGMDELYK | |
| G4S linker | GGGGS | 94 |
| T2A linker | GSGEGRGSLLTCGDVEENPGP | 95 |

TABLE 7
Exemplary CH2 P329G chimeric
receptor DNA sequences:

| | | SEQ |
|---|---|---|
| | | ID |
| Construct | DNA sequence | NO |
| CH2- | GCACCTGAAGCTGCAGGGGGACCGTCAGTC | 96 |
| CD8a_stalk- | TTCCTCTTCCCCCCAAAACCCAAGGACACC | |
| CD8a_TD- | CTCATGATCTCCCGGACCCCTGAGGTCACA | |
| CD137CSD- | TGCGTGGTGGTGGACGTGAGCCACGAAGAC | |
| CD3zSSD | CCTGAGGTCAAGTTCAACTGGTACGTGGAC | |
| fusion | GGCGTGGAGGTGCATAATGCCAAGACAAAG | |
| | CCGCGGGAGGAGCAGTACAACAGCACGTAC | |
| | CGTGTGGTCAGCGTCCTCACCGTCCTGCAC | |
| | CAGGACTGGCTGAATGGCAAGGAGTACAAG | |
| | TGCAAGGTCTCCAACAAAGCCCTCGGCGCC | |
| | CCCATCGAGAAAACCATCTCCAAAGCCAAA | |
| | GGCGGGGGAGGATCCGCCAAGCCCACCACG | |
| | ACGCCAGCGCCGCGACCACCAACACCGGCG | |
| | CCCACCATCGCGTCGCAGCCCCTGTCCCTG | |
| | CGCCCAGAGGCGTGCCGGCCAGCGGCGGGG | |
| | GGCGCAGTGCACACGAGGGGGCTGGACTTC | |
| | GCCTGTGATATCTACATCTGGGCGCCCCTG | |
| | GCCGGGACTTGTGGGGTCCTTCTCCTGTCA | |
| | CTGGTTATCACCAAACGGGGCAGAAAGAAA | |
| | CTCCTGTATATATTCAAACAACCATTTATG | |
| | AGACCAGTACAAACTACTCAAGAGGAAGAT | |
| | GGCTGTAGCTGCCGATTTCCAGAAGAAGAA | |
| | GAAGGAGGATGTGAACTGCGGGTGAAATTC | |
| | TCCCGTTCAGCTGACGCACCCGCGTATCAA | |
| | CAGGGCCAGAACCAACTGTACAATGAGCTC | |
| | AACCTGGGTCGTAGAGAAGAGTACGATGTG | |
| | CTGGACAAGCGCCGGGGCCGCGACCCTGAG | |
| | ATGGGTGGAAAGCCCCAGCGCCGGAAGAAT | |
| | CCCCAAGAGGGACTGTATAACGAGCTGCAG | |
| | AAGGACAAGATGGCTGAGGCCTACAGCGAG | |
| | ATCGGTATGAAGGGCGAGAGACGTAGAGGC | |
| | AAGGGCCATGACGGGCTGTACCAAGGGCTT | |
| | AGTACCGCTACAAAGGATACCTACGACGCC | |
| | CTGCACATGCAGGCCCTGCCCCCTCGT | |
| CD8a_ | GCCAAGCCCACCACGACGCCAGCGCCGCGA | 97 |
| stalk | CCACCAACACCGGCGCCCACCATCGCGTCG | |
| | CAGCCCCTGTCCCTGCGCCCAGAGGCGTGC | |
| | CGGCCAGCGGCGGGGGGCGCAGTGCACACG | |
| | AGGGGGCTGGACTTCGCCTGTGAT | |
| CD8a_TD | ATCTACATCTGGGCGCCCCTGGCCGGGACT | 98 |
| | TGTGGGGTCCTTCTCCTGTCACTGGTTATC | |
| | ACC | |
| CD137CSD | AAACGGGGCAGAAAGAAACTCCTGTATATA | 99 |
| | TTCAAACAACCATTTATGAGACCAGTACAA | |
| | ACTACTCAAGAGGAAGATGGCTGTAGCTGC | |
| | CGATTTCCAGAAGAAGAAGAAGGAGGATGT | |
| | GAACTG | |
| CD3zSSD | CGGGTGAAATTCTCCCGTTCAGCTGACGCA | 100 |
| | CCCGCGTATCAACAGGGCCAGAACCAACTG | |
| | TACAATGAGCTCAACCTGGGTCGTAGAGAA | |
| | GAGTACGATGTGCTGGACAAGCGCCGGGGC | |
| | CGCGACCCTGAGATGGGTGGAAAGCCCCAG | |
| | CGCCGGAAGAATCCCCAAGAGGGACTGTAT | |
| | AACGAGCTGCAGAAGGACAAGATGGCTGAG | |
| | GCCTACAGCGAGATCGGTATGAAGGGCGAG | |
| | AGACGTAGAGGCAAGGGCCATGACGGGCTG | |
| | TACCAAGGGCTTAGTACCGCTACAAAGGAT | |
| | ACCTACGACGCCCTGCACATGCAGGCCCTG | |
| | CCCCCTCGT | |
| CD8a_stalk- | GCCAAGCCCACCACGACGCCAGCGCCGCGA | 101 |
| CD8a_TD- | CCACCAACACCGGCGCCCACCATCGCGTCG | |
| CD137CSD- | CAGCCCCTGTCCCTGCGCCCAGAGGCGTGC | |
| CD3zSSD | CGGCCAGCGGCGGGGGGCGCAGTGCACACG | |
| | AGGGGGCTGGACTTCGCCTGTGATATCTAC | |
| | ATCTGGGCGCCCCTGGCCGGGACTTGTGGG | |
| | GTCCTTCTCCTGTCACTGGTTATCACCAAA | |
| | CGGGGCAGAAAGAAACTCCTGTATATATTC | |
| | AAACAACCATTTATGAGACCAGTACAAACT | |
| | ACTCAAGAGGAAGATGGCTGTAGCTGCCGA | |
| | TTTCCAGAAGAAGAAGAAGGAGGATGTGAA | |
| | CTGCGGGTGAAATTCTCCCGTTCAGCTGAC | |
| | GCACCCGCGTATCAACAGGGCCAGAACCAA | |
| | CTGTACAATGAGCTCAACCTGGGTCGTAGA | |
| | GAAGAGTACGATGTGCTGGACAAGCGCCGG | |
| | GGCCGCGACCCTGAGATGGGTGGAAAGCCC | |
| | CAGCGCCGGAAGAATCCCCAAGAGGGACTG | |
| | TATAACGAGCTGCAGAAGGACAAGATGGCT | |
| | GAGGCCTACAGCGAGATCGGTATGAAGGGC | |
| | GAGAGACGTAGAGGCAAGGGCCATGACGGG | |
| | CTGTACCAAGGGCTTAGTACCGCTACAAAG | |
| | GATACCTACGACGCCCTGCACATGCAGGCC | |
| | CTGCCCCCTCGT | |
| T2A element | GGTTCCGGAGAGGGCAGAGGCTCTCTGCTC | 102 |
| | ACCTGCGGAGATGTGGAGGAAAACCCAGGA | |
| | CCT | |
| eGFP | GTGAGCAAGGGCGAGGAGCTGTTCACCGGG | 103 |
| | GTGGTGCCCATCCTGGTCGAGCTGGACGGC | |
| | GACGTAAACGGCCACAAGTTCAGCGTGTCC | |
| | GGCGAGGGCGAGGGCGATGCCACCTACGGC | |
| | AAGCTGACCCTGAAGTTCATCTGCACCACC | |
| | GGCAAGCTGCCCGTGCCCTGGCCCACCCTC | |
| | GTGACCACCCTGACCTACGGCGTGCAGTGC | |
| | TTCAGCCGCTACCCCGACCACATGAAGCAG | |
| | CACGACTTCTTCAAGTCCGCCATGCCCGAA | |
| | GGCTACGTCCAGGAGCGCACCATCTTCTTC | |
| | AAGGACGACGGCAACTACAAGACCCGCGCC | |
| | GAGGTGAAGTTCGAGGGCGACACCCTGGTG | |
| | AACCGCATCGAGCTGAAGGGCATCGACTTC | |
| | AAGGAGGACGGCAACATCCTGGGGCACAAG | |
| | CTGGAGTACAACTACAACAGCCACAACGTC | |
| | TATATCATGGCCGACAAGCAGAAGAACGGC | |
| | ATCAAGGTGAACTTCAAGATCCGCCACAAC | |
| | ATCGAGGACGGCAGCGTGCAGCTCGCCGAC | |
| | CACTACCAGCAGAACACCCCCATCGGCGAC | |
| | GGCCCCGTGCTGCTGCCCGACAACCACTAC | |
| | CTGAGCACCCAGTCCGCCCTGAGCAAAGAC | |
| | CCCAACGAGAAGCGCGATCACATGGTCCTG | |
| | CTGGAGTTCGTGACCGCCGCCGGGATCACT | |
| | CTCGGCATGGACGAGCTGTACAAGTGA | |
| CH2- | GCACCTGAAGCTGCAGGGGGACCGTCAGTC | 104 |
| CD8a_stalk- | TTCCTCTTCCCCCCAAAACCCAAGGACACC | |
| CD8a_TD- | CTCATGATCTCCCGGACCCCTGAGGTCACA | |
| CD137CSD- | TGCGTGGTGGTGGACGTGAGCCACGAAGAC | |
| CD3zSSD | CCTGAGGTCAAGTTCAACTGGTACGTGGAC | |
| GFP fusion | GGCGTGGAGGTGCATAATGCCAAGACAAAG | |
| | CCGCGGGAGGAGCAGTACAACAGCACGTAC | |
| | CGTGTGGTCAGCGTCCTCACCGTCCTGCAC | |
| | CAGGACTGGCTGAATGGCAAGGAGTACAAG | |
| | TGCAAGGTCTCCAACAAAGCCCTCGGCGCC | |
| | CCCATCGAGAAAACCATCTCCAAAGCCAAA | |
| | GGCGGGGGAGGATCCGCCAAGCCCACCACG | |
| | ACGCCAGCGCCGCGACCACCAACACCGGCG | |
| | CCCACCATCGCGTCGCAGCCCCTGTCCCTG | |
| | CGCCCAGAGGCGTGCCGGCCAGCGGCGGGG | |
| | GGCGCAGTGCACACGAGGGGGCTGGACTTC | |
| | GCCTGTGATATCTACATCTGGGCGCCCCTG | |
| | GCCGGGACTTGTGGGGTCCTTCTCCTGTCA | |
| | CTGGTTATCACCAAACGGGGCAGAAAGAAA | |
| | CTCCTGTATATATTCAAACAACCATTTATG | |
| | AGACCAGTACAAACTACTCAAGAGGAAGAT | |
| | GGCTGTAGCTGCCGATTTCCAGAAGAAGAA | |
| | GAAGGAGGATGTGAACTGCGGGTGAAATTC | |
| | TCCCGTTCAGCTGACGCACCCGCGTATCAA | |
| | CAGGGCCAGAACCAACTGTACAATGAGCTC | |
| | AACCTGGGTCGTAGAGAAGAGTACGATGTG | |
| | CTGGACAAGCGCCGGGGCCGCGACCCTGAG | |
| | ATGGGTGGAAAGCCCCAGCGCCGGAAGAAT | |
| | CCCCAAGAGGGACTGTATAACGAGCTGCAG | |
| | AAGGACAAGATGGCTGAGGCCTACAGCGAG | |
| | ATCGGTATGAAGGGCGAGAGACGTAGAGGC | |
| | AAGGGCCATGACGGGCTGTACCAAGGGCTT | |
| | AGTACCGCTACAAAGGATACCTACGACGCC | |
| | CTGCACATGCAGGCCCTGCCCCCTCGTGGT | |
| | TCCGGAGAGGGCAGAGGCTCTCTGCTCACC | |
| | TGCGGAGATGTGGAGGAAAACCCAGGACCT | |
| | GTGAGCAAGGGCGAGGAGCTGTTCACCGGG | |
| | GTGGTGCCCATCCTGGTCGAGCTGGACGGC | |
| | GACGTAAACGGCCACAAGTTCAGCGTGTCC | |
| | GGCGAGGGCGAGGGCGATGCCACCTACGGC | |
| | AAGCTGACCCTGAAGTTCATCTGCACCACC | |
| | GGCAAGCTGCCCGTGCCCTGGCCCACCCTC | |
| | GTGACCACCCTGACCTACGGCGTGCAGTGC | |
| | TTCAGCCGCTACCCCGACCACATGAAGCAG | |
| | CACGACTTCTTCAAGTCCGCCATGCCCGAA | |
| | GGCTACGTCCAGGAGCGCACCATCTTCTTC | |
| | AAGGACGACGGCAACTACAAGACCCGCGCC | |
| | GAGGTGAAGTTCGAGGGCGACACCCTGGTG | |
| | AACCGCATCGAGCTGAAGGGCATCGACTTC | |
| | AAGGAGGACGGCAACATCCTGGGGCACAAG | |
| | CTGGAGTACAACTACAACAGCCACAACGTC | |
| | TATATCATGGCCGACAAGCAGAAGAACGGC | |
| | ATCAAGGTGAACTTCAAGATCCGCCACAAC | |
| | ATCGAGGACGGCAGCGTGCAGCTCGCCGAC | |
| | CACTACCAGCAGAACACCCCCATCGGCGAC | |
| | GGCCCCGTGCTGCTGCCCGACAACCACTAC | |
| | CTGAGCACCCAGTCCGCCCTGAGCAAAGAC | |
| | CCCAACGAGAAGCGCGATCACATGGTCCTG | |
| | CTGGAGTTCGTGACCGCCGCCGGGATCACT | |
| | CTCGGCATGGACGAGCTGTACAAGTGA | |

TABLE 8
Exemplary CH2-CH3 AAA chimeric
receptor amino acid sequences:

| | | SEQ ID |
|---|---|---|
| Construct | Amino acid sequence | NO |
| CH2/CH3 | AAPEAAGGPSVFLFPPKPKDTLMASRTPEV | 105 |
| (LALA, | TCVVVDVSHEDPEVKFNWYVDGVEVHNAKT | |
| P329R, | KPREEQYNSTYRVVSVLTVLAQDWLNGKEY | |
| I253A, | KCKVSNKALRAPIEKTISKAKGQPREPQVY | |
| H310A, | TLPPSRDELTKNQVSLTCLVKGFYPSDIAV | |
| H435A) | EWESNGQPENNYKTTPPVLDSDGSFFLYSK | |
| | LTVDKSRWQQGNVFSCSVMHEALHNAYTQK | |
| | SLSLSPGK | |
| CH2/CH3- | AAPEAAGGPSVFLFPPKPKDTLMASRTPEV | 106 |
| CD8a_stalk- | TCVVVDVSHEDPEVKFNWYVDGVEVHNAKT | |
| CD8a_TD- | KPREEQYNSTYRVVSVLTVLAQDWLNGKEY | |
| CD137CSD- | KCKVSNKALRAPIEKTISKAKGQPREPQVY | |
| CD3zSSD | TLPPSRDELTKNQVSLTCLVKGFYPSDIAV | |
| fusion | EWESNGQPENNYKTTPPVLDSDGSFFLYSK | |
| | LTVDKSRWQQGNVFSCSVMHEALHNAYTQK | |
| | SLSLSPGKGGGGSLKPTTTPAPRPPTPAP | |
| | TIASQPLSLRPEACRPAAGGAVHTRGLDFA | |
| | CDIYIWAPLAGTCGVLLLSLVITKRGRKKL | |
| | LYIFKQPFMRPVQTTQEEDGCSCRFPEEEE | |
| | GGCELRVKFSRSADAPAYQQGQNQLYNELN | |
| | LGRREEYDVLDKRRGRDPEMGGKPRRKNPQ | |
| | EGLYNELQKDKMAEAYSEIGMKGERRRGKG | |
| | HDGLYQGLSTATKDTYDALHMQALPPR | |

TABLE 9
Exemplary CH2-CH3 AAA chimeric receptor
DNA sequences:

| | | SEQ ID |
|---|---|---|
| Construct | DNA sequence | NO |
| CH2/CH3- | GCCCCTGAAGCTGCTGGCGGCCCTTCCGTG | 107 |
| CD8a_stalk- | TTCCTGTTCCCCCCAAAGCCCAAGGACACC | |
| CD8a_TD- | CTGATGGCCTCCCGGACCCCCGAAGTGACC | |
| CD137CSD- | TGCGTGGTGGTGGACGTGTCCCACGAGGAC | |
| CD3zSSD | CCTGAAGTGAAGTTCAATTGGTACGTGGAC | |
| fusion | GGCGTGGAAGTGCACAACGCCAAGACCAAG | |
| | CCCAGAGAGGAACAGTACAACTCCACCTAC | |
| | CGGGTGGTGTCCGTGCTGACCGTGCTGGCC | |
| | CAGGACTGGCTGAACGGCAAAGAGTACAAG | |
| | TGCAAGGTGTCCAACAAGGCCCTGCGGGCT | |
| | CCCATCGAAAAGACCATCTCCAAGGCCAAG | |
| | GGCCAGCCCCGCGAGCCTCAGGTGTACACC | |
| | CTGCCTCCTAGCCGGGACGAGCTGACCAAG | |
| | AACCAGGTGTCCCTGACCTGCCTGGTGAAG | |
| | GGCTTCTACCCCTCCGATATCGCCGTGGAA | |
| | TGGGAGTCCAACGGCCAGCCCGAGAACAAC | |
| | TACAAGACCACCCCCCCTGTGCTGGACTCC | |
| | GACGGCTCATTCTTCCTGTACTCTAAGCTG | |
| | ACCGTGGACAAGTCCCGGTGGCAGCAGGGC | |
| | AACGTGTTCTCCTGCTCCGTGATGCACGAG | |
| | GCCCTGCACAATGCCTACACCCAGAAGTCC | |
| | CTGTCCCTGTCTCCCGGCAAAGGAGGGGGC | |
| | GGATCCTTGAAGCCCACCACGACGCCAGCG | |
| | CCGCGACCACCAACACCGGCGCCCACCATC | |
| | GCGTCGCAGCCCCTGTCCCTGCGCCCAGAG | |
| | GCGTGCCGGCCAGCGGCGGGGGGCGCAGTG | |
| | CACACGAGGGGGCTGGACTTCGCCTGTGAT | |
| | ATCTACATCTGGGCGCCCCTGGCCGGGACT | |
| | TGTGGGGTCCTTCTCCTGTCACTGGTTATC | |
| | ACCAAACGGGGCAGAAAGAAACTCCTGTAT | |
| | ATATTCAAACAACCATTTATGAGACCAGTA | |
| | CAAACTACTCAAGAGGAAGATGGCTGTAGC | |
| | TGCCGATTTCCAGAAGAAGAAGAAGGAGGA | |
| | TGTGAACTGAGAGTGAAGTTCAGCAGGAGC | |
| | GCAGACGCCCCCGCGTACCAGCAGGGCCAG | |
| | AACCAGCTCTATAACGAGCTCAATCTAGGA | |
| | CGAAGAGAGGAGTACGATGTTTTGGACAAG | |
| | AGACGTGGCCGGGACCCTGAGATGGGGGGA | |
| | AAGCCGAGAAGGAAGAACCCTCAGGAAGGC | |
| | CTGTACAATGAACTGCAGAAAGATAAGATG | |
| | GCGGAGGCCTACAGTGAGATTGGGATGAAA | |
| | GGCGAGCGCCGGAGGGGCAAGGGGCACGAT | |
| | GGCCTTTACCAGGGTCTCAGTACAGCCACC | |
| | AAGGACACCTACGACGCCCTTCACATGCAG | |
| | GCCCTGCCCCCTCGC | |

TABLE 10
Exemplary CD28 costimulatory signaling domain

| | | SEQ ID |
|---|---|---|
| Construct | Amino acid sequence | NO |
| CD28CSD | RSKRSRLLHSDYMNMTPRRPGPTRKHYQPY | 128 |
| | APPRDFAAYRS | |
| CD28CSD | AGGAGTAAGAGGAGCAGGCTCCTGCACAGT | 129 |
| | GACTACATGAACATGACTCCCCGCCGCCCC | |
| | GGGCCCACCCGCAAGCATTACCAGCCCTAT | |
| | GCCCCACCACGCGACTTCGCAGCCTATCGC | |
| | TCC | |

TABLE 11
Exemplary bispecific antibody
anti-PG × FolR1 2 + 1

| | | SEQ ID |
|---|---|---|
| Construct | Amino acid sequence | NO |
| Anti | EVQLVESGGGLVKPGGSLRLSCAASGFTFS | 108 |
| FolR1- | NAWMSWVRQAPGKGLEWVGRIKSKTDGGTT | |
| VHCL | DYAAPVKGRFTISRDDSKNTLYLQMNSLKT | |
| | EDTAVYYCTTPWEWSWYDYWGQGTLVTVSS | |
| | ASVAAPSVFIFPPSDEQLKSGTASVVCLLN | |
| | NFYPREAKVQWKVDNALQSGNSQESVTEQD | |
| | SKDSTYSLSSTLTLSKADYEKHKVYACEVT | |
| | HQGLSSPVTKSFNRGEC | |
| Anti FolR1- | QAVVTQEPSLTVSPGGTVTLTCGSSTGAVT | 109 |
| VLCH1- | TSNYANWVQEKPGQAFRGLIGGTNKRAPGT | |
| CH2(P329R) | PARFSGSLLGGKAALTLSGAQPEDEAEYYC | |
| -CH3 | ALWYSNLWVFGGGTKLTVLSSASTKGPSVF | |
| | PLAPSSKSTSGGTAALGCLVKDYFPEPVTV | |
| | SWNSGALTSGVHTFPAVLQSSGLYSLSSVV | |
| | TVPSSSLGTQTYICNVNHKPSNTKVDKKVE | |
| | PKSCDKTHTCPPCPAPEAAGGPSVFLFPPK | |
| | PKDTLMISRTPEVTCVVVDVSHEDPEVKFN | |
| | WYVDGVEVHNAKTKPREEQYNSTYRVVSVL | |
| | TVLHQDWLNGKEYKCKVSNKALRAPIEKTI | |
| | SKAKGQPREPQVCTLPPSRDELTKNQVSLS | |
| | CAVKGFYPSDIAVEWESNGQPENNYKTTPP | |
| | VLDSDGSFFLVSKLTVDKSRWQQGNVFSCS | |
| | VMHEALHNHYTQKSLSLSPGK | |
| Anti FolR1- | QAVVTQEPSLTVSPGGTVTLTCGSSTGAVT | 110 |
| VLCH1- | TSNYANWVQEKPGQAFRGLIGGTNKRAPGT | |
| Anti PG- | PARFSGSLLGGKAALTLSGAQPEDEAEYYC | |
| VHCH1- | ALWYSNLWVFGGGTKLTVLSSASTKGPSVF | |
| CH2(P329R) | PLAPSSKSTSGGTAALGCLVKDYFPEPVTV | |
| -CH3 | SWNSGALTSGVHTFPAVLQSSGLYSLSSVV | |
| | TVPSSSLGTQTYICNVNHKPSNTKVDKKVE | |
| | PKSCDGGGGSGGGGGEVQLVESGGGLVQPG | |
| | GSLRLSCAASGFTFSRYWMNWVRQAPGKGL | |
| | EWVGEITPDSSTINYAPSLKGRFTISRDNA | |
| | KNSLYLQMNSLRAEDTAVYYCARPYDYGAW | |
| | FASWGQGTLVTVSSASTKGPSVFPLAPSSK | |
| | STSGGTAALGCLVEDYFPEPVTVSWNSGAL | |
| | TSGVHTFPAVLQSSGLYSLSSVVTVPSSSL | |
| | GTQTYICNVNHKPSNTKVDEKVEPKSCDKT | |
| | HTCPPCPAPEAAGGPSVFLFPPKPKDTLMI | |
| | SRTPEVTCVVVDVSHEDPEVKFNWYVDGVE | |
| | VHNAKTKPREEQYNSTYRVVSVLTVLHQDW | |
| | LNGKEYKCKVSNKALRAPIEKTISKAKGQP | |
| | REPQVYTLPPCRDELTKNQVSLWCLVKGFY | |
| | PSDIAVEWESNGQPENNYKTTPPVLDSDGS | |
| | FFLYSKLTVDKSRWQQGNVFSCSVMHEALH | |
| | NHYTQKSLSLSPGK | |
| Anti PG- | QAVVTQEPSLTVSPGGTVTLTCRSSTGAVT | 111 |
| VLCL | TSNYANWVQEKPDHLFTGLIGGTNKRAPGT | |
| | PARFSGSLLGGKAALTLSGAQPEDEAEYYC | |
| | ALWYSNHWVFGGGTKLTVLGQPKAAPSVTL | |
| | FPPSSKKLQANKATLVCLISDFYPGAVTVA | |
| | WKADSSPVKAGVETTTPSKQSNNKYAASSY | |
| | LSLTPEQWKSHRSYSCQVTHEGSTVEKTVA | |
| | PTECS | |

TABLE 12
Exemplary bispecific antibody
anti-AAA × FolR1 2 + 1

| | | SEQ ID |
|---|---|---|
| Construct | Amino acid sequence | NO |
| Anti FolR1- | EVQLVESGGGLVKPGGSLRLSCAASGFTFS | 108 |
| VHCL | NAWMSWVRQAPGKGLEWVGRIKSKTDGGTT | |
| | DYAAPVKGRFTISRDDSKNTLYLQMNSLKT | |
| | EDTAVYYCTTPWEWSWYDYWGQGTLVTVSS | |
| | ASVAAPSVFIFPPSDEQLKSGTASVVCLLN | |
| | NFYPREAKVQWKVDNALQSGNSQESVTEQD | |
| | SKDSTYSLSSTLTLSKADYEKHKVYACEVT | |
| | HQGLSSPVTKSFNRGEC | |
| Anti FolR1- | QAVVTQEPSLTVSPGGTVTLTCGSSTGAVT | 112 |
| VLCH1- | TSNYANWVQEKPGQAFRGLIGGTNKRAPGT | |
| CH2(P329G) | PARFSGSLLGGKAALTLSGAQPEDEAEYYC | |
| -CH3 | ALWYSNLWVFGGGTKLTVLSSASTKGPSVF | |
| | PLAPSSKSTSGGTAALGCLVKDYFPEPVTV | |
| | SWNSGALTSGVHTFPAVLQSSGLYSLSSVV | |
| | TVPSSSLGTQTYICNVNHKPSNTKVDKKVE | |
| | PKSCDKTHTCPPCPAPEAAGGPSVFLFPPK | |
| | PKDTLMISRTPEVTCVVVDVSHEDPEVKFN | |
| | WYVDGVEVHNAKTKPREEQYNSTYRVVSVL | |
| | TVLHQDWLNGKEYKCKVSNKALGAPIEKTI | |
| | SKAKGQPREPQVCTLPPSRDELTKNQVSLS | |
| | CAVKGFYPSDIAVEWESNGQPENNYKTTPP | |
| | VLDSDGSFFLVSKLTVDKSRWQQGNVFSCS | |
| | VMHEALHNHYTQKSLSLSPG | |
| Anti FolR1- | QAVVTQEPSLTVSPGGTVTLTCGSSTGAVT | 113 |
| VLCH1- | TSNYANWVQEKPGQAFRGLIGGTNKRAPGT | |
| | PARFSGSLLGGKAALTLSGAQPEDEAEYYC | |
| Anti AAA | ALWYSNLWVFGGGTKLTVLSSASTKGPSVF | |
| VLCH1- | PLAPSSKSTSGGTAALGCLVKDYFPEPVTV | |
| | SWNSGALTSGVHTFPAVLQSSGLYSLSSVV | |
| | TVPSSSLGTQTYICNVNHKPSNTKVDKKVE | |
| | PKSCDGGGGSGGGGGEVQLVESGGDLVKPG | |
| | GSLKLSCAASGFTFSSYGMSWVRQTPDKRL | |
| | EWVATISSGGSYIYYPDSVKGRFTISRDNA | |
| | KNTLYLQMSSLKSEDTAMYYCARLGMITTG | |
| | YAMDYWGQGTSVTVSSASTKGPSVFPLAPS | |
| | SKSTSGGTAALGCLVEDYFPEPVTVSWNSG | |
| | ALTSGVHTFPAVLQSSGLYSLSSVVTVPSS | |
| | SLGTQTYICNVNHKPSNTKVDEKV | |
| CH2(P329G)- | EPKSCDKTHTCPPCPAPEAAGGPSVFLFPP | |
| CH3-Avi Tag | KPKDTLMISRTPEVTCVVVDVSHEDPEVKF | |
| | NWYVDGVEVHNAKTKPREEQYNSTYRVVSV | |
| | LTVLHQDWLNGKEYKCKVSNKALGAPIEKT | |
| | ISKAKGQPREPQVYTLPPCRDELTKNQVSL | |
| | WCLVKGFYPSDIAVEWESNGQPENNYKTTP | |
| | PVLDSDGSFFLYSKLTVDKSRWQQGNVFSC | |
| | SVMHEALHNHYTQKSLSLSPGGSGGLNDIF | |
| | EAQKIEWHE | |
| Anti AAA | DVLMTQTPLSLPVSLGDQASISCRSSQTIV | 114 |
| VLCL | HSTGHTYLEWFLQKPGQSPKLLIYKVSNRF | |
| | SGVPDRFSGSGSGTDFTLKISRVEAEDLGV | |
| | YYCFQGSHVPYTFGGGTKLEIKRTVAAPSV | |
| | FIFPPSDRKLKSGTASVVCLLNNFYPREAK | |
| | VQWKVDNALQSGNSQESVTEQDSKDSTYSL | |
| | SSTLTLSKADYEKHKVYACEVTHQGLSSPV | |
| | TKSFNRGEC | |

TABLE 13
Exemplary bispecific antibody
anti-PG × FolR1 1 + 1

| | | SEQ ID |
|---|---|---|
| Construct | Amino acid sequence | NO |
| Anti FolR1- | EVQLVESGGGLVKPGGSLRLSCAASGFTFS | 108 |
| VHCL | NAWMSWVRQAPGKGLEWVGRIKSKTDGGTT | |
| | DYAAPVKGRFTISRDDSKNTLYLQMNSLKT | |
| | EDTAVYYCTTPWEWSWYDYWGQGTLVTVSS | |
| | ASVAAPSVFIFPPSDEQLKSGTASVVCLLN | |
| | NFYPREAKVQWKVDNALQSGNSQESVTEQD | |
| | SKDSTYSLSSTLTLSKADYEKHKVYACEVT | |
| | HQGLSSPVTKSFNRGEC | |
| Anti FolR1- | QAVVTQEPSLTVSPGGTVTLTCGSSTGAVT | 109 |
| VLCH1- | TSNYANWVQEKPGQAFRGLIGGTNKRAPGT | |
| CH2(P329R) | PARFSGSLLGGKAALTLSGAQPEDEAEYYC | |
| -CH3 | ALWYSNLWVFGGGTKLTVLSSASTKGPSVF | |
| | PLAPSSKSTSGGTAALGCLVKDYFPEPVTV | |
| | SWNSGALTSGVHTFPAVLQSSGLYSLSSVV | |
| | TVPSSSLGTQTYICNVNHKPSNTKVDKKVE | |
| | PKSCDKTHTCPPCPAPEAAGGPSVFLFPPK | |
| | PKDTLMISRTPEVTCVVVDVSHEDPEVKFN | |
| | WYVDGVEVHNAKTKPREEQYNSTYRVVSVL | |
| | TVLHQDWLNGKEYKCKVSNKALRAPIEKTI | |
| | SKAKGQPREPQVCTLPPSRDELTKNQVSLS | |
| | CAVKGFYPSDIAVEWESNGQPENNYKTTPP | |
| | VLDSDGSFFLVSKLTVDKSRWQQGNVFSCS | |
| | VMHEALHNHYTQKSLSLSPGK | |
| Anti PG- | QAVVTQEPSLTVSPGGTVTLTCRSSTGAVT | 111 |
| VLCL | TSNYANWVQEKPDHLFTGLIGGTNKRAPGT | |
| | PARFSGSLLGGKAALTLSGAQPEDEAEYYC | |
| | ALWYSNHWVFGGGTKLTVLGQPKAAPSVTL | |
| | FPPSSKKLQANKATLVCLISDFYPGAVTVA | |
| | WKADSSPVKAGVETTTPSKQSNNKYAASSY | |
| | LSLTPEQWKSHRSYSCQVTHEGSTVEKTVA | |
| | PTECS | |
| Anti PG- | EVQLVESGGGLVQPGGSLRLSCAASGFTFS | 115 |
| VHCH1- | RYWMNWVRQAPGKGLEWVGEITPDSSTINY | |
| CH2(P329R) | APSLKGRFTISRDNAKNSLYLQMNSLRAED | |
| -CH3 | TAVYYCARPYDYGAWFASWGQGTLVTVSSA | |
| | STKGPSVFPLAPSSKSTSGGTAALGCLVED | |
| | YFPEPVTVSWNSGALTSGVHTFPAVLQSSG | |
| | LYSLSSVVTVPSSSLGTQTYICNVNHKPSN | |
| | TKVDEKVEPKSCDKTHTCPPCPAPEAAGGP | |
| | SVFLFPPKPKDTLMISRTPEVTCVVVDVSH | |
| | EDPEVKFNWYVDGVEVHNAKTKPREEQYNS | |
| | TYRVVSVLTVLHQDWLNGKEYKCKVSNKAL | |
| | RAPIEKTISKAKGQPREPQVYTLPPCRDEL | |
| | TKNQVSLWCLVKGFYPSDIAVEWESNGQPE | |
| | NNYKTTPPVLDSDGSFFLYSKLTVDKSRWQ | |
| | QGNVFSCSVMHEALHNHYTQKSLSLSPGK | |

TABLE 14
Exemplary bispecific antibody
anti-AAA × FolR1 1 + 1

| | | SEQ ID |
|---|---|---|
| Construct | Amino acid sequence | NO |
| Anti FolR1- | EVQLVESGGGLVKPGGSLRLSCAASGFTFS | 108 |
| VHCL | NAWMSWVRQAPGKGLEWVGRIKSKTDGGTT | |
| | DYAAPVKGRFTISRDDSKNTLYLQMNSLKT | |
| | EDTAVYYCTTPWEWSWYDYWGQGTLVTVSS | |
| | ASVAAPSVFIFPPSDEQLKSGTASVVCLLN | |
| | NFYPREAKVQWKVDNALQSGNSQESVTEQD | |
| | SKDSTYSLSSTLTLSKADYEKHKVYACEVT | |
| | HQGLSSPVTKSFNRGEC | |
| Anti FolR1- | QAVVTQEPSLTVSPGGTVTLTCGSSTGAVT | 112 |
| VLCH1- | TSNYANWVQEKPGQAFRGLIGGTNKRAPGT | |
| CH2(P329G) | PARFSGSLLGGKAALTLSGAQPEDEAEYYC | |
| -CH3 | ALWYSNLWVFGGGTKLTVLSSASTKGPSVF | |
| | PLAPSSKSTSGGTAALGCLVKDYFPEPVTV | |
| | SWNSGALTSGVHTFPAVLQSSGLYSLSSVV | |
| | TVPSSSLGTQTYICNVNHKPSNTKVDKKVE | |
| | PKSCDKTHTCPPCPAPEAAGGPSVFLFPPK | |
| | PKDTLMISRTPEVTCVVVDVSHEDPEVKFN | |
| | WYVDGVEVHNAKTKPREEQYNSTYRVVSVL | |
| | TVLHQDWLNGKEYKCKVSNKALGAPIEKTI | |
| | SKAKGQPREPQVCTLPPSRDELTKNQVSLS | |
| | CAVKGFYPSDIAVEWESNGQPENNYKTTPP | |
| | VLDSDGSFFLVSKLTVDKSRWQQGNVFSCS | |
| | VMHEALHNHYTQKSLSLSPG | |
| Anti AAA | DVLMTQTPLSLPVSLGDQASISCRSSQTIV | 114 |
| VLCL | HSTGHTYLEWFLQKPGQSPKLLIYKVSNRF | |
| | SGVPDRFSGSGSGTDFTLKISRVEAEDLGV | |
| | YYCFQGSHVPYTFGGGTKLEIKRTVAAPSV | |
| | FIFPPSDRKLKSGTASVVCLLNNFYPREAK | |
| | VQWKVDNALQSGNSQESVTEQDSKDSTYSL | |
| | SSTLTLSKADYEKHKVYACEVTHQGLSSPV | |
| | TKSFNRGEC | |
| Anti AAA | EVQLVESGGDLVKPGGSLKLSCAASGFTFS | 116 |
| VLCH1- | SYGMSWVRQTPDKRLEWVATISSGGSYIYY | |
| CH2(P329G) | PDSVKGRFTISRDNAKNTLYLQMSSLKSED | |
| -CH3-Avi | TAMYYCARLGMITTGYAMDYWGQGTSVTVS | |
| Tag | SASTKGPSVFPLAPSSKSTSGGTAALGCLV | |
| | EDYFPEPVTVSWNSGALTSGVHTFPAVLQS | |
| | SGLYSLSSVVTVPSSSLGTQTYICNVNHKP | |
| | SNTKVDEKVEPKSCDKTHTCPPCPAPEAAG | |
| | GPSVFLFPPKPKDTLMISRTPEVTCVVVDV | |
| | SHEDPEVKFNWYVDGVEVHNAKTKPREEQY | |
| | NSTYRVVSVLTVLHQDWLNGKEYKCKVSNK | |
| | ALGAPIEKTISKAKGQPREPQVYTLPPCRD | |
| | ELTKNQVSLWCLVKGFYPSDIAVEWESNGQ | |
| | PENNYKTTPPVLDSDGSFFLYSKLTVDKSR | |
| | WQQGNVFSCSVMHEALHNHYTQKSLSLSPG | |
| | GSGGLNDIFEAQKIEWHE | |

TABLE 15
Exemplary bispecific antibody
anti-PG × CEA 2 + 1

| | | SEQ ID |
|---|---|---|
| Construct | Amino acid sequence | NO |
| Anti CEA- | EVQLQQSGAELVEPGASVKLSCTASGFNIK | 117 |
| VHCL | DTYMHWVKQRPEQGLEWIGRIDPANGNSKY | |
| | VPKFQGKATITADTSSNTAYLQLTSLTSED | |
| | TAVYYCAPFGYYVSDYAMAYWGQGTSVTVS | |
| | SASVAAPSVFIFPPSDEQLKSGTASVVCLL | |
| | NNFYPREAKVQWKVDNALQSGNSQESVTEQ | |
| | DSKDSTYSLSSTLTLSKADYEKHKVYACEV | |
| | THQGLSSPVTKSFNRGEC | |
| Anti CEA- | DIVLTQSPASLAVSLGQRATMSCRAGESVD | 118 |
| VLCH1- | IFGVGFLHWYQQKPGQPPKLLIYRASNLES | |
| CH2(P329R) | GIPVRFSGTGSRTDFTLIIDPVEADDVATY | |
| -CH3 | YCQQTNEDPYTFGGGTKLEIKSSASTKGPS | |
| | VFPLAPSSKSTSGGTAALGCLVKDYFPEPV | |
| | TVSWNSGALTSGVHTFPAVLQSSGLYSLSS | |
| | VVTVPSSSLGTQTYICNVNHKPSNTKVDKK | |
| | VEPKSCDKTHTCPPCPAPEAAGGPSVFLFP | |
| | PKPKDTLMISRTPEVTCVVVDVSHEDPEVK | |
| | FNWYVDGVEVHNAKTKPREEQYNSTYRVVS | |
| | VLTVLHQDWLNGKEYKCKVSNKALRAPIEK | |
| | TISKAKGQPREPQVCTLPPSRDELTKNQVS | |
| | LSCAVKGFYPSDIAVEWESNGQPENNYKTT | |
| | PPVLDSDGSFFLVSKLTVDKSRWQQGNVFS | |
| | CSVMHEALHNHYTQKSLSLSPG | |
| Anti CEA- | DIVLTQSPASLAVSLGQRATMSCRAGESVD | 119 |
| VLCH1- | IFGVGFLHWYQQKPGQPPKLLIYRASNLES | |
| Anti PG- | GIPVRFSGTGSRTDFTLIIDPVEADDVATY | |
| VHCH1- | YCQQTNEDPYTFGGGTKLEIKSSASTKGPS | |
| CH2(P329R) | VFPLAPSSKSTSGGTAALGCLVKDYFPEPV | |
| -CH3-Avi | TVSWNSGALTSGVHTFPAVLQSSGLYSLSS | |
| Tag | VVTVPSSSLGTQTYICNVNHKPSNTKVDKK | |
| | VEPKSCGGGGSGGGGEVQLVESGGGLVQPG | |
| | GSLRLSCAASGFTFSRYWMNWVRQAPGKGL | |
| | EWVGEITPDSSTINYAPSLKGRFTISRDNA | |
| | KNSLYLQMNSLRAEDTAVYYCARPYDYGAW | |
| | FASWGQGTLVTVSSASTKGPSVFPLAPSSK | |
| | STSGGTAALGCLVEDYFPEPVTVSWNSGAL | |
| | TSGVHTFPAVLQSSGLYSLSSVVTVPSSSL | |
| | GTQTYICNVNHKPSNTKVDEKVEPKSCDKT | |
| | HTCPPCPAPEAAGGPSVFLFPPKPKDTLMI | |
| | SRTPEVTCVVVDVSHEDPEVKFNWYVDGVE | |
| | VHNAKTKPREEQYNSTYRVVSVLTVLHQDW | |
| | LNGKEYKCKVSNKALRAPIEKTISKAKGQP | |
| | REPQVYTLPPCRDELTKNQVSLWCLVKGFY | |
| | PSDIAVEWESNGQPENNYKTTPPVLDSDGS | |
| | FFLYSKLTVDKSRWQQGNVFSCSVMHEALH | |
| | NHYTQKSLSLSPGGSGGLNDIFEAQKIEWH | |
| | E | |
| Anti PG- | QAVVTQEPSLTVSPGGTVTLTCRSSTGAVT | 120 |
| VLCL | TSNYANWVQEKPDHLFTGLIGGTNKRAPGT | |
| | PARFSGSLLGGKAALTLSGAQPEDEAEYYC | |
| | ALWYSNHWVFGGGTKLTVLGQPKAAPSVTL | |
| | FPPSSKKLQANKATLVCLISDFYPGAVTVA | |
| | WKADSSPVKAGVETTTPSKQSNNKYAASSY | |
| | LSLTPEQWKSHRSYSCQVTHEGSTVEKTVA | |
| | PTECS | |

TABLE 16
Exemplary bispecific antibody
anti-AAA × CEA 2 + 1

| | | SEQ ID |
|---|---|---|
| Construct | Amino acid sequence | NO |
| Anti CEA- | EVQLQQSGAELVEPGASVKLSCTASGFNIK | 121 |
| VHCL | DTYMHWVKQRPEQGLEWIGRIDPANGNSKY | |
| | VPKFQGKATITADTSSNTAYLQLTSLTSED | |
| | TAVYYCAPFGYYVSDYAMAYWGQGTSVTVS | |
| | SASVAAPSVFIFPPSDEQLKSGTASVVCLL | |
| | NNFYPREAKVQWKVDNALQSGNSQESVTEQ | |
| | DSKDSTYSLSSTLTLSKADYEKHKVYACEV | |
| | THQGLSSPVTKSFNRGEC | |
| Anti CEA- | DIVLTQSPASLAVSLGQRATMSCRAGESVD | 122 |
| VLCH1- | IFGVGFLHWYQQKPGQPPKLLIYRASNLES | |
| CH2(P329G) | GIPVRFSGTGSRTDFTLIIDPVEADDVATY | |
| -CH3 | YCQQTNEDPYTFGGGTKLEIKSSASTKGPS | |
| | VFPLAPSSKSTSGGTAALGCLVKDYFPEPV | |
| | TVSWNSGALTSGVHTFPAVLQSSGLYSLSS | |
| | VVTVPSSSLGTQTYICNVNHKPSNTKVDKK | |
| | VEPKSCDKTHTCPPCPAPEAAGGPSVFLFP | |
| | PKPKDTLMISRTPEVTCVVVDVSHEDPEVK | |
| | FNWYVDGVEVHNAKTKPREEQYNSTYRVVS | |
| | VLTVLHQDWLNGKEYKCKVSNKALGAPIEK | |
| | TISKAKGQPREPQVCTLPPSRDELTKNQVS | |
| | LSCAVKGFYPSDIAVEWESNGQPENNYKTT | |
| | PPVLDSDGSFFLVSKLTVDKSRWQQGNVFS | |
| | CSVMHEALHNHYTQKSLSLSPG | |
| Anti CEA- | DIVLTQSPASLAVSLGQRATMSCRAGESVD | 123 |
| VLCH1- | IFGVGFLHWYQQKPGQPPKLLIYRASNLES | |
| Anti AAA- | GIPVRFSGTGSRTDFTLIIDPVEADDVATY | |
| VHCH1- | YCQQTNEDPYTFGGGTKLEIKSSASTKGPS | |
| CH2(P329G) | VFPLAPSSKSTSGGTAALGCLVKDYFPEPV | |
| -CH3-Avi | TVSWNSGALTSGVHTFPAVLQSSGLYSLSS | |
| Tag | VVTVPSSSLGTQTYICNVNHKPSNTKVDKK | |
| | VEPKSCGGGGSGGGGEVQLVESGGDLVKPG | |
| | GSLKLSCAASGFTFSSYGMSWVRQTPDKRL | |
| | EWVATISSGGSYIYYPDSVKGRFTISRDNA | |
| | KNTLYLQMSSLKSEDTAMYYCARLGMITTG | |
| | YAMDYWGQGTSVTVSSASTKGPSVFPLAPS | |
| | SKSTSGGTAALGCLVEDYFPEPVTVSWNSG | |
| | ALTSGVHTFPAVLQSSGLYSLSSVVTVPSS | |
| | SLGTQTYICNVNHKPSNTKVDEKVEPKSCD | |
| | KTHTCPPCPAPEAAGGPSVFLFPPKPKDTL | |
| | MISRTPEVTCVVVDVSHEDPEVKFNWYVDG | |
| | VEVHNAKTKPREEQYNSTYRVVSVLTVLHQ | |
| | DWLNGKEYKCKVSNKALGAPIEKTISKAKG | |
| | QPREPQVYTLPPCRDELTKNQVSLWCLVKG | |
| | FYPSDIAVEWESNGQPENNYKTTPPVLDSD | |
| | GSFFLYSKLTVDKSRWQQGNVFSCSVMHEA | |
| | LHNHYTQKSLSLSPGGSGGLNDIFEAQKIE | |
| | WHE | |
| Anti AAA- | DVLMTQTPLSLPVSLGDQASISCRSSQTIV | 114 |
| VLCL | HSTGHTYLEWFLQKPGQSPKLLIYKVSNRF | |
| | SGVPDRFSGSGSGTDFTLKISRVEAEDLGV | |
| | YYCFQGSHVPYTFGGGTKLEIKRTVAAPSV | |
| | FIFPPSDRKLKSGTASVVCLLNNFYPREAK | |
| | VQWKVDNALQSGNSQESVTEQDSKDSTYSL | |
| | SSTLTLSKADYEKHKVYACEVTHQGLSSPV | |
| | TKSFNRGEC | |

Exemplary CH2-CH3 AAA P329R chimeric
receptor amino acid sequences:

| | | SEQ ID |
|---|---|---|
| Construct | Amino acid sequence | NO |
| CH2/CH3 | APEAAGGPSVFLFPPKPKDTLMASRTPEVT | 124 |
| (LALA, | CVVVDVSHEDPEVKFNWYVDGVEVHNAKTK | |
| P329R, | PREEQYNSTYRVVSVLTVLAQDWLNGKEYK | |
| I253A, | CKVSNKALRAPIEKTISKAKGQPREPQVYT | |
| H310A, | LPPSRDELTKNQVSLTCLVKGFYPSDIAVE | |
| H435A) | WESNGQPENNYKTTPPVLDSDGSFFLYSKL | |
| | TVDKSRWQQGNVFSCSVMHEALHNAYTQKS | |
| | LSLSPGK | |
| CH2/CH3 | APEAAGGPSVFLFPPKPKDTLMASRTPEVT | 125 |
| (LALA, | CVVVDVSHEDPEVKFNWYVDGVEVHNAKTK | |
| P329R, | PREEQYNSTYRVVSVLTVLAQDWLNGKEYK | |
| I253A, | CKVSNKALRAPIEKTISKAKGQPREPQVYT | |
| H310A, | LPPSRDELTKNQVSLTCLVKGFYPSDIAVE | |
| H435A) | WESNGQPENNYKTTPPVLDSDGSFFLYSKL | |
| CD8a_stalk- | TVDKSRWQQGNVFSCSVMHEALHNAYTQKS | |
| CD8a_TD- | LSLSPGKGGGGSAKPTTTPAPRPPTPAPTI | |
| CD137CSD- | ASQPLSLRPEACRPAAGGAVHTRGLDFACD | |
| CD3zSSD | IYIWAPLAGTCGVLLLSLVITKRGRKKLLY | |
| fusion | IFKQPFMRPVQTTQEEDGCSCRFPEEEEGG | |
| | CELRVKFSRSADAPAYQQGQNQLYNELNLG | |
| | RREEYDVLDKRRGRDPEMGGKPQRRKNPQE | |
| | GLYNELQKDKMAEAYSEIGMKGERRRGKGH | |
| | DGLYQGLSTATKDTYDALHMQALPPR | |

TABLE 18
Exemplary CH2-CH3 AAA P329G chimeric
receptor amino acid sequences:

| | | SEQ ID |
|---|---|---|
| Construct | Amino acid sequence | NO |
| CH2/CH3 | APEAAGGPSVFLFPPKPKDTLMASRTPEVT | 126 |
| (LALA, | CVVVDVSHEDPEVKFNWYVDGVEVHNAKTK | |
| P329G, | PREEQYNSTYRVVSVLTVLAQDWLNGKEYK | |
| I253A, | CKVSNKALGAPIEKTISKAKGQPREPQVYT | |
| H310A, | LPPSRDELTKNQVSLTCLVKGFYPSDIAVE | |
| H435A) | WESNGQPENNYKTTPPVLDSDGSFFLYSKL | |
| | TVDKSRWQQGNVFSCSVMHEALHNAYTQKS | |
| | LSLSPGKAPEAAGGPSVFLFPPKPKDTLMA | |
| | SRTPEVTCVVVDVSHEDPEVKFNWYVDGVE | |
| | VHNAKTKPREEQYNSTYRVVSVLTVLAQDW | |
| CH2/CH3 | LNGKEYKCKVSNKALGAPIEKTISKAKGQP | 127 |
| (LALA, | REPQVYTLPPSRDELTKNQVSLTCLVKGFY | |
| P329G, | PSDIAVEWESNGQPENNYKTTPPVLDSDGS | |
| I253A, | FFLYSKLTVDKSRWQQGNVFSCSVMHEALH | |
| H310A, | NAYTQKSLSLSPGKGGGGSAKPTTTPAPRP | |
| H435A) | PTPAPTIASQPLSLRPEACRPAAGGAVHTR | |
| CD8a_stalk- | GLDFACDIYIWAPLAGTCGVLLLSLVITKR | |
| CD8a_TD- | GRKKLLYIFKQPFMRPVQTTQEEDGCSCRF | |
| CD137CSD- | PEEEEGGCELRVKFSRSADAPAYQQGQNQL | |
| CD3zSSD | YNELNLGRREEYDVLDKRRGRDPEMGGKPQ | |
| fusion | RRKNPQEGLYNELQKDKMAEAYSEIGMKGE | |
| | RRRGKGHDGLYQGLSTATKDTYDALHMQAL | |
| | PPR | |

TABLE 19
Exemplary anti-AAA antigen
binding moiety

| | | SEQ ID |
|---|---|---|
| Construct | Amino acid sequence | NO |
| HCDR1 | GFTFSSYG | 130 |
| HCDR2 | ISSGGSYI | 131 |
| HCDR3 | ARLGMITTGY AMDY | 132 |
| LCDR1 | QTIVHSTGHTY | 133 |
| LCDR2 | KVS | 134 |
| LCDR3 | FQGSHVPYT | 135 |
| VH | EVQLVESGGDLVKPGGSLKLSCAASGFTFS | 136 |
| | SYGMSWVRQTPDKRLEWVATISSGGSYIYY | |
| | PDSVKGRFTISRDNAKNTLYLQMSSLKSED | |
| | TAMYYCARLGMITTGYAMDYWGQGTSVTVS | |
| | S | |
| VL | DVLMTQTPLSLPVSLGDQASISCRSSQTIV | 35 |
| | HSTGHTYLEWFLQKPGQSPKLLIYKVSNRF | |
| | SGVPDRFSGSGSGTDFTLKISRVEAEDLGV | |
| | YYCFQGSHVPYTFGGGTKLEIK | |

TABLE 20
Exemplary anti-P329G-IL2v

| | | SEQ ID |
|---|---|---|
| Construct | Amino acid sequence | NO |
| Anti PG- | QAVVTQESALTTSPGETVTLTCRSSTGAVT | 137 |
| VLCL | TSNYANWVQEKPDHLFTGLIGGTNKRAPGV | |
| | PARFSGSLIGDKAALTITGAQTEDEAIYFC | |
| | ALWYSNHWVFGGGTKLTVLGQPKAAPSVTL | |
| | FPPSSKKLQANKATLVCLISDFYPGAVTVA | |
| | WKADSSPVKAGVETTTPSKQSNNKYAASSY | |
| | LSLTPEQWKSHRSYSCQVTHEGSTVEKTVA | |
| | PTECS | |
| Anti PG- | EVKLLESGGGLVQPGGSLKLSCAASGFDFS | 138 |
| VHCH1- | RYWMNWVRQAPGKGLEWIGEITPDSSTINY | |
| CH2(LA)- | TPSLKDKFIISRDNAKNTLYLQMIKVRSED | |
| CH3 | TALYYCVRPYDYGAWFASWGQGTLVTVSAA | |
| | STKGPSVFPLAPSSKSTSGGTAALGCLVED | |
| | YFPEPVTVSWNSGALTSGVHTFPAVLQSSG | |
| | LYSLSSVVTVPSSSLGTQTYICNVNHKPSN | |
| | TKVDEKVEPKSCDKTHTCPPCPAPEAAGGP | |
| | SVFLFPPKPKDTLMISRTPEVTCVVVDVSH | |
| | EDPEVKFNWYVDGVEVHNAKTKPREEQYNS | |
| | TYRVVSVLTVLHQDWLNGKEYKCKVSNKAL | |
| | PAPIEKTISKAKGQPREPQVCTLPPSRDEL | |
| | TKNQVSLSCAVKGFYPSDIAVEWESNGQPE | |
| | NNYKTTPPVLDSDGSFFLVSKLTVDKSRWQ | |
| | QGNVFSCSVMHEALHNHYTQKSLSLSPGK | |
| CH2(LA)- | DKTHTCPPCPAPEAAGGPSVFLFPPKPKDT | 139 |
| CH3-(G4S)3- | LMISRTPEVTCVVVDVSHEDPEVKFNWYVD | |
| IL2v | GVEVHNAKTKPREEQYNSTYRVVSVLTVLH | |
| | QDWLNGKEYKCKVSNKALPAPIEKTISKAK | |
| | GQPREPQVYTLPPCRDELTKNQVSLWCLVK | |
| | GFYPSDIAVEWESNGQPENNYKTTPPVLDS | |
| | DGSFFLYSKLTVDKSRWQQGNVFSCSVMHE | |
| | ALHNHYTQKSLSLSPGGGGGSGGGGSGGGG | |
| | SAPASSSTKKTQLQLEHLLLDLQMILNGIN | |
| | NYKNPKLTRMLTAKFAMPKKATELKHLQCL | |
| | EEELKPLEEVLNGAQSKNFHLRPRDLISNI | |
| | NVIVLELKGSETTFMCEYADETATIVEFLN | |
| | RWITFAQSIISTLT | |

### EXAMPLES

The following are examples of methods and compositions of the invention. It is understood that various other embodiments may be practiced, given the general description provided above.

### Recombinant DNA Techniques

Standard methods were used to manipulate DNA as described in Sambrook et al., Molecular cloning: A laboratory manual: Cold Spring Harbor Laboratory Press, Cold Spring Harbor, New York, 1989. The molecular biological reagents were used according to the manufacturers' instructions. General information regarding the nucleotide sequences of human immunoglobulins light and heavy chains is given in: Kabat, E. A. et al., (1991) Sequences of Proteins of Immunological Interest, 5th ed., NIH Publication No. 91-3242.

### DNA Sequencing

DNA sequences were determined by double strand Sanger sequencing.

### Gene Synthesis

Desired gene segments where required were either generated by PCR using appropriate templates or were synthesized by Genscript Biotech (New Jersey, US) from synthetic oligonucleotides and PCR products by automated gene synthesis. The gene segments flanked by single restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

### Production of IgG-Like Proteins in Expi293F Cells

Antibodies and bispecific antibodies were generated by transient transfection of Expi293F cells. Cells were seeded in Expi293 media (Gibco, Cat. No 1435101) at a density of 2.5×106/ml. Expression vectors and ExpiFectamine (Gibco, ExpiFectamine transfection kit, Cat. No 13385544) were separately mixed in OptiMEM (Gibco, Cat. No 11520386). After 5 minutes both solutions were combined, mixed by pipetting and incubated for 25 minutes at room temperature. Cells were added to the vector/ExpiFectamine solution and incubated for 24 hours at 37° C. in a shaking incubator with a 5% CO2 atmosphere. One day post transfection, supplements (Enhancer 1+2, ExpiFectamine transfection kit) were added. Cell supernatants were harvested after 4-5 days by centrifugation and subsequent filtration (0.2 μm filter), and proteins were purified from the harvested supernatant by standard methods as indicated below.

Production of IgG-like proteins in CHO K1 cells

Alternatively, the antibodies and bispecific antibodies described herein were prepared by Evitria using their proprietary vector system with conventional (non-PCR based) cloning techniques and using suspension-adapted CHO K1 cells (originally received from ATCC and adapted to serum-free growth in suspension culture at Evitria). For the production, Evitria used its proprietary, animal-component free and serum-free media (eviGrow and eviMake2) and its proprietary transfection reagent (eviFect). Supernatant was harvested by centrifugation and subsequent filtration (0.2 μm filter) and, proteins were purified from the harvested supernatant by standard methods.

### Purification of IgG-Like Proteins

Proteins were purified from filtered cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. The protein was concentrated by centrifugation (Millipore Amicon® ULTRA-15 (Art.Nr.: UFC903096), and aggregated protein was separated from monomeric protein by size exclusion chromatography in 20 mM histidine, 140 mM sodium chloride, pH 6.0.

### Analytics of IgG-Like Proteins

The concentrations of purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII or LabChip GX Touch (Perkin Elmer) (Perkin Elmer). Determination of the aggregate content was performed by HPLC chromatography at 25° C. using analytical size-exclusion column (TSKgel G3000 SW XL or UP-SW3000) equilibrated in running buffer (200 mM KH2PO4, 250 mM KCl pH 6.2, 0.02% NaN3).

### Preparation of Lentivirus Supernatants and Transduction of Jurkat Cells

Lipofectamine LTX™-based transfection was performed using ˜70% confluent Lenti-X™ 293T cells (Takara, #632180) and chimeric receptor encoding transfer vectors as well as packaging vectors pCAG-VSVG and psPAX2 at a 2:1:2 molar ratio (Giry-Laterriere M, et al Methods Mol Biol. 2011; 737:183-209, Myburgh R, et al Mol Ther Nucleic Acids. 2014). As control for every experiment also mock virus like particles (VLPs) using only the packaging vectors, but no transfer vector, were produced. After 48 h, the supernatant was collected and centrifuged for 5 min at 350×g to remove remaining cells and purify the virus particles. VLPs were used directly or concentrated 10-fold (Lenti-x-Concentrator, Takara, #631231) and added together with 8 μg/ml Polybrene (Sigma Aldrich) and Lentiboost P (1:100) (Sirion Biotech, #SB-P-LV-101-12) to a 24 well plate for spinfection of Jurkat NFAT (GloResponse Jurkat NFAT-RE-luc2P, Promega #CS176501) or Jurkat TCRab-KO CD4+ (T Cell Activation Bioassay (TCRaβ-KO), Promega #GA1172) cells at 1100×g for 99 min and 31° C. The cells were incubated for at least 72 hours before the transduction was checked by flow cytometry.

### Sorting of Transduced Cells

If the rate of transduced cells was lower than 90%, cells were sorted for eGFP positive cells. Between 3-10 million cells were collected and spun down (400×g, 4 min). The media was removed and the cells were resuspended in MACS buffer (Miltenyi Biotec, #130-091-222) supplemented with 5% BSA (Miltenyi Biotec, #130-091-376). The cells were pool sorted for eGFP positive cells on the BD FACSArialII. For sorting of Jurkat cells, the 100 micron nozzle was used and the 4-way purity precision mode was applied.

### Jurkat NFAT Activation Assay

The Jurkat activation assay measures T cell activation of a human acute lymphatic leukemia reporter cell line (GloResponse Jurkat NFAT-RE-luc2P, Promega #CS176501 or T Cell Activation Bioassay TCRαβ-KO CD4+, Promega #GA1172)). This immortalized T cell line is genetically engineered to stably express a luciferase reporter driven by T cell activation via CD3. After transduction, the cell line expresses a chimeric antigen receptor construct possessing a CD3z signaling domain. Binding of the construct to an immobilized adapter molecule (e.g. a tumor antigen bound adapter molecule) leads to crosslinking resulting in T cell activation and in the expression of luciferase. After addition of a substrate, the cellular changes of the NFAT activity can be measured as relative luminescence units. The assay was performed in a 384 plate (Falcon #353988 flat white, white bottom). Target cells (transduced Jurkat cells) and effector cells were seeded in a 2.5:1 ratio (20 000 effector cells and 8000 target cells) in 20 μl or 10 ul respectively, in RPMI-1640+10% FCS+1% Glutamax (growth medium) in triplicates. For the Jurkat NFAT assay the readout was performed using GloSensor CAMP Reagent (Promega, #E1291) and 2% of the end volume (here: 40 ul, resulting in 0.8 ul/well) were added to the wells. Further, a serial dilution of the antibody of interest was prepared in growth medium and 10 ul were added to the wells to obtain a final concentrations ranging from 20 nM to 0.00122 nM in the assay plate with a final volume of 40 μl per well in total. The 384 well plate was centrifuged for 1 min at 300 g at RT and incubated at 37° C. and 5% CO₂ in a humidified atmosphere. After 4-6 h incubation, the plates were adjusted to room temperature for 10 minutes before measurement. For the Jurkat TCRab-KO CD4+ cells 100% of the final volume of Bio-Glo-NL reagent (Promega, #J3082) was added, and plates were centrifuged for 1 min at 350×g and incubated for 5-10 minutes. In the assay using the Jurkat NFAT cells the CAMP reagent was already added when the cells were seeded (see above). Afterwards, the relative luminescence units (RLU) per s/well were measured immediately using a Tecan microplate reader. Concentration-response curves were fitted and EC₅₀ values were calculated using GraphPadPrism version 8.

Isolation of Primary T Cells from Buffy Coats

Buffy coats were ordered from Blutspende Zürich (Rütistrasse 19, 8952 Schlieren). A Leucosep tube with 15 mL of room temperature Histopaque density gradient medium (Sigma-Aldrich, #10771) was prepared and centrifuged at 400×g for 5 minutes, until the Histopaque passed the filter. The blood was transferred to a T75 flask and an equal volume of DPBS was added, 30 ml of the blood/buffer mixture was added to the Leucosep tubes and they were centrifuged 1200×g for 20 minutes with acceleration and breaks off. The band containing the peripheral blood mononuclear cells (PBMCs) was carefully pipetted into a fresh 50 ml falcon tube and topped up to 50 ml with DPBS. The tubes were centrifuged at 350×g for 10 minutes (with low breaks), then the supernatant was discarded. The cells were washed three more times with DPBS (1, 250×g, 10 minutes: 2, 250×g, 10 minutes: 3, 120×g, 10 minutes), before the cells were resuspended and counted. CD4+ and CD8+ T cell isolation was performed by negative selection according to the manufacturer's instructions using the CD4+ or CD8+ T cell isolation kit (Miltenyi, #130-096-533, #130-096-495). The cells were either frozen or used directly after isolation. Cells were cultured in advanced RPMI (Gibco, #11530446). 10% FBS (Sigma, #F4135-500 ML). 1% Glutamax (Gibco, #35050-038). 50 IU/Proleukin (Novartis), 25 ng/ml IL-7 (Miltenyi, #130-095-364) and 50 ng/ml IL-15 (Miltenyi, #130-095-766) (T cell medium).

### Preparation of Virus Like Particles (VLPs)

Lipofectamine LTX™-based transfection (Thermo Fisher, #15338-100) was performed using ˜70% confluent Lenti-X™ 293T cells (Takara, #632180) and the construct encoding transfer vectors as well as packaging vectors pCAG-VSVG and psPAX2 at a 2:1:2 molar ratio (Giry-Laterriere M, et al Methods Mol Biol. 2011; 737:183-209, Myburgh R, et al Mol Ther Nucleic Acids. 2014). As control for every experiment, mock virus-like particles (VLPs) using only the packaging vectors, but no transfer vector, were produced. After 48 hours, the supernatant was collected and centrifuged for 10 minutes at 500×g to remove remaining cells. The VLPs were concentrated 10-fold by using Lenti-X Concentrator (Takara, #631231) according to the manufacturer's instructions. For storage, the VLPs were aliquoted in Eppendorf tubes and snap frozen in liquid nitrogen, before being stored at −80° C.

### Transduction of Primary T Cells

CD4+ and CD8+ T cells were thawed, mixed 1:1 and directly activated for 24 hours using ImmunoCult™ Human CD3/CD28/CD2 T Cell Activator (StemCell, #10990) according to the manufacturer's manual. Then the activated cells were resuspended, counted and 1 million cells/well/ml were seeded in a 24-well plate. VLPs were thawed at 37° C. and 150 μl were added together with 8 μg/ml Polybrene (Sigma Aldrich) and Lentiboost P (1:100) (Sirion Biotech, #SB-P-LV-101-12) to the activated cells in the 24-well plate. The cells were incubated for 24 hours at 37° C., 5% CO2, before they were resuspended in 4 ml of fresh T cell medium and added to a gREX plate (Wilson Wolf, #MSPP-80192M) for expansion. Expression of the constructs was checked by flow cytometry from 72 hours after transduction.

### STAT5 Phosphorylation Assay

Non-transduced and transduced T cells were spun down, washed with DPBS and seeded for starvation (4 hours to overnight) in medium without cytokines (advanced RPMI, 10% FBS, 1× Glutamax (assay medium)). The fix buffer I (BD, #557870) was placed at 37° C. and the Perm Buffer III (BD, #558050) was placed at −20° C. before the assay. The starved T cells were spun down and resuspended in assay medium. Depending on the expression rate of the transduced T cell pool, the T cells were mixed with non-transduced cells to ensure a similar distribution of non-transduced cells (eGFP−) and construct expressing cells (eGFP+) in each well. 150,000-300,000 cells were seeded in 25 μl in a 96-well V-bottom plate. Cytokines or molecules were diluted in assay medium and 50 μl of the dilutions were added to the T cells in duplicates. The plate was incubated for 15 minutes at 37° C., before 75 μl Fix Buffer I were added to the wells. After incubating the plate an additional 30 minutes at 37° C., the plate was spun down (400×g, 3 min) and the supernatant discarded. The cells were resuspended in 100 μl ice cold Perm Buffer III and incubated on ice for 30 minutes. At this stage, the cells were either stained directly or stored at −20° C. for up to two weeks.

For the staining, the plate was washed twice with DPBS (400×g, 3 min) before the cells were resuspended in 50 μl diluted AF647 mouse anti-Stat5 (pY694) staining antibody (1:20 in FACS buffer, BD, #562076) and incubated at 4° C. for 1 hour. Then the cells were washed twice with FACS buffer, before the samples were acquired on the BD FACS Fortessa. In order to compare the effect of the cytokine fusion molecules on the non-transduced (eGFP−) cells with the effect on the transduced (eGFP+) cells, the median fluorescence intensity of the two populations was analyzed.

### Incucyte® Immune Cell Killing Assay

Cancer cell lines (HeLa and MKN45) were in house transduced with the Incucyte® NucLight Red Lentivirus (EF1α, Puro, #4476) and stable cell lines were created under puromycin selection (HeLa NLR, MKN45 NLR). Human CD4+/CD8+ T cells were isolated and transduced with the desired constructs as described above. For the assay, HeLa NLR or MKN45 NLR cells were resuspended in RPMI-1640 (Gibco, #42401-018)+2% FCS (Sigma, #F4135-500 ML)+1% Glutamax (Gibco, #35050-038) (killing medium) and 10,000 cells seeded in 100 μl in each well of a flat bottom 96-well plate. The plate was incubated for 4-24 hours at 37° C., 5% CO2, until the cells were attached. The transduced T cells were counted, adjusted to 10,000 eGFP+ cells/50 μl in killing medium and added to the attached cancer cells. Adaptor and control antibodies were diluted in killing medium to the desired concentrations and 50 μl were added to the wells. Every condition was pipetted in duplicates. Bubbles were removed from the wells surface and the plates were placed in the Incucyte® S3 machine. Five images of each well were captured every 4 hours over a course of 5 to 6 days. The reduction in cancer cell numbers was quantified using an analysis mask counting the amount of red cells.

### Example 1

### Generation and Characterization of Humanized Anti-P329G Antibodies

Parental and humanized anti-P329G antibodies were produced in HEK cells and purified by ProteinA affinity chromatography and size exclusion chromatography. All antibodies were purified in good quality (Table 13).

TABLE 13
Biochemical analysis of anti-P329G antibodies. Monomer content
determined by analytical size exclusion chromatography. Purity
determined by non-reducing SDS capillary electrophoresis.

| Molecule | Monomer [%] | Purity [%] |
|---|---|---|
| Anti-P329G (M-1.7.24) huIgG1 | 100 | 85 |
| Anti-P329G (VH1VL1) huIgG1 | 100 | 97 |
| Anti-P329G (VH2VL1) huIgG1 | 100 | 87 |
| Anti-P329G (VH3VL1) huIgG1 | 100 | 97 |

### Binding of Parental and Six Humanization Variants of Anti-P329G Binder M-1.7.24 to Human Fc (P329G)

### Instrumentation: Biacore T200

### Chip: CM5 (#772)

Fc1 to 4: anti-human Fab specific (GE Healthcare 28-9583-25)

### Capture: 50 nM IgGs for 60 s

Analyte: human Fc (P329G) (P1AD9000-004)
Running buffer: HBS-EP

### To: 25° C.

Dilution: 2-fold dilution in HBS-EP from 0.59 to 37.5 nM
Flow: 30 μl/min

### Association: 240 sec

### Dissociation: 800 sec

Regeneration: 10 mM glycine pH 2.1 for 2×60 sec

SPR experiments were performed on a Biacore T200 with HBS-EP+ as running buffer (0.01 M HEPES pH 7.4, 0.15 M NaCl, 0.005% Surfactant P20 (BR-1006-69, GE Healthcare)). Anti-human Fab specific antibodies (GE Healthcare 28-9583-25) were directly immobilized by amine coupling on a CM5 chip (GE Healthcare). The IgGs were captured for 60 s at 50 nM. A two-fold dilution series of the human Fc (P329G) was passed over the ligand at 30 μl/min for 240 sec to record the association phase. The dissociation phase was monitored for 800 s and triggered by switching from the sample solution to HBS-EP+. The chip surface was regenerated after every cycle using two injections of 10 mM glycine pH 2.1 for 60 sec. Bulk refractive index differences were corrected for by subtracting the response obtained on the reference flow cell 1. The affinity constants were derived from the kinetic rate constants by fitting to a 1:1 Langmuir binding using the Biaeval software (GE Healthcare). The measure was performed in triplicate with independent dilution series.

Following samples were analyzed for binding to human Fc (P329G) (Table 14).

TABLE 14
Description of the samples analyzed
for binding to human Fc (P329G).

| Binder | TAPIR ID | Format |
|---|---|---|
| Anti-P329G (M-1.7.24) | P1AE9963 | IgG, supernatant/purified |
| (parental) | | |
| Anti-P329G (VH3VL1) | P1AE9957 | IgG, supernatant/purified |
| Anti-P329G (VH1VL1) | P1AE9955 | IgG, supernatant/purified |
| Anti-P329G (VH2VL1) | P1AE9956 | IgG, supernatant/purified |
| Anti-P329G (VH4VL1) | P1AE9958 | IgG, supernatant |
| Anti-P329G (VH1VL2) | P1AE9959 | IgG, supernatant |
| Anti-P329G (VH1VL3) | P1AE9960 | IgG, supernatant |
| human Fc (P329G) | P1AD9000-004 | Antigen used as analyte |

Human Fc (P329G) was prepared by plasmin digestion of a human IgG1 followed by affinity purification by ProteinA and size exclusion chromatography.

### Binding of Parental and Six Humanization Variants of Anti-P329G Binder M-1.7.24 to Human Fc (P329G)

The dissociation phase was fitted to a single curve to help characterize the off-rate. The ratio between binding to capture response level was calculated. (Table 15).

TABLE 15
Binding assessment of six humanization variants
for binding to human Fc (P329G).

| | | kd | Ratio | |
|---|---|---|---|---|
| Binder | TAPIR ID | (1/s) | binding/capture | Binding |
| Anti-P329G | P1AE9963-001 | 5.73E−03 | 20 | parental |
| (M-1.7.24) | | | | |
| (parental) | | | | |
| Anti-P329G | P1AE9957-001 | 5.49E−03 | 20 | as parental |
| (VH3VL1) | | | | |
| Anti-P329G | P1AE9955-001 | 3.88E−03 | 20 | as parental |
| (VH1VL1) | | | | |
| Anti-P329G | P1AE9956-001 | 2.79E−03 | 23 | as parental |
| (VH2VL1) | | | | |
| Anti-P329G | P1AE9958-001 | 1.11E−02 | 19 | reduced |
| (VH4VL1) | | | | |
| Anti-P329G | P1AE9959-001 | 7.86E−03 | 10 | reduced |
| (VH1VL2) | | | | |
| Anti-P329G | P1AE9960-001 | 1.29E−01 | 3 | reduced |
| (VH1VL3) | | | | |

### Affinity of Parental and Three Humanization Variants of Anti-P329G Binder M-1.7.24 to Human Fc (P329G)

Three humanization variants with binding pattern similar to parental were assessed in more details. The kinetic constants for a 1:1 Langmuir binding are summarized in Table 16.

TABLE 16
Kinetic constants (1:1 Langmuir binding). Average and standard
deviation (in parenthesis) of independent triplicate (independent
dilutions series within the same run).

| Binder | TAPIR ID | ka (1/Ms) | kd (1/s) | KD (M) | Rmax (RU) |
|---|---|---|---|---|---|
| Anti-P329G | P1AE9963-003 | 5.03E+05 | 1.58E−03 | 3.17E−09 | 44 |
| (M-1.7.24) | | (4.75 E+04) | (3.8 E−05) | (3.7 E−10) | (2) |
| (parental) | | | | | |
| Anti-P329G | P1AE9957-003 | 2.74E+05 | 1.44E−03 | 5.27E−09 | 55 |
| (VH3VL1) | | (5.51 E+03) | (7.51 E−05) | (3.3 E−10) | (3) |
| Anti-P329G | P1AE9955-003 | 2.83E+05 | 1.20E−03 | 4.24E−09 | 48 |
| (VH1VL1) | | (7.94 E+03) | (4.73 E−05) | (2.5 E−10) | (2) |
| Anti-P329G | P1AE9956-003 | 2.53E+05 | 1.22E−03 | 4.81E−09 | 54 |
| (VH2VL1) | | (3.79 E+03) | (3.61 E−05) | (2.1 E−10) | (5) |

### CONCLUSION

Six humanization variants were generated. Three of them (VH4VL1, VH1VL2, VH1VL3) showed decreased binding to human Fc (P329G) compared to parental M-1.7.24. The other three humanization variants (VH1VL1, VH2VL1, VH3VL1) have a binding kinetic very similar to the parental binder and did not lose affinity through humanization.

### Example 2

### Preparation of CH2 or CH2-CH3 Chimeric Antigen Receptors

DNA sequences encoding either the constant heavy chain 2 (CH2) with a P329G substitution or the CH2-constant heavy chain 3 (CH3) fusion containing the P329R and the AAA substitutions (1253A, H310A, H435A), were cloned and employed as targeting domain for an adaptor molecule in a chimeric receptor according to the invention.

The C-terminal ends of CH2 or CH2-CH3 are connected via G4S linker to the stalk and transmembrane domain of CD8a (Uniprot P01732 [183-203]), which is fused to an intracellular co-stimulatory signaling domain (CSD) CD137 (Uniprot Q07011AA 214-255), which in turn is fused to a stimulatory signaling domain (SSD) CD3ζ (Uniprot P20963 AA 52-164) (Figure FIGS. 1A and B). A graphical representation of an exemplary expression construct including the eGFP reporter and RQR8 purification tag is shown in FIG. 1C and for the CH2 P329G construct and in FIG. 1D for the CH2-CH3 AAA construct.

### Example 3

### Expression of CH2 or CH2-CH3 Chimeric Antigen Receptors in Jurkat-NFAT or Jurkat TCRab KO CD4+ Cells

The CH2 or CH2-CH3 receptors were virally transduced into Jurkat NFAT (GloResponse Jurkat NFAT-RE-luc2P, Promega #CS176501) or Jurkat TCRab-KO CD4+ (T Cell Activation Bioassay (TCRαβ-KO), Promega #GA1172) cells.

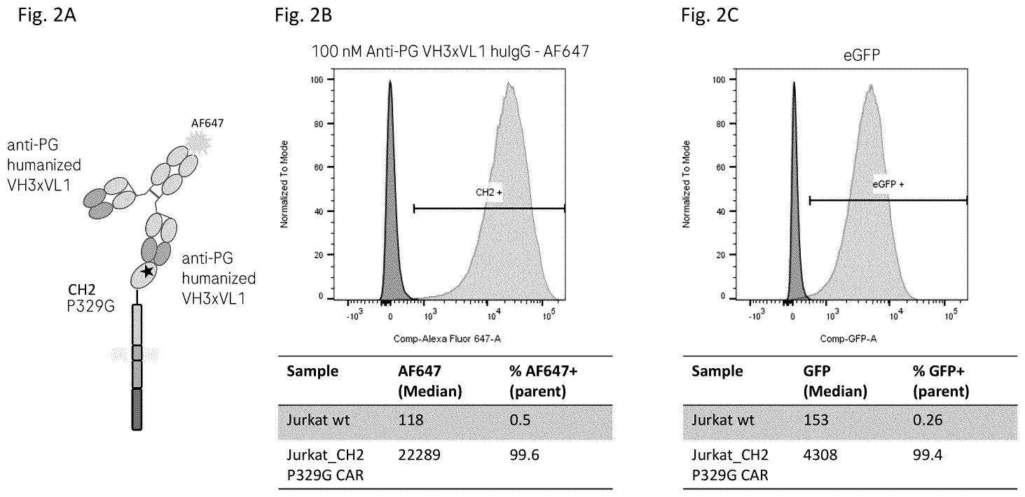

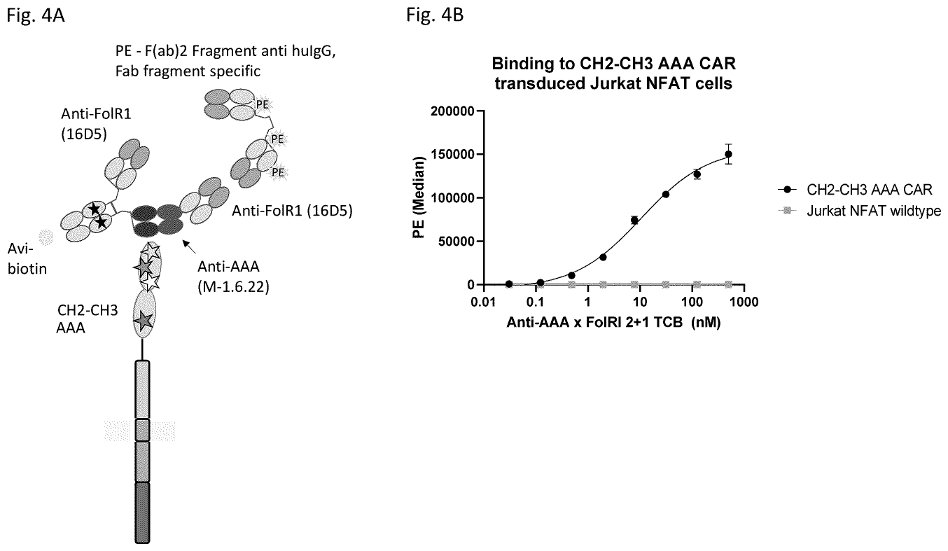

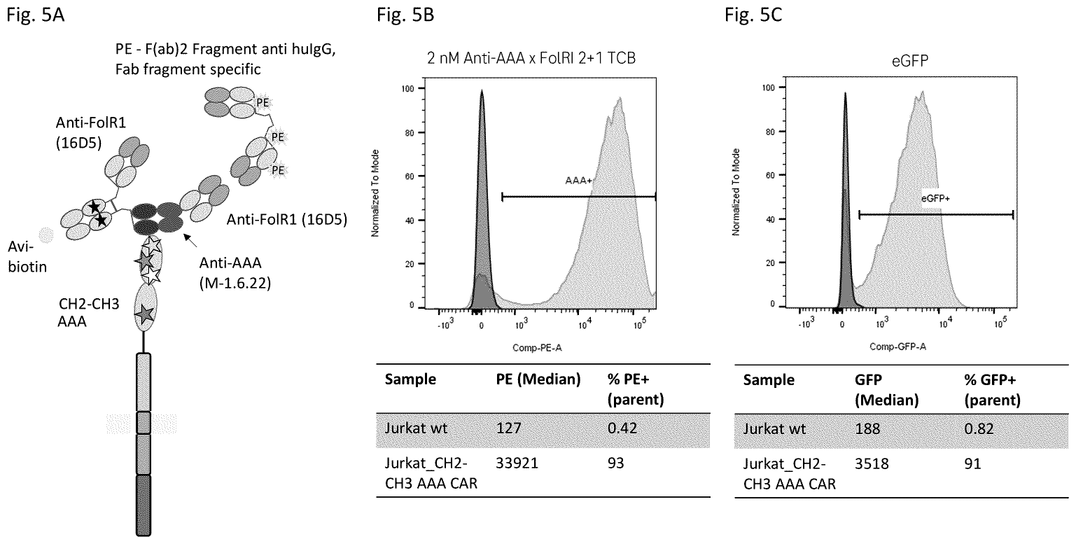

Expression of the chimeric receptors was assessed by flow cytometry. Jurkat cells featuring either the CH2 P329G construct or the CH2-CH3 AAA construct were harvested, washed with PBS and seeded at 100,000 cells per well in a 96 well U bottom plate. The cells were stained with LIVE/DEAD™ Fixable Near-IR Dead (Invitrogen, #L34976) dye (1:1000 in PBS) for 20 minutes at 4° C. and washed twice with FACS-buffer (1×PBS, 2% FBS, 5 mM EDTA pH8.0, 0.05% NaN3). The CH2-CH3 AAA construct transduced cells were incubated with different concentrations (500 nM-0 nM serial dilution of 1:4) of anti-AAA×FolRI 2+1 antibody and incubated for 30 minutes at 4° C. The samples were washed two times with FACS-buffer and then stained with R-Phycoerythrin AffiniPure F(ab′)₂ Fragment Goat Anti-Human IgG, F(ab′)₂ fragment specific antibody (1:50 dilution) for 30 min in the dark at 4° C. (FIGS. 4A and 4B). For the analysis of the receptor expression, a concentration of 2 nM anti-AAA×FolRI 2+1 antibody was chosen and is depicted in FIG. 5B. The CH2 P329G construct expression was also analyzed via FACS-staining. Therefore the cells were harvested, washed and stained for living cells as described above. The CH2 P329G construct was detected by staining with 100 nM anti-P329G VH3×VL1 AF647 labeled IgG (FIGS. 2A and 2B). Additionally, the intracellular eGFP reporter was analyzed (see FIG. 2C and FIG. 5C).

Both, the CH2 P329G construct and the CH2-CH3 AAA construct were shown to be expressed on the surface of the Jurkat cells and the expression correlated with the intracellular eGFP expression.

To test the functionality of the CARs they were assessed in a Jurkat activation assay.

### Example 4

### Specific T Cell Activation in the Presence of Targeting Antibody Comprising the Anti-P329G or Anti-AAA Binder

To assess specific T cell activation, the CH2 P329G Jurkat cells and CH2-CH3 AAA Jurkat cells were evaluated towards their activation in the presence of FolR1-positive target cells and anti-PG×FolRI 2+1 antibody (FIG. 3A) or anti-AAA×FolRI 2+1 antibody (Figure FIG. 6A). The CH2 P329G Jurkat cells were tested with high (HeLa), medium (Ovcar-3) and low (HT29) FolR1-positive target cells. Mock transduced Jurkat cells (transduced with VLPs without transgene vector) served as a negative control. Dose-dependent and also antigen level-dependent activation of the CH2 P329G Jurkat cells was observed. The mock transduced control cells showed no activation by anti-PG×FolRI 2+1 antibody with any of the tested target cells (FIGS. 3B-D). The CH2-CH3 AAA Jurkat cells were tested in an activation assay with high (HeLa) FolR1-positive target cells. Jurkat NFAT wildtype cells served as a negative control. Also here a dose-dependent activation of the CH2-CH3 AAA Jurkat cells was observed, while no activation of the Jurkat NFAT wildtype cells was observed (FIG. 6B).

Those results lead to the conclusion that the CH2 P329G construct as well as the CH2-CH3 AAA construct can be expressed in T cells, are both functional and can be selectively activated by antibodies comprising the cognate antibody binders.

### Example 5

### Expression of CH2-CH3 AAA P329G CAR, CH2-CH3 AAA P329R CAR or CH2 P329G CAR in Primary T Cells

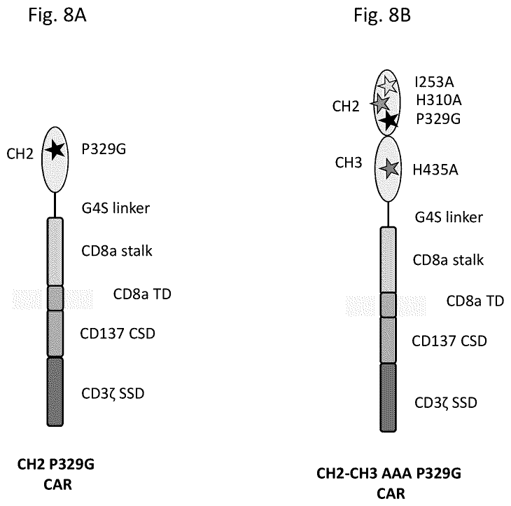

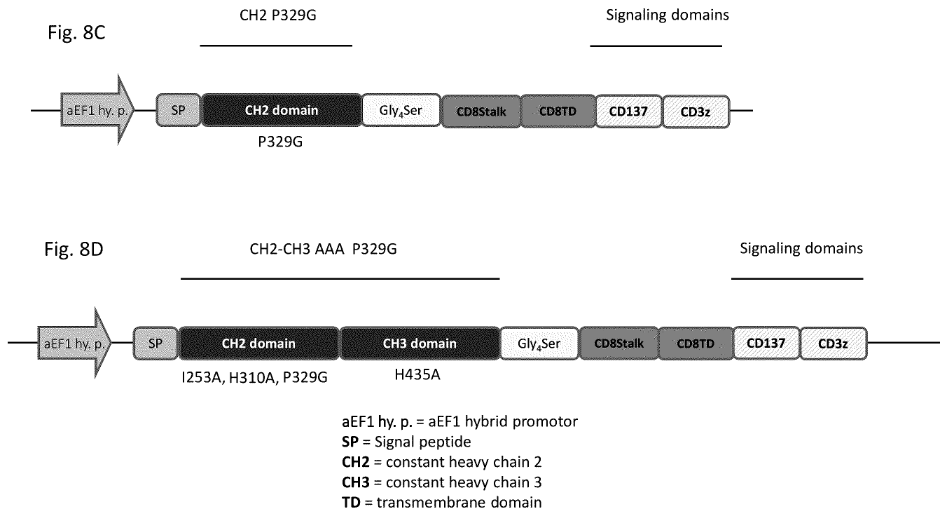

The CH2-CH3 AAA P329G CAR (FIG. 8B, SEQ ID NO: 127), CH2-CH3 AAA P329R CAR (FIG. 1B, SEQ ID NO: 125) or CH2 P329G CAR (FIG. 1A, SEQ ID NO: 87) receptors were transduced with virus-like particles (VLPs) into human T cells (1:1 CD4+:CD8+) as described above.

The expression of the chimeric receptors was assessed and compared by flow cytometry on day 7 after transduction as follows. Transduced T cells were harvested, washed with DPBS and seeded at 100,000 cells per well in a 96-well U bottom plate. The T cells transduced with CARS containing the P329G mutation were stained with LIVE/DEAD™ Fixable Near-IR Dead (Invitrogen, #L34976) dye (1:1000 in DPBS) and 50 nM of anti-PG VH3×VL1 huIgG-AF647 for 20 minutes at 4° C. Then the cells were washed twice with FACS-buffer (1×DPBS. 2% FBS. 5 mM EDTA pH 8.0, 0.05% NaN3), fixed (BD CytoFix, #554655) and analyzed on the FACS.

Cells transduced with CARs containing the AAA mutation were stained with LIVE/DEAD™ Fixable Near-IR Dead (Invitrogen, #L34976) dye (1:1000 in DPBS) and 200 nM of anti-AAA×FolR1 2+1 TCB (20 minutes at 4° C.). Then the cells were washed twice with FACS-buffer. The cell pellets were resuspended in 50 ul secondary antibody diluted in FACS-buffer (1:100, APC-F(ab)₂ Fragment anti-huIgG (Jackson ImmunoResearch, #109-136-097)) and incubated for 30 minutes at 4° C. After another two washing steps, the cells were fixed (BD CytoFix, #554655) and analyzed on the FACS.

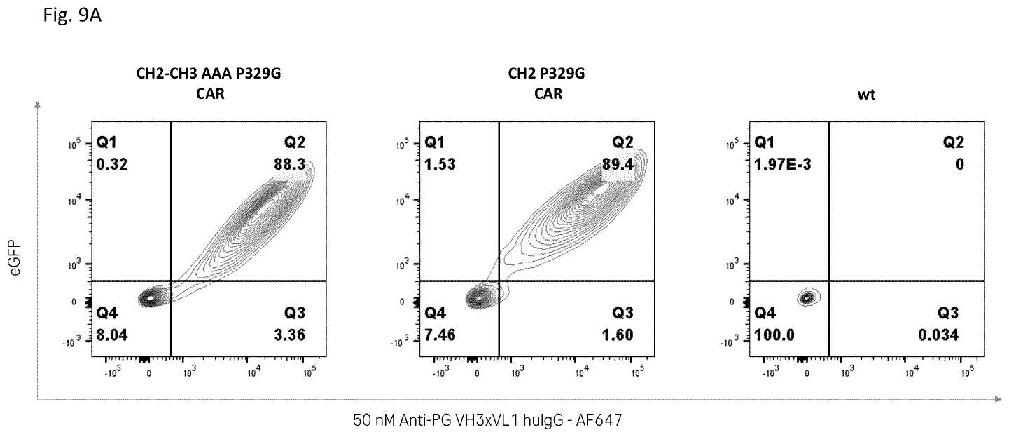

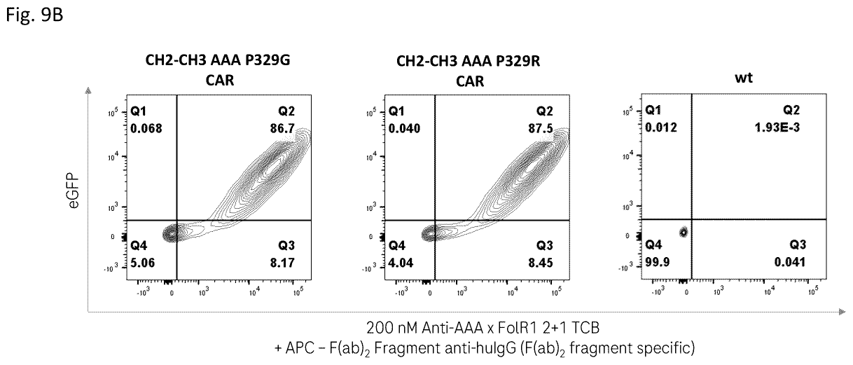

The intracellular eGFP expression and anti-PG/anti-AAA staining is depicted in FIGS. 9A and 9B. Between 87-89% of the cells were eGFP positive and expressing the CAR constructs on the cell surface.

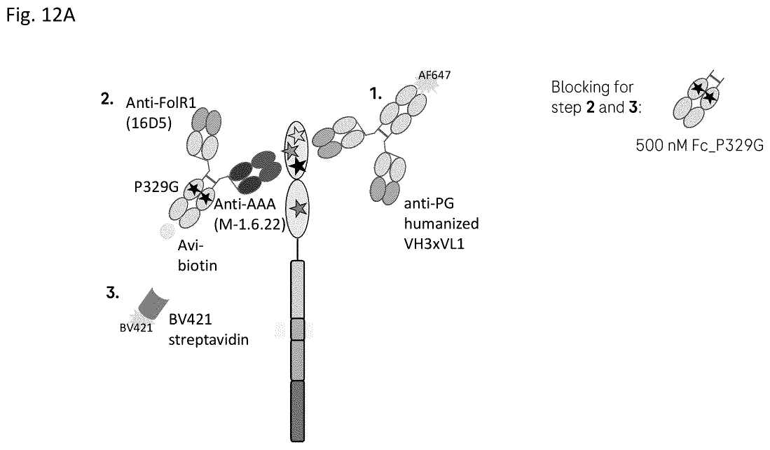

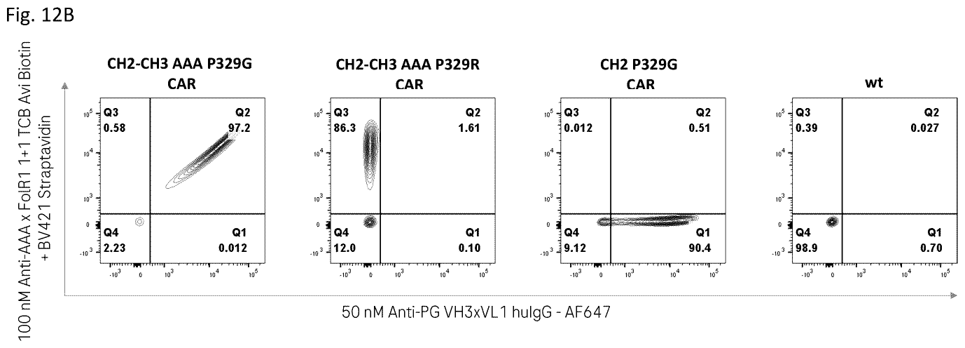

In order to assess if the P329G mutation and AAA mutations can be bound by the respective molecules simultaneously, a second staining was performed as follows (FIG. 12A). The cells were incubated with LIVE/DEAD™ Fixable Near-IR Dead (Invitrogen, #L34976) dye (1:1000 in DPBS) and 50 nM of anti-PG VH3×VL1 huIgG-AF647 for 20 minutes at 4° C.

Then the cells were washed twice with FACS-buffer. In a second step the cells were incubated with 100 nM of anti-AAA×FolR1-Avi-Biotin 1+1 TCB and 500 nM Fc_P329LALA (20 minutes at 4° C.). Fc_P329GLALA served as blocking molecule, to prevent binding of the anti-PG VH3×VL1 huIgG-AF647 to the PG mutation in the anti-AAA×FolR1-Avi-Biotin 1+1 TCB. After the incubation, the cells were washed twice with FACS-buffer. In a third step the cells were resuspended in BV421 Streptavidin (Biolegend, #405225) and 500 nM Fc_P329LALA (20 min, 4° C.). After another two washing steps, the cells were fixed (BD CytoFix, #554655) and analyzed on the FACS.

The CH2-CH3 AAA P329G CAR T cells showed double staining (BV421+, AF647+), indicating that AAA and P329G mutations can be targeted simultaneously in one CAR construct with anti-AAA and anti-PG adaptors (FIG. 12B). The CH2-CH3 AAA P329R CAR or CH2 P329G CAR T cells only showed the respective single staining as expected.

To test the functionality of the constructs, they were assessed in an Incucyte® immune cell killing assay. For further evaluation of targeting cytokines in cis to the CAR T cell via the P329G mutation, a STAT5 phosphorylation assay was performed.

### Example 6

### Incucyte® Immune Cell Killing Assay with CEA+ or FolR1+ Target Cells

To assess the cytotoxicity of the CH2-CH3 AAA P329G CAR (FIG. 8B, SEQ ID NO: 127). CH2-CH3 AAA P329R CAR (FIG. 1B, SEQ ID NO: 125) or CH2 P329G CAR (FIG. 1A, SEQ ID NO: 87) receptors, a killing assay was performed. In order to compare the different constructs, the amount of T cells per well was normalized to the same percentage of eGFP+ cells, resulting in 10,000 target cells and 10.000 eGFP+ T cells/well. The killing assay was performed as described above.

As adaptor molecules for HeLa NLR, anti-AAA×FolRI 2+1, anti-AAA×FolRI 1+1, anti-PG×FolRI 2+1 or anti-PG×FolRI 1+1 antibodies were titrated from 0 pM-10 nM (1:10). As positive control for the killing served 10 nM anti-FolR1 2+1 TCB.

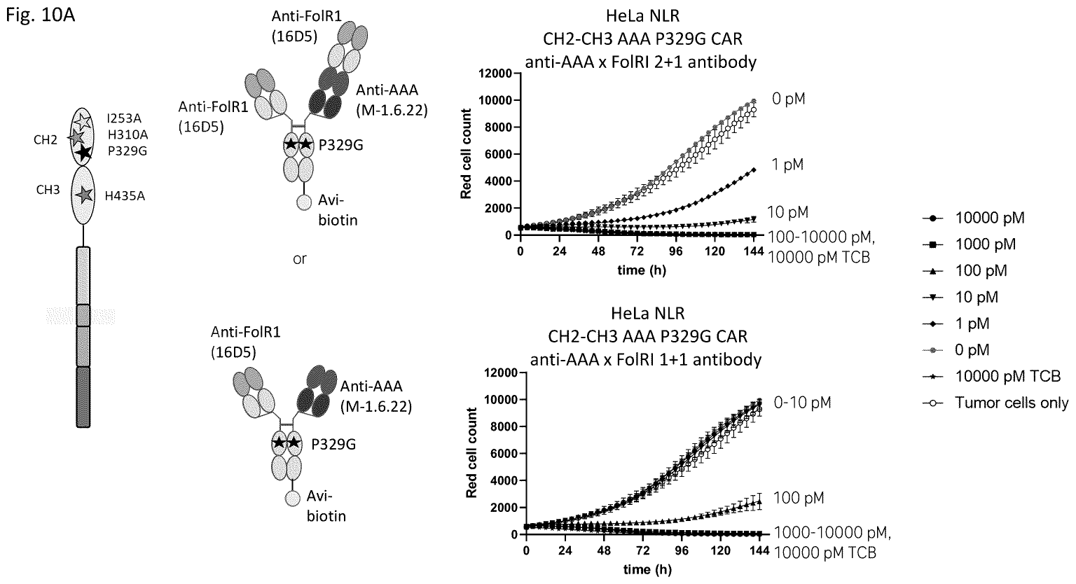

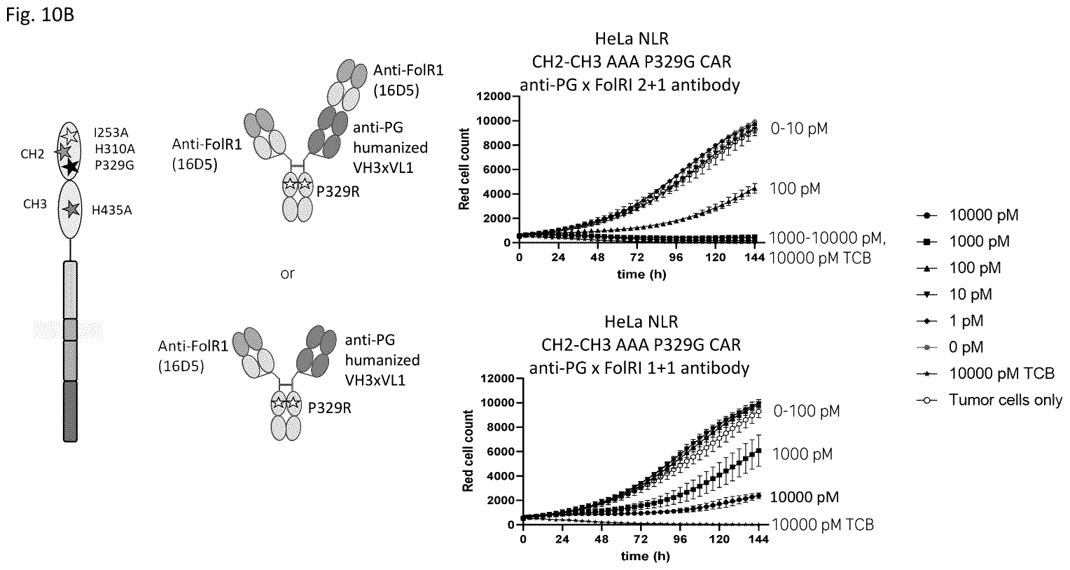

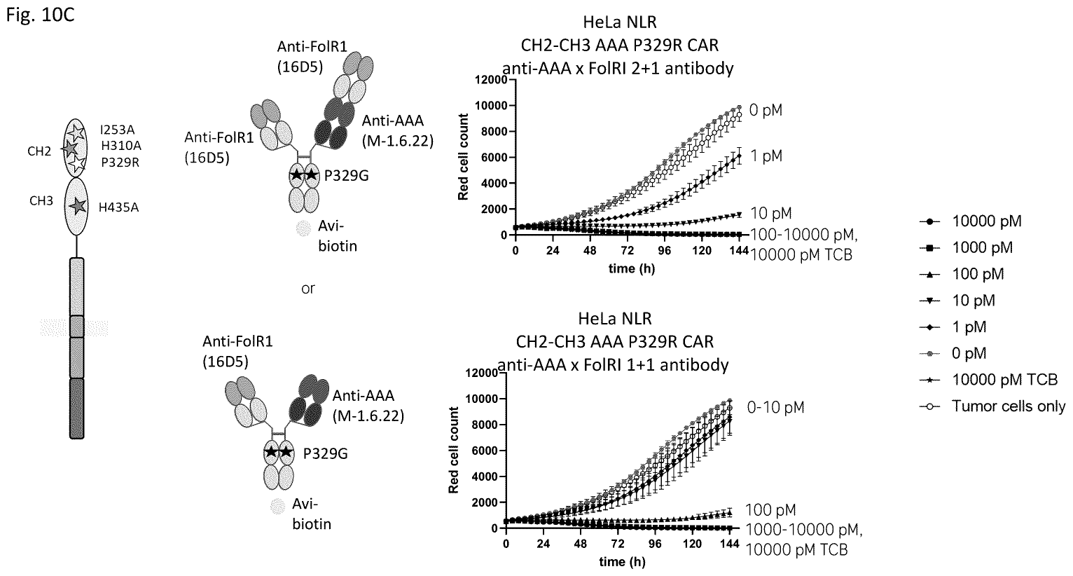

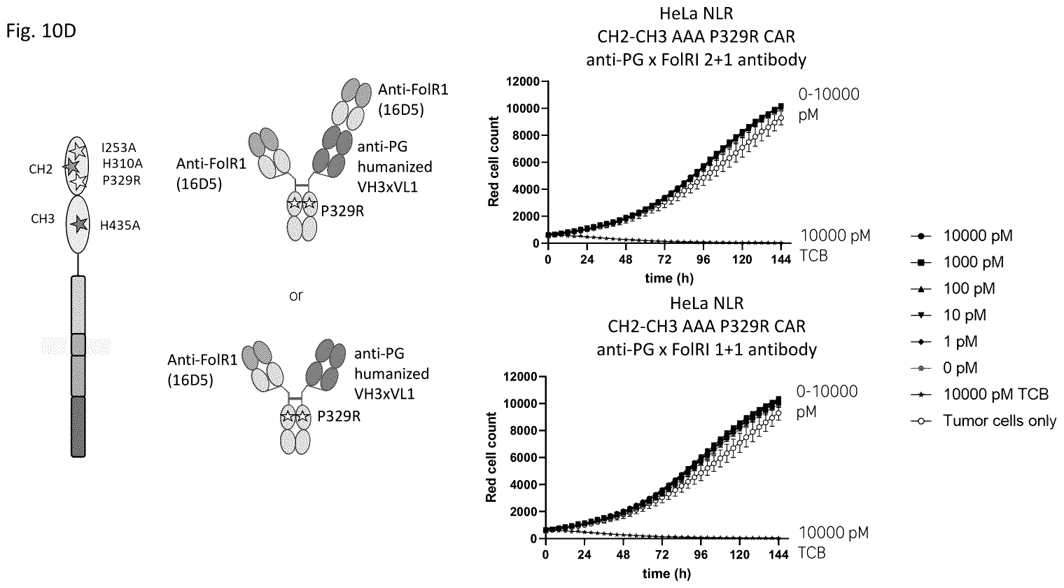

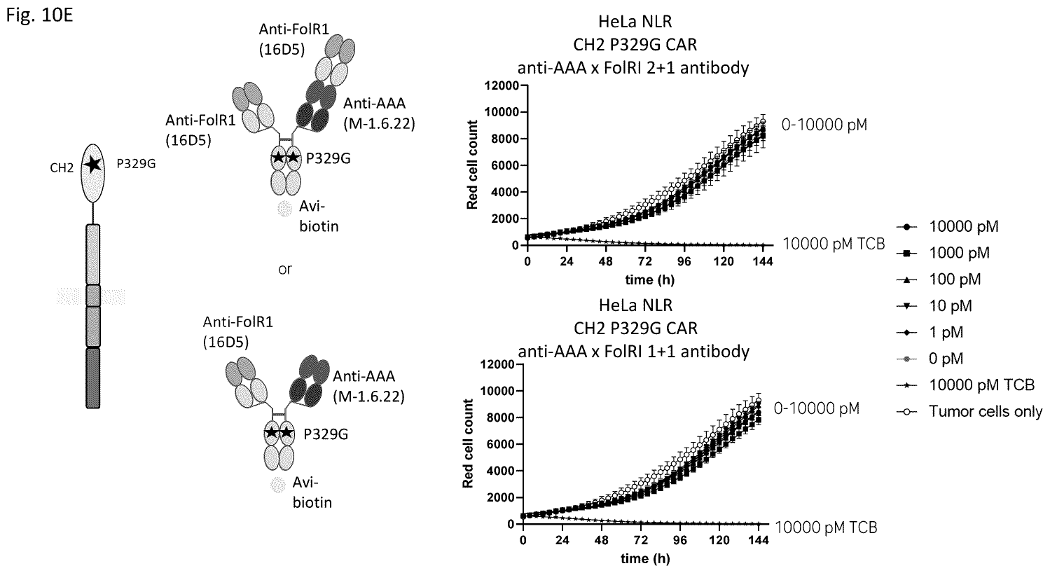

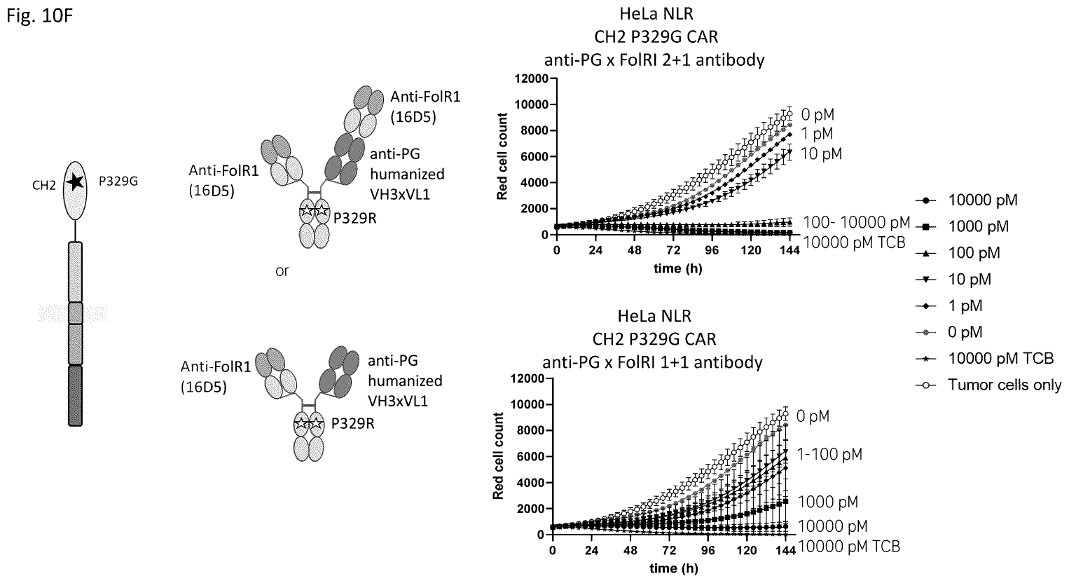

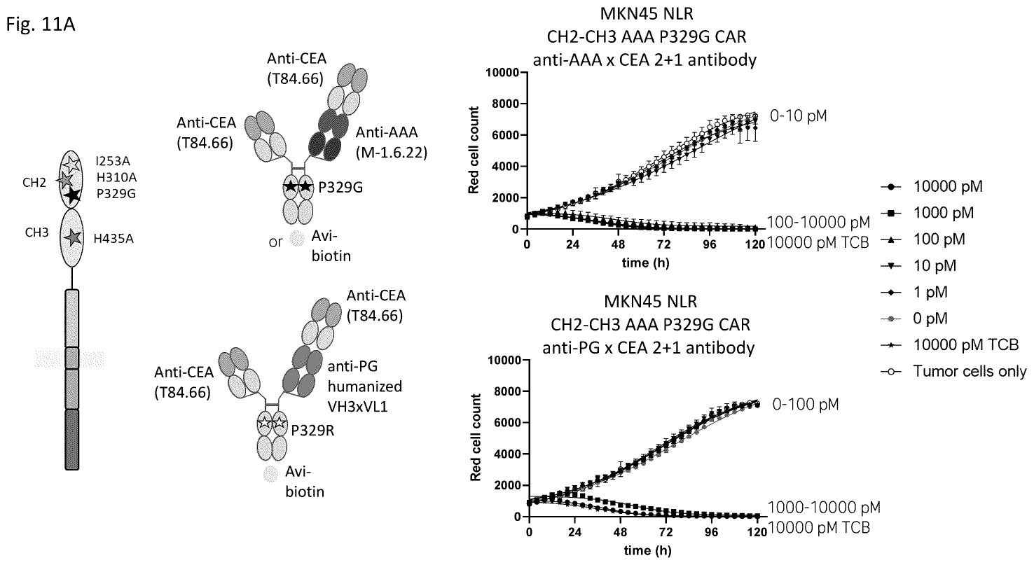

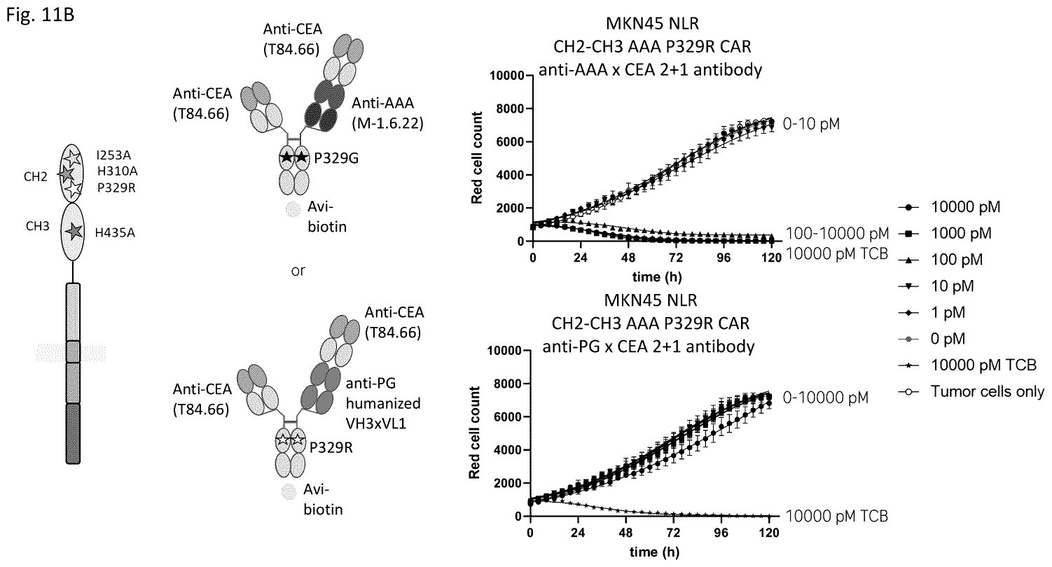

Dose dependent killing was observed with all CAR constructs incubated with their corresponding adaptor molecules (FIGS. 10A, 10B, 10C, 10F). Incubation of T cells expressing CH2-CH3 AAA P329R CAR with anti-PG×FolR1 antibodies did not result in any killing as expected (FIG. 10D). The same was observed for CH2 P329G CAR T cells incubated with anti-AAA×FolR1 antibodies (FIG. 10E).

In both approaches (anti-PG or anti-AAA) the 2+1 format showed more sensitive killing compared to the 1+1 format, independent of the CAR construct (FIGS. 10A, 10B, 10C, 10F). In direct comparison, the anti-AAA×FolR1 molecules showed a higher sensitivity compared to the anti-PG×FolR1 adaptors.

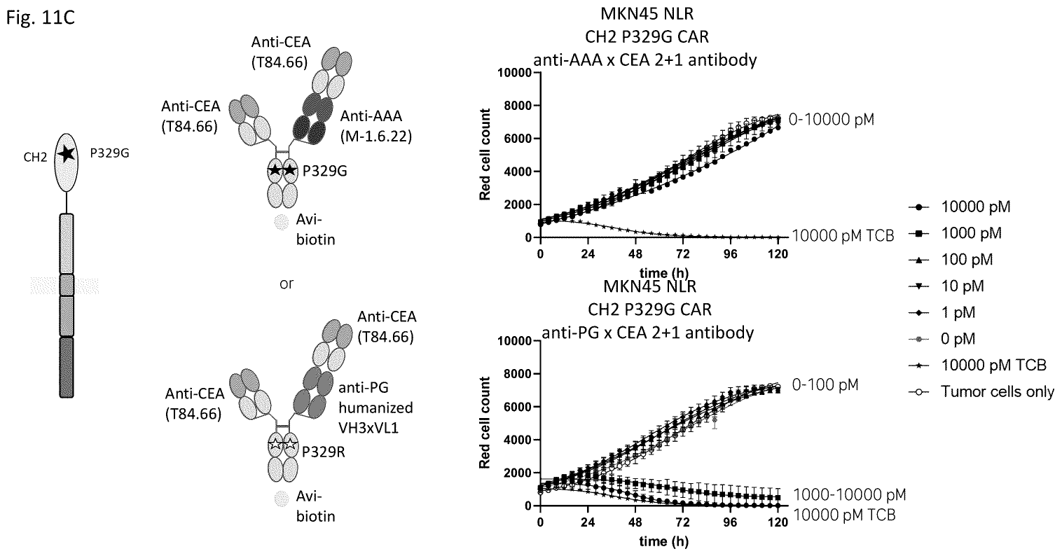

MKN45 NLR cells were targeted with anti-AAA×CEA 2+1 or anti-PG×CEA 2+1 antibodies (0 pM-10 nM. (1:10)). Dose dependent growth inhibition was observed with all CAR constructs incubated with their corresponding adaptor molecules (FIGS. 11A, 11B (top), 11C (bottom)). As observed previously, addition of an adaptor molecule with a binder not matching the mutation present in the CAR construct did not result in any killing (FIGS. 11B (bottom) and 11C (top)). Consistent with the HeLa NLR killing data, the anti-AAA×CEA molecules showed higher sensitivity compared to the anti-PG×CEA adaptors.

### Example 7

### STAT5 Phosphorylation Assay with Anti-P329G Targeted IL2v

In order to assess the selective targeting of a cytokine to the CH2-CH3 AAA P329G CAR (FIG. 8B, SEQ ID NO: 127) or CH2 P329G CAR (FIG. 1A, SEQ ID NO: 87), a STAT5 phosphorylation assay was performed. To ensure a similar distribution of non-transduced cells (eGFP−) and construct expressing cells (eGFP+) in each well, non-transduced cells and transduced cells were mixed 1:1 for this assay (>90% of the transduced cells were expressing the construct). Anti-P329G-IL2v or Proleukin (Novartis) were titrated on the cells, starting with 100 nM (1:10). The assay was performed as described above. The samples were acquired on the BD FACS Fortessa and the median fluorescence intensity (MFI) of the pSTAT5 signal on the non-transduced (eGFP−) population vs. the transduced (eGFP+) population was plotted in GraphPad Prism 8.

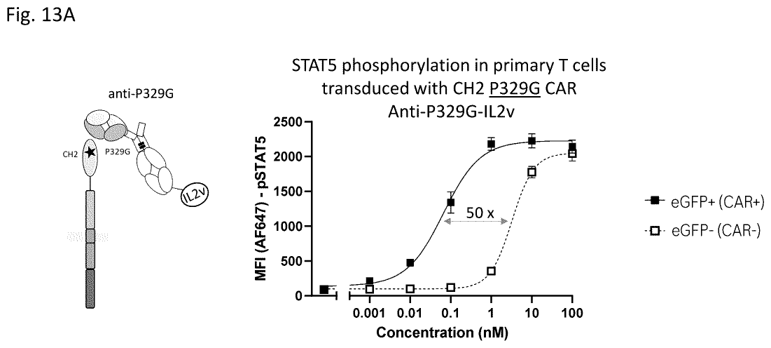

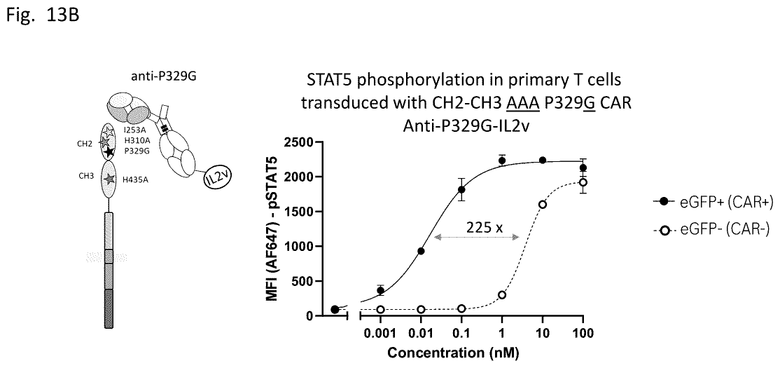

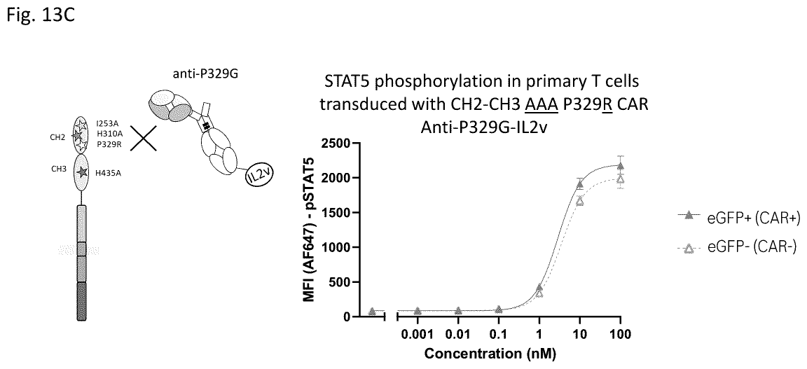

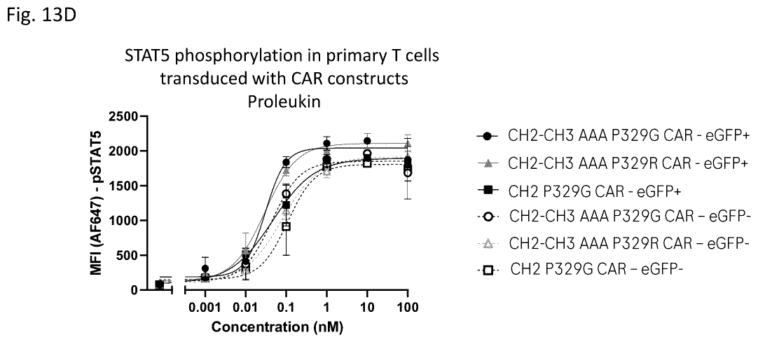

FIG. 13 shows the results of the pSTAT5 assay. In the samples with the CH2 P329G CAR T cells a 50-fold increase in pSTAT5 MFI in the eGFP+ population compared to the eGFP-population was observed (FIG. 13A). This effect was even larger when the T cells were expressing the CH2-CH3 AAA P329G CAR (250-fold, FIG. 13B), showing very strong cis-targeting of IL2v to the eGFP+ population. The cis-effect was not observed in the CH2-CH3 AAA P329R CAR T cell, confirming that the effect is indeed due to P329G targeting. Looking at the data obtained with Proleukin, all samples and populations show comparable STAT5 phosphorylation.

1. A chimeric receptor comprising
(i) an extracellular domain comprising a mutated Fc domain or a fragment thereof, and
(ii) a transmembrane domain.

2. The chimeric receptor of claim 1, further comprising (iii) at least one intracellular stimulatory signaling domain and/or at least one co-stimulatory signaling domain.

3. The chimeric receptor of claim 1 or 2, wherein the extracellular domain comprises a mutated CH2 domain and/or a mutated CH3 domain.

4. The chimeric receptor of any one of claims 1-3, wherein the extracellular domain comprises a mutated CH2 domain and/or a mutated CH3 domain, wherein the mutated CH2 domain and/or the mutated CH3 domain comprises the amino acid substitution (numbering according to Kabat EU index):
(a) I253A, H310A and H435A,
(b) I253A,
(c) I253A and H310A,
(d) I253A and H435A,
(e) H310A and H435A,
(f) H310A,
(g) H435A,
(h) L234A, L235A, and P329G, or
(i) L234A, L235A, I253A, H310A, P329G and H435A.

5. The chimeric receptor of any one of claims 1-4, wherein the transmembrane domain is selected from the group consisting of the CD8, the CD4, the CD3z, the FCGR3A, the NKG2D, the CD27, the CD28, the CD137, the OX40, the ICOS, the DAP10 or the DAP12 transmembrane domain or a fragment thereof.

6. The chimeric receptor of any one of claims 2-5, wherein the stimulatory signaling domain is selected from the group consisting of the intracellular domain of CD3z, of FCGR3A and of NKG2D, or a fragment thereof that retains stimulatory signaling activity.

7. The chimeric receptor of any one of claims 2-6, wherein the co-stimulatory signaling domain is selected from the group consisting of the intracellular domain of CD27, of CD28, of CD137, of OX40, of ICOS, of DAP10 and of DAP12, or a fragment thereof that retain co-stimulatory signaling activity.

8. The chimeric receptor of any one of claims 2-7, wherein the co-stimulatory signaling domain is the CD28 intracellular domain or a fragment thereof that retains CD28 co-stimulatory activity.

9. The chimeric receptor of any one of claims 2-8, wherein the chimeric receptor comprises one stimulatory signaling domain comprising the intracellular domain of CD3z, or a fragment thereof that retains CD3z stimulatory signaling activity, and wherein the chimeric receptor comprises one co-stimulatory signaling domain comprising the intracellular domain of CD137, or a fragment thereof that retains CD137 co-stimulatory signaling activity.

10. The chimeric receptor of any one of claims 2-9, wherein the stimulatory signaling domain comprises the amino acid sequence of SEQ ID NO: 13 and the co-stimulatory signaling domain comprises the amino acid sequence of SEQ ID NO:12.

11. The chimeric receptor of any one of claims 1-10, wherein the chimeric receptor comprises an amino acid sequence that is at least about 95%, 96%, 97%, 98%, 99% or 100% identical to an amino acid sequence selected from the group consisting of SEQ ID NO:87, SEQ ID NO: 106, SEQ ID NO: 125 and SEQ ID NO: 127.

12. A chimeric receptor comprising or consisting of an amino acid sequence selected from the group consisting of SEQ ID NO:87, SEQ ID NO: 106, SEQ ID NO: 125 and SEQ ID NO: 127.

13. An isolated polynucleotide encoding the chimeric receptor of any one of claims 1-12.

14. A polypeptide encoded by the isolated polynucleotide of claim 13.

15. A vector, particularly an expression vector, comprising the polynucleotide of claim 13.

16. A transduced T cell comprising the polynucleotide of claim 13 or the vector of claim 15.

17. A transduced T cell capable of expressing the chimeric receptor of any one of claims 1-12.

18. A kit comprising
(A) a transduced T cell capable of expressing the chimeric receptor of any one of claims 1-12, wherein the chimeric receptor comprises an extracellular domain comprising a mutated Fc domain or a fragment thereof; and
(B) an antibody that comprises at least one antigen binding moiety capable of binding to the mutated Fc domain but not capable of binding to the non-mutated parent Fc domain, wherein the antibody comprises at least one effector moiety.

19. A kit comprising
(A) an isolated polynucleotide encoding the chimeric receptor of any one of claims 1-12, wherein the chimeric receptor comprises an extracellular domain comprising a mutated Fc domain or a fragment thereof; and
(B) an antibody that comprises at least one antigen binding moiety capable of binding to the mutated Fc domain but not capable of binding to the non-mutated parent Fc domain, wherein the antibody comprises at least one effector moiety.

20. The kit of claim 18 or 19, wherein the effector moiety is (i) an antigen binding moiety capable of binding to a target cell antigen, or (ii) and immune activating moiety, in particular a cytokine.

21. The kit of any one of claims 18-20 for use as a medicament.

22. The chimeric receptor of any one of claims 1-12 or the transduced T cell of any one of claim 16 or 17 for use as a medicament, wherein the chimeric receptor comprises an extracellular domain comprising a mutated Fc domain or a fragment thereof, wherein a transduced T cell expressing the chimeric receptor is administered before, simultaneously with or after administration of an antibody that comprises at least one antigen binding moiety capable of binding to a target cell antigen, and at least one antigen binding moiety capable of binding to the mutated Fc domain but not capable of binding to the non-mutated parent Fc domain.

23. The kit of any one of claims 18-20 for use in the treatment of a disease, in particular for use in the treatment of a cancer.

24. A method of treating a disease in a subject, comprising administering to the subject a transduced T cell capable of expressing the chimeric receptor of any one of claims 1-12, wherein the chimeric receptor comprises an extracellular domain comprising a mutated Fc domain or a fragment thereof, and administering before, simultaneously with or after administration of the transduced T cell a therapeutically effective amount of an an antibody that comprises at least one antigen binding moiety capable of binding to the mutated Fc domain but not capable of binding to the non-mutated parent Fc domain, wherein the antibody comprises at least one effector moiety.

25. The method of claim 24, wherein the effector moiety is (i) an antigen binding moiety capable of binding to a target cell antigen, or (ii) and immune activating moiety, in particular a cytokine.

26. Use of the chimeric receptor of any one of claims 1-12, the polynucleotide of claim 13 or the transduced T cell of claim 16 or 17 for the manufacture of a medicament.

27. The use of claim 26, wherein the medicament is for treatment of cancer.

28. The use of claim 27, characterized in that said cancer is selected from cancer of epithelial, endothelial or mesothelial origin and cancer of the blood.


SEQUENCE LISTING (139 sequences)

SEQ ID NO: 1 - protein, 5 aa, synthetic construct
RYWMN

SEQ ID NO: 2 - protein, 17 aa, synthetic construct
EITPDSSTINYTPSLKD

SEQ ID NO: 3 - protein, 10 aa, synthetic construct
PYDYGAWFAS

SEQ ID NO: 4 - protein, 14 aa, synthetic construct
RSSTGAVTTSNYAN

SEQ ID NO: 5 - protein, 7 aa, synthetic construct
GTNKRAP

SEQ ID NO: 6 - protein, 9 aa, synthetic construct
ALWYSNHWV

SEQ ID NO: 7 - protein, 119 aa, synthetic construct
EVKLLESGGGLVQPGGSLKLSCAASGFDFSRYWMNWVRQAPGKGLEWIGEITPDSSTINYTPSLKDKFIISRDNAKNTLYLQMIKVRSEDTALYYCVRPYDYGAWFASWGQGTLVTVSA

SEQ ID NO: 8 - protein, 109 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNHWVFGGGTKLTVL

SEQ ID NO: 9 - protein, 17 aa, synthetic construct
EITPDSSTINYTPSLKG

SEQ ID NO: 10 - protein, 119 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSS

SEQ ID NO: 11 - protein, 109 aa, synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVL

SEQ ID NO: 12 - protein, 17 aa, synthetic construct
EITPDSSTINYAPSLKG

SEQ ID NO: 13 - protein, 119 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSS

SEQ ID NO: 14 - protein, 119 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSS

SEQ ID NO: 15 - protein, 17 aa, synthetic construct
EITPDSSTINYADSVKG

SEQ ID NO: 16 - protein, 119 aa, synthetic construct
EVQLVESGGGLVQPGGSLRLSCAASGFDFSRYWMNWVRQAPGKGLEWVSEITPDSSTINYADSVKGRFTISRDNAKNSLYLQMNSLRAEDTAVYYCARPYDYGAWFASWGQGTLVTVSS

SEQ ID NO: 17 - protein, 109 aa, synthetic construct
QAVVTQEPSLTVSPGGTVTLTCRSSTGAVTTSNYANWFQQKPGQAFTGLIGGTNKRAPGTPARFSGSLLGGKAALTLSGAQPEDEAEYYCALWYSNHWVFGGGTKLTVL

SEQ ID NO: 18 - protein, 14 aa, synthetic construct
GSSTGAVTTSNYAN

SEQ ID NO: 19 - protein, 109 aa, synthetic construct
QAVVTQEPSLTVSPGGTVTLTCGSSTGAVTTSNYANWFQQKPGQAPRTLIGGTNKRAPGTPARFSGSLLGGKAALTLSGAQPEDEAEYYCALWYSNHWVFGGGTKLTVL

SEQ ID NO: 20 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAK

SEQ ID NO: 21 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALLAPIEKTISKAK

SEQ ID NO: 22 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALIAPIEKTISKAK

SEQ ID NO: 23 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAK

SEQ ID NO: 24 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAK

SEQ ID NO: 25 - protein, 111 aa, synthetic construct
AAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALRAPIEKTISKAK

SEQ ID NO: 26 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 27 - protein, 218 aa, synthetic construct
AAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALRAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 28 - protein, 5 aa, synthetic construct
GYYWS

SEQ ID NO: 29 - protein, 16 aa, synthetic construct
EINHGGYVTYNPSLES

SEQ ID NO: 30 - protein, 13 aa, synthetic construct
DYGPGNYDWYFDL

SEQ ID NO: 31 - protein, 11 aa, synthetic construct
RASQSVSSYLA

SEQ ID NO: 32 - protein, 7 aa, synthetic construct
DASNRAT

SEQ ID NO: 33 - protein, 11 aa, synthetic construct
QQRSNWPPALT

SEQ ID NO: 34 - protein, 140 aa, synthetic construct
MNFGLSLVFLALILKGVQCEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSS

SEQ ID NO: 35 - protein, 112 aa, synthetic construct
DVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIK

SEQ ID NO: 36 - DNA, 780 bp, Homo sapiens
  source 1..780: mol_type = other DNA
atggcgcgcccgcatccgtggtggctgtgcgtgctgggcaccctggtgggcctgagcgcgaccccggcgccgaaaagctgcccggaacgccattattgggcgcagggcaaactgtgctgccagatgtgcgaaccgggcacctttctggtgaaagattgcgatcagcatcgcaaagcggcgcagtgcgatccgtgcattccgggcgtgagctttagcccggatcatcatacccgcccgcattgcgaaagctgccgccattgcaacagcggcctgctggtgcgcaactgcaccattaccgcgaacgcggaatgcgcgtgccgcaacggctggcagtgccgcgataaagaatgcaccgaatgcgatccgctgccgaacccgagcctgaccgcgcgcagcagccaggcgctgagcccgcatccgcagccgacccatctgccgtatgtgagcgaaatgctggaagcgcgcaccgcgggccatatgcagaccctggcggattttcgccagctgccggcgcgcaccctgagcacccattggccgccgcagcgcagcctgtgcagcagcgattttattcgcattctggtgatttttagcggcatgtttctggtgtttaccctggcgggcgcgctgtttctgcatcagcgccgcaaatatcgcagcaacaaaggcgaaagcccggtggaaccggcggaaccgtgccattatagctgcccgcgcgaagaagaaggcagcaccattccgattcaggaagattatcgcaaaccggaaccggcgtgcagcccg

SEQ ID NO: 37 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 38 - DNA, 750 bp, Mus musculus
  source 1..750: mol_type = other DNA
atggcgtggccgccgccgtattggctgtgcatgctgggcaccctggtgggcctgagcgcgaccctggcgccgaacagctgcccggataaacattattggaccggcggcggcctgtgctgccgcatgtgcgaaccgggcaccttttttgtgaaagattgcgaacaggatcgcaccgcggcgcagtgcgatccgtgcattccgggcaccagctttagcccggattatcatacccgcccgcattgcgaaagctgccgccattgcaacagcggctttctgattcgcaactgcaccgtgaccgcgaacgcggaatgcagctgcagcaaaaactggcagtgccgcgatcaggaatgcaccgaatgcgatccgccgctgaacccggcgctgacccgccagccgagcgaaaccccgagcccgcagccgccgccgacccatctgccgcatggcaccgaaaaaccgagctggccgctgcatcgccagctgccgaacagcaccgtgtatagccagcgcagcagccatcgcccgctgtgcagcagcgattgcattcgcatttttgtgacctttagcagcatgtttctgatttttgtgctgggcgcgattctgttttttcatcagcgccgcaaccatggcccgaacgaagatcgccaggcggtgccggaagaaccgtgcccgtatagctgcccgcgcgaagaagaaggcagcgcgattccgattcaggaagattatcgcaaaccggaaccggcgttttatccg

SEQ ID NO: 39 - protein, 250 aa, Mus musculus
MAWPPPYWLCMLGTLVGLSATLAPNSCPDKHYWTGGGLCCRMCEPGTFFVKDCEQDRTAAQCDPCIPGTSFSPDYHTRPHCESCRHCNSGFLIRNCTVTANAECSCSKNWQCRDQECTECDPPLNPALTRQPSETPSPQPPPTHLPHGTEKPSWPLHRQLPNSTVYSQRSSHRPLCSSDCIRIFVTFSSMFLIFVLGAILFFHQRRNHGPNEDRQAVPEEPCPYSCPREEEGSAIPIQEDYRKPEPAFYP

SEQ ID NO: 40 - DNA, 660 bp, Homo sapiens
  source 1..660: mol_type = other DNA
atgctgcgcctgctgctggcgctgaacctgtttccgagcattcaggtgaccggcaacaaaattctggtgaaacagagcccgatgctggtggcgtatgataacgcggtgaacctgagctgcaaatatagctataacctgtttagccgcgaatttcgcgcgagcctgcataaaggcctggatagcgcggtggaagtgtgcgtggtgtatggcaactatagccagcagctgcaggtgtatagcaaaaccggctttaactgcgatggcaaactgggcaacgaaagcgtgaccttttatctgcagaacctgtatgtgaaccagaccgatatttatttttgcaaaattgaagtgatgtatccgccgccgtatctggataacgaaaaaagcaacggcaccattattcatgtgaaaggcaaacatctgtgcccgagcccgctgtttccgggcccgagcaaaccgttttgggtgctggtggtggtgggcggcgtgctggcgtgctatagcctgctggtgaccgtggcgtttattattttttgggtgcgcagcaaacgcagccgcctgctgcatagcgattatatgaacatgaccccgcgccgcccgggcccgacccgcaaacattatcagccgtatgcgccgccgcgcgattttgcggcgtatcgcagc

SEQ ID NO: 41 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 42 - DNA, 654 bp, Mus musculus
  source 1..654: mol_type = other DNA
atgaccctgcgcctgctgtttctggcgctgaacttttttagcgtgcaggtgaccgaaaacaaaattctggtgaaacagagcccgctgctggtggtggatagcaacgaagtgagcctgagctgccgctatagctataacctgctggcgaaagaatttcgcgcgagcctgtataaaggcgtgaacagcgatgtggaagtgtgcgtgggcaacggcaactttacctatcagccgcagtttcgcagcaacgcggaatttaactgcgatggcgattttgataacgaaaccgtgacctttcgcctgtggaacctgcatgtgaaccataccgatatttatttttgcaaaattgaatttatgtatccgccgccgtatctggataacgaacgcagcaacggcaccattattcatattaaagaaaaacatctgtgccatacccagagcagcccgaaactgttttgggcgctggtggtggtggcgggcgtgctgttttgctatggcctgctggtgaccgtggcgctgtgcgtgatttggaccaacagccgccgcaaccgcctgctgcagagcgattatatgaacatgaccccgcgccgcccgggcctgacccgcaaaccgtatcagccgtatgcgccggcgcgcgattttgcggcgtatcgcccg

SEQ ID NO: 43 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 44 - DNA, 768 bp, Homo sapiens
  source 1..768: mol_type = other DNA
atgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgtga

SEQ ID NO: 45 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 46 - DNA, 768 bp, Mus musculus
  source 1..768: mol_type = other DNA
atgggcaacaactgctataacgtggtggtgattgtgctgctgctggtgggctgcgaaaaagtgggcgcggtgcagaacagctgcgataactgccagccgggcaccttttgccgcaaatataacccggtgtgcaaaagctgcccgccgagcacctttagcagcattggcggccagccgaactgcaacatttgccgcgtgtgcgcgggctattttcgctttaaaaaattttgcagcagcacccataacgcggaatgcgaatgcattgaaggctttcattgcctgggcccgcagtgcacccgctgcgaaaaagattgccgcccgggccaggaactgaccaaacagggctgcaaaacctgcagcctgggcacctttaacgatcagaacggcaccggcgtgtgccgcccgtggaccaactgcagcctggatggccgcagcgtgctgaaaaccggcaccaccgaaaaagatgtggtgtgcggcccgccggtggtgagctttagcccgagcaccaccattagcgtgaccccggaaggcggcccgggcggccatagcctgcaggtgctgaccctgtttctggcgctgaccagcgcgctgctgctggcgctgatttttattaccctgctgtttagcgtgctgaaatggattcgcaaaaaatttccgcatatttttaaacagccgtttaaaaaaaccaccggcgcggcgcaggaagaagatgcgtgcagctgccgctgcccgcaggaagaagaaggcggcggcggcggctatgaactg

SEQ ID NO: 47 - protein, 256 aa, Mus musculus
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 48 - DNA, 831 bp, Homo sapiens
  source 1..831: mol_type = other DNA
atgtgcgtgggcgcgcgccgcctgggccgcggcccgtgcgcggcgctgctgctgctgggcctgggcctgagcaccgtgaccggcctgcattgcgtgggcgatacctatccgagcaacgatcgctgctgccatgaatgccgcccgggcaacggcatggtgagccgctgcagccgcagccagaacaccgtgtgccgcccgtgcggcccgggcttttataacgatgtggtgagcagcaaaccgtgcaaaccgtgcacctggtgcaacctgcgcagcggcagcgaacgcaaacagctgtgcaccgcgacccaggataccgtgtgccgctgccgcgcgggcacccagccgctggatagctataaaccgggcgtggattgcgcgccgtgcccgccgggccattttagcccgggcgataaccaggcgtgcaaaccgtggaccaactgcaccctggcgggcaaacataccctgcagccggcgagcaacagcagcgatgcgatttgcgaagatcgcgatccgccggcgacccagccgcaggaaacccagggcccgccggcgcgcccgattaccgtgcagccgaccgaagcgtggccgcgcaccagccagggcccgagcacccgcccggtggaagtgccgggcggccgcgcggtggcggcgattctgggcctgggcctggtgctgggcctgctgggcccgctggcgattctgctggcgctgtatctgctgcgccgcgatcagcgcctgccgccggatgcgcataaaccgccgggcggcggcagctttcgcaccccgattcaggaagaacaggcggatgcgcatagcaccctggcgaaaatt

SEQ ID NO: 49 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 50 - DNA, 816 bp, Mus musculus
  source 1..816: mol_type = other DNA
atgtatgtgtgggtgcagcagccgaccgcgctgctgctgctggcgctgaccctgggcgtgaccgcgcgccgcctgaactgcgtgaaacatacctatccgagcggccataaatgctgccgcgaatgccagccgggccatggcatggtgagccgctgcgatcatacccgcgataccctgtgccatccgtgcgaaaccggcttttataacgaagcggtgaactatgatacctgcaaacagtgcacccagtgcaaccatcgcagcggcagcgaactgaaacagaactgcaccccgacccaggataccgtgtgccgctgccgcccgggcacccagccgcgccaggatagcggctataaactgggcgtggattgcgtgccgtgcccgccgggccattttagcccgggcaacaaccaggcgtgcaaaccgtggaccaactgcaccctgagcggcaaacagacccgccatccggcgagcgatagcctggatgcggtgtgcgaagatcgcagcctgctggcgaccctgctgtgggaaacccagcgcccgacctttcgcccgaccaccgtgcagagcaccaccgtgtggccgcgcaccagcgaactgccgagcccgccgaccctggtgaccccggaaggcccggcgtttgcggtgctgctgggcctgggcctgggcctgctggcgccgctgaccgtgctgctggcgctgtatctgctgcgcaaagcgtggcgcctgccgaacaccccgaaaccgtgctggggcaacagctttcgcaccccgattcaggaagaacataccgatgcgcattttaccctggcgaaaatt

SEQ ID NO: 51 - protein, 272 aa, Mus musculus
MYVWVQQPTALLLLALTLGVTARRLNCVKHTYPSGHKCCRECQPGHGMVSRCDHTRDTLCHPCETGFYNEAVNYDTCKQCTQCNHRSGSELKQNCTPTQDTVCRCRPGTQPRQDSGYKLGVDCVPCPPGHFSPGNNQACKPWTNCTLSGKQTRHPASDSLDAVCEDRSLLATLLWETQRPTFRPTTVQSTTVWPRTSELPSPPTLVTPEGPAFAVLLGLGLGLLAPLTVLLALYLLRKAWRLPNTPKPCWGNSFRTPIQEEHTDAHFTLAKI

SEQ ID NO: 52 - DNA, 597 bp, Homo sapiens
  source 1..597: mol_type = other DNA
atgaaaagcggcctgtggtatttttttctgttttgcctgcgcattaaagtgctgaccggcgaaattaacggcagcgcgaactatgaaatgtttatttttcataacggcggcgtgcagattctgtgcaaatatccggatattgtgcagcagtttaaaatgcagctgctgaaaggcggccagattctgtgcgatctgaccaaaaccaaaggcagcggcaacaccgtgagcattaaaagcctgaaattttgccatagccagctgagcaacaacagcgtgagcttttttctgtataacctggatcatagccatgcgaactattatttttgcaacctgagcatttttgatccgccgccgtttaaagtgaccctgaccggcggctatctgcatatttatgaaagccagctgtgctgccagctgaaattttggctgccgattggctgcgcggcgtttgtggtggtgtgcattctgggctgcattctgatttgctggctgaccaaaaaaaaatatagcagcagcgtgcatgatccgaacggcgaatatatgtttatgcgcgcggtgaacaccgcgaaaaaaagccgcctgaccgatgtgaccctg

SEQ ID NO: 53 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 54 - DNA, 600 bp, Mus musculus
  source 1..600: mol_type = other DNA
atgaaaccgtatttttgccgcgtgtttgtgttttgctttctgattcgcctgctgaccggcgaaattaacggcagcgcggatcatcgcatgtttagctttcataacggcggcgtgcagattagctgcaaatatccggaaaccgtgcagcagctgaaaatgcgcctgtttcgcgaacgcgaagtgctgtgcgaactgaccaaaaccaaaggcagcggcaacgcggtgagcattaaaaacccgatgctgtgcctgtatcatctgagcaacaacagcgtgagcttttttctgaacaacccggatagcagccagggcagctattatttttgcagcctgagcatttttgatccgccgccgtttcaggaacgcaacctgagcggcggctatctgcatatttatgaaagccagctgtgctgccagctgaaactgtggctgccggtgggctgcgcggcgtttgtggtggtgctgctgtttggctgcattctgattatttggtttagcaaaaaaaaatatggcagcagcgtgcatgatccgaacagcgaatatatgtttatggcggcggtgaacaccaacaaaaaaagccgcctggcgggcgtgaccagc

SEQ ID NO: 55 - protein, 200 aa, Mus musculus
MKPYFCRVFVFCFLIRLLTGEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 56 - DNA, 279 bp, Homo sapiens
  source 1..279: mol_type = other DNA
atgattcatctgggccatattctgtttctgctgctgctgccggtggcggcggcgcagaccaccccgggcgaacgcagcagcctgccggcgttttatccgggcaccagcggcagctgcagcggctgcggcagcctgagcctgccgctgctggcgggcctggtggcggcggatgcggtggcgagcctgctgattgtgggcgcggtgtttctgtgcgcgcgcccgcgccgcagcccggcgcaggaagatggcaaagtgtatattaacatgccgggccgcggc

SEQ ID NO: 57 - protein, 93 aa, Homo sapiens
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 58 - DNA, 237 bp, Mus musculus
  source 1..237: mol_type = other DNA
atggatccgccgggctatctgctgtttctgctgctgctgccggtggcggcgagccagaccagcgcgggcagctgcagcggctgcggcaccctgagcctgccgctgctggcgggcctggtggcggcggatgcggtgatgagcctgctgattgtgggcgtggtgtttgtgtgcatgcgcccgcatggccgcccggcgcaggaagatggccgcgtgtatattaacatgccgggccgcggc

SEQ ID NO: 59 - protein, 79 aa, Mus musculus
MDPPGYLLFLLLLPVAASQTSAGSCSGCGTLSLPLLAGLVAADAVMSLLIVGVVFVCMRPHGRPAQEDGRVYINMPGRG

SEQ ID NO: 60 - DNA, 342 bp, Homo sapiens
  source 1..342: mol_type = other DNA
atggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatga

SEQ ID NO: 61 - protein, 113 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 62 - DNA, 345 bp, Mus musculus
  source 1..345: mol_type = other DNA
atgggggctctggagccctcctggtgccttctgttccttcctgtcctcctgactgtgggaggattaagtcccgtacaggcccagagtgacactttcccaagatgcgactgttcttccgtgagccctggtgtactggctgggattgttctgggtgacttggtgttgactctgctgattgccctggctgtgtactctctgggccgcctggtctcccgaggtcaagggacagcggaagggacccggaaacaacacattgctgagactgagtcgccttatcaggagcttcagggtcagagaccagaagtatacagtgacctcaacacacagaggcaatattacagatga

SEQ ID NO: 63 - protein, 114 aa, Mus musculus
MGALEPSWCLLFLPVLLTVGGLSPVQAQSDTFPRCDCSSVSPGVLAGIVLGDLVLTLLIALAVYSLGRLVSRGQGTAEGTRKQHIAETESPYQELQGQRPEVYSDLNTQRQYYR

SEQ ID NO: 64 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 65 - DNA, 492 bp, Homo sapiens
  source 1..492: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 66 - protein, 164 aa, Mus musculus
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 67 - DNA, 495 bp, Mus musculus
  source 1..495: mol_type = other DNA
atgaagtggaaagtgtctgttctcgcctgcatcctccacgtgcggttcccaggagcagaggcacagagctttggtctgctggatcccaaactctgctacttgctagatggaatcctcttcatctacggagtcatcatcacagccctgtacctgagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 68 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 69 - DNA, 762 bp, Homo sapiens
  source 1..762: mol_type = other DNA
atgtggcagctgctgctgccgaccgcgctgctgctgctggtgagcgcgggcatgcgcaccgaagatctgccgaaagcggtggtgtttctggaaccgcagtggtatcgcgtgctggaaaaagatagcgtgaccctgaaatgccagggcgcgtatagcccggaagataacagcacccagtggtttcataacgaaagcctgattagcagccaggcgagcagctattttattgatgcggcgaccgtggatgatagcggcgaatatcgctgccagaccaacctgagcaccctgagcgatccggtgcagctggaagtgcatattggctggctgctgctgcaggcgccgcgctgggtgtttaaagaagaagatccgattcatctgcgctgccatagctggaaaaacaccgcgctgcataaagtgacctatctgcagaacggcaaaggccgcaaatattttcatcataacagcgatttttatattccgaaagcgaccctgaaagatagcggcagctatttttgccgcggcctgtttggcagcaaaaacgtgagcagcgaaaccgtgaacattaccattacccagggcctggcggtgagcaccattagcagcttttttccgccgggctatcaggtgagcttttgcctggtgatggtgctgctgtttgcggtggataccggcctgtattttagcgtgaaaaccaacattcgcagcagcacccgcgattggaaagatcataaatttaaatggcgcaaagatccgcaggataaa

SEQ ID NO: 70 - protein, 261 aa, Mus musculus
MFQNAHSGSQWLLPPLTILLLFAFADRQSAALPKAVVKLDPPWIQVLKEDMVTLMCEGTHNPGNSSTQWFHNGRSIRSQVQASYTFKATVNDSGEYRCQMEQTRLSDPVDLGVISDWLLLQTPQRVFLEGETITLRCHSWRNKLLNRISFFHNEKSVRYHHYKSNFSIPKANHSHSGDYYCKGSLGSTQHQSKPVTITVQDPATTSSISLVWYHTAFSLVMCLLFAVDTGLYFYVRRNLQTPREYWRKSLSIRKHQAPQDK

SEQ ID NO: 71 - DNA, 786 bp, Mus musculus
  source 1..786: mol_type = other DNA
atgtttcagaatgcacactctggaagccaatggctacttccaccactgacaattctgctgctgtttgcttttgcagacaggcagagtgcagctcttccgaaggctgtggtgaaactggaccccccatggatccaggtgctcaaggaagacatggtgacactgatgtgcgaagggacccacaaccctgggaactcttctacccagtggttccacaacgggaggtccatccggagccaggtccaagccagttacacgtttaaggccacagtcaatgacagtggagaatatcggtgtcaaatggagcagacccgcctcagcgaccctgtagatctgggagtgatttctgactggctgctgctccagacccctcagcgggtgtttctggaaggggaaaccatcacgctaaggtgccatagctggaggaacaaactactgaacaggatctcattcttccataatgaaaaatccgtgaggtatcatcactacaaaagtaatttctctatcccaaaagccaaccacagtcacagtggggactactactgcaaaggaagtctaggaagtacacagcaccagtccaagcctgtcaccatcactgtccaagatccagcaactacatcctccatctctctagtctggtaccacactgctttctccctagtgatgtgcctcctgtttgcagtggacacgggcctttatttctacgtacggagaaatcttcaaaccccgagggagtactggaggaagtccctgtcaatcagaaagcaccaggctcctcaagacaagtga

SEQ ID NO: 72 - protein, 216 aa, Homo sapiens
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 73 - DNA, 648 bp, Homo sapiens
  source 1..648: mol_type = other DNA
atgggctggattcgcggccgccgcagccgccatagctgggaaatgagcgaatttcataactataacctggatctgaaaaaaagcgattttagcacccgctggcagaaacagcgctgcccggtggtgaaaagcaaatgccgcgaaaacgcgagcccgttttttttttgctgctttattgcggtggcgatgggcattcgctttattattatggtggcgatttggagcgcggtgtttctgaacagcctgtttaaccaggaagtgcagattccgctgaccgaaagctattgcggcccgtgcccgaaaaactggatttgctataaaaacaactgctatcagttttttgatgaaagcaaaaactggtatgaaagccaggcgagctgcatgagccagaacgcgagcctgctgaaagtgtatagcaaagaagatcaggatctgctgaaactggtgaaaagctatcattggatgggcctggtgcatattccgaccaacggcagctggcagtgggaagatggcagcattctgagcccgaacctgctgaccattattgaaatgcagaaaggcgattgcgcgctgtatgcgagcagctttaaaggctatattgaaaactgcagcaccccgaacacctatatttgcatgcagcgcaccgtg

SEQ ID NO: 74 - protein, 232 aa, Mus musculus
MALIRDRKSHHSEMSKCHNYDLKPAKWDTSQEQQKQRLALTTSQPGENGIIRGRYPIEKLKISPMFVVRVLAIALAIRFTLNTLMWLAIFKETFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAV

SEQ ID NO: 75 - DNA, 696 bp, Mus musculus
  source 1..696: mol_type = other DNA
atggcgctgattcgcgatcgcaaaagccatcatagcgaaatgagcaaatgccataactatgatctgaaaccggcgaaatgggataccagccaggaacagcagaaacagcgcctggcgctgaccaccagccagccgggcgaaaacggcattattcgcggccgctatccgattgaaaaactgaaaattagcccgatgtttgtggtgcgcgtgctggcgattgcgctggcgattcgctttaccctgaacaccctgatgtggctggcgatttttaaagaaacctttcagccggtgctgtgcaacaaagaagtgccggtgagcagccgcgaaggctattgcggcccgtgcccgaacaactggatttgccatcgcaacaactgctatcagttttttaacgaagaaaaaacctggaaccagagccaggcgagctgcctgagccagaacagcagcctgctgaaaatttatagcaaagaagaacaggattttctgaaactggtgaaaagctatcattggatgggcctggtgcagattccggcgaacggcagctggcagtgggaagatggcagcagcctgagctataaccagctgaccctggtggaaattccgaaaggcagctgcgcggtgtatggcagcagctttaaagcgtataccgaagattgcgcgaacctgaacacctatatttgcatgaaacgcgcggtg

SEQ ID NO: 76 - protein, 4 aa, synthetic construct
YMNM

SEQ ID NO: 77 - protein, 4 aa, synthetic construct
PYAP

SEQ ID NO: 78 - protein, 4 aa, synthetic construct
FMNM

SEQ ID NO: 79 - protein, 4 aa, synthetic construct
AYAA

SEQ ID NO: 80 - protein, 21 aa, synthetic construct
ATMGWSCIILFLVATATGVHS

SEQ ID NO: 81 - DNA, 57 bp, synthetic construct
  source 1..57: mol_type = other DNA
atgggatggagctgtatcatcctcttcttggtagcaacagctaccggtgtgcactcc

SEQ ID NO: 82 - protein, 276 aa, Homo sapiens
MRNQAPGRPKGATFPPRRPTGSRAPPLAPELRAKQRPGERVMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 83 - DNA, 828 bp, Homo sapiens
  source 1..828: mol_type = other DNA
atgcgcaaccaggcgccgggccgcccgaaaggcgcgacctttccgccgcgccgcccgaccggcagccgcgcgccgccgctggcgccggaactgcgcgcgaaacagcgcccgggcgaacgcgtgatggcgctgccggtgaccgcgctgctgctgccgctggcgctgctgctgcatgcggcgcgcccgagccagtttcgcgtgagcccgctggatcgcacctggaacctgggcgaaaccgtggaactgaaatgccaggtgctgctgagcaacccgaccagcggctgcagctggctgtttcagccgcgcggcgcggcggcgagcccgacctttctgctgtatctgagccagaacaaaccgaaagcggcggaaggcctggatacccagcgctttagcggcaaacgcctgggcgatacctttgtgctgaccctgagcgattttcgccgcgaaaacgaaggctattatttttgcagcgcgctgagcaacagcattatgtattttagccattttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccgccgccgcgtgtgcaaatgcccgcgcccggtggtgaaaagcggcgataaaccgagcctgagcgcgcgctatgtg

SEQ ID NO: 84 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 85 - DNA, 741 bp, Mus musculus
  source 1..741: mol_type = other DNA
atggcgagcccgctgacccgctttctgagcctgaacctgctgctgatgggcgaaagcattattctgggcagcggcgaagcgaaaccgcaggcgccggaactgcgcatttttccgaaaaaaatggatgcggaactgggccagaaagtggatctggtgtgcgaagtgctgggcagcgtgagccagggctgcagctggctgtttcagaacagcagcagcaaactgccgcagccgacctttgtggtgtatatggcgagcagccataacaaaattacctgggatgaaaaactgaacagcagcaaactgtttagcgcggtgcgcgataccaacaacaaatatgtgctgaccctgaacaaatttagcaaagaaaacgaaggctattatttttgcagcgtgattagcaacagcgtgatgtattttagcagcgtggtgccggtgctgcagaaagtgaacagcaccaccaccaaaccggtgctgcgcaccccgagcccggtgcatccgaccggcaccagccagccgcagcgcccggaagattgccgcccgcgcggcagcgtgaaaggcaccggcctggattttgcgtgcgatatttatatttgggcgccgctggcgggcatttgcgtggcgccgctgctgagcctgattattaccctgatttgctatcatcgcagccgcaaacgcgtgtgcaaatgcccgcgcccgctggtgcgccaggaaggcaaaccgcgcccgagcgaaaaaattgtg

SEQ ID NO: 86 - protein, 110 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAK

SEQ ID NO: 87 - protein, 339 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGGGGSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 88 - protein, 48 aa, synthetic construct
AKPTTTPAPRPPTPAPTIASQPLSLRPEACRPAAGGAVHTRGLDFACD

SEQ ID NO: 89 - protein, 21 aa, synthetic construct
IYIWAPLAGTCGVLLLSLVIT

SEQ ID NO: 90 - protein, 42 aa, synthetic construct
KRGRKKLLYIFKQPFMRPVQTTQEEDGCSCRFPEEEEGGCEL

SEQ ID NO: 91 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 92 - protein, 224 aa, synthetic construct
AKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 93 - protein, 238 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 94 - protein, 5 aa, synthetic construct
GGGGS

SEQ ID NO: 95 - protein, 21 aa, synthetic construct
GSGEGRGSLLTCGDVEENPGP

SEQ ID NO: 96 - DNA, 1017 bp, synthetic construct
  source 1..1017: mol_type = other DNA
gcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaaggcgggggaggatccgccaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccaaacggggcagaaagaaactcctgtatatattcaaacaaccatttatgagaccagtacaaactactcaagaggaagatggctgtagctgccgatttccagaagaagaagaaggaggatgtgaactgcgggtgaaattctcccgttcagctgacgcacccgcgtatcaacagggccagaaccaactgtacaatgagctcaacctgggtcgtagagaagagtacgatgtgctggacaagcgccggggccgcgaccctgagatgggtggaaagccccagcgccggaagaatccccaagagggactgtataacgagctgcagaaggacaagatggctgaggcctacagcgagatcggtatgaagggcgagagacgtagaggcaagggccatgacgggctgtaccaagggcttagtaccgctacaaaggatacctacgacgccctgcacatgcaggccctgccccctcgt

SEQ ID NO: 97 - DNA, 144 bp, synthetic construct
  source 1..144: mol_type = other DNA
gccaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 98 - DNA, 63 bp, synthetic construct
  source 1..63: mol_type = other DNA
atctacatctgggcgcccctggccgggacttgtggggtccttctcctgtcactggttatcacc

SEQ ID NO: 99 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 100 - DNA, 339 bp, synthetic construct
  source 1..339: mol_type = other DNA
cgggtgaaattctcccgttcagctgacgcacccgcgtatcaacagggccagaaccaactgtacaatgagctcaacctgggtcgtagagaagagtacgatgtgctggacaagcgccggggccgcgaccctgagatgggtggaaagccccagcgccggaagaatccccaagagggactgtataacgagctgcagaaggacaagatggctgaggcctacagcgagatcggtatgaagggcgagagacgtagaggcaagggccatgacgggctgtaccaagggcttagtaccgctacaaaggatacctacgacgccctgcacatgcaggccctgccccctcgt

SEQ ID NO: 101 - DNA, 672 bp, synthetic construct
  source 1..672: mol_type = other DNA
gccaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccaaacggggcagaaagaaactcctgtatatattcaaacaaccatttatgagaccagtacaaactactcaagaggaagatggctgtagctgccgatttccagaagaagaagaaggaggatgtgaactgcgggtgaaattctcccgttcagctgacgcacccgcgtatcaacagggccagaaccaactgtacaatgagctcaacctgggtcgtagagaagagtacgatgtgctggacaagcgccggggccgcgaccctgagatgggtggaaagccccagcgccggaagaatccccaagagggactgtataacgagctgcagaaggacaagatggctgaggcctacagcgagatcggtatgaagggcgagagacgtagaggcaagggccatgacgggctgtaccaagggcttagtaccgctacaaaggatacctacgacgccctgcacatgcaggccctgccccctcgt

SEQ ID NO: 102 - DNA, 63 bp, synthetic construct
  source 1..63: mol_type = other DNA
ggttccggagagggcagaggctctctgctcacctgcggagatgtggaggaaaacccaggacct

SEQ ID NO: 103 - DNA, 717 bp, synthetic construct
  source 1..717: mol_type = other DNA
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 104 - DNA, 1797 bp, synthetic construct
  source 1..1797: mol_type = other DNA
gcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaaggcgggggaggatccgccaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccaaacggggcagaaagaaactcctgtatatattcaaacaaccatttatgagaccagtacaaactactcaagaggaagatggctgtagctgccgatttccagaagaagaagaaggaggatgtgaactgcgggtgaaattctcccgttcagctgacgcacccgcgtatcaacagggccagaaccaactgtacaatgagctcaacctgggtcgtagagaagagtacgatgtgctggacaagcgccggggccgcgaccctgagatgggtggaaagccccagcgccggaagaatccccaagagggactgtataacgagctgcagaaggacaagatggctgaggcctacagcgagatcggtatgaagggcgagagacgtagaggcaagggccatgacgggctgtaccaagggcttagtaccgctacaaaggatacctacgacgccctgcacatgcaggccctgccccctcgtggttccggagagggcagaggctctctgctcacctgcggagatgtggaggaaaacccaggacctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 105 - protein, 218 aa, synthetic construct
AAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALRAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 106 - protein, 446 aa, synthetic construct
AAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALRAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKGGGGSLKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - DNA, 1335 bp, synthetic construct
  source 1..1335: mol_type = other DNA
gcccctgaagctgctggcggcccttccgtgttcctgttccccccaaagcccaaggacaccctgatggcctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctggcccaggactggctgaacggcaaagagtacaagtgcaaggtgtccaacaaggccctgcgggctcccatcgaaaagaccatctccaaggccaagggccagccccgcgagcctcaggtgtacaccctgcctcctagccgggacgagctgaccaagaaccaggtgtccctgacctgcctggtgaagggcttctacccctccgatatcgccgtggaatgggagtccaacggccagcccgagaacaactacaagaccaccccccctgtgctggactccgacggctcattcttcctgtactctaagctgaccgtggacaagtcccggtggcagcagggcaacgtgttctcctgctccgtgatgcacgaggccctgcacaatgcctacacccagaagtccctgtccctgtctcccggcaaaggagggggcggatccttgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 108 - protein, 227 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 441 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 110 - protein, 674 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGGEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 215 aa, synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGQPKAAPSVTLFPPSSKKLQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 112 - protein, 440 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 113 - protein, 693 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGGEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGLNDIFEAQKIEWHE

SEQ ID NO: 114 - protein, 219 aa, synthetic construct
DVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 449 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 468 aa, synthetic construct
EVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGLNDIFEAQKIEWHE

SEQ ID NO: 117 - protein, 228 aa, synthetic construct
EVQLQQSGAELVEPGASVKLSCTASGFNIKDTYMHWVKQRPEQGLEWIGRIDPANGNSKYVPKFQGKATITADTSSNTAYLQLTSLTSEDTAVYYCAPFGYYVSDYAMAYWGQGTSVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 442 aa, synthetic construct
DIVLTQSPASLAVSLGQRATMSCRAGESVDIFGVGFLHWYQQKPGQPPKLLIYRASNLESGIPVRFSGTGSRTDFTLIIDPVEADDVATYYCQQTNEDPYTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 119 - protein, 691 aa, synthetic construct
DIVLTQSPASLAVSLGQRATMSCRAGESVDIFGVGFLHWYQQKPGQPPKLLIYRASNLESGIPVRFSGTGSRTDFTLIIDPVEADDVATYYCQQTNEDPYTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGLNDIFEAQKIEWHE

SEQ ID NO: 120 - protein, 215 aa, synthetic construct; identical to SEQ ID NO: 111

SEQ ID NO: 121 - protein, 228 aa, synthetic construct; identical to SEQ ID NO: 117

SEQ ID NO: 122 - protein, 442 aa, synthetic construct
DIVLTQSPASLAVSLGQRATMSCRAGESVDIFGVGFLHWYQQKPGQPPKLLIYRASNLESGIPVRFSGTGSRTDFTLIIDPVEADDVATYYCQQTNEDPYTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 123 - protein, 693 aa, synthetic construct
DIVLTQSPASLAVSLGQRATMSCRAGESVDIFGVGFLHWYQQKPGQPPKLLIYRASNLESGIPVRFSGTGSRTDFTLIIDPVEADDVATYYCQQTNEDPYTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGLNDIFEAQKIEWHE

SEQ ID NO: 124 - protein, 217 aa, synthetic construct
APEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALRAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 125 - protein, 446 aa, synthetic construct
APEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALRAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKGGGGSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 126 - protein, 217 aa, synthetic construct
APEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 127 - protein, 446 aa, synthetic construct
APEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKGGGGSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 128 - protein, 41 aa, synthetic construct
RSKRSRLLHSDYMNMTPRRPGPTRKHYQPYAPPRDFAAYRS

SEQ ID NO: 129 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 130 - protein, 8 aa, synthetic construct
GFTFSSYG

SEQ ID NO: 131 - protein, 8 aa, synthetic construct
ISSGGSYI

SEQ ID NO: 132 - protein, 14 aa, synthetic construct
ARLGMITTGYAMDY

SEQ ID NO: 133 - protein, 11 aa, synthetic construct
QTIVHSTGHTY

SEQ ID NO: 134 - skipped

SEQ ID NO: 135 - protein, 9 aa, synthetic construct
FQGSHVPYT

SEQ ID NO: 136 - protein, 121 aa, synthetic construct
EVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSS

SEQ ID NO: 137 - protein, 215 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNHWVFGGGTKLTVLGQPKAAPSVTLFPPSSKKLQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 138 - protein, 449 aa, synthetic construct
EVKLLESGGGLVQPGGSLKLSCAASGFDFSRYWMNWVRQAPGKGLEWIGEITPDSSTINYTPSLKDKFIISRDNAKNTLYLQMIKVRSEDTALYYCVRPYDYGAWFASWGQGTLVTVSAASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 139 - protein, 374 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT